(19) United States
(12) Patent Application Publication
(10) Pub. No.: US 2020/0187851 A1
(43) Pub. Date: Jun. 18, 2020

(54) PERIODONTAL DISEASE STRATIFICATION AND USES THEREOF

(71) Applicant: The University of North Carolina at Chapel Hill, Chapel Hill, NC (US)

(72) Inventors: Steven Offenbacher, Chapel Hill, NC (US); Thiago Morelli, Durham, NC (US); Kevin Lee Moss, Graham, NC (US); James Douglas Beck, Chapel Hill, NC (US)

(21) Appl. No.: 16/713,874

(22) Filed: Dec. 13, 2019

Related U.S. Application Data
(60) Provisional application No. 62/780,675, filed on Dec. 17, 2018.

(51) Int. Cl. A61B 5/00; G16H 20/10

(52) U.S. Cl. CPC A61B 5/4552; G16H 20/10; A61B 5/7275; A61B 5/7264

(57) ABSTRACT

Described herein are methods of classifying periodontal patients and individual teeth. For example, disclosed is a method of diagnosing periodontal disease and/or risk of tooth loss in a subject that involves classifying teeth into one of 7 classes of periodontal disease. The method can include the step of performing a dental examination on a patient and determining a periodontal profile class (PPC). The method can further include the step of determining for each tooth a Tooth Profile Class (TPC). The PPC and TPC can be used together to generate a composite risk score for an individual, which is referred to herein as the Index of Periodontal Risk (IPR). In some embodiments, each stage of the disclosed PPC system is characterized by unique single nucleotide polymorphisms (SNPs) associated with unique pathways, identifying unique druggable targets for each stage.

### CROSS-REFERENCE TO RELATED APPLICATIONS

This application claims benefit of U.S. Provisional Application No. 62/780,675, filed Dec. 17, 2018, which is hereby incorporated herein by reference in its entirety.

### STATEMENT REGARDING FEDERALLY SPONSORED RESEARCH OR DEVELOPMENT

This invention was made with government support under Grant Numbers R01-DE021418, R01-DE021986, UL1-TR001111, HHSN268201100005C, HHSN268201100006C, HHSN268201100007C, HHSN268201100008C, HHSN268201100009C, HHSN268201100010C, HHSN268201100011C, and HHSN268201100012C awarded by the National Institutes of Health. The government has certain rights in the invention.

### BACKGROUND

The notion of precision dentistry as it relates to precision medicine is relatively new to the field of oral health. A search for the term ‘precision dentistry’ almost exclusively brings up articles that focus on the importance of being ‘precise’ in patient treatment procedures (precision attachments, high-precision digitizing, digital dentistry, minimally invasive dentistry). A search for the term ‘personalized dentistry’ results in articles about providing care based on patient characteristics as well as some articles on the same topic under the rubric of precision dentistry. Thus, it seems that just using the term ‘precision dentistry’ results in some confusion as to what this is all about. To add to that confusion, precision dentistry differs from personalized dentistry. Precision dentistry is a contemporary, multifaceted, data-driven approach to oral health care that uses individual characteristics to stratify alike patients into phenotypic groups. The goal is to provide clinicians the information that will allow them to improve treatment planning and patient response to treatment. Providers that use a precision oral health approach would move away from using an ‘average treatment’ for all people with a particular diagnosis and toward more specific treatments for patients within each diagnostic subgroup.

Precision oral health requires a method or model that places each individual in a subgroup where each member is the same as every other member in relation to the disease of interest. Precision dentistry is a paradigm shift that requires a new way of thinking about diagnostic categories. This approach uses patients' risk factor data (including but not limited to genetic, environmental and health behavioral), rather than expert opinion or clinical presentation alone, to redefine traditional categories of health and disease.

### SUMMARY

Disclosed herein is a method of diagnosing periodontal disease and/or risk of tooth loss in a subject, the method comprising classifying the patient and/or each individual tooth of a patient into one of 7 classes of periodontal disease. The classes can indicate the risk of tooth loss in a subject and/or by individual tooth. The method can include the step of performing a dental examination on a patient and determining a periodontal profile class (PPC). The PPC is a patient stratification system based on seven clinical parameters that defines distinct categories of members (people) with previously “hidden” combinations of clinical characteristics, to create mutually exclusive latent classes. The seven PPCs are designated herein as PPC-A, PPC-B, PPC-C, PPC-D), PPC-E, PPC-F, and PPC-G. PPC-A designates a healthy/Incidental disease subject. PPC-B designates a subject with mild periodontal disease. PPC-C designates a subject with high GI (gingival inflammation index). PPC-D designates a subject with some tooth loss (about 50% tooth loss). PPC-E designates a subject with posterior disease. PPC-F indicates a subject with severe tooth loss (about 75% tooth loss). PPC-G indicates a subject with severe disease. The description of clinical parameters for each PPC appears in Table 9. There were significant differences among all seven PPCs, and these values were provided for descriptive and comparative purposes. PPC-A (Health) had the lowest mean extent of BOP, GI≥1, and PI≥1. The mean extent of IAL≥3 mm of 8% and a mean extent of PD≥4 mm of 2% were the lowest among all 7 periodontal profile classes. PPC-B (Mild Disease) was mainly characterized by a slight increase in IAL≥3 mm and PD≥4 mm mean extent scores, and significant higher BOP (3-fold) and GI (9-fold) when compared to PPC-A. PPC-C (High GI) was notably marked by the highest mean extent GI score among all periodontal profile classes and was seen in 10% of the population. PPC-D (Tooth Loss) was characterized by fewer teeth. PPC-E (Posterior Disease) was marked by a moderate mean extent of IAL≥3 mm of 33% mainly located at the posterior dentition. PPC-F (Severe Tooth Loss) was characterized by the lowest mean number of teeth (8 teeth), where the remaining teeth were mainly mandibular anterior teeth with an edentulous maxilla and reflected 13% of the population. Finally, PPG-G (Severe Disease) was characterized by the highest mean extent of IAL≥3 mm of 54% and PD≥4 mm of about 25%. Higher BOP, GI, and PI extent scores were also found in this generalized severe disease profile.

The method can further include the step of performing a dental examination on a patient and determining for each tooth a Tooth Profile Class (TPC). The TPC is a stratification that is applied to each individual tooth in a patient that defines distinct categories of teeth based on clinical parameters. The seven TPCs are designated herein as TPC-A, TPC-B, TPC-C, TPC-D, TPC-E, TPC-F, and TPC-G. The tooth-level LCA procedure enabled us to identify 7 TPCs (A-G), in the DARIC population. The description of the 14 clinical parameters for each TPC is described in Table 12. As described in the Examples, significant differences among all seven TPCs were observed, and these values are provided for descriptive and comparative purposes. For example, TPC-A can include teeth with the least attachment loss, PD, BOP, recession, GI, PI, caries, and number of crowns. TPC-G can include teeth with signs of periodontitis represented by substantial attachment loss, deep PD, high GI and PI.

The PPC and TPC can be used together to generate a composite risk score for an individual, which is referred to herein as the Index of Periodontal Risk (IPR). The IPR can range from 4 to 46 and can be calculated for each individual based on tooth loss risks. The analytical approach used to calculate IPR can be based on a 7×7 table (PPC×TPC) of predicted probabilities for 10-year tooth loss. First, each individual can be assigned to one of the 7 PPCs and then, each tooth can be classified to one of the 7 TPCs. The IPR can then be calculated as the mean predicted probability for 10-year tooth loss across all teeth present for each individual. The development of the IPR can include one or more risk factors for periodontal disease, such as age, sex, race, diabetes, and smoking status. Tooth loss and disease progression risk estimates based on classes developed specific IPR cut—can support 3 levels or classes of risk for tooth loss denominated Index of Periodontal Classes (IPC): IPC-“Low” (IPR 0-10), IPC-“Moderate” (IPR 10-20), and IPC-“High” (IPR>20). In some embodiments, the method can include the step of performing a dental examination on a patient and classifying the subject into one of the seven PPCs and determining for each tooth a TPC. The method can then include the step of calculating the mean predicted probability for 10-year tooth loss across all teeth present in the individual. The IPR can fall into one of three risk of tooth loss categories (Low, Moderate, or High) referred to herein as an Index of Periodontal Class as previously described.

In some embodiments, each stage of the disclosed PPC system is characterized by unique single nucleotide polymorphisms (SNPs) as described in Table 1. These SNPs are associated with unique pathways, identifying unique druggable targets for each stage. Therefore, disclosed herein is a method of classifying a subject into PPC stages that involves assaying a biological sample from the subject for one or more SNPs disclosed herein. This can be done using a saliva sample.

For example, in some embodiments PPC-A is associated with rs12970437. In some embodiments PPC-B is associated with one or more SNPs selected from the group consisting of rs10881157, rs11011168, rs1915979, rs17660815, rs10411431, rs282669, rs17054017, rs9616028, rs12439077, rs12270207, rs2030737, rs8002848, rs8132, rs11574632, rs2245768, rs7195261, rs3853178, rs368380, rs2527073, rs985211, rs10244526, rs476154, rs311433, rs17141570, rs10773345, rs11852677, rs9872121, and rs947089.

In some embodiments PPC-C is associated with one or more SNPs selected from the group consisting of rs10163673, rs10762418, rs10786967, rs11591738, rs11605897, rs11671501, rs11688237, rs11730793, rs11936468, rs12149031, rs12157940, rs12287284, rs12437774, rs12524678, rs13140329, rs1328710, rs1374624, rs1383795, rs1440634, rs1484126, rs1530239, rs1559836, rs1574733, rs1706226, rs17081845, rs17168014, rs17784714, rs1794520, rs1850937, rs1864268, rs1971643, rs1997316, rs2012362, rs205402, rs2165693, rs2290983, rs2302373, rs2311120, rs2389911, rs2443743, rs2862478, rs4437385, rs4475, rs4586547, rs4758347, rs6031077, rs6462623, rs6476553, rs663578, rs6662013, rs6852438, rs6887840, rs6891234, rs6991003, rs7164558, rs7192715, rs7209032, rs724845, rs7263547, rs7532243, rs7720860, rs7839820, rs7908425, rs7924855, rs8038015, rs8097637, rs8109263, rs85431, rs9325009, rs9514947, and rs965919.

In some embodiments PPC-D is associated with one or more SNPs selected from the group consisting of rs10005793, rs10018581, rs10225336, rs10227357, rs10280585, rs10417806, rs10776634, rs10819125, rs10833328, rs10893450, rs10985401, rs11017774, rs11033288, rs11047355, rs11122200, rs11129475, rs11210982, rs1148546, rs1153536, rs11721807, rs11780899, rs11854996, rs11866630, rs12055381, rs12101334, rs12108434, rs12217983, rs12340257, rs12346949, rs12512358, rs12522180, rs12568660, rs12629984, rs12927176, rs13032979, rs13110356, rs13166852, rs1328247, rs1348467, rs1464609, rs1538432, rs1588935, rs165110, rs16841998, rs16908206, rs16940467, rs16991843, rs17228469, rs17252387, rs17275995, rs17397004, rs17404332, rs17405875, rs17607190, rs17765204, rs1833, rs1843371, rs1860613, rs1931084, rs1994379, rs2025935, rs2065617, rs2098883, rs2129209, rs2169856, rs2186903, rs2226179, rs2243879, rs2287410, rs2290717, rs2359376, rs2404645, rs2571236, rs2617101, rs2634511, rs2682551, rs2713621, rs27567, rs2828792, rs2858590, rs299499, rs3010311, rs3050, rs3101796, rs325369, rs358734, rs3744744, rs3759317, rs376200, rs3785817, rs37974, rs428311, rs4318271, rs4330511, rs4445711, rs4641033, rs4653109, rs4659708, rs4726340, rs473172, rs4780337, rs4798470, rs4859966, rs4899265, rs4924590, rs4965520, rs501290, rs5019079, rs534904, rs558706, rs572491, rs5770661, rs6098802, rs6134073, rs6134489, rs6448755, rs6561712, rs6564672, rs6601751, rs669310, rs6718569, rs673282, rs6756721, rs6804188, rs6844333, rs6923429, rs6950173, rs7017336, rs7028263, rs7076143, rs7081956, rs7164199, rs7285846, rs7336914, rs7406992, rs741223, rs747838, rs7500162, rs7578859, rs758971, rs7613532, rs773312, rs7740170, rs7804, rs7808523, rs7904936, rs8022554, rs827080, rs867677, rs888092, rs915180, rs9428536, rs9457640, rs949741, rs955901, rs959083, rs9701452, rs9830768, rs984984, and rs9972232.

In some embodiments PPC-E is associated with one or more SNPs selected from the group consisting of rs10055033, rs1013261, rs10170697, rs10171384, rs10224959, rs10795862, rs10799145, rs10853171, rs10960440, rs11180563, rs11237082, rs11595862, rs11774301, rs11982297, rs12135329, rs12191170, rs12193727, rs12220989, rs12521638, rs12546876, rs12587917, rs12592259, rs12936464, rs1420364, rs1472969, rs1492658, rs1516920, rs1613, rs167237, rs16950392, rs16992846, rs17105275, rs17261780, rs17310798, rs17554439, rs1793767, rs1945146, rs2028270, rs2040784, rs2117817, rs2140634, rs2153226, rs2184925, rs2499914, rs2639989, rs2775941, rs2822127, rs2822129, rs2916635, rs318373, rs4413520, rs4432731, rs465947, rs4659558, rs4783961, rs4809947, rs4842345, rs4848521, rs485345, rs5016898, rs550445, rs625492, rs648731, rs6535497, rs6586508, rs6665694, rs7015174, rs7219527, rs7235201, rs726237, rs7275448, rs7330295, rs7642023, rs7687468, rs7761181, rs7818703, rs7847940, rs8192856, rs878171, rs893787, rs9306, rs9375794, rs9514840, and rs9946639.

In some embodiments PPC-F is associated with one or more SNPs selected from the group consisting of rs1001486, rs10036878, rs10217492, rs1024445, rs10806844, rs10861932, rs10876555, rs10899610, rs10921094, rs10949356, rs11161058, rs11237081, rs11257948, rs11601125, rs11662579, rs11663290, rs11722347, rs11743051, rs11764731, rs11816208, rs11817679, rs12202642, rs12414476, rs12425668, rs12430698, rs12436090, rs12480427, rs12517647, rs12815089, rs12867851, rs12883143, rs12998780, rs13246891, rs1385398, rs1386269, rs1434123, rs1496650, rs1680580, rs16851463, rs16857576, rs16931840, rs16948939, rs17042804, rs17057184, rs1707981, rs17171742, rs17193494, rs17230650, rs17272228, rs17286152, rs17336166, rs17365678, rs17585733, rs17687542, rs17710623, rs17764155, rs17773903, rs1867714, rs1878705, rs1893452, rs1902431, rs1967505, rs1998058, rs2012586, rs2039052, rs2055141, rs215941, rs228146, rs2303059, rs2331494, rs2345191, rs2603731, rs2798776, rs2823042, rs299958, rs3017366, rs3755845, rs4148202, rs4273108, rs4309286, rs4396981, rs4534931, rs4684503, rs4841663, rs4858184, rs4884948, rs4910237, rs4941366, rs5997400, rs6071100, rs6471325, rs6488099, rs6575926, rs6576504, rs6600382, rs6703917, rs6729815, rs6744726, rs6757665, rs6820473, rs6911915, rs6997097, rs6997500, rs7042041, rs7141908, rs7285551, rs7302663, rs731945, rs7327336, rs753127, rs7613298, rs7753922, rs7764197, rs7806488, rs7921396, rs7952254, rs8012983, rs9267853, rs930851, rs9369583, rs9462426, rs9530506, rs9537303, rs9601679, rs9690040, rs978493, rs9814936, rs9832396, and rs9964434.

In some embodiments PPC-G is associated with one or more SNPs selected from the group consisting of rs10002158, rs10008703, rs1011058, rs10281536, rs1030038, rs10448335, rs10499129, rs10769990, rs10775437, rs10797752, rs10947850, rs11003132, rs11067587, rs11122949, rs1112919, rs11133161, rs1116008, rs11629965, rs11728254, rs11758068, rs11789281, rs11843657, rs11869615, rs11984109, rs12029285, rs12091354, rs12101383, rs12200300, rs12418774, rs12465864, rs12471370, rs12504119, rs12551283, rs12612309, rs12677687, rs12801239, rs13131866, rs13265778, rs13267206, rs1331472, rs13425677, rs1358882, rs1376605, rs1449542, rs1486816, rs1502276, rs1507869, rs1533344, rs1542371, rs1585775, rs16838572, rs16875331, rs16912660, rs16947580, rs17043278, rs17062397, rs17064357, rs170827, rs17096074, rs17113689, rs17137637, rs17140677, rs1720228, rs17676820, rs17729851, rs1775919, rs17792047, rs179885, rs1820825, rs1879671, rs188178, rs1994317, rs201033, rs2039957, rs2062312, rs2103304, rs2159472, rs2189099, rs2193875, rs223110, rs2236891, rs2246356, rs2254996, rs2255273, rs2305804, rs2315578, rs2320214, rs2324499, rs2392285, rs2424300, rs2736874, rs2899576, rs3024851, rs35405, rs3801899, rs3812389, rs4140872, rs4290517, rs4362420, rs4371785, rs4561982, rs4595351, rs4648955, rs4673287, rs4715277, rs4754687, rs4862622, rs4896502, rs4940002, rs4952539, rs558917, rs6052782, rs6456180, rs6465149, rs6484998, rs6485513, rs6533101, rs6764156, rs6789415, rs6814251, rs6862, rs693442, rs7048256, rs709143, rs7242593, rs7313672, rs7446448, rs756958, rs7697424, rs771177, rs7742386, rs7859003, rs7864, rs8086522, rs8104456, rs8114348, rs831784, rs888804, rs913585, rs9313719, rs9350031, rs9376791, rs9416628, rs9460898, rs9478243, rs9531387, rs9601292, rs966423, and rs9821929.

Also disclosed herein is a method of treating and/or preventing periodontal disease and/or tooth loss in a subject that involves classifying a subject into one of seven PPCs based on information on each tooth present in the subject obtained via a dental examination; and treating the subject with an effective amount of a therapeutic agent that targets one or more genes associated with the classified PPC, wherein the treatment and/or preventive therapy provided to the subject is different as compared to the treatment and/or preventive therapy that would have been provided if a traditional periodontal disease/tooth loss classification system were used. The instances where a dentist could change the treatment plan are displayed in top part of FIG. 17 along with the number of people who could be affected with that change.

In addition to the above changes in treatment plan, in some embodiments, wherein the subject is classified as PPC-B and has the appropriate genes, the subject can be treated with a therapeutic agent selected from the group consisting of dextrose, pyroglutamic acid, streptol, vandetanib, progesterone, acarbose, miglitol, celgosivir, duvoglustat hydrochloride, duvoglustat, phorbol myristate acetate, leucovorin, and irinotecan.

Similarly, in some embodiments, wherein the subject is classified as PPC-C, the subject can be treated with a therapeutic agent selected from the group consisting of quercetin, metaproterenol sulfate, copanlisib, alpelisib, pictilisib, apitolisib, pf-04691502, gedatolisib, sonolisib, sf-1126, voxtalisib, pilaralisib (chembl3218575), dactolisib, pi-103, gsk-2636771, buparlisib, chembl1229535, hydrogen peroxide, clavulanic acid, amoxicillin, me-344, nv-128, metformin hydrochloride, chembl1161866, capsaicin, chembl1213492, nintedanib esylate, su-014813, krn-633, l-21649, tak-593, ag-13958, bms-690514, foretinib, mgcd-265, brivanib alaninate, semaxanib, cediranib, sorafenib, anlotinib, cc-223, dovitinib, fruquintinib, lenvatinib, linifanib, mk-2461, motesanib, nintedanib, pazopanib, sunitinib, tesevatinib, tivozanib, vatalanib, axitinib, regorafenib, lenvatinib mesylate, ilorasertib, cep-11981, cep-7055, chiauranib, jnj-26483327, osi-930, rg-1530, telatinib, famitinib, xl-999, brivanib, sunitinib malate, vandetanib, sorafenib tosylate, pazopanib hydrochloride, lucitanib, enmd-2076, orantinib, x-82, xl-820, cep-5214, su-14813, cp-459632, sulfatinib, 4sc-203, chembl313417, imc-3c5, taberminogene vadenovec, tg100-801, imc-1c11, pegpleranib sodium, chembl384759, guanosine triphosphate, glycine, tezampanel, butabarbital, butalbital, talbutal, secobarbital, metharbital, thiopental, primidone, mephobarbital, phenobarbital, chembl301536, 2s,4r-4-methylglutamate, domoic acid, kainic acid, mesalamine, methyprylon, amobarbital, aprobarbital, butethal, heptabarbital, hexobarbital, barbital, selurampanel, topiramate, pentobarbital, quisqualate, l-glutamate, protirelin, taltirelin, ganitumab, picropodophyllotoxin, bms-754807, linsitinib, aew-541, dalotuzumab, insulin glargine, emactuzumab, azd-4547, chembl401930, brigatinib, ceritinib, chembl464552, chembl458997, dimethisterone, xl-228, biib-022, ave-1642, cixutumumab, insm-18, kw-2450, figitumumab, robatumumab, mecasermin, pl-225b, teprotumumab, mecasermin rinfabate, chembl1230989, acetylcysteine, thrombin, insulin human, chembl263143, chembl397666, raloxifene, erlotinib, rinfabate, staurosporine, and hydrochlorothiazide.

In some embodiments, wherein the subject is classified as PPC-D, the subject can be treated with a therapeutic agent selected from the group consisting of temazepam, adinazolam, halazepam, diazepam, oxazepam, triazolam, estazolam, bromazepam, clotiazepam, fludiazepam, ketazolam, prazepam, quazepam, cinolazepam, nitrazepam, clorazepate dipotassium, chlordiazepoxide, eszopiclone, meprobamate, clobazam, alprazolam, butalbital, clonazepam, desflurane, talbutal, butabarbital sodium, lorazepam, metharbital, methyprylon, midazolam hydrochloride, primidone, propofol, pentobarbital sodium, secobarbital sodium, sevoflurane, thiamylal sodium, flurazepam hydrochloride, halothane, isoflurane, adipiplon, lorediplon, resequinil, pf-06372865, methohexital sodium, thiopental sodium, chlordiazepoxide hydrochloride, pentobarbital, ethchlorvynol, glutethimide, flumazenil, clorazepic acid, methoxyflurane, midazolam, triclofos sodium, topiramate, gaboxadol, etomidate, acamprosate calcium, flurazepam, ocinaplon, enflurane, etazolate, chembl2325441, pregnenolone, s-adenosylhomocysteine, odanacatib, proscillaridin, cyproheptadine, cyclopentolate, eletriptan, methysergide, zolmitriptan, ergotamine, rizatriptan, mianserin, eletriptan hydrobromide, almotriptan malate, lasmiditan, chembl266591, serotonin, 5-meo-dmt, 5-methoxytryptamine, 8-oh-dpat, chembl1256797, clozapine, dihydroergotamine, chembl1332062, donitriptan, chembl101690, chembl3186179, naratriptan, olanzapine, quetiapine, sumatriptan, tryptamine, xanomeline, brl-15,572, gr-127935, chembl1256701, chembl277120, metergoline, metitepine, methylergonovine, risperidone, sertindole, yohimbine, adenosine triphosphate, azd-5438, pha-793887, at-7519, roniciclib, colforsin, papaverine hydrochloride, methicillin, adenosine phosphate, hesperidin, chembl578514, lipoic acid, methacholine chloride, leucine, chembl506495, galmic, galnon, chembl541253, chembl450441, methylene blue, bretylium tosylate, chembl604991, methoxamine, temazepam, galanin, diphemanil, galantide, androstenedione, testosterone, vandetanib, sorafenib, cabozantinib, regorafenib, ponatinib, ast-487, cep-11981, linifanib, lucitanib, quizartinib, sunitinib, tamatinib, at-9283, motesanib, amuvatinib, lenvatinib, alectinib hydrochloride, cep-32496, lestaurtinib, sorafenib tosylate, sunitinib malate, cep-2563, cetuximab, chembl1213492, ink-128, everolimus, dexamethasone, azd-1480, chembl306380, dovitinib, genistein, imatinib, chembl126955, enmd-2076, alectinib, phorbol myristate acetate, xl-999, tretinoin, nintedanib, pyrimethamine, cobalt (ii) ion, verapamil, tetanus toxoid, ceritinib, medronic acid, quercetin, metaproterenol sulfate, copanlisib, alpelisib, pictilisib, apitolisib, pf-04691502, gedatolisib, sonolisib, sf-1126, voxtalisib, pilaralisib (chembl3218575), dactolisib, pi-103, gsk-2636771, buparlisib, chembl1229535, arsenic trioxide, motexafin gadolinium, chembl449269, flavin adenine dinucleotide, spermidine, fotemustine, cerivastatin, pseudoephedrine hydrochloride, bosutinib, dasatinib, hydrocortisone, lithium carbonate, lithium citrate hydrate, acetylcysteine, sunitinib, aripiprazole, naphthalene, chembl435278, chembl122264, chembl17639, chembl21283, monoethanolamine, quercetin, prednisolone, and puromycin.

In some embodiments, wherein the subject is classified as PPC-E, the subject can be treated with a therapeutic agent selected from the group consisting of vantictumab, flanvotumab, docetaxel, paclitaxel, acalabrutinib, ibrutinib, inositol, retinol, genistein, leuprolide acetate, dexamethasone, halofuginone, dihydrospingosine, sphingosine, nifedipine, chembl1179605, mefenamic acid, pioglitazone, rosiglitazone, troglitazone, chembl169233, aspirin, chembl1161866, stanolone, chembl566340, colforsin, darotropium bromide, tridihexethyl chloride, tolterodine tartrate, propantheline bromide, oxyphenonium bromide, aripiprazole, olanzapine, methixene, terfenadine, clozapine, oxyphencyclimine, procyclidine, loxapine, promazine, hyoscyamine, darifenacin, tridihexethyl, anisotropine methylbromide, diphemanil methylsulfate, scopolamine, benzquinamide, propiomazine, tropicamide, brompheniramine, glycopyrrolate, tolterodine, pilocarpine, mivacurium, diphenidol, chlorprothixene, pipecuronium, levomepromazine, isopropamide, mepenzolate, fesoterodine, methacholine, aclidinium, umeclidinium, acetylcholine, arecaidine propargyl ester, arecoline, bethanechol, furtrethonium, 5-methylfurmethiodide, chembl99521, eribaxaban, oxotremorine, chembl130715, chembl74300, milameline, sabcomeline, xanomeline, alcuronium, brucine, chembl343796, strychnine, chembl2206331, chembl343357, vinburnine, vincamine, chembl139677, chembl523685, chembl1256845, 4-damp, chembl279453, amitriptyline, benztropine mesylate, atropine, biperiden (chembl1101), clidinium, dicyclomine, dothiepin (chembl1492500), chembl580785, hexocyclium, himbacine, chembl1256682, ipratropium, methoctramine, hydrochloric acid, otenzepad, oxybutynin, pirenzepine, propantheline, solifenacin, coenzyme_a, quinuclidinyl benzilate, tiotropium (chembl1900528), tripitramine, chembl1233686, chembl1628667, revatropate, umeclidinium bromide, las190792, afacifenacin, tarafenacin, solifenacin succinate, aclidinium bromide, methacholine chloride, tiotropium bromide, acetylcholine chloride, carbachol (chembl14), pilocarpine hydrochloride, cevimeline hydrochloride, suxamethonium, batefenterol, mepenzolate bromide, hexocyclium methylsulfate, dicyclomine hydrochloride, bethanechol chloride, atropine sulfate, oxybutynin chloride, darifenacin hydrobromide, ipratropium bromide hydrate, methscopolamine bromide, oxyphencyclimine hydrochloride, glycopyrrolate bromide, isopropamide iodide, trospium chloride, fesoterodine fumarate, cyclopentolate hydrochloride, asm-024, azd8683, clidinium bromide, thiethylperazine, cevimeline, doxepin, promethazine, itopride, homatropine methylbromide, anacetrapib, dalcetrapib, torcetrapib, chembl67129, evacetrapib, cerivastatin, tamoxifen, cep-2563, ticlopidine, gsk-690693, sotrastaurin, (7s)-hydroxyl-staurosporine, midostaurin, quercetin, sotrastaurin acetate, staurosporine, dexfosfoserine, ingenol mebutate, chembl369507, bryostatin, medronic acid, insulin human, vandetanib, chembl552425, sulfasalazine, ridogrel, dazoxiben, phorbol myristate acetate, belimumab, briobacept, atacicept, tabalumab, blisibimod, dioxane, etoposide, and citric acid.

In some embodiments, wherein the subject is classified as PPC-F, the subject can be treated with a therapeutic agent selected from the group consisting of isradipine, nimodipine, nisoldipine, verapamil, felodipine, nitrendipine, nifedipine, mibefradil, nilvadipine, salsalate, bepridil hydrochloride, pregabalin, gabapentin, gabapentin enacarbil, imagabalin, atagabalin, magnesium sulfate, amlodipine, celecoxib, dronedarone, chembl566340, go-6976, quercetin, gsk-690693, (7s)-hydroxyl-staurosporine, cep-2563, midostaurin, sotrastaurin, bryostatin, resveratrol, vasopressin, bosutinib, chembl359482, paroxetine, ocriplasmin, hydrochlorothiazide, chembl384759, insulin human, busulfan, zonisamide, fructose, trichostatin, ascorbate, ocriplasmin, guanidine hydrochloride, dalfampridine, tedisamil, nerispirdine, tretinoin, etretinate, zoledronic acid, chembl300914, pyridoxal phosphate, threonine, nirogacestat, regn-421, and cyclophosphamide.

In some embodiments, wherein the subject is classified as PPC-G, the subject can be treated with a therapeutic agent selected from the group consisting of danazol, chembl195368, chembl1234621, conbercept, sorafenib tosylate, lenalidomide, bevacizumab, nimodipine, progesterone, spironolactone, eplerenone, felodipine, desoxycorticosterone pivalate, drospirenone, aldosterone, hydrocortisone, desoxycorticosterone, dexamethasone, fludrocortisone, prednisolone, finerenone, onapristone, pf-03882845, oxprenoate potassium, x1550, mt-3995, ly2623091, desoxycorticosterone acetate, fludrocortisone acetate, corticosterone, rizatriptan, chembl482796, lipoxin a4, lithium, gadobenate dimeglumine, chembl185515, amiloride, pyrimethamine, nafamostat, pregnenolone, genistein, leuprolide acetate, dexamethasone, halofuginone, davalintide, pramlintide, pramlintide acetate, calcitonin salmon recombinant, doxorubicin, chembl574817, clozapine, odanacatib, palmitic acid, flanvotumab, ingenol mebutate, ellagic acid, aprinocarsen sodium, chembl1236539, balanol, enzastaurin, go-6976, sebacic acid, ruboxistaurin, sotrastaurin, midostaurin, quercetin, sotrastaurin acetate, gsk-690693, cep-2563, (7s)-hydroxyl-staurosporine, bryostatin, tamoxifen, dexfosfoserine, vitamin e, fructose, trichostatin, sacituzumab govitecan, teglarinad chloride, fluorouracil, ocriplasmin, halothane, proxyphylline, lisofylline, caffeine, tretinoin, urokinase, carboquone, insulin human, mesalamine, l-glutamate, pyrimethamine, pyridostigmine, pyrilamine, peginterferon lambda-1a, rintatolimod, pyroxamide, hydroxychloroquine, eledoisin, kassinin, neurokinin a, neurokinin b, chembl69367, senktide, substance p, isoflurane, azd2624, chembl480249, chembl221445, osanetant, chembl44229, saredutant, chembl9843, chembl1991816, amcinonide, talnetant, sb-222200, chembl275544, pyrimethamine, cobalt (ii) ion, verapamil, vasopressin tannate, famoxadone, azoxystrobin, coenzyme q2, proxyphylline, cholic acid, afimoxifene (chembl489), diarylpropionitrile, diethylstilbestrol, chlorotrianisene, estrogens, conjugated, etonogestrel, desogestrel, levonorgestrel, progesterone, toremifene, medroxyprogesterone acetate, estrone, tamoxifen, dienestrol, fulvestrant, norgestimate, ethinyl estradiol, melatonin, trilostane, fluoxymesterone, estramustine, estriol, prinaberel, propylpyrazoletriol, raloxifene, chembl282489, chembl188528, lasofoxifene, bazedoxifene, clomiphene, chembl201013, hexestrol, chembl520107, mestranol, danazol, allylestrenol, prasterone, estropipate, quinestrol, ospemifene, tibolone, estrogens, conjugated synthetic a, synthetic conjugated estrogens, b, estradiol, mitotane, sr16234 (chembl3545210), fispemifene, ly2245461, idoxifene, gtx-758, afimoxifene (chembl10041), droloxifene, acolbifene, tamoxifen citrate, estradiol valerate, clomiphene citrate, estradiol cypionate, estrogens, esterified, diethylstilbestrol diphosphate, toremifene citrate, bazedoxifene acetate, chf4227, gdc-0810, estradiol acetate (chembl1200430), polyestradiol phosphate, mk-6913, iodine, vintafolide, custirsen, chembl304552, everolimus, chembl181936, chembl391910, pertuzumab, chembl180300, dienogest, estrogen, rad1901, chembl222501, arzoxifene, chembl180071, chembl193676, ethynodiol diacetate, genistein, raloxifen, ribociclib, raloxifene core, trastuzumab, chembl236718, abemaciclib, 2-amino-1-methyl-6-phenylimidazo[4, 5-b]pyridine, erteberel, exemestane, endoxifen, megestrol acetate, sivifene, palbociclib, leflunomide, estrone sodium sulfate, letrozole, chembl236086, chembl184151, chembl223026, lapatinib, chembl180517, norelgestromin, anastrozole, carboquone, gonadorelin, chembl1213270, norgestrel, and medronic acid.

As an example, if a subject comes into a dental office for a screening exam the subject would receive a full mouth examination, e.g. consisting of probing depths, attachment loss, bleeding on probing, gingival index assessment, plaque score assessment, missing teeth and crowns. The full mouth exam would be entered into a charting program and the data could be entered into a computer where PPC classification would be returned. If the subject's PPC assignment is PPC-C (for this example) a DNA sample would be taken and polymorphisms (SNP's) would be determined. The subject may receive non-surgical periodontal treatment, local delivery of an antimicrobial, possibly a systemic antibiotic a recall frequency of 1-2×/year or possibly 3-4/× year would be recommended. This subject may be referred to a periodontist and a medical consultation may be recommended. One of the SNP's identified in the PPC-C class is rs1945146 as being related to having more periodontal disease. In addition, aspirin is one of the therapeutic agents that is listed as a possible treatment for PPC-C. FIG. 28 shows that people who have one or more copies of the minor allele and are not currently taking Aspirin have a significantly higher extent of interproximal attachment loss >=3 mm than people who have one or more copies of the minor allele who are currently taking Aspirin (p=0.01). In fact, the level of attachment loss is like people who do not have a copy of the minor allele and are taking aspirin. In this real-world example using the Dental ARIC dataset, it is important to note that Aspirin appears to be beneficial to people who have one or more copies of the minor allele for rs1945146 and are in PPC-C. Aspirin does not appear to be beneficial for any other PPC class.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows computed probabilities for 10-year tooth loss (≥3 teeth) using the DARIC dataset stratified by Periodontal Profile Classes (PPC) and Tooth Profile Classes (TPC).

FIGS. 2A-2H show graphs that can demonstrate predicted probability for the Index of Periodontal Risk (IPR) score associated with tooth loss, attachment loss, and edentulism. FIG. 2A shows predicted probability for the Index of Periodontal Risk (IPR) score associated with tooth loss in the DARIC dataset. FIG. 2B shows Area under the Receiver Operator Curve (ROC) for 10-year tooth loss (≥3 teeth) in the DARIC population. FIG. 2C shows predicted probability for the Index of Periodontal Risk (IPR) score associated with tooth loss in the Piedmont (PDS) dataset. FIG. 2D shows Area under the Receiver Operator Curve (ROC) for 10-year tooth loss (≥3 teeth) in the PDS dataset. FIG. 2E can show the association of the IPR score and 3-year attachment loss in the PDS dataset. FIG. 2F can demonstrate the area under the ROC for 3-year attachment loss in the PDS dataset. FIG. 2G can demonstrate the association of the IPR score and edentulism in the PDS dataset. FIG. 2H can demonstrate the area under the ROC for edentulism in the PDS dataset.

FIG. 3 can demonstrate the Index of Periodontal Risk (IPR) calculation for a hypothetical person classified under the Periodontal Profile Class E (PPC-E) having teeth representing multiple Tooth Profile Classes (TPC). The computed probabilities values are presented for each tooth according to the TPC classification. The IPR value for this hypothetical person is 18.5, which represents the sum of the computed probabilities divided by the total number of teeth. M represents missing tooth.

FIGS. 4A-4G can demonstrate item response probabilities conditional on class membership for FIG. 4A can show Tooth Status (presence or absence), FIG. 4B can show Prosthetic Crowns (presence or absence), FIG. 4C can show Interproximal Attachment Loss≥3 mm, FIG. 4D can demonstrate Pocket Depth≥4 mm, FIG. 4E can show the Gingival Index (GI, dichotomized as ≥1 sites with GI≥1 vs none), FIG. 4F can show the Plaque Index (PI, dichotomized at ≥1 sites with PI≥1), FIG. 4G can show bleeding on Probing (BOP, dichotomized at 50% or ≥3 sites per tooth). Probabilities are illustrated for each tooth type (1-32) representing both arches graphically in a heatmap for each clinical parameter in the DARIC sample. The upper and lower arch are represented for each Periodontal Profile Class (PPC) for each tooth.

FIG. 5 can demonstrate the Distribution of the seven Tooth Profile Classes (TPC) for each of the seven Periodontal Profile Classes (PPC). TPC-A (Health), TPC-B (Recession), TPC-C (Crown), TPC-D (GI), TPC-E (Interproximal Disease), TPC-F (Reduced Periodontium), and TPC-G (Severe Disease).

FIGS. 6A-6G can demonstrate the percentage distribution of each Tooth Profile Class (TPC) by tooth for all Periodontal Profile Classes (PPC). The percentage distribution is shown for each tooth type (1-32) representing both arches graphically in a heatmap for each PPC in the DARIC population. Both the upper and lower arch are represented for each TPC for each tooth. FIG. 6A. PPC-A (Health), FIG. 6B. PPC-B (Mild Disease), FIG. 6C. PPC-C (High GI), FIG. 6D. PPC-D (Tooth Loss), FIG. 6E. PPC-E (Posterior Disease), FIG. 6F. PPC-F (Severe Tooth Loss), FIG. 6G. PPC-G (Severe Disease).

FIG. 7 shows clinical utility of the UNC Periodontal Profile Phenotype (P³) System described herein. This figure provides a conceptual framework for the development and clinical utility of the P³ system containing the three major domains of Diagnosis, Risk and Outcome. This figure is a framework for a system that can assist clinicians in relating patient classification, risk for tooth loss, and treatments. Components of the P³ that have been demonstrated include: the calculation of PPC and TPC using LCA as described by Morelli et al.³; the development of risk models and impact on future tooth loss and attachment loss, also described by Morelli et al.¹; and the associations of PPC with prevalent systemic diseases and inflammatory markers compared to associations generated by CDC/AAP and European indices described by Beck, et al.²⁴

FIG. 8 shows clinical measures for each tooth profile class. TPC=Tooth Profile Class, GI=Gingival Inflammation, Interprox=Interproximal Disease, DimPerio=Diminished Periodontium; iAL=mean interproximal (i.e. adjoining tooth surfaces, 4 sites) attachment loss in mm; dAL=mean direct attachment loss in mm (buccal and lingual surfaces, 2 sites); iPD=mean interproximal probing depth in mm; dPD=mean direct probing depth in mm (buccal and lingual surfaces); iCEJ=mean distance from free gingival margin to cementoenamel junction (CEJ) in mm measured interproximally, negative indicates recession, i.e. CEJ exposed and above the free gingival margin; dCEJ=mean distance from free gingival margin to cementoenamel junction (CEJ) in mm measured directly at buccal and lingual surfaces (2 sites), negative indicates recession, i.e. CEJ exposed and above the free gingival margin; BOP=mean Bleeding on probing as percent of sites (6 sites per tooth); GI=mean Gingival Index score as measured across buccal sites. GI is scored 0-3; PQ=mean Plaque index score as measured across buccal sites. PQ is scored 0-3; Crown=proportion of teeth with crowns

FIG. 9 shows distribution of Tooth Profile Classes (TPC) by Person Profile Class (PPC) in the Dental ARIC Study. Sev=Severe Disease; iDis=interproximal disease; Dim Per=diminished periodontium; GI=gingival inflammation; Crn=crown on tooth; Rec=recession.

FIG. 10 shows intraoral distribution of Tooth Profile Class (TPC) by Periodontal Profile Class (PPC) by Tooth Type.

FIG. 11 shows Mean Interproximal Attachment Level, Mean Probing Depth, Extent Bleeding on Probing and Mean Gingival Index by Periodontal Profile Class (PPC) and Tooth Type.

FIG. 12 shows Distribution of Tooth Profile Classes (TPC) Posterior Probabilities in Dental ARIC Study Participants for all Teeth According to Arch. Each of the TPC descriptors, lower levels of the condition and higher levels for the upper [ 1/16 tooth numbers] and lower [ 17/32 tooth numbers] arches are represented. For example, Tooth #8 has a 61% probability of being a Healthy TPC in an individual, considering all teeth among all Dental ARIC participants.

FIGS. 13A-13C show example P³ system charts for (FIG. 13A) P³ System Chart for Healthy Patient; (FIG. 13B) P³ System Chart for High GI Patient; (FIG. 13C) P³ System Chart for Severe Periodontitis Patient.

FIGS. 14A-14B show risk for incident ischemic stroke in the Dental ARIC cohort depicted as crude (FIG. 14A) and adjusted (FIG. 14B) Hazard Ratios for the various classes of periodontal disease: PPC-A or reference healthy group without periodontal disease, PPC-B, or mild periodontal disease, PPC-C or high GI score, PPC-D or tooth loss, PPC-E or posterior disease, PPC-F or severe tooth loss and PPC-G or severe periodontal disease. In FIG. 14B Hazard Ratios are adjusted for Race/Center, Age, Gender, BMI, Hypertension, Diabetes, LDL Level, Smoking (3-levels), Pack Years, Education (3-levels).

FIGS. 15A-15B show Risk reduction in incident ischemic stroke in the main ARIC cohort depicted as crude (FIG. 15A) and adjusted (FIG. 15B) Hazards Ratio for episodic and regular (reference group for comparison) dental care users, determined at visit 4. In FIG. 15A Hazards Ratio is adjusted for Race/Center, Age, Gender, BMI, Hypertension, Diabetes, LDL Level, Smoking (3-levels), Pack Years, Education (3-levels).

FIGS. 16A-16D show Kaplan Meier curves depicting 15 years' outcome of (FIG. 16A) incident ischemic stroke (overall), (FIG. 16B) lacunar, (FIG. 16C) cardioembolic and (FIG. 16D) thrombotic stroke subtypes. Inset: Crude Hazard Ratios for ischemic stroke (overall) and stroke subtypes. Hazards Ratio adjusted for Race/Center, Age, Gender, BMI, Hypertension, Diabetes, LDL Level, Smoking (3-levels), Pack Years, Education (3-levels).

FIG. 17 shows a table that can demonstrate treatment recommendations based on the traditional classification system (CDC/AAP) as compared to the PPC classification system described herein and can demonstrate the change in treatment recommendations as a result of the PPC classification system.

FIGS. 18A and 18B show traditional disease classifications. FIG. 18A is a traditional method where experts draw one or more lines in the disease continuum to create disease classifications. FIG. 18B shows a type of computer modeling to create overlapping clusters of individuals who could be in more than one disease classification.

FIG. 19 shows a newer method for disease classification. Latent Class Analysis or Deep Learning models are used to place individuals with similar characteristics related to the disease in mutually exclusive, classes on an agnostic basis.

FIG. 20 shows adaptation of PPC Nomenclature to 2017 World Workshop Guidelines¹. This Figure depicts the adaptation of the Periodontal Profile Class (PPC) nomenclature and the Index of Periodontal Risk (IPR) score to the Stage and Grade nomenclature adopted by the 2017 World Workshop on Disease Classifications.¹ PPCs are designated as Stages (I-VII) identifying Health-Incidental through Severe Disease in individuals who are largely dentate as (I-IV) and those with increasing edentulism as V-VII. Stage V has mild tooth loss (Mild TL) and high extent gingival index scores (high gingival inflammation or Hi GI), Stage VI has moderate tooth loss (Mod TL) with many teeth with reduced periodontium (Reduced) and Stage VII (Severe TL) represents individuals with few remaining teeth. Grades A-C are assigned based upon the computed IPR score, which adjusts individual scores for age, race, gender, smoking and diabetes. This Table demonstrates that most individuals fall within one of fifteen distinct bins.

FIGS. 21A and 21B show mean percent of clinical measures within each stage. FIG. 21A shows mean percent of clinical measures within each WW17 Stage. Stage I is Initial Disease, Stage II is Moderate Disease, Stage III is Severe Disease, and Stage IV is Advanced Disease. Risk of Tooth Loss for each Stage is the probability of losing three or more teeth over a 10-year period. ALPD=extent of CAL≥3mm&PD4≥mm; Posterior=Percent of posterior teeth with ALPD; BOP=extent bleeding on probing; GI=extent of gingival inflammation; PQ=extent of plaque; Missing=number of missing teeth. FIG. 21B shows mean percent of clinical measures within each PPC Stage. Stage I is Health/Incidental Disease, Stage II is Mild Disease, Stage III is Moderate Disease, and Stage IV is Severe Disease, Stage V is Mild Tooth Loss/High Gingival Inflammation (TL/Hi GI), Stage VI is Moderate Tooth Loss/Reduced Periodontium (Moderate TL), Stage VII is Severe Tooth Loss (Severe TL). Risk of Tooth Loss for each Stage is the probability of losing three or more teeth over a 10-year period. ALPD=extent of CAL≥3 mm&PD4≥mm; Posterior=Percent of posterior teeth with ALPD; BOP=extent bleeding on probing; GI=extent of gingival inflammation; PQ=extent of plaque; Missing=number of missing teeth

FIGS. 22A and 22B show percent of participants who have risk factors for tooth loss due to periodontal disease. FIG. 22A shows perrcent of participants with risk factors for tooth loss due to periodontal disease by WW17 Stage. Risk factors are Male, African-American (AA), having Diabetes, Cigarette Smoking, and Age≥65 years. Stage I is Initial Disease, Stage II is Moderate Disease, Stage III is Severe Disease, and Stage IV is Advanced Disease. Risk of Tooth Loss for each Stage is the probability of losing three or more teeth over a 10-year period. FIG. 22B shows percent of participants with Risk Factors for Tooth Loss Due to Periodontal Disease by PPC Stage. Stage I is Health/Incidental Disease, Stage II is Mild Disease, Stage III is Moderate Disease, and Stage IV is Severe Disease, Stage V is Mild Tooth Loss/High Gingival Inflammation (TL/Hi GI), Stage VI is Moderate Tooth Loss/Reduced Periodontium (Moderate TL), Stage VII is Severe Tooth Loss (Severe TL). Risk of Tooth Loss for each Stage is the probability of losing three or more teeth over a 10-year period.

FIG. 23 shows risk for 10-Year Tooth Loss by PPC Stage and Grade. Stage I is Health/Incidental Disease, Stage II is Mild Disease, Stage III is Moderate Disease, and Stage IV is Severe Disease, Stage V is Mild Tooth Loss/High Gingival Inflammation (TL/Hi GI), Stage VI is Moderate Tooth Loss/Reduced Periodontium (Moderate TL), Stage VII is Severe Tooth Loss (Severe TL). Risk of Tooth Loss for each Stage is the probability of losing three or more teeth over a 10-year period. Cells without numbers have <10 study participants. It is unlikely to have participants in Stages IV to VII who would be classified as Grade A as well as having participants classified into Stages Ito III who would be Grade C.

FIGS. 24A and 24B show Piedmont 65+ Dental Study: Attachment Loss over Three Years of ≥3 mm FIG. 24A: Risk for 3-year Attachment Loss by WW17 and PPC Stage (n=363). Attachment loss increase is defined by 10% of sites increasing by 3 mm. WW17 Stages are: Stage I is Initial Disease, Stage II is Moderate Disease, Stage III is Severe Disease, and Stage IV is Advanced Disease. PPC Stages are: Stage I is Health/Incidental Disease, Stage II is Mild Disease, Stage III is Moderate Disease, and Stage IV is Severe Disease, Stage V is Mild Tooth Loss/High Gingival Inflammation (TL/Hi GI), Stage VI is Moderate Tooth Loss/Reduced Periodontium (Moderate TL), Stage VII is Severe Tooth Loss (Severe TL). FIG. 24B: Risk for 3-Year Attachment Loss by PPC Grade (n=363). The sample size was small enough that data were too sparse to present information on Grade within Stage. This Figure just presents Grade alone. Attachment loss increase is defined by 10% of sites increasing by 3 mm. Grade A is an Index of Periodontal Risk Score (IPR) of <10, Grade B is an IPR between 10 and <30, and Grade C is an IPR of ≥30.

FIG. 25 shows odds of having prevalent diabetes according to WW17 and PPC Stages. Odds for prevalent diabetes in the dental ARIC (Atherosclerosis Risk in Communities) cohort depicted as adjusted odds ratios (ORs) using the WW17 Supervised Approach (A) and adjusted ORs for the WW17 Unsupervised Approach (B). For A, Stage I or Initial Disease is the reference while Stage II or Moderate; Stage III or Severe; and Stage IV or Advanced are compared to Stage I. For B, PPC Stage I or Health-Incidental is the reference and the other periodontal profile classes PPC Stage II or Mild disease, PPC Stage III or Moderate disease, PPC Stage IV or Severe disease, PPC Stage V or Mild Tooth Loss/High Gingival Inflammation (Mild TL/Hi GI), PPC Stage VI or Moderate Tooth Loss/Reduced Periodontium (Mod TL/Reduced), or PPC Stage VII or Severe Tooth Loss (Severe TL). In (B) ORs are adjusted for race, examination center, age, gender, body mass index (BMI), smoking (three levels) and education (three levels). LCL indicates lower confidence limit; and UCL, upper confidence limit.

FIG. 26 shows hazard ratios for incident stroke according to WW17 and PPC Stages. Risk for incident ischemic stroke in the dental ARIC (Atherosclerosis Risk in Communities) cohort depicted as adjusted hazard ratios (HRs) using the WW17 Supervised Approach (A) and adjusted HRs for the WW17 Unsupervised Approach (B). For A, Stage I or Initial Disease is the reference while Stage II or Moderate; Stage III or Severe; and Stage IV or Advanced are compared to Stage I. For B, PPC Stage I or Health-Incidental is the reference and the other periodontal profile classes PPC Stage II or Mild disease, PPC Stage III or Moderate disease, PPC Stage IV or Severe disease, PPC Stage V or Mild Tooth Loss/High Gingival Inflammation (Mild TL/Hi GI), PPC Stage VI or Moderate Tooth Loss/Reduced Periodontium (Mod TL/Reduced), or PPC Stage VII or Severe Tooth Loss (Severe TL). In (B) HRs are adjusted for race/center, age, sex, body mass index, hypertension, diabetes mellitus, low-density lipoprotein level, smoking (3 levels), pack years, education (3 levels). LCL indicates lower confidence limit; and UCL, upper confidence limit.

FIG. 27 shows odds of having high C-Reactive Protein (CRP) level according to WW17 and PPC stages. Odds for having high C-Reactive Protein Level (CRP) in the dental ARIC (Atherosclerosis Risk in Communities) cohort depicted as adjusted odds ratios (ORs) using the WW17 Supervised Approach (A) and adjusted ORs for the WW17 Unsupervised Approach (B). For A, Stage I or Initial Disease is the reference while Stage II or Moderate; Stage III or Severe; and Stage IV or Advanced are compared to Stage I. For B, PPC Stage I or Health-Incidental is the reference and the other periodontal profile classes PPC Stage II or Mild disease, PPC Stage III or Moderate disease, PPC Stage IV or Severe disease, PPC Stage V or Mild Tooth Loss/High Gingival Inflammation (Mild TL/Hi GI), PPC Stage VI or Moderate Tooth Loss/Reduced Periodontium (Mod TL/Reduced), or PPC Stage VII or Severe Tooth Loss (Severe TL). In (B) ORs are adjusted for race, examination center, age, gender, BMI, smoking (three levels), diabetes, hypertension, and education (three levels). LCL indicates lower confidence limit; and UCL, upper confidence limit.

FIG. 28 shows adjusted mean extent of interproximal attachment loss by PPC and aspirin use in an ARIC study (n=6793). * P=0.01 (Aspirin vs. No Aspirin for the 1.2+2.2 group). Adjusted for Field Center, Age, and Sex. Individuals classified as PPC-C who take aspirin have significantly lower levels of attachment loss, but aspirin makes no difference in the other PPC groups.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

It must be noted that, as used in the specification and the appended claims, the singular forms “a,” “an,” and “the” include plural referents unless the context clearly dictates otherwise.

As used herein, “administering” refers to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques.

As used herein, “control” refers to an alternative subject or sample used in an experiment for comparison purpose and included to minimize or distinguish the effect of variables other than an independent variable.

As used herein, “preventative” and “prevent” refers to hindering or stopping a disease or condition before it occurs, even if undiagnosed, or while the disease or condition is still in the sub-clinical phase.

As used interchangeably herein, “subject,” “individual,” participant, or “patient” refers to a vertebrate organism, such as a mammal (e.g. human). “Subject” can also refer to a cell, a population of cells, a tissue, an organ, or an organism, preferably to human and constituents thereof.

As used herein, the terms “treating” and “treatment” refers generally to obtaining a desired pharmacological and/or physiological effect. The effect can be, but does not necessarily have to be, prophylactic in terms of preventing or partially preventing a disease, symptom or condition thereof, such as periodontal disease. The effect can be therapeutic in terms of a partial or complete cure of a disease, condition, symptom or adverse effect attributed to the disease, disorder, or condition. The term “treatment” as used herein covers any treatment of periodontal disease, in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

A number of embodiments of the invention have been described. Nevertheless, it will be understood that various modifications may be made without departing from the spirit and scope of the invention. Accordingly, other embodiments are within the scope of the following claims.

The recently introduced concept of precision medicine offers a new vision for the prevention and treatment of disease, as well as for biomedical research. Along the lines of the “personalized medicine” paradigm, precision medicine entails prevention and treatment strategies that take individual variability into account. A complete account of environmental and innate influences of disease susceptibility is certainly a daunting task. Nevertheless, recent advances in the biomedical sciences have made possible the comprehensive characterization of individuals' genomes, transcriptomes, proteomes and metabolomes, and the superimposition of this “panomics” information with detailed health and disease endpoints. There is promise that “precision dentistry” will emerge from this new wave of systems biology and big data-driven science and practice and will bring about meaningful improvements in individuals' and populations' health.

Recent efforts in periodontal medicine have built upon the principles of precision medicine to refine periodontal health and disease classifications and dissect the biological basis of disease susceptibility, with the ultimate goal of tailoring or targeting prevention and treatment strategies. Along these lines, the development of a precise stratification system that reflects distinct periodontal disease patterns can serve as the basis for precise risk assessment (at the population-level) and estimation of individual susceptibilities (at the person-level), with disease progression and tooth loss being the main endpoints of interest. However, current periodontal disease taxonomies have limited utility for predicting disease progression and tooth loss; in fact, tooth loss itself can undermine precise person-level periodontal disease classifications.

To date, periodontal disease risk assessment tools have used clinical measurements and known risk factors to predict tooth loss or periodontal disease progression with the goal of establishing more specific prognoses and to optimize treatment choices. Although some prediction models incorporate well-established risk factors, such as smoking history, diabetes, age, race and sex, there are currently no validated risk assessment tools utilizing clinical parameters including tooth-specific patterns. Most models utilize subject-level summary variables of clinical parameters, such as mean or extent scores for various signs of disease including plaque scores, gingival indices, probing depths, and clinical attachment levels, that reflect subject-level disease and are not always linked to tooth type or tooth loss patterns. Most prediction models do not explicitly classify missing teeth, which can be lost for a variety of reasons, and are not informed by existing tooth loss patterns when considering risk of natural disease progression, which has important prognostic value¹² Individual tooth-specific measures of crown to root ratios, mobility, tooth position and other factors can be used to improve estimates of individual tooth-level prognoses, but irrespective of the model the final estimates of risk are qualitative in nature based upon clinical impressions. In sum, no predictive models exist that provide quantitative tooth-based risk estimates and account for the informative heterogeneity in clinical presentation by patterns of tooth loss.

Wth that said, described herein are methods of diagnosing periodontal disease and/or assessing risk of tooth loss in a subject that can include the step of classifying the patient and/or each individual tooth of a patient into one of 7 classes of periodontal disease and uses thereof. The methods can further include the step(s) of treating periodontal disease in a patient and/or each individual tooth based on which of the seven classes the patient and/or the individual tooth is classified into step of classifying the patient and/or each individual tooth of a patient into one of 7 classes of periodontal disease. Also described herein are methods of treating periodontal disease in a patient that can include the step of diagnosing periodontal disease and/or assessing risk of tooth loss in a subject that can include the step of classifying the patient and/or each individual tooth of a patient into one of 7 classes of periodontal disease. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Described herein are methods of diagnosing periodontal disease in a subject that can include the step of classifying the patient and/or each individual tooth of a patient into one of 7 classes of periodontal disease. The classes can indicate the risk of tooth loss in a subject and/or by individual tooth. The method can include the step of performing a dental examination on a patient and determining a periodontal profile class (PPC). The PPC is a stratification system based on seven clinical parameters that defines distinct categories of members (people) with previously “hidden” combinations of clinical characteristics, to create mutually exclusive latent classes. In combination with the tooth profile class (TPC) assignment, a computed score can be calculated, which can indicate the risk of tooth loss and periodontal disease progression in the individual patient. The seven PPCs are designated herein as PPC-A, PPC-B, PPC-C, PPC-D, PPC-E, PPC-F, and PPC-G. PPC-A designates a healthy or incidental diseased subject. PPC-B designates a subject with mild periodontal disease. PPC-C designates a subject with high GI (gingival inflammation index). PPC-D designates a subject with some tooth loss (about 50% tooth loss). PPC-E designates a subject with posterior disease. PPC-F indicates a subject with severe tooth loss (about 75% tooth loss). PPC-G indicates a subject with severe periodontal disease. The description of clinical parameters for each PPC appears in Table 9. There were significant differences among all seven PPCs, and these values were provided for descriptive and comparative purposes. PPC-A (Health) had the lowest mean extent of BOP, GI≥1, and PI≥1. The mean extent of IAL≥3 mm of 8% and a mean extent of PD≥4 mm of 2% were the lowest among all 7 periodontal profile classes. PPC-B (Mild Disease) was mainly characterized by a slight increase in IAL≥3 mm and PD≥4 mm mean extent scores, and significant higher BOP (3-fold) and GI (9-fold) when compared to Stage I. PPC-C (High GI) was notably marked by the highest mean extent GI score among all periodontal profile classes and was seen in 10% of the population. PPC-D (Tooth Loss) was characterized by fewer teeth. PPC-E (Posterior Disease) was marked by a moderate mean extent of IAL≥3 mm of 33% mainly located at the posterior dentition. PPC-F (Severe Tooth Loss) was characterized by the lowest mean number of teeth (8 teeth), where the remaining teeth were mainly mandibular anterior teeth with an edentulous maxilla and reflected 13% of the population. Finally, PPC-G (Severe Disease) was characterized by the highest mean extent of IAL≥3 mm of 54% and PD≥4 mm of about 25%. Higher BOP, GI, and PI extent scores were also found in this generalized severe disease profile.

The method can include the step of performing a dental examination on a patient and determining for each tooth a Tooth Profile Class (TPC). The TPC is a stratification that is applied to each individual tooth in a patient that defines distinct categories of teeth based on clinical parameters. The seven TPCs are designated herein as TPC-A, TPC-B, TPC-C, TPC-D, TPC-E, TPC-F, and TPC-G. The tooth-level LCA procedure enabled us to identify 7 TPCs (A-G), in the DARIC population. The description of the 14 clinical parameters for each TPC is described in Table 12. As described in the Examples, significant differences among all seven TPCs were observed, and these values are provided for descriptive and comparative purposes. For example, TPC-A can include teeth with the least attachment loss, PD, BOP, recession, GI, PI, caries, and number of crowns. TPC-G can include teeth with signs of periodontitis represented by substantial attachment loss, deep PD, high GI and PI.

The PPC and TPC can be used together to generate a composite risk score for an individual, which is referred to herein as the Index of Periodontal Risk (IPR). The IPR can range from 4 to 46 and can be calculated for each individual based on tooth loss risks. The analytical approach used to calculate IPR can be based on a 7×7 table (PPC×TPC) of predicted probabilities for 10-year tooth loss. First, each individual can be assigned to one of the 7 PPCs and then, each tooth can be classified to one of the 7 TPCs. The IPR can then be calculated as the mean predicted probability for 10-year tooth loss across all teeth present for each individual. The development of the IPR can include one or more risk factors for periodontal disease, such as age, sex, race, diabetes, and smoking status. Tooth loss and disease progression risk estimates based on classes developed specific IPR cut—can support 3 levels or classes of risk for tooth loss denominated Index of Periodontal Classes (IPC): IPC-“Low” (IPR 0-10), IPC-“Moderate” (IPR 10-20), and IPC-“High” (IPR>20). In some embodiments, the method can include the step of performing a dental examination on a patient and classifying the subject into one of the seven PPCs and determining for each tooth a TPC. The method can then include the step of calculating the mean predicted probability for 10-year tooth loss across all teeth present in the individual. The IPR can fall into one of three risk of tooth loss categories (Low, Moderate, or High) referred to herein as an Index of Periodontal Class as previously described.

The method can further include the step of treating the subject and/or one or more teeth in a subject. The treatment and/or preventive therapy carried can include, but are not limited to, tooth extraction, non-surgical periodontal therapy, surgical periodontal therapy, administration of medicated tooth paste and/or oral mouth rinse, administration of a pharmaceutical agent (e.g. an antibiotic), specialist referral, maintenance frequency recalls, and any combination thereof. Additional therapies that might affect periodontal conditions can include, but are not limited to, restorative dental procedures (e.g. crowns and restorations), endodontic treatment (root canal), orthodontic treatment, dental implant therapy, and/or combinations thereof. Therapy for tooth loss can include, but is not limited to, placement of partial dentures, full dentures, bridges, dental implants, and/or combinations thereof. Assessing the risk for tooth loss by and periodontal disease progression any of the methodology and can be used by a practitioner in treating patients. For example, in some embodiments subjects classified into the PPC-G (Severe Disease) and High IPC, can be treated with non-surgical and/or surgical periodontal therapy in combination with the administration of systemic antibiotics, and more frequent maintenance recalls. This is different than for subjects classified under the PPC-A (Healthy) and Low IPC level, which can be treated using a regular adult prophylaxis with a 6-month treatment recall schedule. The method used to assess the risk for tooth loss and periodontal disease progression using PPC, TPC, and IPC was based on the clinical data from over 7,000 subjects from the DARIC dataset that includes a 10-year assessment on tooth loss. Further, it was validated using the longitudinal data from the Piedmont study and 2 national databases (NHANES 2009-2010 and NHANES 2011-2012).

In some aspects, a subject's PPC, IPR, and/or IPC can be used to determine the subject's risk of systemic conditions such as diabetes, coronary heart disease (CHD), and stroke, as well as systemic measures of high sensitivity such as serum C-Reactive Protein (hs-CRP) and Interleukin-6 (IL-6). Thus, described herein are methods of determine the risk of a subject to a systemic condition that can include the step of determining the subject's PPC, IPR, and/or IPC as previously described.

More specifically, associations between the PPC and prevalent systemic conditions of diabetes, CHD, stroke, hs-CRP, and serum IL-6 for ARIC study participants is presented in Table 22. The number of study participants with these diseases and conditions vary and are shown at the top of each column. All models were adjusted for relevant confounders and covariates including race/center, age, sex, BMI, smoking (three levels), education, as well as lipids and other systemic conditions relevant to the disease. These models each served as a reference model for computing the BIC that permitted comparison of having periodontal disease in the model to not having periodontal disease. It can be seen that many BIC improvement scores are above 10 (very strong contribution) for the PPC classification, with the CDC/AAP and European classifications having one BIC score above 10 and many below 2.0.

As discussed in greater detail in the Examples, for diabetes (see e.g. Table 22), most categories showed significant odds ratios for the PPC, while nothing was significant for the CDC/AAP and European indices. Individuals with periodontal disease who have retained most of their teeth generally are classified as having mild, high GI, posterior disease, or severe disease. Of these classes, the high GI and severe disease classes are associated with prevalent diabetes. Greater odds of having prevalent diabetes with both tooth loss classes and severe disease were also observed. The PPC for severe disease was observed to have the highest odds ratio (1.88) for diabetes and the entire PPC model showed the greatest BIC Improvement. All the high GI, tooth loss, severe tooth loss, and severe disease classes were significant for CHD. Only the severe disease class was significant for prevalent stroke. Individuals classified as having high GI, tooth loss, severe tooth loss, or severe disease were significantly more likely to be in the highest quartile for serum CRP and IL-6. There were no significant associations for the CDC/AAP classification, and European index associations were between CHD and IL-6 for severe disease. Cardiovascular disease is divided into CHD and stroke because they share some risk factors, but have differing mechanisms of pathogenesis. Among the European classifications, only the severe categories show significant associations with CHD, and none of the associations with stroke were significant. Several of the PPC classes were significantly associated with CHD, with high GI being the strongest followed by mild disease and the two tooth loss classes. However, severe disease was not significant. The PPC severe disease class was significantly associated with stroke.

The analyses shown in Table 22 and Table 23 for the three combined NHANES cohorts (2009-2010, 2011-2012, and 2013-2014). IL-6 information was not available in NHANES, so it is absent in these tables. BIC improvement scores for having periodontal disease classifications in the models were very strong for all PPC models, but were observed to be weaker for most CDC/AAP- and European-based models. The NHANES results in Table 23 show all PPC classifications except posterior disease were significantly associated with diabetes as were the CDC/AAP moderate and severe and the European incipient and severe categories. The PPC models can demonstrate that posterior disease and severe tooth loss are the only categories associated with CHD, and only tooth loss is associated with stroke. The two tooth loss categories and posterior disease were associated with CRP. The CDC/AAP and European models were associated with CHD and CRP, but not with stroke. The models for CHD showed that moderate and severe disease were significantly associated for the CDC/AAP and that incipient and severe disease were significant for the European index. The BIC scores for the PPC, CDC/AAP, and European models indicated that periodontitis made a very strong contribution.

Similar to the periodontal treatment recommendations for each PPC/TPC, the treatment related to systemic conditions, such as diabetes, CHD, and stroke depends on the clinician's decision. But, with the information acquired by using the PPC/TPC/IPC stratification guides provides dentists and physicians to assess patient's risk in a more precisely and elaborate a treatment plan and recall frequency in a way that best fit the patient's needs.

Importantly, the PPC system of patient stratification results in different treatment recommendations for some patients as compared to the traditional classification system (CDC/AAP). FIG. 17 illustrates the changes in treatment plans that could occur by using the PPC system rather than a traditional system (CDC/AAP) to determine an individual's periodontal status. Data from the US National Health and Nutrition Survey (NHANES 2009-2014) was used to indicate how many individuals' treatment might be impacted by using the PPC system. The grey colored cells indicate situations where the PPC would differ from the CDC/AAP designation and some change in treatment would be recommended as compared to the traditional treatments recommended and/or used for individuals now falling in these categories. The third row in the table shows where the CDC/AAP classification and PPC names would align with each other and which PPC categories do not have a corresponding CDC/AAP classification.

Using latent class analysis to create PPC classes of disease that reflect risk has can better determine periodontal treatment needs and appropriate treatments for subjects based in a data driven approach that classifies an individual into a group where all members of a group have similar risk for contracting disease, disease progression, or response to treatment. The treatment recommendations according to the different PPCs are demonstrated below. The recommendations include different active periodontal treatment options, the recommended recall visit frequency, and the recommendation for a specialist referral.

In comparison to the CDC/AAP classification it was demonstrated that with the PPC classification described herein almost 10 million Americans would not be identified using the CDC/AAP classification and consequently not receive the most recommended periodontal treatment. Regarding the mild/moderate CDC/AAP classification the numbers are extremely higher with approximately 37 million Americans being misclassified according to their risk and consequently not receiving the appropriate treatment recommendations. When evaluating the Severe CDC/AAP category the number of Americans being misclassified are approximately 6 million, which also ultimately influence the treatment recommendations.

A couple examples can be illustrative in understanding the import of these differences. Considering the 89.4 million people who were classified as being healthy by the CDC/AAP index, 79.8 million would receive the traditionally recommended treatment that is displayed under the PPC-A column (FIG. 17, Part 1). However, the millions (about 9.6 million) of people that under the traditional method would have been considered Healthy fell into different categories under the PPC classification system, which resulted in a different treatment recommendation that what they would have received had they been considered under the traditional classification system. More specifically, different treatments can include delivery of delivery of antibiotics (local or systemic), increased recall visits, surgical intervention and specialty consultations. Furthermore, Additional therapeutic agents therapy based on genetic tests may be indicated. Importantly, the traditional system fails to classify some patients as a high risk for tooth loss or disease and as such, many subjects may not receive a treatment that may save a tooth and/or reduce other tooth issues. In other words earlier intervention can be missed to the detriment of the subject.

A second example involves the 43.3 million individuals who fall into the CDC/AAP Mild/Moderate category. About 6 million fall into the comparable PPC-B group and would receive the treatment indicated in that column (FIG. 17, Part 1). However, 13.9 million were classified as PPC-A and could have received less treatment with a corresponding reduction in cost. In addition, millions more fell into other PPC categories, including 2.7 million who were classified as having severe disease and need a more extensive treatment plan to deal with their greatly increased risk of disease progression and tooth loss. A similar situation is seen with those traditionally classified as having severe disease in which many under the PPC system could receive less treatment, which can have a corresponding reduction in total treatment cost and in many cases avoid tooth removal.

In some aspects, after a patient is classified using the PPC system, a treatment option can be recommended and/or performed by a practitioner. As described above, the treatment can be different than what would have been recommended/performed if the patient had been diagnosed using a traditional classification system (FIG. 17, Part 2). In some aspects, the treatment/preventive therapies that can be recommended and/or performed include, but are not limited to, adult prophylactic dental care, providing non-surgical periodontal therapy, administering local antibiotics, administering systemic antibiotics, providing surgical periodontal therapy, or any combination thereof. Furthermore, Additional therapeutic agents based on genetic tests may be indicated. Additional therapies that might affect periodontal conditions can include, but are not limited to, restorative dental procedures (e.g. crowns and restorations), endodontic treatment (root canal), orthodontic treatment, dental implant therapy, and/or combinations thereof. Therapy for tooth loss can include, but is not limited to, placement of partial dentures, full dentures, bridges, dental implants, and/or combinations thereof. In some aspects, after a patient is classified using the PPC system, a practitioner can recommend recall visits. In some aspects, the number of recall visits can be different as compared to the number that would have been recommended for the same patient if they were diagnosed using a traditional classification system. In some aspects, the number of recall visits can be increased as a result of using the PPC system instead of a traditional system. In some aspects, the number of recall visits can be decreased as a result of using the PPC system instead of a traditional system. Traditional classification systems can include, but are not limited to, the Centers for Disease Control/American Academy of Periodontology (CDC/AAP) index¹⁷ along with the European Periodontal index. Other traditionally used classification systems are the 1999 AAP classification (see e.g. Armitage GC. Development of a classification system for periodontal diseases and conditions. Ann Periodontol 1999; 4:1-6.) and the American Dental Association/American Academy of Periodontology Association (ADA/AAP) classification (Sweeting L A, Davis K, Cobb C M. Periodontal treatment protocol (PTP) for the general dental practice. J Dent Hyg 2008; 82:16-26.).

In some embodiments, each state of the disclosed PPC system is characterized by unique single nucleotide polymorphisms (SNPs) as described in Table 1. These SNPs are associated with unique pathways, identifying unique druggable targets for each stage. Therefore, disclosed herein is a method of classifying a subject into PPC stages that involves assaying a biological sample from the subject for one or more SNPs identified in Table 1.

Also disclosed herein is a method of treating and/or preventing periodontal disease and/or tooth loss in a subject, the method comprising: classifying a subject into one of seven Periodontal Profile Classes (PPCs) based on information on each tooth present in the subject obtained via a dental examination; and treating the subject with an effective amount of a therapeutic agent that targets one or more genes associated with the classified PPC.

Therefore, in some embodiments, a subject classified as PPC-B is treated with a therapeutic agent selected from the group consisting of dextrose, pyroglutamic acid, streptol, vandetanib, progesterone, acarbose, miglitol, celgosivir, duvoglustat hydrochloride, duvoglustat, phorbol myristate acetate, leucovorin, and irinotecan.

In some embodiments, a subject classified as PPC-C is treated with a therapeutic agent selected from the group consisting of VANTICTUMAB, FLANVOTUMAB, DOCETAXEL, PACLITAXEL, ACALABRUTINIB, IBRUTINIB, INOSITOL, RETINOL, GENISTEIN, LEUPROLIDE ACETATE, DEXAMETHASONE, HALOFUGINONE, DIHYDROSPINGOSINE, SPHINGOSINE, NIFEDIPINE, CHEMBL1179605, MEFENAMIC ACID, PIOGLITAZONE, ROSIGLITAZONE, TROGLITAZONE, CHEMBL169233, ASPIRIN, CHEMBL1161866, STANOLONE, CHEMBL566340, COLFORSIN, DAROTROPIUM BROMIDE, TRIDIHEXETHYL CHLORIDE, TOLTERODINE TARTRATE, PROPANTHELINE BROMIDE, OXYPHENONIUM BROMIDE, ARIPIPRAZOLE, OLANZAPINE, METHIXENE, TERFENADINE, CLOZAPINE, OXYPHENCYCLIMINE, PROCYCLIDINE, LOXAPINE, PROMAZINE, HYOSCYAMINE, DARIFENACIN, TRIDIHEXETHYL, ANISOTROPINE METHYLBROMIDE, DIPHEMANIL METHYLSULFATE, SCOPOLAMINE, BENZQUINAMIDE, PROPIOMAZINE, TROPICAMIDE, BROMPHENIRAMINE, GLYCOPYRROLATE, TOLTERODINE, PILOCARPINE, MIVACURIUM, DIPHENIDOL, CHLORPROTHIXENE, PIPECURONIUM, LEVOMEPROMAZINE, ISOPROPAMIDE, MEPENZOLATE, FESOTERODINE, METHACHOLINE, ACLIDINIUM, UMECLIDINIUM, ACETYLCHOLINE, ARECAIDINE PROPARGYL ESTER, ARECOLINE, BETHANECHOL, FURTRETHONIUM, 5-METHYLFURMETHIODIDE, CHEMBL99521, ERIBAXABAN, OXOTREMORINE, CHEMBL130715, CHEMBL74300, MILAMELINE, SABCOMELINE, XANOMELINE, ALCURONIUM, BRUCINE, CHEMBL343796, STRYCHNINE, CHEMBL2206331, CHEMBL343357, VINBURNINE, VINCAMINE, CHEMBL139677, CHEMBL523685, CHEMBL1256845, 4-DAMP, CHEMBL279453, AMITRIPTYLINE, BENZTROPINE MESYLATE, ATROPINE, BIPERIDEN (CHEMBL1101), CLIDINIUM, DICYCLOMINE, DOTHIEPIN (CHEMBL1492500), CHEMBL580785, HEXOCYCLIUM, HIMBACINE, CHEMBL1256682, IPRATROPIUM, METHOCTRAMINE, HYDROCHLORIC ACID, OTENZEPAD, OXYBUTYNIN, PIRENZEPINE, PROPANTHELINE, SOLIFENACIN, COENZYME_A, QUINUCLIDINYL BENZILATE, TIOTROPIUM (CHEMBL1900528), TRIPITRAMINE, CHEMBL1233686, CHEMBL1628667, REVATROPATE, UMECLIDINIUM BROMIDE, LAS190792, Afacifenacin, Tarafenacin, SOLIFENACIN SUCCINATE, ACLIDINIUM BROMIDE, METHACHOLINE CHLORIDE, TIOTROPIUM BROMIDE, ACETYLCHOLINE CHLORIDE, CARBACHOL (CHEMBL14), PILOCARPINE HYDROCHLORIDE, CEVIMELINE HYDROCHLORIDE, SUXAMETHONIUM, BATEFENTEROL, MEPENZOLATE BROMIDE, HEXOCYCLIUM METHYLSULFATE, DICYCLOMINE HYDROCHLORIDE, BETHANECHOL CHLORIDE, ATROPINE SULFATE, OXYBUTYNIN CHLORIDE, DARIFENACIN HYDROBROMIDE, IPRATROPIUM BROMIDE HYDRATE, METHSCOPOLAMINE BROMIDE, OXYPHENCYCLIMINE HYDROCHLORIDE, GLYCOPYRROLATE BROMIDE, ISOPROPAMIDE IODIDE, TROSPIUM CHLORIDE, FESOTERODINE FUMARATE, CYCLOPENTOLATE HYDROCHLORIDE, ASM-024, AZD8683, CLIDINIUM BROMIDE, THIETHYLPERAZINE, CEVIMELINE, DOXEPIN, PROMETHAZINE, ITOPRIDE, HOMATROPINE METHYLBROMIDE, ANACETRAPIB, DALCETRAPIB, TORCETRAPIB, CHEMBL67129, EVACETRAPIB, CERIVASTATIN, TAMOXIFEN, CEP-2563, TICLOPIDINE, GSK-690693, SOTRASTAURIN, (7S)-HYDROXYL-STAUROSPORINE, MIDOSTAURIN, QUERCETIN, SOTRASTAURIN ACETATE, STAUROSPORINE, DEXFOSFOSERINE, INGENOL MEBUTATE, CHEMBL369507, BRYOSTATIN, MEDRONIC ACID, INSULIN HUMAN, VANDETANIB, CHEMBL552425, SULFASALAZINE, RIDOGREL, DAZOXIBEN, PHORBOL MYRISTATE ACETATE, BELIMUMAB, BRIOBACEPT, ATACICEPT, TABALUMAB, BLISIBIMOD, DIOXANE, ETOPOSIDE, CITRIC ACID

In some embodiments, a subject classified as PPC-D is treated with a therapeutic agent selected from the group consisting of ISRADIPINE, NIMODIPINE, NISOLDIPINE, VERAPAMIL, FELODIPINE, NITRENDIPINE, NIFEDIPINE, MIBEFRADIL, NILVADIPINE, SALSALATE, BEPRIDIL HYDROCHLORIDE, PREGABALIN, GABAPENTIN, GABAPENTIN ENACARBIL, IMAGABALIN, ATAGABALIN, MAGNESIUM SULFATE, AMLODIPINE, CELECOXIB, DRONEDARONE, CHEMBL566340, GO-6976, QUERCETIN, GSK-690693, (7S)-HYDROXYL-STAUROSPORINE, CEP-2563, MIDOSTAURIN, SOTRASTAURIN, BRYOSTATIN, RESVERATROL, VASOPRESSIN, BOSUTINIB, CHEMBL359482, PAROXETINE, OCRIPLASMIN, HYDROCHLOROTHIAZIDE, CHEMBL384759, INSULIN HUMAN, BUSULFAN, ZONISAMIDE, FRUCTOSE, TRICHOSTATIN, ASCORBATE, OCRIPLASMIN, GUANIDINE HYDROCHLORIDE, DALFAMPRIDINE, TEDISAMIL, NERISPIRDINE, TRETINOIN, ETRETINATE, ZOLEDRONIC ACID, CHEMBL300914, PYRIDOXAL PHOSPHATE, THREONINE, NIROGACESTAT, REGN-421, CYCLOPHOSPHAMIDE

In some embodiments, a subject classified as PPC-E is treated with a therapeutic agent selected from the group consisting of QUERCETIN, METAPROTERENOL SULFATE, COPANLISIB, ALPELISIB, PICTILISIB, APITOLISIB, PF-04691502, GEDATOLISIB, SONOLISIB, SF-1126, VOXTALISIB, PILARALISIB (CHEMBL3218575), DACTOLISIB, PI-103, GSK-2636771, BUPARLISIB, CHEMBL1229535, HYDROGEN PEROXIDE, CLAVULANIC ACID, AMOXICILLIN, ME-344, NV-128, METFORMIN HYDROCHLORIDE, CHEMBL1161866, CAPSAICIN, CHEMBL1213492, NINTEDANIB ESYLATE, SU-014813, KRN-633, L-21649, TAK-593, AG-13958, BMS-690514, FORETINIB, MGCD-265, BRIVANIB ALANINATE, SEMAXANIB, CEDIRANIB, SORAFENIB, Anlotinib, CC-223, DOVITINIB, Fruquintinib, LENVATINIB, LINIFANIB, MK-2461, MOTESANIB, NINTEDANIB, PAZOPANIB, SUNITINIB, TESEVATINIB, TIVOZANIB, VATALANIB, AXITINIB, REGORAFENIB, LENVATINIB MESYLATE, ILORASERTIB, CEP-11981, CEP-7055, Chiauranib, JNJ-26483327, OSI-930, RG-1530, TELATINIB, Famitinib, XL-999, BRIVANIB, SUNITINIB MALATE, VANDETANIB, SORAFENIB TOSYLATE, PAZOPANIB HYDROCHLORIDE, LUCITANIB, ENMD-2076, ORANTINIB, X-82, XL-820, CEP-5214, SU-14813, CP-459632, Sulfatinib, 4SC-203, CHEMBL313417, IMC-3C5, TABERMINOGENE VADENOVEC, TG100-801, IMC-1C11, PEGPLERANIB SODIUM, CHEMBL384759, GUANOSINE TRIPHOSPHATE, GLYCINE, TEZAMPANEL, BUTABARBITAL, BUTALBITAL, TALBUTAL, SECOBARBITAL, METHARBITAL, THIOPENTAL, PRIMIDONE, MEPHOBARBITAL, PHENOBARBITAL, CHEMBL301536, 2S,4R-4-METHYLGLUTAMATE, DOMOIC ACID, KAINIC ACID, MESALAMINE, METHYPRYLON, AMOBARBITAL, APROBARBITAL, BUTETHAL, HEPTABARBITAL, HEXOBARBITAL, BARBITAL, Selurampanel, TOPIRAMATE, PENTOBARBITAL, QUISQUALATE, L-GLUTAMATE, PROTIRELIN, TALTIRELIN, GANITUMAB, PICROPODOPHYLLOTOXIN, BMS-754807, LINSITINIB, AEW-541, DALOTUZUMAB, INSULIN GLARGINE, EMACTUZUMAB, AZD-4547, CHEMBL401930, BRIGATINIB, CERITINIB, CHEMBL464552, CHEMBL458997, DIMETHISTERONE, XL-228, BUB-022, AVE-1642, CIXUTUMUMAB, INSM-18, KW-2450, FIGITUMUMAB, ROBATUMUMAB, MECASERMIN, PL-225B, TEPROTUMUMAB, MECASERMIN RINFABATE, CHEMBL1230989, ACETYLCYSTEINE, THROMBIN, INSULIN HUMAN, CHEMBL263143, CHEMBL397666, RALOXIFENE, ERLOTINIB, RINFABATE, STAUROSPORINE, HYDROCHLOROTHIAZIDE

In some embodiments, a subject classified as PPC-F is treated with a therapeutic agent selected from the group consisting of DANAZOL, CHEMBL195368, CHEMBL1234621, CONBERCEPT, SORAFENIB TOSYLATE, LENALIDOMIDE, BEVACIZUMAB, NIMODIPINE, PROGESTERONE, SPIRONOLACTONE, EPLERENONE, FELODIPINE, DESOXYCORTICOSTERONE PIVALATE, DROSPIRENONE, ALDOSTERONE, HYDROCORTISONE, DESOXYCORTICOSTERONE, DEXAMETHASONE, FLUDROCORTISONE, PREDNISOLONE, FINERENONE, ONAPRISTONE, PF-03882845, OXPRENOATE POTASSIUM, XL550, MT-3995, LY2623091, DESOXYCORTICOSTERONE ACETATE, FLUDROCORTISONE ACETATE, CORTICOSTERONE, RIZATRIPTAN, CHEMBL482796, LIPDXIN A4, LITHIUM, GADOBENATE DIMEGLUMINE, CHEMBL185515, AMILORIDE, PYRIMETHAMINE, NAFAMOSTAT, PREGNENOLONE, GENISTEIN, LEUPROLIDE ACETATE, DEXAMETHASONE, HALOFUGINONE, DAVALINTIDE, PRAMLINTIDE, PRAMLINTIDE ACETATE, CALCITONIN SALMON RECOMBINANT, DOXORUBICIN, CHEMBL574817, CLOZAPINE, ODANACATIB, PALMITIC ACID, FLANVOTUMAB, INGENOL MEBUTATE, ELLAGIC ACID, APRINOCARSEN SODIUM, CHEMBL1236539, BALANOL, ENZASTAURIN, GO-6976, SEBACIC ACID, RUBOXISTAURIN, SOTRASTAURIN, MIDOSTAURIN, QUERCETIN, SOTRASTAURIN ACETATE, GSK-690693, CEP-2563, (7S)-HYDROXYL-STAUROSPORINE, BRYOSTATIN, TAMOXIFEN, DEXFOSFOSERINE, VITAMIN E, FRUCTOSE, TRICHOSTATIN, SACITUZUMAB GOVITECAN, TEGLARINAD CHLORIDE, FLUOROURACIL, OCRIPLASMIN, HALOTHANE, PROXYPHYLLINE, LISOFYLLINE, CAFFEINE, TRETINOIN, UROKINASE, CARBOQUONE, INSULIN HUMAN, MESALAMINE, L-GLUTAMATE, PYRIMETHAMINE, PYRIDOSTIGMINE, PYRILAMINE, PEGINTERFERON LAMBDA-1A, RINTATOLIMOD, PYROXAMIDE, HYDROXYCHLOROQUINE, ELEDOISIN, KASSININ, NEUROKININ A, NEUROKININ B, CHEMBL69367, SENKTIDE, SUBSTANCE P, ISOFLURANE, AZD2624, CHEMBL480249, CHEMBL221445, OSANETANT, CHEMBL44229, SAREDUTANT, CHEMBL9843, CHEMBL1991816, AMCINONIDE, TALNETANT, SB-222200, CHEMBL275544, PYRIMETHAMINE, COBALT (II) ION, VERAPAMIL, VASOPRESSIN TANNATE, FAMOXADONE, AZOXYSTROBIN, Coenzyme Q2, PROXYPHYLLINE, CHOLIC ACID, AFIMOXIFENE (CHEMBL489), DIARYLPROPIONITRILE, DIETHYLSTILBESTROL, CHLOROTRIANISENE, ESTROGENS, CONJUGATED, ETONOGESTREL, DESOGESTREL, LEVONORGESTREL, PROGESTERONE, TOREMIFENE, MEDROXYPROGESTERONE ACETATE, ESTRONE, TAMOXIFEN, DIENESTROL, FULVESTRANT, NORGESTIMATE, ETHINYL ESTRADIOL, MELATONIN, TRILOSTANE, FLUOXYMESTERONE, ESTRAMUSTINE, ESTRIOL, PRINABEREL, PROPYLPYRAZOLETRIOL, RALOXIFENE, CHEMBL282489, CHEMBL188528, LASOFOXIFENE, BAZEDOXIFENE, CLOMIPHENE, CHEMBL201013, HEXESTROL, CHEMBL520107, MESTRANOL, DANAZOL, ALLYLESTRENOL, PRASTERONE, ESTROPIPATE, QUINESTROL, OSPEMIFENE, TIBOLONE, ESTROGENS, CONJUGATED SYNTHETIC A, SYNTHETIC CONJUGATED ESTROGENS, B, ESTRADIOL, MITOTANE, SR16234 (CHEMBL3545210), FISPEMIFENE, LY2245461, IDOXIFENE, GTx-758, AFIMOXIFENE (CHEMBL10041), DROLOXIFENE, ACOLBIFENE, TAMOXIFEN CITRATE, ESTRADIOL VALERATE, CLOMIPHENE CITRATE, ESTRADIOL CYPIONATE, ESTROGENS, ESTERIFIED, DIETHYLSTILBESTROL DIPHOSPHATE, TOREMIFENE CITRATE, BAZEDOXIFENE ACETATE, CHF4227, GDC-0810, ESTRADIOL ACETATE (CHEMBL1200430), POLYESTRADIOL PHOSPHATE, MK-6913, IODINE, VINTAFOLIDE, CUSTIRSEN, CHEMBL304552, EVEROLIMUS, CHEMBL181936, CHEMBL391910, PERTUZUMAB, CHEMBL180300, DIENOGEST, ESTROGEN, RAD1901, CHEMBL222501, ARZOXIFENE, CHEMBL180071, CHEMBL193676, ETHYNODIOL DIACETATE, GENISTEIN, RALOXIFEN, Ribociclib, RALOXIFENE CORE, TRASTUZUMAB, CHEMBL236718, ABEMACICLIB, 2-AMINO-1-METHYL-6-PHENYLIMIDAZO[4,5-B]PYRIDINE, ERTEBEREL, EXEMESTANE, ENDOXIFEN, MEGESTROL ACETATE, SIVIFENE, PALBOCICLIB, LEFLUNOMIDE, ESTRONE SODIUM SULFATE, LETROZOLE, CHEMBL236086, CHEMBL184151, CHEMBL223026, LAPATINIB, CHEMBL180517, NORELGESTROMIN, ANASTROZOLE, CARBOQUONE, GONADORELIN, CHEMBL1213270, NORGESTREL, MEDRONIC ACID

In some embodiments, a subject classified as PPC-G is treated with a therapeutic agent selected from the group consisting of TEMAZEPAM, ADINAZOLAM, HALAZEPAM, DIAZEPAM, OXAZEPAM, TRIAZOLAM, ESTAZOLAM, BROMAZEPAM, CLOTIAZEPAM, FLUDIAZEPAM, KETAZOLAM, PRAZEPAM, QUAZEPAM, CINOLAZEPAM, NITRAZEPAM, CLORAZEPATE DIPOTASSIUM, CHLORDIAZEPDXIDE, ESZOPICLONE, MEPROBAMATE, CLOBAZAM, ALPRAZOLAM, BUTALBITAL, CLONAZEPAM, DESFLURANE, TALBUTAL, BUTABARBITAL SODIUM, LORAZEPAM, METHARBITAL, METHYPRYLON, MIDAZOLAM HYDROCHLORIDE, PRIMIDONE, PROPOFOL, PENTOBARBITAL SODIUM, SECOBARBITAL SODIUM, SEVOFLURANE, THIAMYLAL SODIUM, FLURAZEPAM HYDROCHLORIDE, HALOTHANE, ISOFLURANE, ADIPIPLON, Lorediplon, RESEQUINIL, PF-06372865, METHOHEXITAL SODIUM, THIOPENTAL SODIUM, CHLORDIAZEPDXIDE HYDROCHLORIDE, PENTOBARBITAL, ETHCHLORVYNOL, GLUTETHIMIDE, FLUMAZENIL, CLORAZEPIC ACID, METHOXYFLURANE, MIDAZOLAM, TRICLOFOS SODIUM, TOPIRAMATE, GABOXADOL, ETOMIDATE, ACAMPROSATE CALCIUM, FLURAZEPAM, OCINAPLON, ENFLURANE, ETAZOLATE, CHEMBL2325441, PREGNENOLONE, S-ADENOSYLHOMOCYSTEINE, ODANACATIB, PROSCILLARIDIN, CYPROHEPTADINE, CYCLOPENTOLATE, ELETRIPTAN, METHYSERGIDE, ZOLMITRIPTAN, ERGOTAMINE, RIZATRIPTAN, MIANSERIN, ELETRIPTAN HYDROBROMIDE, ALMOTRIPTAN MALATE, LASMIDITAN, CHEMBL266591, SEROTONIN, 5-MEO-DMT, 5-METHOXYTRYPTAMINE, 8-OH-DPAT, CHEMBL1256797, CLOZAPINE, DIHYDROERGOTAMINE, CHEMBL1332062, DONITRIPTAN, CHEMBL101690, CHEMBL3186179, NARATRIPTAN, OLANZAPINE, QUETIAPINE, SUMATRIPTAN, TRYPTAMINE, XANOMELINE, BRL-15,572, GR-127935, CHEMBL1256701, CHEMBL277120, METERGOLINE, METITEPINE, METHYLERGONOVINE, RISPERIDONE, SERTINDOLE, YOHIMBINE, ADENOSINE TRIPHOSPHATE, AZD-5438, PHA-793887, AT-7519, Roniciclib, COLFORSIN, PAPAVERINE HYDROCHLORIDE, METHICILLIN, ADENOSINE PHOSPHATE, HESPERIDIN, CHEMBL578514, LIPOIC ACID, METHACHOLINE CHLORIDE, LEUCINE, CHEMBL506495, GALMIC, GALNON, CHEMBL541253, CHEMBL450441, METHYLENE BLUE, BRETYLIUM TOSYLATE, CHEMBL604991, METHOXAMINE, TEMAZEPAM, GALANIN, DIPHEMANIL, GALANTIDE, ANDROSTENEDIONE, TESTOSTERONE, VANDETANIB, SORAFENIB, CABOZANTINIB, REGORAFENIB, PONATINIB, AST-487, CEP-11981, LINIFANIB, LUCITANIB, QUIZARTINIB, SUNITINIB, TAMATINIB, AT-9283, MOTESANIB, AMUVATINIB, LENVATINIB, ALECTINIB HYDROCHLORIDE, CEP-32496, LESTAURTINIB, SORAFENIB TOSYLATE, SUNITINIB MALATE, CEP-2563, CETUXIMAB, CHEMBL1213492, INK-128, EVEROLIMUS, DEXAMETHASONE, AZD-1480, CHEMBL306380, DOVITINIB, GENISTEIN, IMATINIB, CHEMBL126955, ENMD-2076, ALECTINIB, PHORBOL MYRISTATE ACETATE, XL-999, TRETINOIN, NINTEDANIB, PYRIMETHAMINE, COBALT (II) ION, VERAPAMIL, TETANUS TOXOID, CERITINIB, MEDRONIC ACID, QUERCETIN, METAPROTERENOL SULFATE, COPANLISIB, ALPELISIB, PICTILISIB, APITOLISIB, PF-04691502, GEDATOLISIB, SONOLISIB, SF-1126, VOXTALISIB, PILARALISIB (CHEMBL3218575), DACTOLISIB, PI-103, GSK-2636771, BUPARLISIB, CHEMBL1229535, ARSENIC TRIOXIDE, MOTEXAFIN GADOLINIUM, CHEMBL449269, FLAVIN ADENINE DINUCLEOTIDE, SPERMIDINE, FOTEMUSTINE, CERIVASTATIN, PSEUDOEPHEDRINE HYDROCHLORIDE, BOSUTINIB, DASATINIB, HYDROCORTISONE, LITHIUM CARBONATE, LITHIUM CITRATE HYDRATE, ACETYLCYSTEINE, SUNITINIB, ARIPIPRAZOLE, NAPHTHALENE, CHEMBL435278, CHEMBL122264, CHEMBL17639, CHEMBL21283, MONOETHANOLAMINE, QUERCETIN, PREDNISOLONE, PUROMYCIN

TABLE 1
Significant (p < 5.00E−08) SNPs Identified by Pleiotropic GWAS Stratified by PPC-Stage

| | | | | | | | | |
|---|---|---|---|---|---|---|---|---|
| npid | Allele1 | Allele2 | Zscore | P | Direction | chr | pos | closestRefGene |

PPC-A

| | | | | | | | | |
|---|---|---|---|---|---|---|---|---|
| rs12970437 | a | c | −6.003 | 1.93E−09 | −−−−−− | 18 | 47905999 | DCC |

PPC-B

| | | | | | | | | |
|---|---|---|---|---|---|---|---|---|
| rs10881157 | a | g | −5.706 | 1.16E−08 | −−−−−− | 1 | 1E+08 | AMY1A |
| rs11011168 | c | g | −5.623 | 1.88E−08 | −−−−−− | 10 | 3.8E+07 | ANKRD30A |
| rs1915979 | c | g | 5.686 | 1.30E−08 | ++++++ | 7 | 6.8E+07 | AUTS2 |
| rs17660815 | g | t | 6.443 | 1.17E−10 | ++++++ | 5 | 1.8E+07 | BASP1 |
| rs10411431 | a | g | 6.252 | 4.06E−10 | ++++++ | 19 | 6.2E+07 | BC37295_3 |
| rs282669 | c | t | −5.664 | 1.48E−08 | −−−−+− | 10 | 6E+07 | BICC1 |
| rs17054017 | a | g | 5.512 | 3.54E−08 | ++++++ | 8 | 2.6E+07 | CDCA2 |
| rs9616028 | c | t | 5.721 | 1.06E−08 | +++++− | 22 | 4.5E+07 | CELSR1 |
| rs12439077 | a | g | −5.57 | 2.55E−08 | −−−−−− | 15 | 6.2E+07 | DAPK2 |
| rs12270207 | c | g | 5.892 | 3.82E−09 | ++++++ | 11 | 8.4E+07 | DLG2 |
| rs2030737 | c | t | 5.965 | 2.44E−09 | ++++++ | 3 | 1.4E+08 | EPHB1 |
| rs8002848 | c | t | −5.614 | 1.98E−08 | −−−−−− | 13 | 1E+08 | FGF14 |
| rs8132 | c | t | −5.53 | 3.19E−08 | −−−−−− | 17 | 7.6E+07 | GAA |
| rs11574632 | c | t | −5.474 | 4.40E−08 | −−−−−− | 16 | 3.1E+07 | ITGAX |
| rs2245768 | a | t | 6.022 | 1.72E−09 | ++++++ | 11 | 1.3E+08 | KIRREL3 |
| rs7195261 | a | g | 5.477 | 4.33E−08 | ++++++ | 16 | 7.2E+07 | LOC283902 |
| rs3853178 | a | g | 6.208 | 5.38E−10 | ++++−+ | 16 | 3.4E+07 | LOC729355 |
| rs368380 | c | t | −6.013 | 1.82E−09 | −−−−−− | 20 | 1.5E+07 | MACROD2 |
| rs2527073 | a | c | −5.677 | 1.37E−08 | −−−−−− | 8 | 5586807 | MCPH1 |
| rs985211 | g | t | 5.498 | 3.83E−08 | ++++++ | 21 | 2.4E+07 | NCAM2 |
| rs10244526 | a | g | 5.481 | 4.24E−08 | ++++++ | 7 | 3.3E+07 | NT5C3 |
| rs476154 | c | g | −5.462 | 4.72E−08 | −−−−−− | 11 | 1.3E+08 | OPCML |
| rs311433 | c | g | 5.65 | 1.61E−08 | ++++++ | 1 | 2.5E+07 | RUNX3 |
| rs17141570 | a | g | 5.552 | 2.82E−08 | ++++++ | 10 | 1.8E+07 | STAM |
| rs10773345 | g | t | 5.585 | 2.34E−08 | ++++++ | 12 | 1.3E+08 | TMEM132B |
| rs11852677 | c | t | 5.584 | 2.35E−08 | ++++++ | 15 | 6.9E+07 | UACA |
| rs9872121 | a | g | −6.081 | 1.20E−09 | −−−−−− | 3 | 4.6E+07 | XCR1 |
| rs947089 | a | t | −5.698 | 1.21E−08 | −−−−−− | 10 | 3.1E+07 | ZNF438 |

PPC-C

| | | | | | | | | |
|---|---|---|---|---|---|---|---|---|
| rs10055033 | c | t | 5.942 | 2.81E−09 | ++++++ | 5 | 8E+07 | SERINC5 |
| rs1013261 | a | g | −5.466 | 4.60E−08 | −−−−−− | 2 | 1.2E+08 | DPP10 |
| rs10170697 | c | t | −5.853 | 4.81E−09 | −−−−−− | 2 | 2E+08 | FZD7 |
| rs10171384 | a | g | 5.764 | 8.19E−09 | +++−++ | 2 | 2E+07 | LAPTM4A |
| rs10224959 | c | t | 5.73 | 1.01E−08 | ++++++ | 7 | 1.5E+08 | CNTNAP2 |
| rs10795862 | c | g | 5.467 | 4.58E−08 | ++++++ | 10 | 1.1E+07 | CUGBP2 |
| rs10799145 | g | t | −5.668 | 1.45E−08 | −−−−−− | 1 | 4921060 | AJAP1 |
| rs10853171 | a | g | −5.495 | 3.90E−08 | −−−−−− | 17 | 3E+07 | TMEM132E |
| rs10960440 | g | t | 6.432 | 1.26E−10 | ++++++ | 9 | 1.2E+07 | TYRP1 |
| rs11180563 | c | t | 5.631 | 1.79E−08 | ++++++ | 12 | 7.4E+07 | KRR1 |
| rs11237082 | a | g | 5.471 | 4.47E−08 | ++++++ | 11 | 7E+07 | SHANK2 |
| rs11595862 | c | t | 6.113 | 9.80E−10 | ++++++ | 10 | 7.7E+07 | DUSP13 |
| rs11774301 | g | t | 6.055 | 1.40E−09 | ++++−+ | 8 | 5.2E+07 | PXDNL |
| rs11982297 | a | g | −5.492 | 3.97E−08 | −−−−+− | 7 | 2.4E+07 | STK31 |
| rs12135329 | c | t | 5.895 | 3.76E−09 | ++++−+ | 1 | 2.4E+08 | PLD5 |
| rs12191170 | a | t | 5.58 | 2.41E−08 | ++++++ | 6 | 1.3E+08 | C6orf191 |
| rs12193727 | c | t | −5.913 | 3.35E−09 | −−−−−− | 6 | 8329576 | SLC35B3 |
| rs12220989 | a | c | −5.592 | 2.24E−08 | −−−−−− | 10 | 3E+07 | LYZL1 |
| rs12521638 | a | g | −5.498 | 3.85E−08 | −−−−−− | 5 | 1.7E+08 | ODZ2 |
| rs12546876 | a | g | 6.019 | 1.75E−09 | ++++++ | 8 | 6E+07 | TOX |
| rs12587917 | c | g | 6.366 | 1.94E−10 | ++++++ | 14 | 7.6E+07 | VASH1 |
| rs12592259 | c | t | 5.909 | 3.44E−09 | ++++++ | 15 | 6.8E+07 | TLE3 |
| rs12936464 | a | c | 5.469 | 4.53E−08 | ++++++ | 17 | 7344666 | POLR2A |
| rs1420364 | a | g | −5.552 | 2.83E−08 | −−−−−− | 2 | 6.7E+07 | ETAA1 |
| rs1472969 | c | g | −6.077 | 1.22E−09 | −−−−+− | 4 | 4.8E+07 | TEC |
| rs1492658 | a | g | 6.454 | 1.09E−10 | ++++++ | 8 | 9.6E+07 | C8orf37 |
| rs1516920 | a | g | 5.882 | 4.05E−09 | ++++++ | 2 | 1.8E+07 | RDH14 |
| rs1613 | c | t | 5.864 | 4.52E−09 | ++++++ | 11 | 9.3E+07 | SLC36A4 |
| rs167237 | a | t | −6.176 | 6.59E−10 | −−−−−− | 5 | 1.7E+07 | BASP1 |
| rs16950392 | a | g | 5.919 | 3.23E−09 | ++++++ | 15 | 6.5E+07 | SMAD3 |
| rs16992846 | a | g | −5.839 | 5.25E−09 | −−−−−− | 20 | 1.2E+07 | BTBD3 |
| rs17105275 | c | t | −5.456 | 4.88E−08 | −−−−−− | 5 | 1.5E+08 | PPP2R2B |
| rs17261780 | a | c | 5.678 | 1.37E−08 | ++++−+ | 4 | 1.1E+08 | DKK2 |
| rs17310798 | c | t | 5.578 | 2.43E−08 | ++++++ | 3 | 1.4E+08 | RAB6B |
| rs17554439 | a | c | 5.774 | 7.74E−09 | ++++++ | 9 | 7.3E+07 | TRPM3 |
| rs1793767 | a | g | 6.643 | 3.08E−11 | ++++++ | 11 | 1.3E+08 | HNT |
| rs1945146 | a | g | 5.816 | 6.01E−09 | ++++++ | 11 | 8.6E+07 | ME3 |
| rs2028270 | c | g | 5.539 | 3.04E−08 | ++++++ | 5 | 7.9E+07 | SERINC5 |
| rs2040784 | a | g | 5.973 | 2.33E−09 | ++++++ | 7 | 2.6E+07 | NFE2L3 |
| rs2117817 | a | t | −5.8 | 6.65E−09 | −−−−−− | 4 | 1.8E+08 | FBXO8 |
| rs2140634 | c | t | 6.072 | 1.26E−09 | ++++++ | 7 | 1.6E+08 | RNF32 |
| rs2153226 | c | t | −5.792 | 6.95E−09 | −−−−−− | 9 | 7.8E+07 | PCSK5 |
| rs2184925 | g | t | −5.88 | 4.12E−09 | −−−−+− | 6 | 7.6E+07 | COL12A1 |
| rs2499914 | a | c | −5.469 | 4.53E−08 | −−−−−− | 10 | 1.2E+07 | USP6NL |
| rs2639989 | c | t | −6.364 | 1.97E−10 | −−−−−− | 18 | 7.1E+07 | ZADH2 |
| rs2775941 | c | t | −5.897 | 3.70E−09 | −−−−+− | 21 | 1.4E+07 | LOC441956 |
| rs2822127 | a | g | 5.845 | 5.06E−09 | ++++−+ | 21 | 1.4E+07 | LOC441956 |
| rs2822129 | a | g | 5.856 | 4.75E−09 | ++++−+ | 21 | 1.4E+07 | LOC441956 |
| rs2916635 | c | t | −5.591 | 2.26E−08 | −−−−−− | 5 | 1.4E+08 | SPOCK1 |
| rs318373 | c | t | −5.777 | 7.61E−09 | −−−−−− | 5 | 1.4E+08 | HMHB1 |
| rs4413520 | a | c | −5.764 | 8.19E−09 | −−−−−− | 5 | 1.1E+08 | EFNA5 |
| rs4432731 | c | t | −6.134 | 8.58E−10 | −−−−+− | 4 | 1.6E+08 | DCHS2 |
| rs465947 | c | g | −5.895 | 3.74E−09 | −−−−−− | 5 | 7868237 | ADCY2 |
| rs4659558 | c | g | 5.952 | 2.66E−09 | ++++++ | 1 | 2.4E+08 | CHRM3 |
| rs4783961 | a | g | 5.715 | 1.09E−08 | ++++++ | 16 | 5.6E+07 | CETP |
| rs4809947 | c | t | 5.516 | 3.48E−08 | ++++++ | 20 | 5.2E+07 | BCAS1 |
| rs4842345 | c | t | 5.456 | 4.88E−08 | ++++++ | 12 | 7.9E+07 | C12orf64 |
| rs4848521 | c | t | 5.601 | 2.13E−08 | ++++−+ | 2 | 1.2E+08 | EN1 |
| rs485345 | a | g | −5.619 | 1.92E−08 | −−−−+− | 11 | 1.2E+08 | ASAM |
| rs5016898 | c | t | 5.518 | 3.43E−08 | ++++++ | 1 | 8.1E+07 | LPHN2 |
| rs550445 | c | t | −6.73 | 1.69E−11 | −−−−−− | 11 | 3E+07 | MPPED2 |
| rs625492 | a | c | −5.499 | 3.83E−08 | −−−−−− | 2 | 1.7E+08 | GRB14 |
| rs648731 | a | g | 5.77 | 7.92E−09 | ++++++ | 10 | 6508737 | PRKCQ |
| rs6535497 | a | g | 5.684 | 1.32E−08 | ++++++ | 4 | 8.5E+07 | MAG1 |
| rs6586508 | a | g | −5.672 | 1.41E−08 | −−−−−− | 1 | 1.7E+07 | CROCC |
| rs6665694 | c | t | −5.635 | 1.75E−08 | −−−−−− | 1 | 1.6E+08 | LMX1A |
| rs7015174 | g | t | 5.535 | 3.11E−08 | ++++++ | 8 | 7.4E+07 | TERF1 |
| rs7219527 | c | t | 6.004 | 1.92E−09 | ++++−+ | 17 | 5.7E+07 | NACA2 |
| rs7235201 | a | g | 5.669 | 1.44E−08 | ++++++ | 18 | 2.1E+07 | ZNF521 |
| rs726237 | c | g | −5.584 | 2.35E−08 | −−−−−− | 2 | 7.9E+07 | CTNNA2 |
| rs7275448 | c | t | 5.915 | 3.33E−09 | ++++++ | 21 | 2.3E+07 | NCAM2 |
| rs7330295 | g | t | 5.758 | 8.50E−09 | ++++++ | 13 | 6.7E+07 | PCDH9 |
| rs7642023 | c | t | 6.106 | 1.02E−09 | ++++++ | 3 | 5.7E+07 | ARHGEF3 |
| rs7687468 | a | g | −5.469 | 4.53E−08 | −−−−−− | 4 | 5.9E+07 | IGFBP7 |
| rs7761181 | a | t | 5.853 | 4.84E−09 | ++++++ | 6 | 9.4E+07 | EPHA7 |
| rs7818703 | g | t | −5.633 | 1.77E−08 | −−−−−− | 8 | 1.2E+08 | SNTB1 |
| rs7847940 | c | g | −5.64 | 1.70E−08 | −−−−+− | 9 | 7.3E+07 | TRPM3 |
| rs8192856 | a | t | −6.021 | 1.73E−09 | −−−−−− | 7 | 1.4E+08 | TBXAS1 |
| rs878171 | c | t | −5.543 | 2.98E−08 | −−−−+− | 17 | 4.3E+07 | OSBPL7 |
| rs893787 | c | t | −6.138 | 8.38E−10 | −−−−−− | 2 | 1.2E+07 | LPIN1 |
| rs9306 | c | t | 5.72 | 1.06E−08 | ++++++ | 6 | 1.6E+08 | PACRG |
| rs9375794 | c | t | −5.657 | 1.54E−08 | −−−−−− | 6 | 1.3E+08 | EPB41L2 |
| rs9514840 | c | g | −5.8 | 6.63E−09 | −−−−−− | 13 | 1.1E+08 | TNFSF13B |
| rs9946639 | a | g | 5.497 | 3.87E−08 | ++++++ | 18 | 2.2E+07 | SS18 |

PPC-D

| | | | | | | | | |
|---|---|---|---|---|---|---|---|---|
| rs1001486 | a | g | −6.64 | 3.13E−11 | −−−−− | 10 | 1.9E+07 | CACNB2 |
| rs10036878 | a | g | 5.574 | 2.49E−08 | +++++ | 5 | 5.1E+07 | ISL1 |
| rs10217492 | c | g | −5.774 | 7.74E−09 | −−−−− | 9 | 7.8E+07 | PCSK5 |
| rs1024445 | a | g | −5.477 | 4.32E−08 | −−−−− | 19 | 1.9E+07 | UPF1 |
| rs10806844 | a | t | 6.195 | 5.84E−10 | +++++ | 6 | 1.7E+08 | T |
| rs10861932 | a | t | −5.898 | 3.69E−09 | −−−−− | 12 | 1.1E+08 | HYPE |
| rs10876555 | g | t | −5.846 | 5.03E−09 | −−−−− | 12 | 3.8E+07 | KIF21A |
| rs10899610 | a | g | 5.459 | 4.80E−08 | +++−+ | 11 | 7.9E+07 | ODZ4 |
| rs10921094 | a | g | −5.923 | 3.16E−09 | −−−−− | 1 | 1.9E+08 | RGS18 |
| rs10949356 | a | g | 5.5 | 3.80E−08 | +++++ | 6 | 1.7E+07 | ATXN1 |
| rs11161058 | c | t | 5.77 | 7.94E−09 | +++++ | 14 | 2.9E+07 | PRKD1 |
| rs11237081 | c | g | −5.675 | 1.38E−08 | −−−−− | 11 | 7.6E+07 | CAPN5 |
| rs11257948 | a | g | −5.776 | 7.66E−09 | −−−−− | 10 | 1.3E+07 | CAMK1D |
| rs11601125 | c | g | 5.912 | 3.39E−09 | +++++ | 11 | 9852501 | SBF2 |
| rs11662579 | c | t | −5.591 | 2.26E−08 | −−−−− | 18 | 6976611 | LAMA1 |
| rs11663290 | a | t | 5.611 | 2.02E−08 | +++++ | 18 | 5.4E+07 | NEDD4L |
| rs11722347 | c | t | 5.568 | 2.57E−08 | +++++ | 4 | 8.4E+07 | COQ2 |
| rs11743051 | a | g | 5.713 | 1.11E−08 | +++++ | 5 | 1.8E+08 | TSPAN17 |
| rs11764731 | c | g | 6.833 | 8.35E−12 | +++++ | 7 | 1.5E+08 | DPP6 |
| rs11816208 | a | t | 6.776 | 1.24E−11 | +++++ | 10 | 7500961 | SFMBT2 |
| rs11817679 | a | g | −5.619 | 1.92E−08 | −−−−− | 10 | 1.9E+07 | ARL5B |
| rs12202642 | c | t | −6.572 | 4.95E−11 | −−−−− | 6 | 3.7E+07 | FGD2 |
| rs12414476 | a | g | −6.666 | 2.64E−11 | −−−−− | 10 | 7.3E+07 | C10orf54 |
| rs12425668 | c | t | −5.573 | 2.50E−08 | −−−−− | 12 | 1.1E+08 | MED13L |
| rs12430698 | a | t | 5.869 | 4.38E−09 | +++++ | 13 | 9E+07 | GPC5 |
| rs12436090 | a | g | 5.483 | 4.17E−08 | +++++ | 14 | 5.6E+07 | PELI2 |
| rs12480427 | a | t | −5.811 | 6.21E−09 | −−−−− | 20 | 1.7E+07 | PCSK2 |
| rs12517647 | g | t | 6.332 | 2.42E−10 | +++++ | 5 | 1.7E+08 | ODZ2 |
| rs12815089 | c | g | 6.078 | 1.22E−09 | +++++ | 12 | 2.3E+07 | ETNK1 |
| rs12867851 | c | t | −5.458 | 4.82E−08 | −−−−− | 13 | 2.6E+07 | WASF3 |
| rs12883143 | c | t | 5.742 | 9.34E−09 | +++++ | 14 | 9.7E+07 | VRK1 |
| rs12998780 | a | g | −5.667 | 1.45E−08 | −−−−− | 2 | 5.6E+07 | SMEK2 |
| rs13246891 | c | t | −5.96 | 2.52E−09 | −−−−− | 7 | 4.3E+07 | HECW1 |
| rs1385398 | a | g | −5.562 | 2.67E−08 | −−−−− | 14 | 8.7E+07 | GALC |
| rs1386269 | a | c | 6.134 | 8.57E−10 | +++++ | 17 | 4.8E+07 | CA10 |
| rs1434123 | c | t | 6.034 | 1.60E−09 | +++++ | 2 | 8735254 | ID2 |
| rs1496650 | c | t | 5.761 | 8.35E−09 | +++++ | 3 | 6E+07 | FHIT |
| rs1680580 | c | t | −5.649 | 1.61E−08 | −−−−− | 5 | 1.7E+08 | DOCK2 |
| rs16851463 | g | t | −5.527 | 3.26E−08 | −−−−− | 1 | 1.5E+07 | TMEM51 |
| rs16857576 | a | g | 5.674 | 1.39E−08 | +++++ | 3 | 1.5E+08 | PLOD2 |
| rs16931840 | a | g | −6.162 | 7.19E−10 | −−−−− | 9 | 1.4E+07 | ZDHHC21 |
| rs16948939 | c | g | 6.091 | 1.13E−09 | +++++ | 15 | 9.3E+07 | MCTP2 |
| rs17042804 | c | t | 5.754 | 8.71E−09 | +++++ | 3 | 5536429 | EDEM1 |
| rs17057184 | c | g | −5.629 | 1.81E−08 | −−−−− | 6 | 1.3E+08 | LAMA2 |
| rs1707981 | g | t | −5.511 | 3.57E−08 | −−−−− | 3 | 3.9E+07 | MOBP |
| rs17171742 | a | g | 5.8 | 6.64E−09 | +++++ | 7 | 3.6E+07 | HERPUD2 |
| rs17193494 | a | t | 6.378 | 1.80E−10 | +++++ | 12 | 9E+07 | C12orf12 |
| rs17230650 | a | g | 5.571 | 2.54E−08 | +++++ | 10 | 6.2E+07 | ANK3 |
| rs17272228 | a | c | 5.854 | 4.80E−09 | +++++ | 15 | 6.5E+07 | SMAD6 |
| rs17286152 | a | g | 5.462 | 4.70E−08 | +++++ | 20 | 4E+07 | PTPRT |
| rs17336166 | a | g | 5.477 | 4.33E−08 | +++++ | 6 | 1.2E+08 | SLC35F1 |
| rs17365678 | c | t | 5.594 | 2.23E−08 | +++++ | 2 | 1.1E+07 | KCNF1 |
| rs17585733 | a | c | 5.582 | 2.38E−08 | +++++ | 2 | 6E+07 | BCL11A |
| rs17687542 | c | t | −5.495 | 3.91E−08 | −−−−− | 3 | 6.1E+07 | FHIT |
| rs17710623 | a | g | −5.849 | 4.94E−09 | −−−−− | 20 | 3544845 | ATRN |
| rs17764155 | c | t | 5.765 | 8.15E−09 | +++++ | 14 | 6.9E+07 | ERH |
| rs17773903 | a | g | −5.788 | 7.12E−09 | −−−−− | 8 | 1E+08 | KLF10 |
| rs1867714 | a | g | 5.703 | 1.18E−08 | +++++ | 5 | 3.5E+07 | RAI14 |
| rs1878705 | a | g | −6.297 | 3.04E−10 | −−−−− | 14 | 2E+07 | OSGEP |
| rs1893452 | a | g | −5.614 | 1.98E−08 | −−−−− | 18 | 4.6E+07 | MYO5B |
| rs1902431 | a | t | −6.052 | 1.43E−09 | −−−−− | 8 | 1.3E+08 | FAM84B |
| rs1967505 | c | t | −5.486 | 4.11E−08 | −−−−− | 7 | 5.7E+07 | ZNF479 |
| rs1998058 | a | g | 5.69 | 1.27E−08 | +++++ | 22 | 4.7E+07 | TBC1D22A |
| rs2012586 | c | t | 5.647 | 1.64E−08 | +++++ | 21 | 3.7E+07 | CLDN14 |
| rs2039052 | a | c | −5.456 | 4.86E−08 | −−−−− | 9 | 1.4E+07 | NFIB |
| rs2055141 | a | g | 6.058 | 1.38E−09 | +++++ | 5 | 5.7E+07 | DKFZp686D0972 |
| rs215941 | c | t | −6.533 | 6.46E−11 | −−−−− | 6 | 8.6E+07 | TBX18 |
| rs228146 | a | c | −5.456 | 4.86E−08 | −−−−− | 6 | 5.6E+07 | BMP5 |
| rs2303059 | c | t | 5.904 | 3.54E−09 | +++++ | 5 | 1.6E+08 | CCNJL |
| rs2331494 | c | t | −6.214 | 5.16E−10 | −−−−− | 14 | 2.2E+07 | DAD1 |
| rs2345191 | a | g | 6.196 | 5.80E−10 | +++++ | 10 | 8.3E+07 | SH2D4B |
| rs2603731 | c | t | −5.73 | 1.01E−08 | −−−−− | 4 | 1.9E+08 | SORBS2 |
| rs2798776 | c | t | 5.464 | 4.65E−08 | +++++ | 10 | 2.9E+07 | BAMBI |
| rs2823042 | c | t | 5.616 | 1.96E−08 | +++++ | 21 | 1.5E+07 | NRIP1 |
| rs299958 | g | t | −5.762 | 8.32E−09 | −−−−− | 16 | 8.5E+07 | FOXL1 |
| rs3017366 | a | g | −5.59 | 2.28E−08 | −−−−− | 18 | 5.8E+07 | TNFRSF11A |
| rs3755845 | c | t | −5.596 | 2.20E−08 | −−−−− | 4 | 5764485 | EVC |
| rs4148202 | g | t | −5.747 | 9.06E−09 | −−−−− | 2 | 4.4E+07 | ABCG8 |
| rs4273108 | a | g | −5.864 | 4.51E−09 | −−−−− | 17 | 7.4E+07 | DNAH17 |
| rs4309286 | a | t | 5.957 | 2.57E−09 | +++++ | 13 | 8.3E+07 | SLITRK1 |
| rs4396981 | c | t | −5.483 | 4.19E−08 | −−−−− | 4 | 8.3E+07 | RASGEF1B |
| rs4534931 | a | g | −5.94 | 2.85E−09 | −−−−− | 18 | 2.6E+07 | DSC3 |
| rs4684503 | c | g | 6.597 | 4.18E−11 | +++++ | 3 | 5698569 | EDEM1 |
| rs4841663 | a | c | 6.166 | 7.00E−10 | +++++ | 8 | 1.2E+07 | DEFB134 |
| rs4858184 | a | t | 5.522 | 3.35E−08 | +++++ | 3 | 2E+07 | SGOL1 |
| rs4884948 | a | g | 5.532 | 3.17E−08 | +++++ | 13 | 7.1E+07 | DACH1 |
| rs4910237 | c | t | −6.43 | 1.28E−10 | −−−−− | 11 | 1.1E+07 | GALNTL4 |
| rs4941366 | g | t | 5.558 | 2.72E−08 | +++++ | 18 | 5.4E+07 | NEDD4L |
| rs5997400 | a | g | −5.487 | 4.08E−08 | −−−−− | 22 | 2.7E+07 | CCDC117 |
| rs6071100 | c | t | −5.467 | 4.58E−08 | −−−−− | 20 | 5.8E+07 | C20orf197 |
| rs6471325 | a | g | 5.91 | 3.41E−09 | +++++ | 8 | 9.3E+07 | RUNX1T1 |
| rs6488099 | a | g | 5.988 | 2.12E−09 | +++++ | 12 | 3.3E+07 | PKP2 |
| rs6575926 | a | g | −5.596 | 2.19E−08 | −−−−− | 14 | 1E+08 | RCOR1 |
| rs6576504 | c | t | −6.796 | 1.08E−11 | −−−−− | 15 | 2.4E+07 | ATP10A |
| rs6600382 | a | g | −7.248 | 4.22E−13 | −−−−− | 1 | 4.2E+07 | HIVEP3 |
| rs6703917 | c | g | −6.483 | 9.01E−11 | −−−−− | 1 | 8.4E+07 | TTLL7 |
| rs6729815 | c | t | 5.491 | 3.99E−08 | +++++ | 2 | 6.1E+07 | BCL11A |
| rs6744726 | a | g | −5.963 | 2.48E−09 | −−−−− | 2 | 1.3E+08 | RAB6C |
| rs6757665 | a | g | −5.714 | 1.11E−08 | −−−−− | 2 | 2.4E+08 | SH3BP4 |
| rs6820473 | c | t | −5.553 | 2.81E−08 | −−−−− | 4 | 2.9E+07 | PCDH7 |
| rs6911915 | c | t | 5.522 | 3.35E−08 | +++++ | 6 | 1.2E+08 | DCBLD1 |
| rs6997097 | c | t | 5.767 | 8.09E−09 | +++++ | 8 | 1.2E+07 | NEIL2 |
| rs6997500 | c | t | −5.654 | 1.57E−08 | −−−−− | 8 | 9.4E+07 | RUNX1T1 |
| rs7042041 | c | t | −6.183 | 6.28E−10 | −−−−− | 9 | 1.2E+08 | OR1L8 |
| rs7141908 | a | g | −5.606 | 2.07E−08 | −−−−− | 14 | 9.8E+07 | C14orf177 |
| rs7285551 | a | g | 5.552 | 2.83E−08 | +++++ | 22 | 4.6E+07 | TBC1D22A |
| rs7302663 | a | c | 5.557 | 2.74E−08 | +++++ | 12 | 9.3E+07 | CRADD |
| rs731945 | c | t | 5.495 | 3.91E−08 | +++++ | 19 | 1.8E+07 | ELL |
| rs7327336 | c | t | 6.011 | 1.84E−09 | +++++ | 13 | 2.4E+07 | SPATA13 |
| rs753127 | c | t | −6.244 | 4.27E−10 | −−−−− | 14 | 9.2E+07 | SLC24A4 |
| rs7613298 | c | g | 5.77 | 7.92E−09 | +++++ | 3 | 7.5E+07 | CNTN3 |
| rs7753922 | c | t | −5.553 | 2.80E−08 | −−−−− | 6 | 8.9E+07 | C6orf166 |
| rs7764197 | c | t | −5.533 | 3.16E−08 | −−−−− | 6 | 1.1E+07 | GCNT2 |
| rs7806488 | a | g | −5.771 | 7.86E−09 | −−−−− | 7 | 2E+07 | ITGB8 |
| rs7921396 | g | t | −5.518 | 3.42E−08 | −−−−− | 10 | 2.5E+07 | THNSL1 |
| rs7952254 | c | t | −6.935 | 4.06E−12 | −−−−− | 11 | 1.6E+07 | SOX6 |
| rs8012983 | a | g | −6.194 | 5.86E−10 | −−−−− | 14 | 5.1E+07 | FRMD6 |
| rs9267853 | c | t | −6.016 | 1.78E−09 | −−−−− | 6 | 3.2E+07 | NOTCH4 |
| rs930851 | a | g | 6.433 | 1.25E−10 | +++++ | 1 | 1.8E+07 | IGSF21 |
| rs9369583 | a | g | −5.928 | 3.06E−09 | −−−−− | 6 | 4.6E+07 | CLIC5 |
| rs9462426 | a | g | 6.427 | 1.30E−10 | +++++ | 6 | 3.8E+07 | BTBD9 |
| rs9530506 | c | t | 5.688 | 1.28E−08 | +++++ | 13 | 7.5E+07 | LMO7 |
| rs9537303 | a | g | 6.16 | 7.28E−10 | +++++ | 13 | 5.5E+07 | FLJ40296 |
| rs9601679 | g | t | −5.719 | 1.07E−08 | −−−−− | 13 | 8.1E+07 | SPRY2 |
| rs9690040 | c | t | 5.501 | 3.78E−08 | +++++ | 7 | 1.5E+08 | INSIG1 |
| rs978493 | a | g | 5.731 | 1.00E−08 | +++++ | 5 | 1.2E+08 | SEMA6A |
| rs9814936 | a | g | −5.531 | 3.18E−08 | −−−−− | 3 | 1.5E+08 | GPR87 |
| rs9832396 | a | g | 5.932 | 3.00E−09 | +++++ | 3 | 7.9E+07 | ROBO1 |
| rs9964434 | c | g | −5.552 | 2.82E−08 | −−−−− | 18 | 5.8E+07 | RNF152 |

PPC-E

| | | | | | | | | |
|---|---|---|---|---|---|---|---|---|
| rs10163673 | a | t | 6.352 | 2.13E−10 | ++++++ | 18 | 3.6E+07 | PIK3C3 |
| rs10762418 | c | g | 5.484 | 4.16E−08 | +++−++ | 10 | 7.2E+07 | PCBD1 |
| rs10786967 | c | t | 5.802 | 6.54E−09 | ++++++ | 10 | 1.1E+08 | SORCS1 |
| rs11591738 | a | g | 5.513 | 3.53E−08 | ++++++ | 10 | 1.3E+08 | LHPP |
| rs11605897 | a | g | −5.903 | 3.56E−09 | −−−−−− | 11 | 9.8E+07 | CNTN5 |
| rs11671501 | c | t | 5.702 | 1.18E−08 | ++++++ | 19 | 1.5E+07 | FLJ45910 |
| rs11688237 | a | g | −5.535 | 3.10E−08 | −−−−−− | 2 | 1.4E+08 | ARHGAP15 |
| rs11730793 | c | t | −5.64 | 1.70E−08 | −−−−−− | 4 | 5243434 | STK32B |
| rs11936468 | c | t | 5.525 | 3.29E−08 | ++++++ | 4 | 2E+07 | SLIT2 |
| rs12149031 | c | t | −5.528 | 3.24E−08 | −−−−−− | 16 | 5.5E+07 | NUP93 |
| rs12157940 | c | t | 5.573 | 2.50E−08 | ++++++ | 22 | 3.2E+07 | LARGE |
| rs12287284 | a | t | −5.865 | 4.50E−09 | −−−−−− | 11 | 9E+07 | CHORDC1 |
| rs12437774 | c | t | 6.063 | 1.34E−09 | ++++++ | 15 | 3.6E+07 | TMCO5 |
| rs12524678 | a | t | −5.738 | 9.55E−09 | −−−−−− | 6 | 2.1E+07 | CDKAL1 |
| rs13140329 | c | t | −5.877 | 4.17E−09 | −−−−−− | 4 | 2E+07 | SLIT2 |
| rs1328710 | a | g | 5.469 | 4.52E−08 | ++++++ | 6 | 7.3E+07 | RIMS1 |
| rs1374624 | a | c | −6.077 | 1.22E−09 | −−−−−− | 4 | 5196334 | STK32B |
| rs1383795 | c | t | 5.511 | 3.56E−08 | ++++++ | 14 | 8.4E+07 | FLRT2 |
| rs1440634 | a | g | −6.034 | 1.60E−09 | −−−−−− | 3 | 1.5E+08 | CPA3 |
| rs1484126 | g | t | −5.667 | 1.45E−08 | −−−−−− | 8 | 5.2E+07 | SNTG1 |
| rs1530239 | c | t | −5.644 | 1.66E−08 | −−−−−− | 2 | 2.1E+08 | IKZF2 |
| rs1559836 | c | t | 5.67 | 1.43E−08 | ++++++ | 12 | 1.1E+08 | RBM19 |
| rs1574733 | c | t | −5.516 | 3.47E−08 | −−−−−− | 3 | 1.3E+08 | HEG1 |
| rs1706226 | c | t | −7.281 | 3.31E−13 | −−−−−− | 4 | 3.5E+07 | CENTD1 |
| rs17081845 | a | g | 5.607 | 2.06E−08 | ++++++ | 13 | 2.5E+07 | FAM123A |
| rs17168014 | c | t | −5.552 | 2.82E−08 | −−−−−− | 7 | 1.3E+08 | CALD1 |
| rs17784714 | c | t | 5.536 | 3.10E−08 | ++++++ | 3 | 4.7E+07 | SCAP |
| rs1794520 | a | t | 6.179 | 6.45E−10 | ++++++ | 6 | 3.3E+07 | HLA−DQB1 |
| rs1850937 | c | g | −5.588 | 2.29E−08 | −−−−−− | 19 | 2.2E+07 | ZNF429 |
| rs1864268 | c | g | −5.697 | 1.22E−08 | −−−−−− | 2 | 6.1E+07 | CCDC139 |
| rs1971643 | c | t | −5.979 | 2.25E−09 | −−−−−− | 7 | 1.1E+07 | NDUFA4 |
| rs1997316 | a | c | 5.724 | 1.04E−08 | ++++++ | 15 | 3.1E+07 | SCG5 |
| rs2012362 | c | t | −5.539 | 3.04E−08 | −−−−−− | 5 | 1.1E+07 | ROPN1L |
| rs205402 | c | t | −6.041 | 1.54E−09 | −−−−−− | 16 | 2.8E+07 | XPO6 |
| rs2165693 | c | t | 5.67 | 1.43E−08 | ++++++ | 14 | 8.5E+07 | FLRT2 |
| rs2290983 | a | g | −5.57 | 2.55E−08 | −−−−−− | 5 | 1.8E+08 | FLT4 |
| rs2302373 | c | t | −5.957 | 2.56E−09 | −−−−−− | 10 | 1.2E+08 | NRAP |
| rs2311120 | a | g | 6.144 | 8.06E−10 | ++++++ | 18 | 5.1E+07 | RAB27B |
| rs2389911 | c | t | −6.031 | 1.63E−09 | −−−−−− | 13 | 9.7E+07 | RAP2A |
| rs2443743 | c | t | 5.512 | 3.54E−08 | ++++++ | 5 | 1.2E+08 | LOC340069 |
| rs2862478 | g | t | 6.604 | 3.99E−11 | ++++++ | 3 | 1.8E+08 | TBL1XR1 |
| rs4437385 | c | t | 5.554 | 2.80E−08 | ++++++ | 5 | 2.8E+07 | CDH9 |
| rs4475 | c | g | −6.496 | 8.23E−11 | −−−−−− | 22 | 4.6E+07 | TBC1D22A |
| rs4586547 | c | t | −5.938 | 2.89E−09 | −−−−−− | 18 | 4835603 | ZFP161 |
| rs4758347 | c | t | −5.639 | 1.71E−08 | −−−−−− | 11 | 8316071 | STK33 |
| rs6031077 | a | g | 5.641 | 1.69E−08 | ++++++ | 20 | 4.2E+07 | FAM112A |
| rs6462623 | c | t | −5.984 | 2.17E−09 | −−−−−− | 7 | 3.6E+07 | HERPUD2 |
| rs6476553 | a | g | −6.014 | 1.81E−09 | −−−−−− | 9 | 3633012 | RFX3 |
| rs663578 | a | g | −5.716 | 1.09E−08 | −−−−−− | 11 | 5.8E+07 | GLYATL2 |
| rs6662013 | c | t | 5.688 | 1.29E−08 | ++++++ | 1 | 1.6E+08 | FAM78B |
| rs6852438 | c | t | 5.619 | 1.92E−08 | ++++++ | 4 | 1.8E+08 | ODZ3 |
| rs6887840 | a | c | 5.736 | 9.72E−09 | ++++++ | 5 | 1.7E+08 | FAM44B |
| rs6891234 | a | c | 5.493 | 3.96E−08 | ++++++ | 5 | 1.2E+08 | PRR16 |
| rs6991003 | a | t | 6.361 | 2.00E−10 | ++++++ | 8 | 2.6E+07 | BNIP3L |
| rs7164558 | a | g | −5.525 | 3.29E−08 | −−−−−− | 15 | 7.6E+07 | TBC1D2B |
| rs7192715 | c | t | −5.523 | 3.34E−08 | −−−−−− | 16 | 6.1E+07 | CDH8 |
| rs7209032 | c | t | 5.662 | 1.49E−08 | ++++++ | 17 | 2.6E+07 | CRLF3 |
| rs724845 | c | t | −5.582 | 2.38E−08 | −−−−−− | 6 | 1E+08 | GRIK2 |
| rs7263547 | c | g | −5.632 | 1.78E−08 | −−−−−− | 20 | 2E+07 | C20orf74 |
| rs7532243 | a | c | 5.631 | 1.79E−08 | ++++++ | 1 | 6.2E+07 | TM2D1 |
| rs7720860 | c | t | −5.607 | 2.06E−08 | −−−−−− | 5 | 1.5E+08 | JAKMIP2 |
| rs7839820 | a | g | 6.653 | 2.86E−11 | ++++++ | 8 | 1.1E+08 | TRHR |
| rs7908425 | c | g | −5.904 | 3.54E−09 | −−−−−− | 10 | 2734213 | PFKP |
| rs7924855 | a | g | −5.711 | 1.12E−08 | −−−−−− | 11 | 2.1E+07 | NELL1 |
| rs8038015 | c | t | 5.989 | 2.12E−09 | ++++++ | 15 | 9.7E+07 | IGF1R |
| rs8097637 | a | g | 5.855 | 4.78E−09 | ++++++ | 18 | 5.4E+07 | NEDD4L |
| rs8109263 | g | t | −5.469 | 4.53E−08 | −−−−−− | 19 | 2.1E+07 | ZNF626 |
| rs85431 | a | g | 6.494 | 8.35E−11 | ++++++ | 14 | 5.8E+07 | DACT1 |
| rs9325009 | c | t | 5.704 | 1.17E−08 | ++++++ | 5 | 1.5E+08 | TCERG1 |
| rs9514947 | c | t | 5.48 | 4.26E−08 | ++++++ | 13 | 1.1E+08 | MYO16 |
| rs965919 | c | t | −6.313 | 2.73E−10 | −−−−−− | 5 | 1.4E+08 | NRG2 |

PPC-F

| | | | | | | | | |
|---|---|---|---|---|---|---|---|---|
| rs10002158 | c | g | 5.916 | 3.29E−09 | ++++++ | 4 | 6573539 | PPP2R2C |
| rs10008703 | a | g | 6.074 | 1.24E−09 | ++++++ | 4 | 32560006 | PCDH7 |
| rs1011058 | a | g | 5.602 | 2.11E−08 | ++++++ | 13 | 73254684 | KLF12 |
| rs10281536 | g | t | −5.552 | 2.83E−08 | −−−−−− | 7 | 135456942 | MTPN |
| rs1030038 | a | g | 6.358 | 2.05E−10 | ++++++ | 2 | 70782175 | ADD2 |
| rs10448335 | c | t | 5.831 | 5.53E−09 | ++++++ | 9 | 70372768 | C9orf71 |
| rs10499129 | a | g | −5.477 | 4.34E−08 | −−−−−− | 6 | 124975906 | TCBA1 |
| rs10769990 | a | c | 5.804 | 6.46E−09 | ++++++ | 11 | 9102383 | RAB6IP1 |
| rs10775437 | a | g | 6.34 | 2.30E−10 | ++++++ | 18 | 6833259 | ARHGAP28 |
| rs10797752 | c | t | −5.864 | 4.52E−09 | −−−−−− | 1 | 180319837 | ZNF648 |
| rs10947850 | c | t | 5.988 | 2.12E−09 | ++++++ | 6 | 12332702 | HIVEP1 |
| rs11003132 | c | t | −6.191 | 5.98E−10 | −−−−−− | 10 | 54207453 | MBL2 |
| rs11067587 | c | t | −6.22 | 4.97E−10 | −−−−−− | 12 | 114338107 | MED13L |
| rs11122949 | a | g | 5.599 | 2.16E−08 | ++++++ | 2 | 123734743 | CNTNAP5 |
| rs1112919 | a | g | −5.644 | 1.66E−08 | −−−−−− | 2 | 54618678 | SPTBN1 |
| rs11133161 | a | g | −5.472 | 4.44E−08 | −−−−−− | 4 | 177703983 | VEGFC |
| rs1116008 | a | t | −5.933 | 2.97E−09 | −−−−−+ | 4 | 32181660 | PCDH7 |
| rs11629965 | c | t | 5.462 | 4.71E−08 | ++++++ | 15 | 82405649 | ADAMTSL3 |
| rs11728254 | a | g | 5.643 | 1.67E−08 | ++++++ | 4 | 149521029 | NR3C2 |
| rs11758068 | a | g | −5.508 | 3.64E−08 | −−−−−− | 6 | 82636270 | FAM46A |
| rs11789281 | a | t | −5.778 | 7.56E−09 | −−−−−− | 9 | 3746597 | GLIS3 |
| rs11843657 | c | t | 5.523 | 3.34E−08 | ++++++ | 13 | 39757504 | FOXO1 |
| rs11869615 | c | t | −6.158 | 7.36E−10 | −−−−−− | 17 | 29269981 | ACCN1 |
| rs11984109 | c | t | −6.435 | 1.24E−10 | −−−−−− | 7 | 105600422 | SYPL1 |
| rs12029285 | a | g | 5.986 | 2.15E−09 | ++++++ | 1 | 106364369 | PRMT6 |
| rs12091354 | c | t | 5.885 | 3.99E−09 | ++++++ | 1 | 243608399 | KIF26B |
| rs12101383 | a | g | 5.726 | 1.03E−08 | ++++++ | 15 | 65055257 | SMAD3 |
| rs12200300 | c | g | 5.504 | 3.71E−08 | +++++− | 6 | 80339370 | LCA5 |
| rs12418774 | a | g | −5.85 | 4.90E−09 | −−−−−− | 11 | 67530807 | UNC93B1 |
| rs12465864 | a | g | −5.604 | 2.10E−08 | −−−−−− | 2 | 238438444 | RAMP1 |
| rs12471370 | c | t | −5.681 | 1.34E−08 | −−−−−− | 2 | 142389275 | LRP1B |
| rs12504119 | g | t | 6.047 | 1.48E−09 | ++++++ | 4 | 11903918 | HS3ST1 |
| rs12551283 | a | g | 6.486 | 8.83E−11 | ++++++ | 9 | 121631118 | DBC1 |
| rs12612309 | g | t | −5.739 | 9.53E−09 | −−−−−− | 2 | 65694589 | SPRED2 |
| rs12677687 | c | t | −6.416 | 1.40E−10 | −−−−−− | 8 | 20956830 | GFRA2 |
| rs12801239 | c | t | −6.056 | 1.39E−09 | −−−−−− | 11 | 125828538 | KIRREL3 |
| rs13131866 | c | t | 5.492 | 3.97E−08 | ++++++ | 4 | 157402080 | CTSO |
| rs13265778 | c | t | 5.818 | 5.97E−09 | ++++++ | 8 | 58840748 | FAM110B |
| rs13267206 | c | t | 5.691 | 1.27E−08 | ++++++ | 8 | 76938348 | HNF4G |
| rs1331472 | c | t | −6.543 | 6.02E−11 | −−−−−− | 9 | 70404705 | C9orf71 |
| rs13425677 | g | t | 5.701 | 1.19E−08 | ++++++ | 2 | 6868616 | LOC129607 |
| rs1358882 | a | g | 5.564 | 2.64E−08 | ++++++ | 6 | 167387091 | FGFR1OP |
| rs1376605 | a | c | −5.526 | 3.27E−08 | −−−−−− | 3 | 32096983 | GPD1L |
| rs1449542 | a | g | −5.701 | 1.19E−08 | −−−−−− | 8 | 76770523 | HNF4G |
| rs1486816 | a | c | 5.692 | 1.26E−08 | ++++++ | 9 | 11773923 | TYRP1 |
| rs1502276 | a | g | −5.757 | 8.55E−09 | −−−−−− | 11 | 99936149 | CNTN5 |
| rs1507869 | a | g | 5.649 | 1.61E−08 | ++++++ | 3 | 36805250 | LBA1 |
| rs1533344 | c | t | −6.313 | 2.74E−10 | −−−−−− | 17 | 61976025 | PRKCA |
| rs1542371 | a | t | 5.589 | 2.28E−08 | ++++++ | 4 | 187169995 | SORBS2 |
| rs1585775 | c | t | 5.739 | 9.50E−09 | ++++++ | 3 | 103481815 | ZPLD1 |
| rs16838572 | c | t | −6.234 | 4.55E−10 | −−−−−− | 1 | 163292382 | LMX1A |
| rs16875331 | a | g | −5.459 | 4.80E−08 | −−−−−− | 8 | 108008872 | ABRA |
| rs16912660 | c | g | 6.273 | 3.55E−10 | ++++++ | 12 | 17393034 | FLJ22655 |
| rs16947580 | a | g | 5.634 | 1.76E−08 | ++++++ | 13 | 91872650 | GPC5 |
| rs17043278 | a | c | −5.541 | 3.01E−08 | −−−−−− | 3 | 5689934 | EDEM1 |
| rs17062397 | c | g | 5.95 | 2.68E−09 | ++++++ | 9 | 71007765 | TJP2 |
| rs17064357 | a | t | 6.063 | 1.34E−09 | ++++++ | 3 | 61014858 | FHIT |
| rs170827 | g | t | −6.756 | 1.41E−11 | −−−−−+ | 1 | 58854121 | TACSTD2 |
| rs17096074 | g | t | −7.366 | 1.76E−13 | −−−−−− | 14 | 58859645 | DAAM1 |
| rs17113689 | c | t | 6.352 | 2.13E−10 | ++++++ | 1 | 56537999 | PPAP2B |
| rs17137637 | a | g | 5.861 | 4.59E−09 | ++++++ | 5 | 114706552 | CCDC112 |
| rs17140677 | c | t | 6.8 | 1.05E−11 | ++++++ | 5 | 116227021 | SEMA6A |
| rs1720228 | c | g | −5.543 | 2.97E−08 | −−−−−− | 3 | 173588593 | FNDC3B |
| rs17676820 | c | t | −6.352 | 2.13E−10 | −−−−−− | 4 | 71431578 | AMTN |
| rs17729851 | c | t | 5.597 | 2.19E−08 | ++++++ | 13 | 89065858 | GPC5 |
| rs1775919 | a | g | −6.742 | 1.56E−11 | −−−−−− | 10 | 29213108 | BAMBI |
| rs17792047 | a | g | −6.916 | 4.65E−12 | −−−−−− | 20 | 58403602 | C20orf197 |
| rs179885 | c | g | 5.498 | 3.85E−08 | ++++++ | 7 | 105809184 | PBEF1 |
| rs1820825 | c | t | −5.49 | 4.01E−08 | −−−−−− | 8 | 15507610 | TUSC3 |
| rs1879671 | g | t | 5.961 | 2.51E−09 | ++++++ | 11 | 30311283 | C11orf46 |
| rs188178 | a | g | 6.587 | 4.48E−11 | ++++++ | 16 | 76189432 | ADAMTS18 |
| rs1994317 | a | t | −5.612 | 2.01E−08 | −−−−−− | 3 | 106723236 | ALCAM |
| rs201033 | a | c | 5.99 | 2.10E−09 | ++++++ | 6 | 6638442 | LY86 |
| rs2039957 | c | t | −5.617 | 1.95E−08 | −−−−−− | 1 | 219432395 | HLX |
| rs2062312 | a | c | 6.018 | 1.76E−09 | ++++++ | 18 | 68638440 | NETO1 |
| rs2103304 | a | g | 5.564 | 2.64E−08 | ++++++ | 15 | 53153611 | C15orf15 |
| rs2159472 | c | t | −6.012 | 1.83E−09 | −−−−−− | 7 | 146374539 | CNTNAP2 |
| rs2189099 | c | t | −5.685 | 1.31E−08 | −−−−−− | 7 | 82757757 | SEMA3E |
| rs2193875 | c | t | 5.869 | 4.39E−09 | ++++++ | 3 | 191804150 | IL1RAP |
| rs223110 | a | g | −6.03 | 1.64E−09 | −−−−−− | 14 | 23042883 | NGDN |
| rs2236891 | g | t | −5.596 | 2.20E−08 | −−−−−+ | 1 | 207871122 | LAMB3 |
| rs2246356 | c | t | 6.249 | 4.12E−10 | ++++++ | 9 | 85975801 | SLC28A3 |
| rs2254996 | a | g | 5.712 | 1.11E−08 | ++++++ | 5 | 153409251 | MFAP3 |
| rs2255273 | c | t | 5.495 | 3.92E−08 | ++++++ | 5 | 5200376 | ADAMTS16 |
| rs2305804 | c | g | 5.536 | 3.09E−08 | ++++++ | 19 | 15899365 | CYP4F11 |
| rs2315578 | c | t | −6.006 | 1.91E−09 | −−−−−− | 17 | 9644108 | DHRS7C |
| rs2320214 | a | c | −5.595 | 2.21E−08 | −−−−−− | 18 | 4410250 | DLGAP1 |
| rs2324499 | c | g | −5.815 | 6.07E−09 | −−−−−− | 13 | 39560001 | COG6 |
| rs2392285 | a | t | 6.17 | 6.84E−10 | ++++++ | 7 | 34861267 | NPSR1 |
| rs2424300 | c | t | 5.662 | 1.50E−08 | ++++++ | 20 | 20186950 | C20orf26 |
| rs2736874 | a | c | 5.759 | 8.45E−09 | ++++++ | 8 | 134551372 | ST3GAL1 |
| rs2899576 | c | t | 6.991 | 2.74E−12 | ++++++ | 15 | 53107909 | C15orf15 |
| rs3024851 | a | t | −6.474 | 9.54E−11 | −−−−−− | 2 | 191637067 | STAT4 |
| rs35405 | c | t | −5.57 | 2.54E−08 | −−−−−− | 5 | 33981515 | SLC45A2 |
| rs3801899 | a | g | 5.865 | 4.49E−09 | ++++++ | 7 | 155460238 | SHH |
| rs3812389 | c | t | 5.999 | 1.99E−09 | ++++++ | 7 | 29211284 | CHN2 |
| rs4140872 | c | t | 5.799 | 6.68E−09 | ++++++ | 2 | 169734842 | LRP2 |
| rs4290517 | a | g | −5.92 | 3.23E−09 | −−−−−− | 17 | 68196182 | SLC39A11 |
| rs4362420 | a | g | −6.475 | 9.46E−11 | −−−−−− | 16 | 75253877 | CNTNAP4 |
| rs4371785 | a | g | 5.462 | 4.71E−08 | ++++++ | 5 | 36557307 | SLC1A3 |
| rs4561982 | c | t | −5.64 | 1.70E−08 | −−−−−− | 4 | 115540465 | UGT8 |
| rs4595351 | a | c | 6.016 | 1.79E−09 | ++++++ | 1 | 163250449 | PBX1 |
| rs4648955 | a | g | 5.966 | 2.43E−09 | ++++++ | 1 | 24440677 | IL28RA |
| rs4673287 | a | g | −5.68 | 1.35E−08 | −−−−−− | 2 | 204744658 | ICOS |
| rs4715277 | a | g | 5.924 | 3.14E−09 | ++++++ | 6 | 52053686 | PKHD1 |
| rs4754687 | a | c | 6.669 | 2.58E−11 | ++++++ | 11 | 99935263 | CNTN5 |
| rs4862622 | a | g | −5.476 | 4.35E−08 | −−−−−− | 4 | 187176543 | TLR3 |
| rs4896502 | a | g | −5.569 | 2.56E−08 | −−−−−− | 6 | 140333871 | CITED2 |
| rs4940002 | a | g | 5.49 | 4.03E−08 | ++++++ | 18 | 46449193 | MAPK4 |
| rs4952539 | c | t | −6.11 | 9.95E−10 | −−−−−− | 2 | 41995056 | LOC91461 |
| rs558917 | a | g | −5.469 | 4.53E−08 | −−−−−− | 3 | 74432987 | CNTN3 |
| rs6052782 | a | c | −5.452 | 4.98E−08 | −−−−−− | 20 | 4640201 | PRNP |
| rs6456180 | c | t | 5.475 | 4.38E−08 | ++++++ | 6 | 170385059 | DLL1 |
| rs6465149 | c | t | −5.934 | 2.96E−09 | −−−−−− | 7 | 88139850 | ZNF804B |
| rs6484998 | c | t | 5.69 | 1.27E−08 | ++++++ | 11 | 11547337 | GALNTL4 |
| rs6485513 | c | t | 5.739 | 9.53E−09 | ++++++ | 11 | 44326716 | ALX4 |
| rs6533101 | c | t | 5.485 | 4.14E−08 | ++++++ | 4 | 104760456 | TACR3 |
| rs6764156 | c | t | 5.601 | 2.13E−08 | ++++++ | 3 | 74579805 | CNTN3 |
| rs6789415 | g | t | −5.738 | 9.56E−09 | −−−−−− | 3 | 85395324 | CADM2 |
| rs6814251 | a | t | −5.639 | 1.71E−08 | −−−−−+ | 4 | 122535336 | GPR103 |
| rs6862 | g | t | −5.54 | 3.02E−08 | −−−−−− | 7 | 5078634 | LOC389458 |
| rs693442 | c | t | −5.483 | 4.19E−08 | −−−−−− | 13 | 100725498 | NALCN |
| rs7048256 | a | g | −5.815 | 6.05E−09 | −−−−−− | 9 | 129172213 | GARNL3 |
| rs709143 | a | g | −5.473 | 4.41E−08 | −−−−−− | 3 | 192601529 | CCDC50 |
| rs7242593 | c | t | −5.899 | 3.65E−09 | −−−−−− | 18 | 9323937 | TWSG1 |
| rs7313672 | c | t | −5.777 | 7.59E−09 | −−−−−− | 12 | 128361357 | TMEM132D |
| rs7446448 | a | g | 5.605 | 2.08E−08 | ++++++ | 5 | 52452452 | MOCS2 |
| rs756958 | a | g | 6.078 | 1.22E−09 | ++++++ | 11 | 44006770 | LOC390110 |
| rs7697424 | g | t | 6.775 | 1.25E−11 | ++++++ | 4 | 181705931 | ODZ3 |
| rs771177 | c | t | −5.685 | 1.31E−08 | −−−−−− | 2 | 6866166 | LOC129607 |
| rs7742386 | c | t | 5.525 | 3.30E−08 | ++++++ | 6 | 21509740 | CDKAL1 |
| rs7859003 | c | t | −5.956 | 2.58E−09 | −−−−−− | 9 | 7262552 | JMJD2C |
| rs7864 | c | t | 5.687 | 1.29E−08 | ++++++ | 8 | 417913 | FBXO25 |
| rs8086522 | a | c | 5.996 | 2.03E−09 | +++++− | 18 | 341004 | COLEC12 |
| rs8104456 | c | t | 6.198 | 5.73E−10 | ++++++ | 19 | 34464045 | UQCRFS1 |
| rs8114348 | c | t | −5.91 | 3.42E−09 | −−−−−− | 20 | 45944879 | SULF2 |
| rs831784 | g | t | 5.593 | 2.24E−08 | ++++++ | 5 | 56384032 | MIER3 |
| rs888804 | a | g | 5.92 | 3.21E−09 | ++++++ | 5 | 85446937 | COX7C |
| rs913585 | c | t | −6.131 | 8.75E−10 | −−−−−− | 9 | 7278847 | JMJD2C |
| rs9313719 | c | t | 5.722 | 1.05E−08 | ++++++ | 5 | 157092264 | THG1L |
| rs9350031 | c | t | 6.493 | 8.43E−11 | ++++++ | 6 | 17259744 | RBM24 |
| rs9376791 | c | t | −5.89 | 3.86E−09 | −−−−−− | 6 | 144170379 | PHACTR2 |
| rs9416628 | a | t | 5.541 | 3.01E−08 | ++++++ | 10 | 59234259 | IPMK |
| rs9460898 | a | c | 6.401 | 1.54E−10 | ++++++ | 6 | 23699140 | NRSN1 |
| rs9478243 | a | g | 5.624 | 1.86E−08 | ++++++ | 6 | 152159151 | ESR1 |
| rs9531387 | a | g | 5.486 | 4.12E−08 | ++++++ | 13 | 82333973 | SLITRK1 |
| rs9601292 | g | t | −5.979 | 2.24E−09 | −−−−−− | 13 | 79384164 | NDFIP2 |
| rs966423 | c | t | 6.553 | 5.63E−11 | ++++++ | 2 | 218018585 | TNS1 |
| rs9821929 | c | t | 6.196 | 5.80E−10 | ++++++ | 3 | 29778509 | RBMS3 |

PPC-G

| | | | | | | | | |
|---|---|---|---|---|---|---|---|---|
| rs10005793 | a | g | −5.496 | 3.89E−08 | −−−−−− | 4 | 45633144 | GABRG1 |
| rs10018581 | c | t | 5.471 | 4.46E−08 | ++++++ | 4 | 82641457 | RASGEF1B |
| rs10225336 | c | t | 5.782 | 7.39E−09 | ++++++ | 7 | 15199866 | FLJ16237 |
| rs10227357 | c | g | 5.504 | 3.72E−08 | ++++++ | 7 | 21768673 | CDCA7L |
| rs10280585 | c | t | 6.797 | 1.07E−11 | ++++++ | 7 | 6139180 | USP42 |
| rs10417806 | a | c | 5.565 | 2.62E−08 | ++++++ | 19 | 17349141 | PLVAP |
| rs10776634 | c | t | 5.62 | 1.91E−08 | ++++++ | 10 | 49585662 | ARHGAP22 |
| rs10819125 | c | t | 7.07 | 1.55E−12 | ++++++ | 9 | 127921957 | PBX3 |
| rs10833328 | c | t | −5.842 | 5.17E−09 | −−−−−− | 11 | 20432136 | PRMT3 |
| rs10893450 | c | g | 6.32 | 2.61E−10 | ++++++ | 11 | 125323964 | CDON |
| rs10985401 | a | t | −5.614 | 1.98E−08 | −−−−−− | 9 | 123612989 | DAB2IP |
| rs11017774 | a | c | 5.59 | 2.27E−08 | ++++++ | 10 | 132847693 | TCERG1L |
| rs11033288 | a | g | 5.651 | 1.59E−08 | ++++++ | 11 | 35801506 | TRIM44 |
| rs11047355 | c | t | 5.471 | 4.47E−08 | ++++++ | 12 | 24334969 | SOX5 |
| rs11122200 | a | c | 6.046 | 1.48E−09 | ++++++ | 1 | 229245373 | FAM89A |
| rs11129475 | c | t | 5.95 | 2.68E−09 | ++++++ | 3 | 32005548 | OSBPL10 |
| rs11210982 | a | g | −7.327 | 2.36E−13 | −−−−−− | 1 | 44566347 | PRNPIP |
| rs1148546 | a | g | 5.932 | 3.00E−09 | ++++++ | 12 | 56957031 | XRCC6BP1 |
| rs1153536 | a | g | −6.276 | 3.47E−10 | −−−−−− | 3 | 2227530 | CNTN4 |
| rs11721807 | g | t | −5.847 | 5.02E−09 | −−−−−− | 4 | 157313959 | CTSO |
| rs11780899 | c | t | 6.541 | 6.12E−11 | ++++++ | 8 | 18474836 | PSD3 |
| rs11854996 | a | g | −5.641 | 1.69E−08 | −−−−−− | 15 | 35159173 | MEIS2 |
| rs11866630 | a | g | −6.039 | 1.55E−09 | −−−−−− | 16 | 74257482 | TERF2IP |
| rs12055381 | c | g | 5.769 | 7.95E−09 | +++−++ | 6 | 130155962 | C6orf191 |
| rs12101334 | a | t | −6.576 | 4.84E−11 | −−−−−− | 15 | 40008803 | EHD4 |
| rs12108434 | c | t | 5.602 | 2.12E−08 | ++++++ | 4 | 136985576 | PCDH18 |
| rs12217983 | a | t | −5.521 | 3.38E−08 | −−−+−− | 10 | 61734864 | ANK3 |
| rs12340257 | c | t | −5.812 | 6.16E−09 | −−−−−− | 9 | 112395540 | SVEP1 |
| rs12346949 | a | g | −5.991 | 2.09E−09 | −−−−−− | 9 | 112872846 | EDG2 |
| rs12512358 | a | g | 5.968 | 2.40E−09 | ++++++ | 4 | 7203404 | SORCS2 |
| rs12522180 | a | g | −5.529 | 3.22E−08 | −−−+−− | 5 | 5933413 | KIAA0947 |
| rs12568660 | a | c | 6.193 | 5.92E−10 | ++++++ | 1 | 35189347 | ZMYM6 |
| rs12629984 | c | t | 5.688 | 1.29E−08 | ++++++ | 3 | 88102183 | HTR1F |
| rs12927176 | c | g | 5.631 | 1.79E−08 | ++++++ | 16 | 79180342 | CDYL2 |
| rs13032979 | c | g | −5.852 | 4.85E−09 | −−−−−− | 2 | 202445434 | ALS2CR7 |
| rs13110356 | c | t | 5.653 | 1.58E−08 | ++++++ | 4 | 70326826 | UGT2B4 |
| rs13166852 | c | t | 5.526 | 3.27E−08 | ++++++ | 5 | 7648762 | ADCY2 |
| rs1328247 | c | t | 5.896 | 3.72E−09 | ++++++ | 13 | 108917530 | MYO16 |
| rs1348467 | c | t | 5.913 | 3.35E−09 | ++++++ | 13 | 52951373 | OLFM4 |
| rs1464609 | a | g | 5.528 | 3.23E−08 | ++++−+ | 8 | 123766046 | ZHX2 |
| rs1538432 | a | t | −5.524 | 3.31E−08 | −−−−−− | 10 | 86749755 | KIAA1128 |
| rs1588935 | a | g | −6.085 | 1.16E−09 | −−−−−− | 12 | 71771269 | TRHDE |
| rs165110 | c | t | 6.208 | 5.38E−10 | ++++++ | 18 | 73522973 | GALR1 |
| rs16841998 | c | t | 5.458 | 4.80E−08 | ++++++ | 2 | 139751689 | NXPH2 |
| rs16908206 | c | t | −5.948 | 2.71E−09 | −−−−−− | 8 | 139156805 | FAM135B |
| rs16940467 | c | t | −5.619 | 1.93E−08 | −−−−−− | 16 | 74187178 | ADAT1 |
| rs16991843 | g | t | −6.241 | 4.35E−10 | −−−−−− | 4 | 35687788 | CENTD1 |
| rs17228469 | g | t | −5.66 | 1.51E−08 | −−−−−− | 9 | 113073593 | OR2K2 |
| rs17252387 | c | g | 5.608 | 2.04E−08 | ++++++ | 15 | 66398543 | ITGA11 |
| rs17275995 | c | t | −5.677 | 1.37E−08 | −−−−−− | 7 | 24728564 | DFNA5 |
| rs17397004 | c | t | 5.619 | 1.92E−08 | ++++++ | 2 | 33155645 | LTBP1 |
| rs17404332 | c | t | 5.653 | 1.57E−08 | ++++++ | 10 | 7015537 | SFMBT2 |
| rs17405875 | c | t | −7.193 | 6.35E−13 | −−−−−− | 8 | 109156368 | RSPO2 |
| rs17607190 | c | t | −5.785 | 7.23E−09 | −−−−−− | 8 | 8311365 | CLDN23 |
| rs17765204 | c | t | 5.724 | 1.04E−08 | ++++++ | 14 | 79480170 | NRXN3 |
| rs1833 | a | c | 6.581 | 4.68E−11 | ++++++ | 10 | 42875694 | RET |
| rs1843371 | c | g | 5.894 | 3.78E−09 | ++++++ | 12 | 102978172 | HCFC2 |
| rs1860613 | c | t | −5.883 | 4.03E−09 | −−−−−− | 12 | 541931 | B4GALNT3 |
| rs1931084 | c | t | −5.718 | 1.08E−08 | −−−−−− | 13 | 100779104 | NALCN |
| rs1994379 | c | t | −6.419 | 1.37E−10 | −−−−−− | 15 | 37773405 | FSIP1 |
| rs2025935 | a | g | 6.13 | 8.76E−10 | ++++++ | 1 | 205742278 | CR1 |
| rs2065617 | a | g | 5.981 | 2.22E−09 | ++++++ | 20 | 56103090 | C20orf85 |
| rs2098883 | a | g | 5.664 | 1.48E−08 | ++++++ | 7 | 49807889 | VWC2 |
| rs2129209 | a | c | −5.529 | 3.22E−08 | −−−−−− | 6 | 159075986 | SYTL3 |
| rs2169856 | c | g | −5.733 | 9.88E−09 | −−−−−− | 12 | 53858919 | OR10A7 |
| rs2186903 | a | g | 5.526 | 3.28E−08 | ++++++ | 11 | 107261761 | SLC35F2 |
| rs2226179 | g | t | −6.74 | 1.59E−11 | −−−−−− | 1 | 163209800 | PBX1 |
| rs2243879 | c | g | −5.659 | 1.52E−08 | −−−−−− | 10 | 35662564 | CCNY |
| rs2287410 | a | t | 5.554 | 2.79E−08 | ++++++ | 2 | 186312736 | FLJ44048 |
| rs2290717 | a | g | 6 | 1.97E−09 | ++++++ | 12 | 10674996 | STYK1 |
| rs2359376 | a | g | 5.784 | 7.30E−09 | ++++++ | 1 | 196253748 | LHX9 |
| rs2404645 | a | g | −5.597 | 2.18E−08 | −−−−−− | 4 | 136302168 | PCDH10 |
| rs2571236 | a | g | −5.683 | 1.33E−08 | −−−−−− | 18 | 53607672 | ATP8B1 |
| rs2617101 | c | g | −5.884 | 4.00E−09 | −−−−−− | 8 | 4449958 | CSMD1 |
| rs2634511 | a | c | −6.485 | 8.88E−11 | −−−−−− | 8 | 59837041 | TOX |
| rs2682551 | c | t | −6.038 | 1.56E−09 | −−−−−− | 19 | 48695548 | PHLDB3 |
| rs2713621 | g | t | −5.965 | 2.44E−09 | −−−−−− | 3 | 130706821 | IFT122 |
| rs27567 | c | t | 5.608 | 2.05E−08 | ++++++ | 5 | 112769697 | TSSK1B |
| rs2828792 | c | g | 6.367 | 1.93E−10 | ++++++ | 21 | 24371534 | MRPL39 |
| rs2858590 | a | c | −6.767 | 1.31E−11 | −−−−−− | 22 | 47984977 | FAM19A5 |
| rs299499 | a | g | 5.773 | 7.78E−09 | ++++++ | 1 | 8215348 | SLC45A1 |
| rs3010311 | c | t | −5.726 | 1.03E−08 | −−−−−− | 6 | 139801888 | CITED2 |
| rs3050 | a | g | −5.773 | 7.78E−09 | −−−−−− | 6 | 150964808 | PLEKHG1 |
| rs3101796 | c | t | 6.357 | 2.06E−10 | ++++++ | 10 | 23265630 | ARMC3 |
| rs325369 | a | g | −5.773 | 7.78E−09 | −−−−−− | 18 | 36524196 | PIK3C3 |
| rs358734 | a | c | 6.035 | 1.59E−09 | ++++++ | 3 | 156966013 | C3orf33 |
| rs3744744 | c | t | −5.675 | 1.39E−08 | −−−−−− | 17 | 673882 | NXN |
| rs3759317 | c | t | 5.591 | 2.26E−08 | ++++++ | 12 | 105894382 | MTERFD3 |
| rs376200 | a | g | 6.056 | 1.39E−09 | ++++++ | 4 | 79496371 | FRAS1 |
| rs3785817 | a | g | 5.518 | 3.42E−08 | ++++++ | 17 | 39779191 | GRN |
| rs37974 | a | g | −6.227 | 4.74E−10 | −−−−−− | 7 | 7976911 | GLCCI1 |
| rs428311 | a | t | 6.105 | 1.03E−09 | ++++++ | 3 | 27956513 | EOMES |
| rs4318271 | c | t | −5.953 | 2.64E−09 | −−−−−− | 17 | 1051470 | ABR |
| rs4330511 | a | g | 5.745 | 9.20E−09 | ++++++ | 6 | 72578292 | RIMS1 |
| rs4445711 | a | g | −6.298 | 3.01E−10 | −−−−−− | 12 | 103160731 | TXNRD1 |
| rs4641033 | c | t | −5.558 | 2.73E−08 | −−−−−− | 8 | 57158229 | RPS20 |
| rs4653109 | a | g | −5.514 | 3.52E−08 | −−−−−− | 1 | 35100832 | DLGAP3 |
| rs4659708 | a | c | 5.644 | 1.66E−08 | ++++++ | 1 | 234965966 | ACTN2 |
| rs4726340 | a | t | −5.477 | 4.34E−08 | −−−−−− | 7 | 153188040 | DPP6 |
| rs473172 | a | g | 6.877 | 6.10E−12 | ++++++ | 11 | 116313180 | KIAA0999 |
| rs4780337 | a | c | 5.621 | 1.90E−08 | ++++++ | 16 | 10938243 | DEXI |
| rs4798470 | c | t | 5.81 | 6.24E−09 | ++++++ | 18 | 6643590 | ARHGAP28 |
| rs4859966 | a | c | −6.035 | 1.59E−09 | −−−−−− | 4 | 76738120 | CDKL2 |
| rs4899265 | a | c | −5.557 | 2.75E−08 | −−−−−− | 14 | 68391314 | ACTN1 |
| rs4924590 | a | t | −5.901 | 3.62E−09 | −−−−−− | 15 | 40020965 | EHD4 |
| rs4965520 | a | g | 6.805 | 1.01E−11 | ++++++ | 15 | 96525461 | ARRDC4 |
| rs501290 | a | c | 5.463 | 4.69E−08 | +++−++ | 9 | 22774110 | DMRTA1 |
| rs5019079 | c | g | 6.396 | 1.60E−10 | ++++++ | 3 | 76753974 | ROBO2 |
| rs534904 | c | t | 6.099 | 1.07E−09 | ++++−+ | 8 | 102567794 | GRHL2 |
| rs558706 | c | g | 6.325 | 2.53E−10 | ++++++ | 9 | 3957879 | GLIS3 |
| rs572491 | a | t | 5.702 | 1.19E−08 | +++−++ | 9 | 3917738 | GLIS3 |
| rs5770661 | a | g | 5.917 | 3.27E−09 | ++++++ | 22 | 47758835 | FAM19A5 |
| rs6098802 | a | g | 5.911 | 3.40E−09 | ++++++ | 20 | 53805479 | CBLN4 |
| rs6134073 | c | g | −5.562 | 2.66E−08 | −−−−−− | 20 | 10814660 | JAG1 |
| rs6134489 | c | t | −5.537 | 3.08E−08 | −−−−−− | 20 | 12182802 | BTBD3 |
| rs6448755 | a | g | −5.461 | 4.72E−08 | −−−−−− | 4 | 30809610 | PCDH7 |
| rs6561712 | c | t | 6.129 | 8.85E−10 | ++++++ | 13 | 52778810 | OLFM4 |
| rs6564672 | a | g | −5.675 | 1.39E−08 | −−−−−− | 16 | 78050238 | MAF |
| rs6601751 | g | t | 5.513 | 3.54E−08 | ++++++ | 10 | 3756696 | KLF6 |
| rs669310 | a | g | 6.125 | 9.08E−10 | ++++++ | 4 | 130752760 | C4orf33 |
| rs6718569 | c | t | 5.54 | 3.03E−08 | ++++++ | 2 | 59458515 | BCL11A |
| rs673282 | c | t | 6.049 | 1.46E−09 | ++++++ | 18 | 4375235 | DLGAP1 |
| rs6756721 | a | g | −5.831 | 5.51E−09 | −−−−−− | 2 | 229565736 | PID1 |
| rs6804188 | c | t | −5.564 | 2.63E−08 | −−−−−− | 3 | 156992357 | C3orf33 |
| rs6844333 | c | t | −5.513 | 3.53E−08 | −−−−−− | 4 | 54351850 | LOC402176 |
| rs6923429 | c | g | 5.574 | 2.49E−08 | ++++++ | 6 | 24834785 | C6orf62 |
| rs6950173 | a | g | −5.526 | 3.27E−08 | −−−−−− | 7 | 22136045 | RAPGEF5 |
| rs7017336 | g | t | −6.388 | 1.68E−10 | −−−−−− | 8 | 82674096 | IMPA1 |
| rs7028263 | a | g | 6.127 | 8.96E−10 | ++++++ | 9 | 31105847 | ACO1 |
| rs7076143 | a | g | −5.452 | 4.99E−08 | −−−−−− | 10 | 129874450 | MKI67 |
| rs7081956 | c | t | −6.039 | 1.56E−09 | −−−−−− | 10 | 58912724 | IPMK |
| rs7164199 | g | t | −6.774 | 1.25E−11 | −−−−−− | 15 | 75939681 | TBC1D2B |
| rs7285846 | c | g | −5.632 | 1.78E−08 | −−−−−− | 22 | 41963344 | SCUBE1 |
| rs7336914 | a | g | 5.704 | 1.17E−08 | +++−−+ | 13 | 29667250 | KATNAL1 |
| rs7406992 | c | t | 5.485 | 4.14E−08 | ++++++ | 17 | 786714 | NXN |
| rs741223 | g | t | −5.66 | 1.52E−08 | −−−−−− | 9 | 112869081 | EDG2 |
| rs747838 | c | g | 5.614 | 1.98E−08 | ++++++ | 18 | 11995695 | IMPA2 |
| rs7500162 | a | g | −5.903 | 3.56E−09 | −−−−−− | 16 | 85044314 | FOXF1 |
| rs7578859 | a | c | −5.782 | 7.39E−09 | −−−−−− | 2 | 208703172 | CRYGC |
| rs758971 | a | g | −5.919 | 3.23E−09 | −−−−−− | 9 | 128215611 | FAM125B |
| rs7613532 | a | g | 5.711 | 1.12E−08 | ++++++ | 3 | 182801980 | SOX2 |
| rs773312 | g | t | 5.494 | 3.94E−08 | ++++−+ | 13 | 104130457 | DAOA |
| rs7740170 | a | g | 6.648 | 2.98E−11 | ++++++ | 6 | 160196917 | PNLDC1 |
| rs7804 | c | t | 5.857 | 4.73E−09 | ++++++ | 7 | 45082298 | CCM2 |
| rs7808523 | a | g | −5.462 | 4.70E−08 | −−−−−− | 7 | 71275165 | CALN1 |
| rs7904936 | c | t | −5.839 | 5.24E−09 | −−−−−− | 10 | 70666198 | HKDC1 |
| rs8022554 | a | g | 5.889 | 3.88E−09 | ++++++ | 14 | 58589906 | DAAM1 |
| rs827080 | a | g | −5.665 | 1.47E−08 | −−−−−− | 6 | 113449386 | RFPL4B |
| rs867677 | a | c | −6.242 | 4.32E−10 | −−−−−− | 4 | 79732631 | ANXA3 |
| rs888092 | a | t | 5.572 | 2.51E−08 | ++++++ | 2 | 74823764 | SEMA4F |
| rs915180 | c | t | −5.578 | 2.43E−08 | −−−−−− | 1 | 154345707 | LMNA |
| rs9428536 | c | t | −5.461 | 4.74E−08 | −−−−−− | 1 | 240299937 | PLD5 |
| rs9457640 | c | t | 5.943 | 2.80E−09 | ++++++ | 6 | 159796374 | FNDC1 |
| rs949741 | a | c | −6.126 | 9.00E−10 | −−−−−− | 13 | 96763781 | MBNL2 |
| rs955901 | a | g | 5.682 | 1.33E−08 | ++++++ | 3 | 20819889 | SGOL1 |
| rs959083 | a | g | 5.792 | 6.97E−09 | ++++++ | 14 | 102892440 | EIF5 |
| rs9701452 | g | t | −5.455 | 4.89E−08 | −−−−−− | 1 | 227357530 | RAB4A |
| rs9830768 | c | t | −6.809 | 9.83E−12 | −−−−−− | 3 | 157722002 | KCNAB1 |
| rs984984 | g | t | −5.661 | 1.51E−08 | −−−−−− | 1 | 206101711 | CD34 |
| rs9972232 | a | g | 5.458 | 4.82E−08 | ++++++ | 14 | 21807431 | DAD1 |

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

Introduction

The recently introduced concept of precision medicine offers a new vision for the prevention and treatment of disease, as well as for biomedical research. Along the lines of the “personalized medicine” paradigm, precision medicine entails prevention and treatment strategies that take individual variability into account.¹ A complete account of environmental and innate influences of disease susceptibility is certainly a daunting task—nevertheless, recent advances in the biomedical sciences have made possible the comprehensive characterization of individuals' genomes, transcriptomes, proteomes and metabolomes, and the superimposition of this “panomics” information with detailed health and disease endpoints.² There is promise that “precision dentistry” will emerge from this new wave of systems biology and big data-driven science and practice, and will bring about meaningful improvements in individuals' and populations' health.^{3, 4}

Recent efforts in periodontal medicine have built upon the principles of precision medicine to refine periodontal health and disease classifications and dissect the biological basis of disease susceptibility, with the ultimate goal of tailoring or targeting prevention and treatment strategies. ⁵ Along these lines, the development of a precise stratification system that reflects distinct periodontal disease patterns can serve as the basis for precise risk assessment (at the population-level) and estimation of individual susceptibilities (at the person-level), with disease progression and tooth loss being the main endpoints of interest. However, current periodontal disease taxonomies have limited utility for predicting disease progression and tooth loss; in fact, tooth loss itself can undermine precise person-level periodontal disease classifications.

To date, periodontal disease risk assessment tools have used clinical measurements and known risk factors to predict tooth loss or periodontal disease progression with the goal of establishing more specific prognoses and to optimize treatment choices.⁶⁻¹¹ Although some prediction models incorporate well-established risk factors, such as smoking history, diabetes, age, race and sex, there are currently no validated risk assessment tools utilizing clinical parameters including tooth-specific patterns. Most models utilize subject-level summary variables of clinical parameters, such as mean or extent scores for various signs of disease including plaque scores, gingival indices, probing depths, and clinical attachment levels, that reflect subject-level disease and are not always linked to tooth type or tooth loss patterns. Most prediction models do not explicitly classify missing teeth, which can be lost for a variety of reasons, and are not informed by existing tooth loss patterns when considering risk of natural disease progression, which has important prognostic value.¹² Individual tooth-specific measures of crown to root ratios, mobility, tooth position and other factors can be used to improve estimates of individual tooth-level prognoses, but irrespective of the model the final estimates of risk are qualitative in nature based upon clinical impressions. In sum, no predictive models exist that provide quantitative tooth-based risk estimates and account for the informative heterogeneity in clinical presentation by patterns of tooth loss. The elements are critical in the realm of precision dentistry, and are necessary for informing personalized periodontal, restorative and prosthodontics plans of care.

To overcome these limitations, a periodontitis stratification system (UNC-PPC) based on latent class analyses of clinical parameters including patterns of missing teeth was developed. Previous work has demonstrated how multiple clinical characteristics can be used to identify and stratify clinically distinct periodontal and tooth profile classes that incorporate these tooth loss patterns.¹³ This Example describes, inter alia, the clinical utility of the UNC-PPC taxonomy for risk assessment and ‘precision periodontal medicine’, specifically for predicting and/or treating periodontal disease progression and incident tooth loss.

Materials and Methods

Study Populations. The analytical sample comprised 4,682 adult participants of two prospective cohort studies [Dental Atherosclerosis Risk in Communities Study (DARIC) and Piedmont Dental Study (PDS)] with information on periodontal disease progression and incident tooth loss. All participants provided written informed consent to a protocol that was reviewed and approved by the Institutional Review Board on research involving human subjects at the University of North Carolina and/or at each study performance site.

The DARIC sample was recruited from the ARIC population study and included dentate participants who did not have contraindications for periodontal probing.¹⁴ The entire DARIC study comprised 6,793 dentate individuals living in four United States communities and received a baseline dental and periodontal examination between 1996-1998. In 2012-13, DARIC participants were asked via follow-up calls to assess their tooth loss in the previous ten years: “Have you lost any teeth in the past ten years?”. Their answers were categorized as none, one or two, three or more, and don't know.¹⁵

The PDS was based on a stratified random clustered sample of all people aged 65 and over in the five adjacent counties in the Piedmont area of North Carolina.¹⁶ The longitudinal study began in 1988 with a random subsample of 697 dentate individuals. Additional study design and population characteristics are described in detail in previous publications.^{17, 18}

Calculation of the Index of Periodontal Classes as a Risk Score. The DARIC 10-year tooth loss data was used to compute the risk of tooth loss for each tooth profile class (TPC) within each periodontal profile class (PPC) assignment. A composite risk score for each individual was then calculated based on tooth loss risks—this continuous score (ranging theoretically between 0 and 100) is referred to as the Index of Periodontal Risk (IPR). The analytical approach used to calculate IPR was based on a 7×7 table (PPC×TPC) of predicted probabilities for 10-year tooth loss. First, each participant was assigned to one of the 7 PPCs and then, each tooth was classified to one of the 7 TPCs. The IPR was then calculated as the mean predicted probability for 10-year tooth loss across all teeth present for each individual. The development of the IPR included traditional risk factors for periodontal disease, such as age, sex, race, diabetes, and smoking status. Information from the DARIC dataset was used to develop an IPR adjustment score for each of these traditional risk factors.

Statistical Analyses. Logistic regression models adjusting for examination center, race, gender, age, diabetes, and smoking were used to quantify the association of the seven PPCs and the seven TPCs with tooth loss (DARIC and PDS datasets) and periodontitis progression (PDS dataset) by obtaining corresponding relative risk (RR) estimates and 95% confidence intervals (CI). Similar models were developed using the Center for Disease Control/American Academy of Periodontology (CDC/AAP) definition of periodontal disease,¹⁹ to allow the contrast of tooth loss and periodontitis estimates of association with the ones obtained from PPC/TPC-based analyses.

Predicted probabilities and 95% confidence intervals were computed to estimate the risk for tooth loss across the observed range of IPR scores using the DARIC dataset. Effect estimates (beta coefficients) for race, age, gender, diabetes, and smoking status were also computed in the DARIC dataset. This IPR-developed model was subsequently applied to the PDS longitudinal dataset for validation. Predicted probabilities were calculated to estimate the risk for tooth loss, periodontitis progression, and incidence of edentulism in the PDS. Periodontitis progression was defined as ≥10% of sites exhibiting ≥3 mm attachment loss in a 3-year period. To evaluate the sensitivity and specificity of the predicted probability model, a receiver operator curve (ROC)²⁰ and C-statistic ²¹ were calculated for each predicted estimate. IPR thresholds for defining risk categories as Index of Periodontal Classes (IPC)-‘Low’, ‘Moderate’ and ‘High’ were identified using the Bayesian Information Criterion (BIC)²² and confirmed with Classification and Regression Trees (CART).²³

Results

The demographic and clinical characteristics of the study participants as well the 7 PPCs were reported in an earlier publication.¹³ There were significant differences between the PPC groups with regard to race, sex, age, diabetes, smoking (history and pack/year), obesity, access to dental care, socio-economic status, and educational level.

Risk Models for Tooth Loss and Periodontal Disease Progression by Periodontal Profile Class. The DARIC dataset was originally used to derive the PPC classification¹³ and in this study was used to estimate associations with incident tooth loss and periodontal disease progression (i.e., clinical attachment loss). The Piedmont dataset was used as an independent validation dataset. Table 2 presents the Relative Risk (RR) and 95% confidence intervals (CI) for a person losing ≥3 teeth over a 10-year period for the DARIC sample stratified by PPC assignment and CDC/AAP disease definition. Estimates from both crude and fully adjusted (for race, gender, age, diabetes, and smoking status) models are presented. Individuals assigned to PPC-D and -G had the highest tooth loss risk: RR=3.8 (95% CI=2.9-5.1) and 3.6 (2.6-5.0), respectively. These estimates, derived from the UNC-PPC system demonstrated a stronger association with tooth loss compared to the CDC/AAP severe disease classification (RR=2.8; 95% CI=2.0-4.0).

TABLE 2
Relative Risk and 95% Confidence Intervals (CI) for incident
loss of ≥3 teeth over a 10-year period (n = 3,985)
among the DARIC participants stratified by Periodontal
Profile Class (PPC) and Center for Disease Control/American
Academy of Periodontology (CDC/AAP) classification.

| Classi- | | | Adjusted |
|---|---|---|---|
| fication | Description | Crude Model | Model* |
| PPC-A | Health | Ref | Ref |
| PPC-B | Mild Disease | 2.10 (1.53-2.87) | 1.84 (1.34-2.53) |
| PPC-C | High GI Scores | 3.84 (2.82-5.23) | 2.15 (1.51-3.05) |
| PPC-D | Tooth Loss | 4.91 (3.71-6.51) | 3.83 (2.87-5.12) |
| PPC-E | Posterior Disease | 2.89 (2.15-3.90) | 2.72 (2.01-3.69) |
| PPC-F | Severe Tooth Loss | 3.32 (2.45-4.50) | 2.29 (1.66-3.15) |
| PPC-G | Severe Disease | 5.91 (4.39-7.96) | 3.62 (2.61-5.01) |
| CDC/AAP | Health | Ref | Ref |
| CDC/AAP | Mild | 1.24 (0.88-1.75) | 1.13 (0.78-1.61) |
| CDC/AAP | Moderate | 1.90 (1.37-2.62) | 1.72 (1.22-2.42) |
| CDC/AAP | Severe | 3.65 (2.63-5.05) | 2.82 (1.97-4.04) |

*Adjusted for examination center, race/center, sex, age, diabetes, smoking (5 levels)

Five-year tooth loss risk estimates (≥2 and ≥3 teeth) are presented in Table 3. Similarly, Table 3 presents periodontal disease progression (3-year attachment loss of ≥3 mm in ≥10% of sites in the PDS) risk estimates according to the UNC-PPC system and the CDC/AAP definition. Individuals assigned to PPC-D, -E, and -G classes showed the highest risk of losing ≥2 teeth, with corresponding estimates of RR=3.5 (95% CI=1.6-7.6), 3.9 (1.0-14.4), and 3.4 (1.4-8.4). As expected, risk estimates for attachment loss were the highest among PPCs associated with disease. For example, for attachment loss, PPC-G had the highest risk RR=7.8 (95% CI=3.0-20.7) followed by PPC-D: RR=6.1 (95% CI=2.4-15.5) and PPC-F: RR=4.2 (95% CI=0.6-11.1). In contrast, the severe disease CDC/AAP group had RR=4.5 (95% CI=2.2-9.2). These results highlight the higher risks of attachment loss associated with PPC-D and PPC-G assignment as compared to the CDC/AAP severe category, even after adjustments for race, gender, age, diabetes, and smoking status. These findings support the utility of the PPC method to identify subjects with elevated disease progression risk in an independent sample (PDS).

TABLE 3
Adjusted* Relative Risk and 95% Confidence Interval (CI) for
losing ≥2 and ≥3 teeth over a 5-year period and 10% of sites with
3+ mm Attachment Loss (AL) increase (n = 363) over a 3-year
period for the PDS (Piedmont Dental Study) population stratified
by Periodontal Profile Class (PPC) and Center for Disease Control/
American Academy of Periodontology (CDC/AAP) classification.

| | | | | |
|---|---|---|---|---|
| | | | | Attachment Loss |
| | | Tooth Loss | Tooth Loss | 10% of sites |
| | | ≥2 teeth | ≥3 teeth | with ≥3 mm |
| Class | Description | over 5 years | over 5 years | AL increase |

PPC

| | | | | |
|---|---|---|---|---|
| PPC-A | Health | Ref | Ref | Ref |
| PPC-B | Mild Disease | 2.59 (1.24-5.40) | 2.54 (0.89-7.29) | 1.34 (0.28-6.54) |
| PPC-C | High GI Scores | 3.02 (1.65-5.50) | 3.87 (1.69-8.89) | 3.05 (1.18-7.89) |
| PPC-D | Tooth Loss | 3.51 (1.91-6.44) | 4.34 (1.87-10.0) | 6.15 (2.44-15.5) |
| PPC-E | Posterior Disease | 4.21 (1.80-9.83) | 3.54 (0.87-14.3) | 4.05 (0.60-27.3) |
| PPC-F | Severe Tooth Loss | 3.08 (1.66-5.70) | 3.89 (1.67-9.06) | 4.16 (1.56-11.1) |
| PPC-G | Severe Disease | 3.32 (1.71-6.45) | 4.72 (1.94-11.5) | 7.82 (2.96-20.7) |

CDC/AAP

| | | | | |
|---|---|---|---|---|
| CDC/AAP | Health | Ref | Ref | Ref |
| CDC/AAP | Mild | 0.69 (0.42-1.12) | 0.73 (0.39-1.38) | 0.68 (0.24-1.84) |
| CDC/AAP | Moderate | 1.20 (0.84-1.70) | 1.16 (0.70-1.89) | 1.91 (0.93-3.93) |
| CDC/AAP | Severe | 1.89 (1.33-2.68) | 2.46 (1.52-3.98) | 4.49 (2.19-9.22) |

*Adjusted for race, sex, age, diabetes and smoking

Risk Models for Tooth Loss and Periodontal Disease Progression by Tooth Profile Class. Tooth loss and disease progression risk estimates in the PDS sample according to TPC are presented in Table 4. Evidently, teeth classified under the TPC associated with periodontal disease showed higher risk for being lost. TPC-G had the highest tooth loss risk RR=3.9 (95% CI=3.4-4.7), followed TPC-F: RR=2.5 (95% CI=2.1-2.9) and TPC-E: RR=2.3 (95% CI=1.9-3.0). Attachment loss estimates were significantly higher for TPC-B (“Recession”), TPC-E (“Interproximal Periodontal Disease”), and TPC-G (“Severe Periodontal Disease”) compared to TPC-A (“Health”).

TABLE 4
Tooth Level Adjusted* Relative Risk and 95% Confidence Interval
(CI) stratified by Tooth Profile Class (TPC) for Observed
5-Year Tooth Loss and Attachment Loss Increase (≥3 mm
increase) for the Piedmont Dental Study (PDS) dataset.

| Tooth | | | |
|---|---|---|---|
| Profile | | | Attachment Loss |
| Class | Description | Tooth Loss | Increase (≥3 mm) |
| TPC-A | Health | Ref | Ref |
| TPC-B | Recession | 1.24 (1.00-1.55) | 1.56 (1.40-1.77) |
| TPC-C | Crown | 0.85 (0.62-1.14) | 0.49 (0.38-0.65) |
| TPC-D | Gingival Inflammation | 2.02 (1.69-2.41) | 1.36 (1.19-1.56) |
| TPC-E | Interproximal Disease | 2.34 (1.89-3.02) | 1.77 (1.49-2.10) |
| TPC-F | Diminished | 2.50 (2.14-2.92) | 1.44 (1.28-1.63) |
| | Periodontium | | |
| TPC-G | Severe Disease | 3.95 (3.35-4.66) | 1.73 (1.46-2.04) |

*Adjusted for race, age, sex, diabetes and smoking

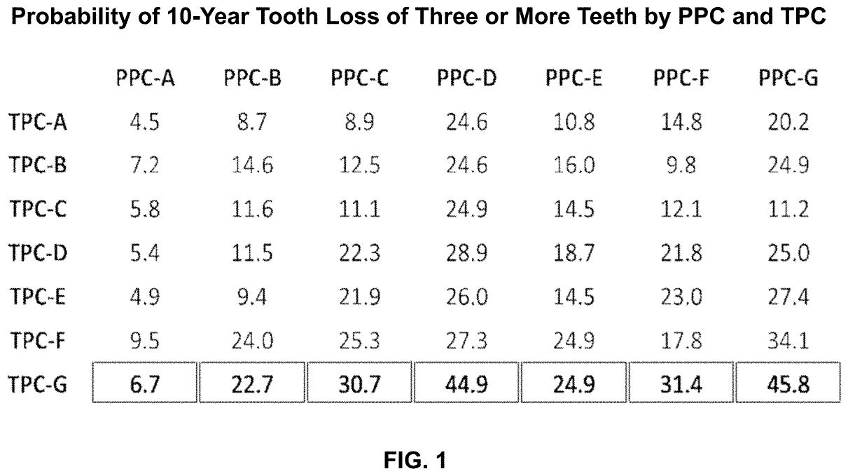

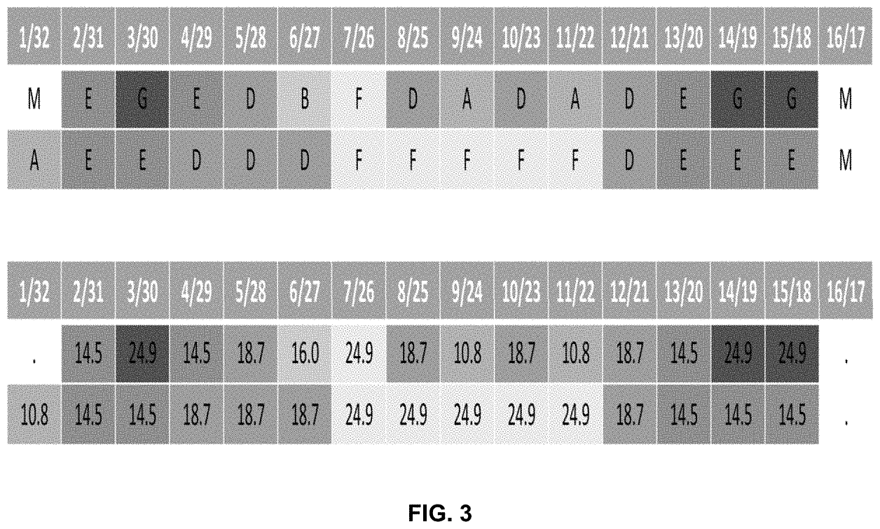

Predicted Probability of 10-Year Tooth Loss Stratified by PPC and TPC. The tooth-level risk scores, which are computed probabilities for 10-year tooth loss (≥3 teeth) using the DARIC dataset are shown in FIG. 1 as a 7×7 table (PPC/TPC). For example, periodontally-healthy teeth, in a periodontally-healthy person (TPC A/PPC A) had a pooled 4.5% probability of belonging to a person who lost teeth over 10 years. As expected, healthy teeth (TPC-A) had lower risk than diseased teeth (TPC-G) to be in a diseased person (PPC-G). Periodontally-healthy teeth in a participant with severe disease (TPCA/PPC G) had an increased probability to be lost (20.2%). In contrast, a severely diseased tooth in a person with severe disease (TPC G/PPC G) had 45.8% probability of being lost (FIG. 1). As mentioned earlier, these pooled-risk tooth-level scores stratified by PPC were averaged across teeth. For example, if a PPC-A (“Health”) person had all teeth classified as TPC-A (“Health”), the IPR would be 4.5. Similarly, a PPC-G (“Severe Disease”) person with all teeth classified as TPC-G (“Severe Disease”) would have an IPR score of 45.8. A clinical example of how to calculate the IPR for a hypothetical PPC-E person having teeth representing multiple TPC classes is presented in FIG. 3. The person-level IPR score is a composite risk score based upon the DARIC dataset that is unadjusted for traditional person-based risk factors. Table 5 provides a numerical adjustment of the IPR score that accounts for the effect of age, race, sex, diabetes and smoking. Thus, a male subject who is a current smoker and is of age 65 will have an IPR score that is increased by 8 points, based upon these risk factors (3+5+0) using estimates described under methods.

TABLE 5
Numerical adjustment of the Index of Periodontal Risk
(IPR) score that accounts for the effect of age, race,
sex, diabetes and smoking based on the DARIC dataset.

| Risk Factor | IPR Adjustment |
|---|---|
| Diabetes | +3 |
| Male | +3 |
| African American | +5 |
| Smokers | +5 |
| Age 30-39 | −7.8 |
| Age 40-49 | −5.2 |
| Age 50-59 | −2.6 |
| Age 60-69 | +0 |
| Age 70-79 | +2.6 |
| Age 80-89 | +5.2 |
| Age 90+ | +7.8 |

If after adjustment IPC < 5 then IPC = 5
If after adjustment IPC > 46 then IPC = 46

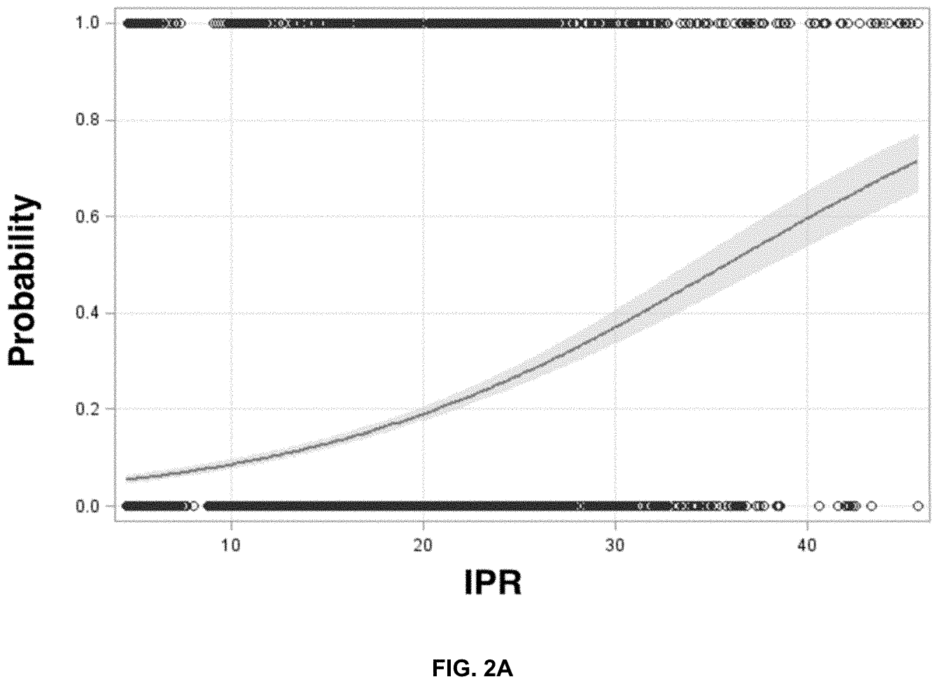

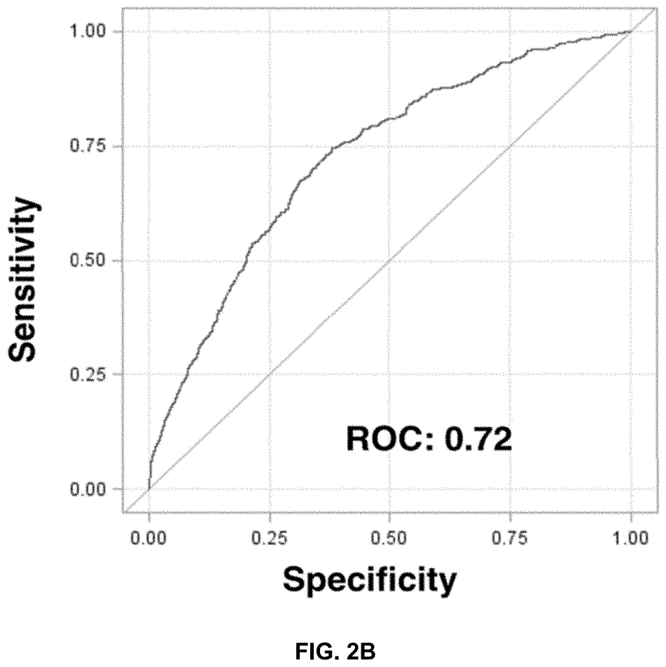

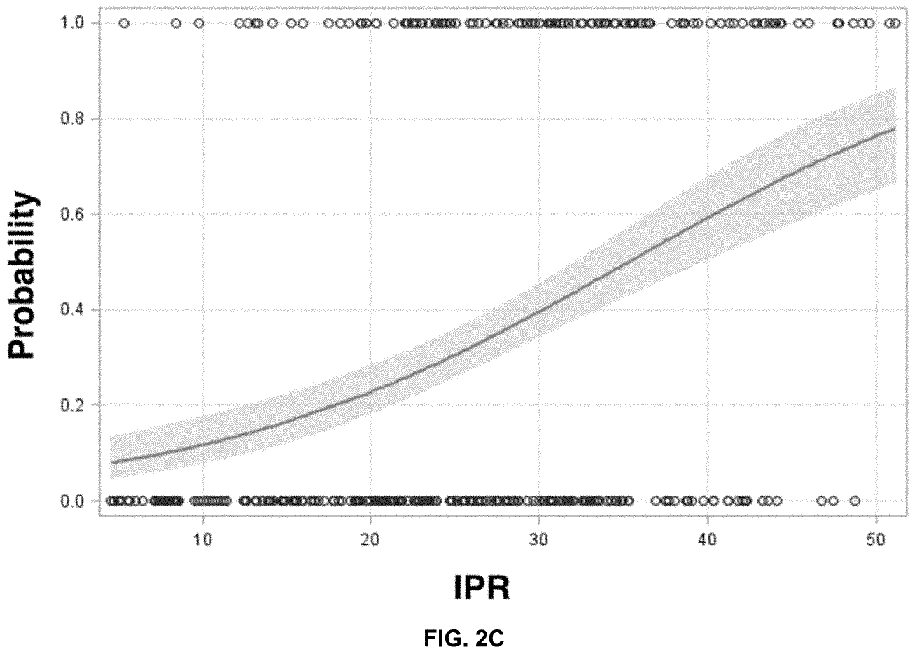

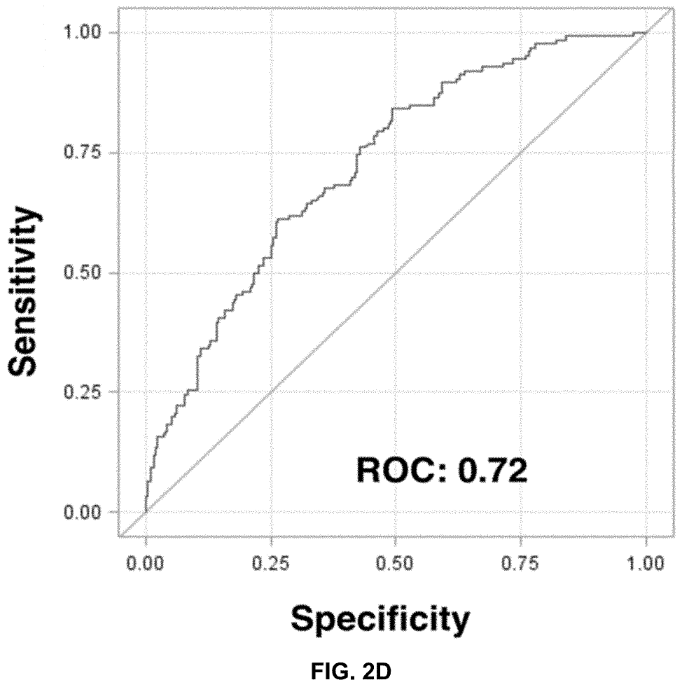

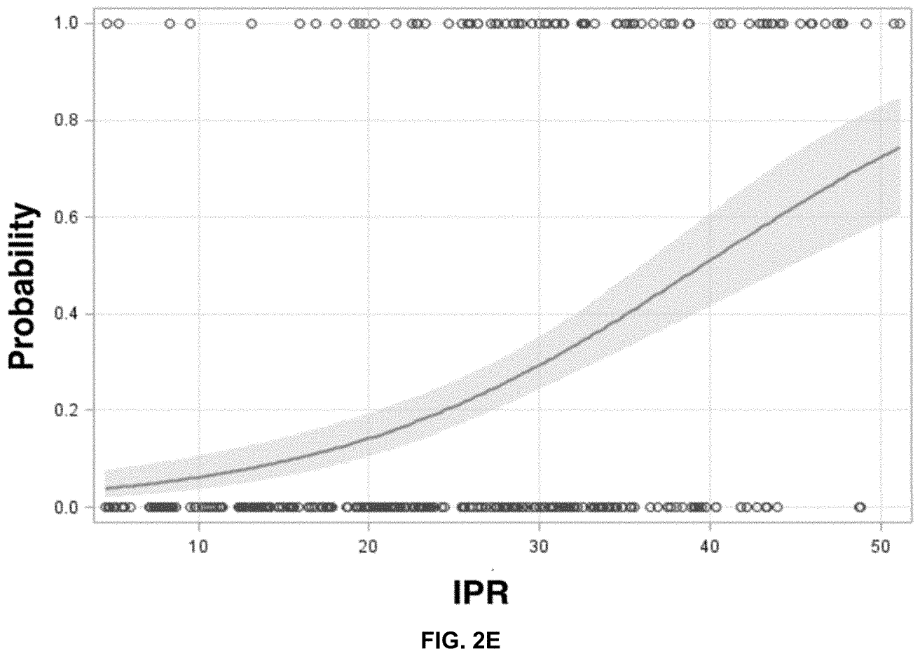

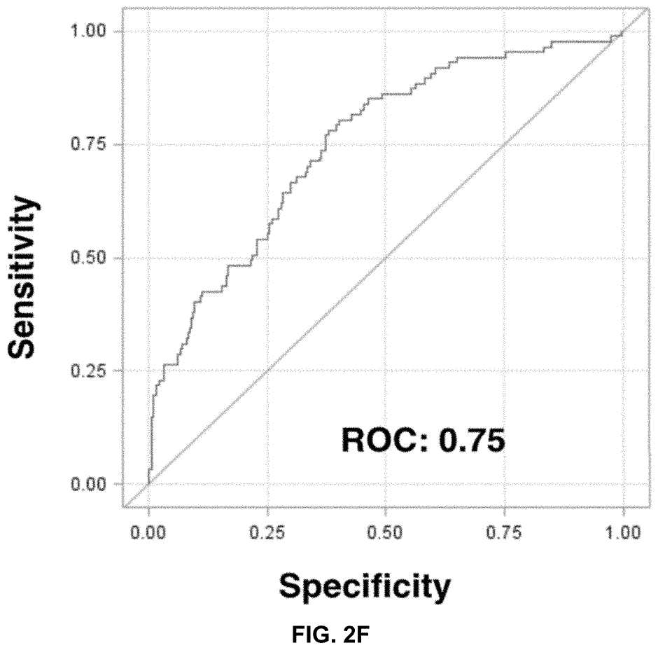

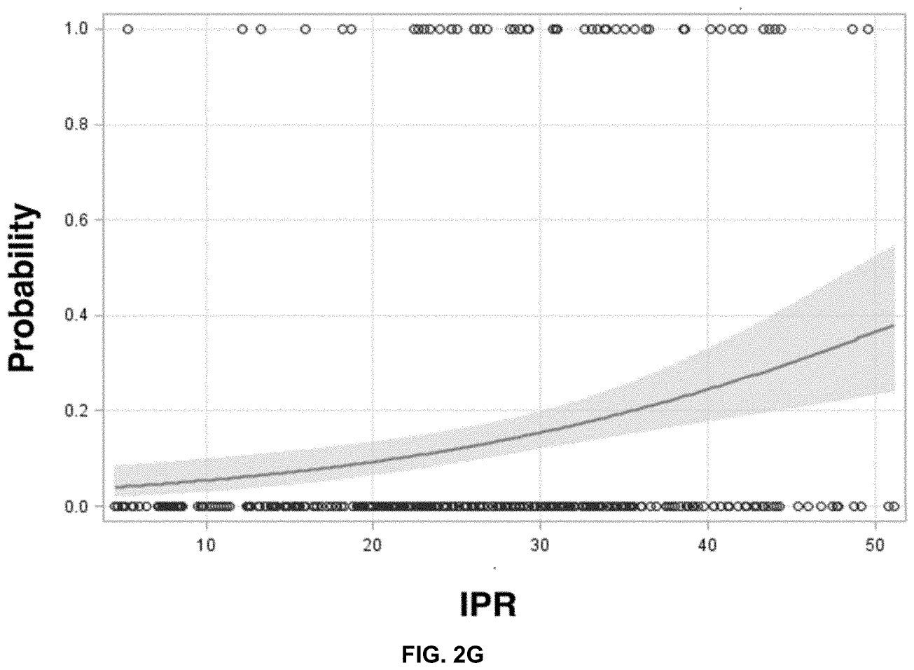

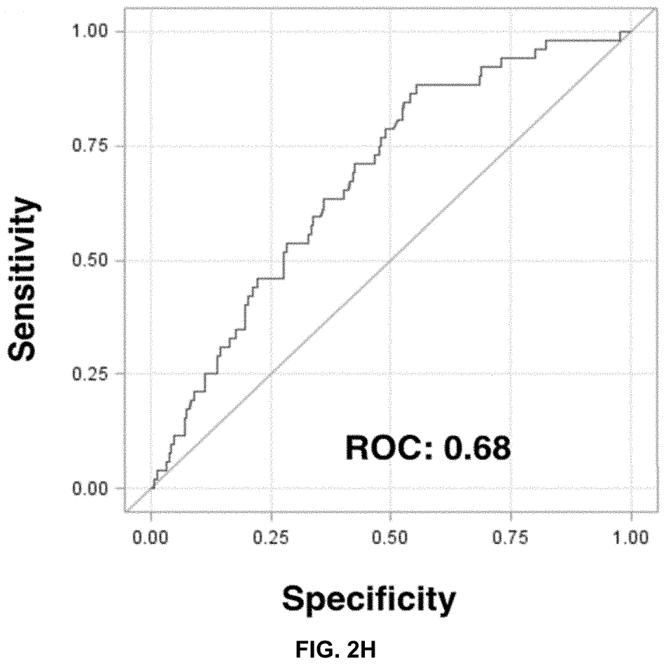

The association of the I PR score and 10-year tooth loss (≥3 teeth) in the DARIC dataset is illustrated in FIG. 2A. The validation of IPR as a predictor of tooth loss was done using PDS 5-year tooth loss (≥3 teeth) data and is shown in FIG. 2C. For example, the predicted probability of tooth loss of IPR of 20, based on the DARIC dataset is 19%. The observed probability in the PDS dataset for an IPC of 20 was 23%. The predicted probability of disease progression in 3 years and incident edentulism using the PDS dataset appear in FIGS. 2E and 2G, respectively. Similarly to the risk for tooth loss, the IPR score is positively associated with periodontitis progression and edentulism. The area under the ROC for all predicted estimates are shown in FIGS. 2A-2H. Both DARIC and PDS predicted tooth loss area under the ROC was 0.72 (FIGS. 2B and 2D). The area under the ROC for attachment loss for the PDS dataset was 0.75 (FIG. 2F). A detailed presentation of predicted probabilities for tooth loss according to IPR scores can be found in Table 6.

TABLE 6
Detailed presentation of the predicted probability for 10-year
risk for tooth loss (≥3 teeth) for the DARIC dataset and 5-
year tooth loss (≥3 teeth) for the Piedmont (PDS) dataset stratified
by the Index of Periodontal Risk (IPR) score. Actual probability
for the 10-year tooth loss (≥3 teeth) for the DARIC dataset.

| | Risk Tooth | Risk Tooth | Actual Probability |
|---|---|---|---|
| IPR | Loss (%) | Loss (%) | (%) of Tooth Loss |
| Score | DARIC | PDS | (5-IPR Running Average) |
| 5 | 6 | 8 | |
| 6 | 6 | 9 | |
| 7 | 7 | 10 | 4.3 |
| 8 | 7 | 10 | 4.5 |
| 9 | 8 | 11 | 4.3 |
| 10 | 9 | 12 | 7.50 |
| 11 | 9 | 13 | 12.7 |
| 12 | 10 | 14 | 18.1 |
| 13 | 11 | 14 | 16.6 |
| 14 | 12 | 15 | 15.2 |
| 15 | 13 | 17 | 18.1 |
| 16 | 14 | 18 | 17.0 |
| 17 | 15 | 19 | 15.6 |
| 18 | 16 | 20 | 20.3 |
| 19 | 18 | 22 | 20.0 |
| 20 | 19 | 23 | 25.0 |
| 21 | 21 | 24 | 26.7 |
| 22 | 22 | 26 | 32.0 |
| 23 | 24 | 27 | 33.3 |
| 24 | 25 | 29 | 31.2 |
| 25 | 27 | 31 | 34.6 |
| 26 | 29 | 32 | 31.6 |
| 27 | 31 | 34 | 35.7 |
| 28 | 33 | 36 | 33.8 |
| 29 | 35 | 38 | 36.8 |
| 30 | 37 | 40 | 39.1 |
| 31 | 39 | 41 | 39.4 |
| 32 | 41 | 43 | 47.2 |
| 33 | 44 | 45 | 45.0 |
| 34 | 46 | 48 | 48.4 |
| 35 | 48 | 50 | 48.1 |
| 36 | 50 | 51 | 50.0 |
| 37 | 52 | 53 | 54.3 |
| 38 | 55 | 55 | 48.6 |
| 39 | 57 | 57 | 53.1 |
| 40 | 60 | 60 | 41.1 |
| 41 | 62 | 61 | 47.2 |
| 42 | 64 | 63 | 54.0 |
| 43 | 66 | 65 | 56.6 |
| 44 | 68 | 67 | 62.9 |
| 45 | 70 | 68 | 58.3 |

Risk Models for Tooth Loss and Periodontal Disease Progression by Index of Periodontal Classes (IPC). Tooth loss and disease progression risk estimates based on classes developed from specific IPR cut-points are presented in Table 7. Rather than defining these from percentiles (e.g., quartiles) of IPR values, which are distribution-based, we used CART methods²³ to select optimal cut-points to establish 3 levels or classes of risk for tooth loss: IPC-“Low” (0-10), IPC-“Moderate” (10-20), and IPC-“High” (>20). The DARIC dataset demonstrated a significant higher RR (CI) for tooth loss for both “Moderate” RR=2.4 (95% CI=1.8-3.1) and “High” RR=5.6 (95% CI=3.5-5.9) relative to “Low”. Similar estimates were found in the PDS dataset. Using IPC-“Low” as a reference, IPC-“Moderate” showed an almost 200% increased risk (RR=2.9; 95% CI=0.9-9.6) for tooth loss, while IPC-“High” had RR=5.8 (95% CI=1.8-18.3). Estimates of attachment loss of ≥3 mm risk in the PDS dataset were low for IPC-“Moderate”: RR=1.1 (95% CI=0.3-3.5) and substantially higher for IPC-“High”: RR=3.7 (95% CI=1.3-10.5). Attachment loss estimates were not calculated in DARIC because there are no available longitudinal data for attachment loss. Of note, exploratory adjustment for dental caries did not influence the risk estimates obtained neither tooth loss nor periodontal disease progression (i.e., attachment loss).

TABLE 7
Relative Risk and 95% Confidence Interval (CI) for losing ≥3 teeth
over a 5-year period and 10% of sites with ≥3 mm Attachment Loss
(ALoss) increase (n = 363) over a 3-year period for the PDS
(Piedmont Dental Study) dataset and for 10 Year Tooth Loss (≥3
teeth) for the DARIC dataset stratified by Index of Profile Classes (IPC)

| PDS Dataset | Tooth Loss | Attachment Loss |
|---|---|---|
| IPC Low (0-10) | Ref | Ref |
| IPC Moderate (11-20) | 2.88 (0.86-9.62) | 1.05 (0.32-3.46) |
| IPC High (>20) | 5.79 (1.83-18.3) | 3.71 (1.31-10.5) |
| DARIC Dataset | Tooth Loss (≥3 teeth) | |
| IPC Low (0-10) | Ref | |
| IPC Moderate (11-20) | 2.36 (1.81-3.07) | |
| IPC High (>20) | 5.55 (3.50-5.92) | |

Discussion

This Example presents, inter alia, the development and application of the Index of Periodontal Risk score (IPR), as a means to inform precise periodontal disease risk assessment, prevention and therapy. The IPR is based upon the novel patient stratification system for periodontal disease classification, the University of North Carolina Periodontal and Tooth Profile Class (UNC-PPC/TPC) system, to provide summary estimates for tooth loss risk and periodontitis progression. This study used two independent population-based cohort samples with over 4,500 participants and demonstrated that the IPR and its derived classes (IPC-Low, Moderate and Severe) offer substantial gains in precise classification and accurate estimation of prospectively-assessed disease endpoints, such as tooth loss and disease progression, compared to existing taxonomies of disease.

There are several strategic advantages of the proposed UNC-PPC/TPC classification that were previously described by our group.¹³ The IPR was generated based on specific PCC/TPC classifications using the DARIC 10-year tooth loss data. For example, in FIG. 1 shows that a severe disease tooth (TPC-G) has a probability of 6.7% for tooth loss when found in PPC-A (Health) individual. This probability increases to 45.8% in a PPC-G (Severe Disease) individual. The IPR simply computes a composite probability based upon the individual's PPC category and TPC values as determined by the specific clinical status of the teeth present. In this study, the application of the IPR score was demonstrated in a manner that offers quantitative assessments for attachment and tooth loss risk rather than broadly-defined, overlapping categories of prognostic terms, such as good, fair, questionable, unfavorable, poor or hopeless. A strength of this study is that this risk tool was developed using the DARIC cohort of 3,985 individuals and validated using the PDS longitudinal study (n=697) representing a total of 4,682 individuals. The validation in the PDS dataset was important as it had a different composition of age, sex, race/ethnicity. The IPR score also accommodates the well-established risk factors, smoking, diabetes, age, sex, race/ethnicity, into the risk models with effect sizes that are comparable to previous publications.^{6, 8, 24} Additionally, this method can be extended to make specific risk inferences at the tooth type level.

This Example can demonstrate that the UNC-PPC system classification enables a more detailed and precise stratification for risk assessment and clinical outcome prediction than the CDC/AAP classification. Admittedly, the CDC/AAP classification was developed for epidemiologic surveys rather than risk assessment, but it has been widely used for this purpose. The AAP classification is based upon presence of attachment/bone loss that reflects history of disease²⁵, which is relatively insensitive to changes in person-level factors, tooth loss or disease activity, but are widely used in healthcare settings. The PPC classification system is similarly insensitive to change over time, either in response to treatment or in the natural history of disease. However, treatment or disease progression does influence the TPC values and therefore can modify the IPR score. This characteristic makes the IPR score useful for clinicians as means for monitoring and illustrating individual patients' disease and specific outcome (e.g., tooth loss) propensity. Once treatment is provided, the IPR score can change at it relates directly to risk for attachment and tooth loss. This Example describes, inter alia, a methodology that can provide a more valid outcome measure and of greater utility than, for example, changes in mean probing depths and bleeding scores. The utility of the IPR score as a measure of clinical outcomes in response to therapy will be explored and presented in future publications. The IPR score incorporates a large set of tooth-specific periodontal clinical indicators, but also includes information on tooth-specific coronal and root caries, that appears to result in excess risk for tooth loss. Nevertheless, exclusion of caries scores did not change the obtained risk estimates for attachment loss.

Previous investigation demonstrated that the LCA model grouped individuals into separate clinical phenotypes that would be collapsed (or hidden) under the CDC/AAP classification.¹³ For example, 46% of individuals in PPC-D (“Tooth Loss”) were classified with moderate periodontal disease (CDC/AAP). The PPC-D class has sites with periodontal disease, but also these individuals have lost about half of their dentition (mean 16.8 teeth present). Nonetheless, here we demonstrated (Table 1) that the adjusted relative risk for ≥3 losing teeth among individuals in that group was 3.8, more than double compared to the CDC/AAP moderate level of disease category (RR=1.7). This is an emphatic demonstration of the gains in precise risk assessment that can be achieved by defining periodontal disease classes that accommodate missing teeth.

The Piedmont Dental Study included tooth status assessment over time that enabled us to measure rates of tooth loss and periodontitis progression as shown in Tables 2 and 3. We defined periodontitis progression as a minimal of 10% of sites with 3mm of attachment loss within 3 years. This is a very stringent definition of disease progression. We found that PPC-G (“Severe Disease”) had a very high relative risk (RR=7.8) to experience periodontitis progression compared to periodontally healthy individuals. Also, PPC-D demonstrated a significant higher adjusted RR for periodontitis progression. Considering that the majority of PPC-D (“Tooth Loss”) individuals were classified into the mild/moderate CDC/AAP disease category¹³, a large proportion of individuals would be unable to receive appropriate preventive care due to the underestimation of risk attributed to the CDC/AAP classification. Interestingly, we found that TPC-C (“Crown”) assignment was protective against attachment loss and had a similar, yet non-significant tooth loss prevention effect (Table 3). This is likely due to the TPC clustering of these teeth within individuals who had and are willing to spend disposable income for restorative dental care.

The predicted values for tooth loss based upon the composite IPR score by individuals in the DARIC dataset is closely reproduced in the PDS dataset. The predicted probabilities of attachment loss for the PDS dataset also demonstrate a similar pattern. For attachment and tooth loss the ROC reflected in the C-statistics were above 70%, which is considered strong for a single clinical score.²¹ The key to the utility of IPC is based upon the incorporation of the longitudinal data in the 7×7 table (FIG. 1). The overall precision of the model and the granularity at a tooth-level of establishing prognosis could be improved with the addition of more prospective clinical data that could refine the scoring. This method can fulfill the requirement of generating a “learning algorithm” that could improve prediction with more data. For example, with more individuals in the dataset the risk for tooth and attachment loss for a second maxillary molar in a specific PPC class individual could be quantified.

In summary, this Example can demonstrate the clinical application and utility of the UNC periodontal and tooth profile class (UNC-PPC/TPC) system, as well as its derived risk score and risk classes (I PR/I PC), for patient stratification, risk assessment and personalized outcome propensity estimation. This newly developed system, upon additional validation can inform and improve patient care decisions and outcomes, consistent with the vision of precision periodontal medicine.

### REFERENCES

1. Collins F S, Varmus H. A new initiative on precision medicine. N Engl J Med 2015; 372:793-795.

2. Voros S, Maurovich-Horvat P, Marvasty I B, et al. Precision phenotyping, panomics, and system-level bioinformatics to delineate complex biologies of atherosclerosis: rationale and design of the “Genetic Loci and the Burden of Atherosclerotic Lesions” study. J Cardiovasc Comput Tomogr 2014; 8:442-451.

3. Kusiak J W, Somerman M. Data science at the National Institute of Dental and Craniofacial Research: Changing dental practice. J Am Dent Assoc 2016; 147:597-599.

4. Sankar P L, Parker L S. The Precision Medicine Initiative's All of Us Research Program: an agenda for research on its ethical, legal, and social issues. Genet Med 2016.

5. Offenbacher S, Divaris K, Barros S P, et al. Genome-wide association study of biologically informed periodontal complex traits offers novel insights into the genetic basis of periodontal disease. Hum Mol Genet 2016; 25:2113-2129.

6. Lang N P, Tonetti M S. Periodontal risk assessment (PRA) for patients in supportive periodontal therapy (SPT). Oral Health Prey Dent 2003; 1:7-16.

7. Chandra R V. Evaluation of a novel periodontal risk assessment model in patients presenting for dental care. Oral Health Prey Dent 2007; 5:39-48.

8. Page R C, Krall E A, Martin J, Mancl L, Garcia R I. Validity and accuracy of a risk calculator in predicting periodontal disease. J Am Dent Assoc 2002; 133:569-576.

9. Trombelli L, Farina R, Ferrari S, Pasetti P, Calura G. Comparison between two methods for periodontal risk assessment. Minerva Stomatol 2009; 58:277-287.

10. Busby M, Chapple L, Matthews R, Burke F J, Chapple I. Continuing development of an oral health score for clinical audit. Br Dent J 2014; 216:E20.

11. Lindskog S, Blomlof J, Persson I, et al. Validation of an algorithm for chronic periodontitis risk assessment and prognostication: analysis of an inflammatory reactivity test and selected risk predictors. Journal of periodontology 2010; 81:837-847.

12. Lang N P, Suvan J E, Tonetti M S. Risk factor assessment tools for the prevention of periodontitis progression a systematic review. J Clin Periodontol 2015; 42 Suppl 16:S59-70.

13. Morelli T, Moss K L, Beck J, et al. Derivation and Validation of the Periodontal and Tooth Profile Classification System for Patient Stratification. Journal of periodontology 2017; 88:153-165.

14. Beck J D, Elter J R, Heiss G, Couper D, Mauriello S M, Offenbacher S. Relationship of periodontal disease to carotid artery intima-media wall thickness: the atherosclerosis risk in communities (ARIC) study. Arterioscler Thromb Vasc Biol 2001; 21:1816-1822.

15. Naorungroj S S G, Divaris K, Beck J D, Heiss G, Offenbacher S. Predictors of 10-year incident tooth loss: the Dental ARIC Study. J Public Health Dent In Press.

16. Beck J D, Sharp T, Koch G G, Offenbacher S. A study of attachment loss patterns in survivor teeth at 18 months, 36 months and 5 years in community-dwelling older adults. J Periodontal Res 1997; 32:497-505.

17. Beck J D, Koch G G, Rozier R G, Tudor G E. Prevalence and risk indicators for periodontal attachment loss in a population of older community-dwelling blacks and whites. Journal of periodontology 1990; 61:521-528.

18. Beck J D, Koch G G, Offenbacher S. Attachment loss trends over 3 years in community-dwelling older adults. Journal of periodontology 1994; 65:737-743.

19. Eke P I, Page R C, Wei L, Thornton-Evans G, Genco R J. Update of the case definitions for population-based surveillance of periodontitis. Journal of periodontology 2012; 83:1449-1454.

20. Griner P F, Mayewski R J, Mushlin Al, Greenland P. Selection and interpretation of diagnostic tests and procedures. Principles and applications. Ann Intern Med 1981; 94:557-592.

21. Hosmer D W LS. Applied Logistic Regression. New York, N.Y.: John Wley & Sons; 2000.

22. Schwarz G. Estimating the Dimension of a Model. 1978:461-464.

23. Breiman L F, J. H.; Olshen, R. A.; Stone, C. J. Classification and regression trees. Monterey, Calif.: Wadsworth & Brooks/Cole Advanced Books & Software; 1984.

24. Giannobile W V, Braun T M, Caplis A K, Doucette-Stamm L, Duff G W, Kornman K S. Patient stratification for preventive care in dentistry. Journal of dental research 2013; 92:694-701.

25. Armitage G C. Development of a classification system for periodontal diseases and conditions. Ann Periodontol 1999; 4:1-6.

### EXAMPLE 2

Introduction

Precise stratification is an important and highly desirable goal, from both clinical and public health standpoints. In the oral health domain, accurate stratification has the promise of optimizing diagnoses, treatment decisions, and overall care. For example, estimating tooth loss propensities at the individual and tooth levels can be highly informative for planning personalized, risk-based care.

Clustering methods based upon principal component analyses have been widely employed to identify microbial community structures and a combination of clinical signs that describe characteristics of the population.¹⁻³ However, most traditional clustering techniques neither categorize individuals to enable person-specific predictions, nor are they sensitive to change in status over time. Most existing models use person-level summary variables of clinical parameters, such as mean or extent scores for various signs of disease including plaque scores, gingival indices, probing depths, and clinical attachment levels, that reflect person-level disease and are not always linked to tooth type or tooth loss patterns. Other classifications are minimalist in nature seeking the fewest number of sites or probing measures to place individuals into mutually exclusive categories of disease status.^{4,5}

Latent class analysis (LCA) is a statistical method used to identify a set of discrete, mutually exclusive latent classes of individuals based on their responses to a set of observed categorical variables.⁶ It is a data-driven, person-centered approach that considers heterogeneity among individuals that can be grouped into relatively homogeneous subclasses with similar clinical patterns or trait endorsements.^{7, 8} LCA can also be used to explore the association between a set of observed categorical variables through assumed unobserved, latent classes. Researchers in numerous areas have been increasingly using LCA to discover hidden (latent) classes of individuals including the behavioral sciences^{9, 10}, autism¹¹, HIV infection¹², and asthma¹³. LCA has not been used before to derive periodontal or tooth profile classes.

This Example describes the development of analytical procedures for implementing person-level LCA to identify discrete classes of individuals that are discriminated by tooth-level clinical parameters. A tooth-level LCA was also applied to discriminate different classes of teeth using tooth/site level clinical parameters. Finally, the resulting estimates were applied as model parameters to systematically examine other large randomly sampled populations to ascertain whether tooth-based clinical parameters could effectively segregate different clinical periodontal classes, even in the presence of incomplete data. This Example describes the derivation and validation of the LCA classes.

Materials and Methods

Analytical Approach for Classification of Subjects into Subgroups. The analytical approach implemented person-level LCA to identify discrete classes of individuals was based upon 7 tooth-level clinical parameters, including: ≥1 site with interproximal attachment level (IAL)24 3 mm, ≥1 site with probing depth (PD)≥4 mm, extent of bleeding on probing (BOP, dichotomized at 50% or ≥3 sites per tooth), gingival inflammation index¹⁴ (GI, dichotomized as GI=0 vs. GI≥1), plaque index¹⁵ (PI, dichotomized as PI=0 vs. PI≥1), the presence/absence of full prosthetic crowns for each tooth, and tooth status presence (present vs. absent). The Dental Atherosclerosis Risk in Community Study (DARIC) cohort (n=6793)¹⁶ was used and applied the resulting estimates as model parameters to systematically examine other large-sample populations to ascertain whether tooth-based clinical parameters associated with baseline status could effectively discriminate between different clinical periodontal classes, even in the presence of incomplete data.

Individuals were classified into mutually exclusive latent classes based on their responses to a set of observed categorical variables. Criteria used to determine the optimal number of classes included the Akaike Information Criterion (AIC) and the Bayesian Information Criterion (BIC), while ensuring that clinically relevant categories were maintained. Milligan and Cooper's²³ recommendation for the maximum number (n) of classes was used, suggesting to stop when the newly-added class (n+1) is not clinically distinct from the previous number (n) of identified classes. Additionally, it was verified that mean posterior probabilities of correct class assignment were >0.7, which according to Nagin²⁴ indicates adequate class separation and membership precision. In the first step of LCA, the person-level LCA was used to classify individuals into seven latent classes based on 224 dichotomous variables (derived from 7 tooth-level variables, using the clinical parameters referred to above for each of 32 teeth). The class membership probabilities represent the overall, unconditional proportions of individuals in each of seven latent classes. The model parameters from the first step were then used to compute the posterior probabilities (the probability of event A occurring given that event B has occurred) of each individual's membership into each class conditional upon the values of the 224 items, or as many of them as were observed for that individual.

Recognizing that individuals with periodontal disease have teeth with diagnoses ranging from health to severe disease, we carried out a tooth-level LCA analysis to capture the distribution of these tooth-specific classes within each person-level subgroup. This tooth-level analysis enabled us to refine the individual tooth status at a person-level within each Periodontal Profile Class (PPC) for risk assessment modeling. The tooth-level LCA classified teeth into 7 latent Tooth Profile Classes (TPC), based on 14 categorical clinical parameters similar to those referenced above. These 14 clinical parameters included IAL (<3 mm=0, ≥1 site with 3 or 4 mm=1, and ≥5 mm=2), direct attachment level [DAL, measured at direct buccal and lingual (<3 mm=0, ≥1 site with 3 or 4 mm=1, and ≥5 mm=2)], interproximal PD (<4 mm=0, ≥1 site with 4 or 5 mm=1, and ≥6 mm=2), direct PD (<4 mm=0, ≥1 site with 4 or 5 mm=1, and ≥6 mm=2), interproximal gingival recession (IGR, dichotomized as IGR≤1 vs. IGR>1), direct GR (measured at direct buccal and lingual, dichotomized as DGR≤1 vs. DGR>1), BOP (dichotomized at <3 vs. ≥3 sites per tooth), GI¹⁴ (dichotomized as GI=0 vs. GI≥1), PI ¹⁵ (dichotomized as PI=0 vs. PI≥1), decayed coronal surface (DCS, dichotomized as DCS=0 vs. DCS≥1), filled coronal surface (FCS, dichotomized as FCS=0 vs. FCS≥1), decayed root surface (DRS, dichotomized as DRS=0 vs. DRS≥1), filled root surface (FRS, dichotomized FRS=0 vs. FRS≥1), and the presence/absence of full prosthetic crowns. These steps were carried out using the SAS PROC LCA procedure^{#}. ⁶

The LCA model parameter estimates obtained from DARIC were used to estimate the posterior class membership probabilities of three additional populations. This process involved the creation of a novel scoring algorithm that directly computed the likelihood of each class membership (using the posterior probabilities). The scoring code creates what we are referring to as the University of North Carolina (UNC) Periodontal and Tooth Profile Classes (PPC/TPC). The underlying statistical model and handling of missing data are presented in some detail in the supplemental methods. In brief, for all examined populations, an individual was classified into the latent class for which he/she had the corresponding largest posterior membership probability. As a measure of the quality of the classification assignments, the percentage of individuals with the largest class membership probability exceeding a certain threshold was determined for each study population.

Study Populations. All participants provided written informed consent to a protocol that was reviewed and approved by the Institutional Review Board on research involving human subjects at the University of North Carolina and/or at each study performance site.

DARIC participants were recruited from the ARIC population study and included dentate participants who did not have contraindications for periodontal probing.¹⁶ The DARIC sample consisted of 6,793 individuals living in four United States communities. These subjects had full-mouth periodontal examinations at six sites per tooth, including third molars, as measured by trained and calibrated examiners.

Two additional datasets from the National Health and Nutrition Examination Survey (NHANES; 2009-2010 and 2011-2012) were used as the second study population. The technical details of the surveys, including sampling design, periodontal data collection protocols, and data availability, have been described elsewhere.^{17, 18} Briefly, periodontal measurements were collected for 3,750 individuals (NHANES 2009-2010) and for 3,338 individuals (NHANES 2011-2012). The third study population was from the Piedmont 65+ Dental Study (PDS), which was based on a stratified random clustered sample of all people aged 65 and over in the five adjacent counties in the Piedmont area of North Carolina.¹⁹ The PDS began in 1988 with a random subsample of 697 dentate individuals with periodontal data available. Although PDS is a longitudinal study, in this report these analysis were conducted using the baseline data. Additional population characteristics are described in detail in previous publications.^{20, 21}

Statistical Analyses for Comparison of Latent Class Subgroups within Populations. The seven latent classes were compared with respect to participants' demographic characteristics in the DARIC population, which facilitated their labeling with monikers that briefly summarize the clinical impression of each class. Pearson chi-square tests were used to test for overall differences in the seven classes with respect to these characteristics and one-way ANOVA F-tests were used to test for differences with respect to periodontal variables. A conventional p<0.05 statistical significance criterion was used for all analyses.

Additional analyses compared periodontal status across the seven classes for each of the three validation datasets with class membership derived from the LCA model developed from the DARIC data. Sensitivity analyses with the DARIC dataset were conducted to assess the utility and performance of the LCA model for assigning members into the seven PPCs when a periodontal measure was entirely missing (e.g., data not collected). Using DARIC as the gold standard when all seven periodontal indices were available for analysis, the average posterior probabilities were calculated for each of the seven person-level and tooth-level LCA classes. The average posterior probabilities were calculated within each of the seven indices omitted singly from the DARIC dataset.

Results

Periodontal and Tooth Profile Classes Derived from Tooth Level Clinical Parameters. The person-level LCA procedure enabled us to select 7 PPCs (A-G), in the DARIC population with distinct clinical phenotypes. The demographic characteristics for the 7 PPCs labeled A-G with class clinical monikers are shown in Table 8. There were significant group differences with regard to race, sex, age, diabetes, smoking (history and pack/year), obesity, access to dental care, socio-economic status, and educational level. In general, the demographics followed expected patterns with regards to the clinical phenotypes. The clinical periodontal phenotype as defined by the 7 PPCs compared to the 4-level Center for Disease Control/American Academy of Periodontology (CDC/AAP) definitions is shown in Table 8 and illustrates the differences in clinical presentation comparing the two classifications. For example, 45% of the CDC/AAP healthy individuals fall under the PPC-A (Healthy) class. While 29% of these CDC/AAP healthy individuals are assigned to the PPC-F (Severe Tooth Loss) class. For the CDC/AAP severe classification 32% and 26% are in PPC-E (Posterior Disease) and PPC-G (Severe), respectively.

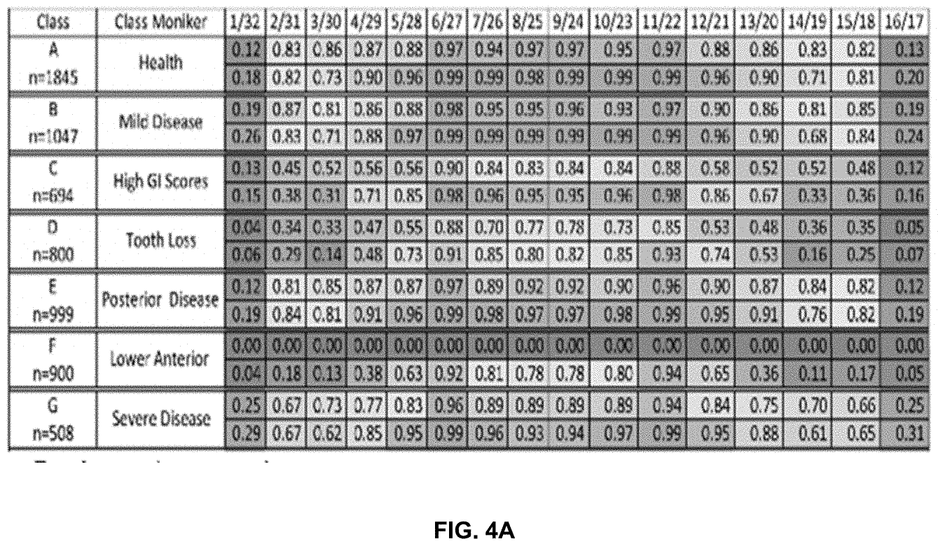

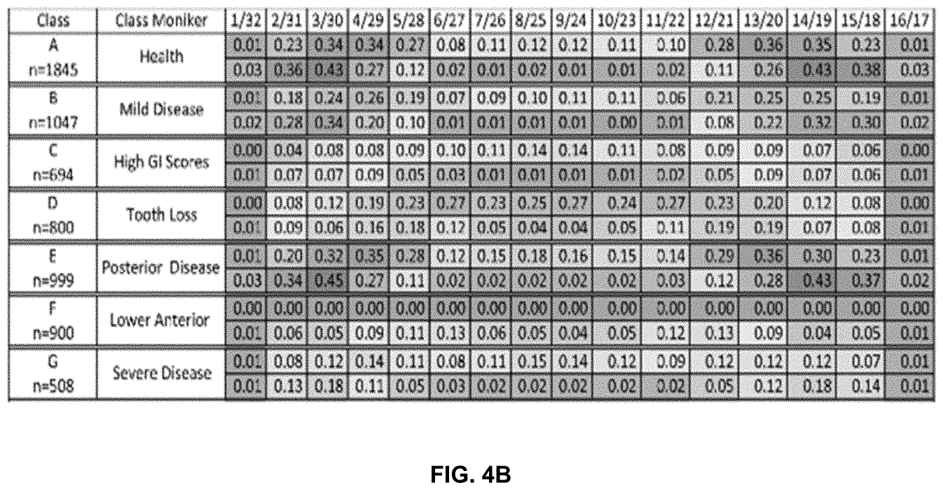

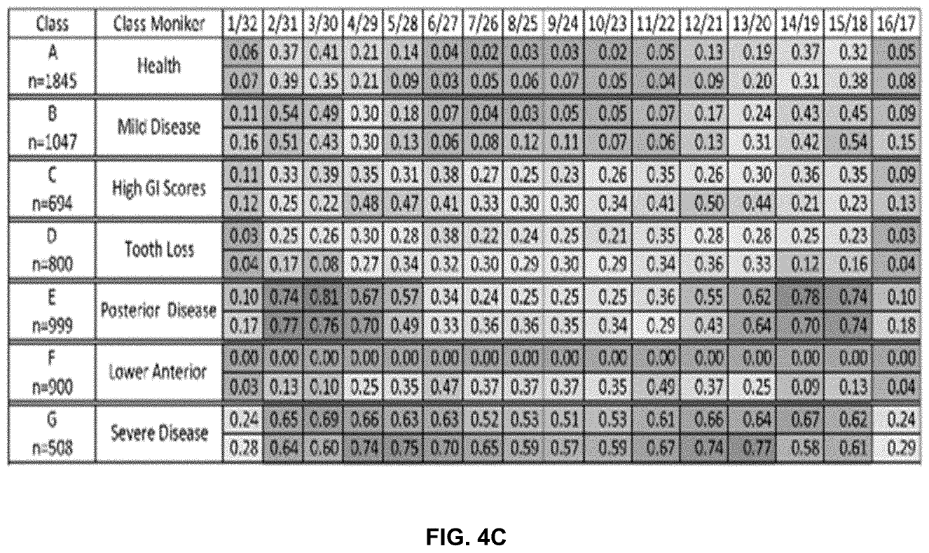

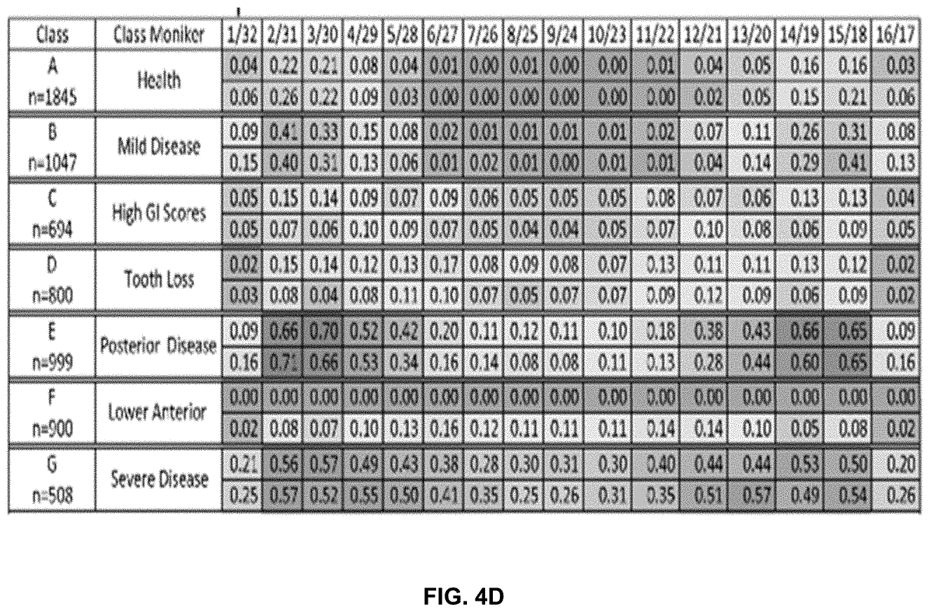

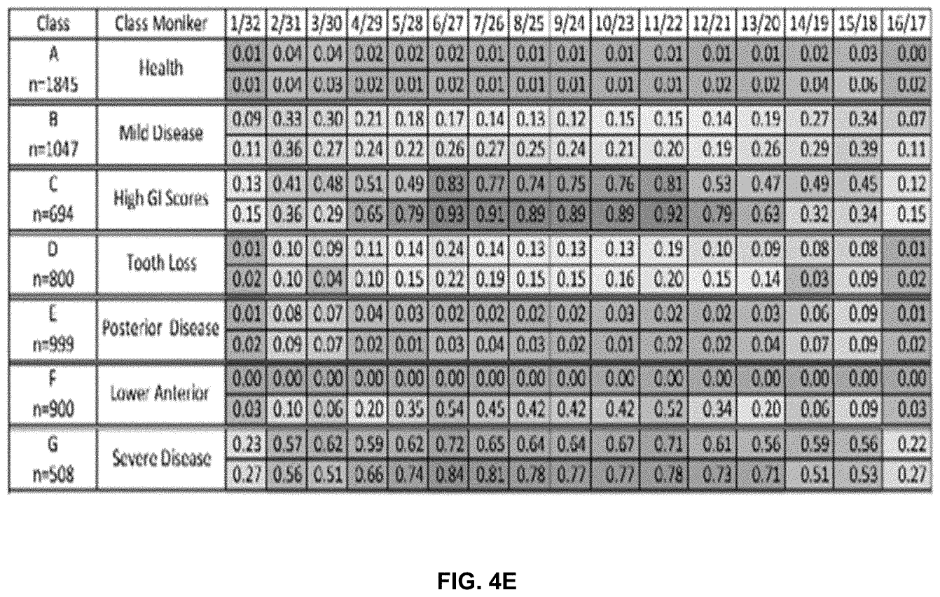

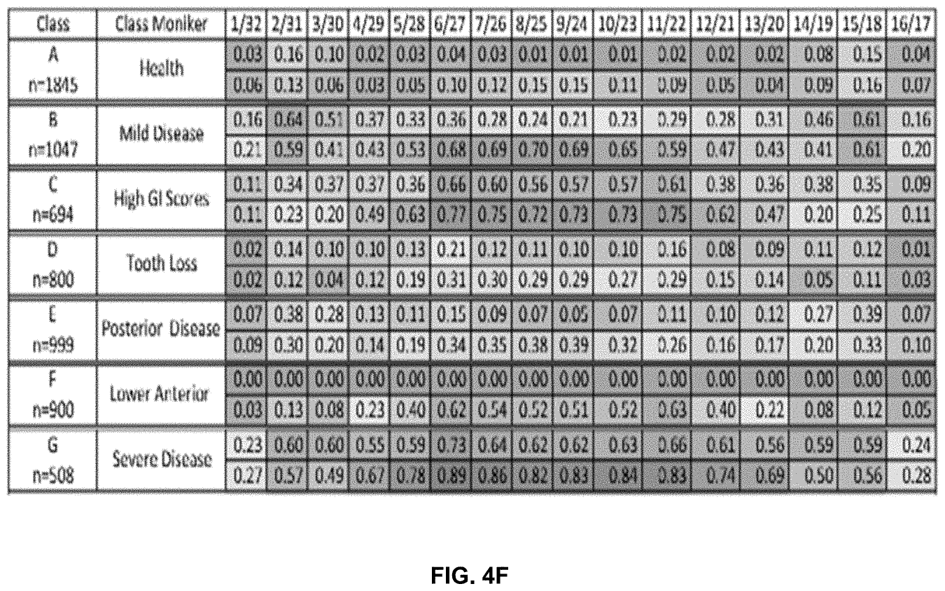

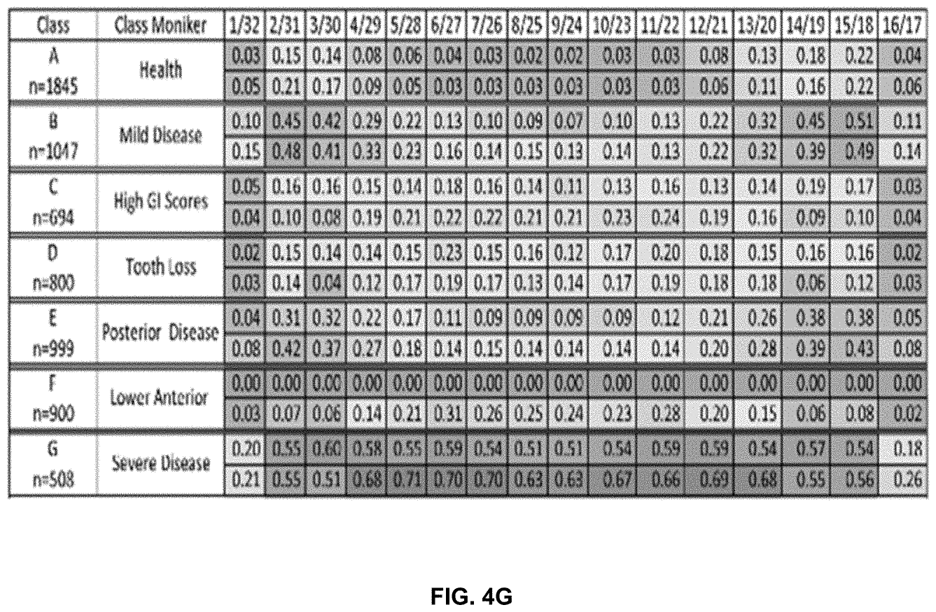

The underlying differences in the PPC classifications based upon the seven clinical measures for all 32 teeth are illustrated in FIGS. 4A-4G. The posterior probabilities (1=present; 0=absence) for tooth presence vs. absence, crown presence vs. absence, IAL≥3 mm, PD≥4 mm, GI≥1, PI≥1, and higher BOP are shown for each tooth type (1-32) representing both arches graphically in a heatmap for each clinical parameter (FIGS. 4A-4G). In this figure, both the upper and lower arch are represented for each PPC for each tooth with green indicating high probability of tooth presence and healthy clinical signs with shifts to yellow and red indicating more disease-associated signs or tooth loss. For example, one can see that most teeth are present (except 3^{rd} molars) with healthy clinical signs in PPC-A (Health), whereas, only mandibular anterior teeth remain with disease in PPC-F (Severe Tooth Loss). Interestingly, the person-level LCA identified a high gingivitis/inflammation group; PPC-C. PPC-E (15% of individuals) displayed posterior disease reflected in probing depths and attachment loss with the most severe disease patterns in PPC-G (7% of individuals). It is readily apparent in this figure that there is marked symmetry in disease patterns, with significant differences between arches. Importantly, these clinical patterns of disease and tooth loss represent typical patterns of disease that clinicians observe and are entirely data-derived.

The description of clinical parameters for each PPC appears in Table 9. As expected, there were significant differences among all seven PPCs, and these values were provided for descriptive and comparative purposes. PPC-A (Health) had the lowest mean extent of BOP, GI≥1, and PI≥1. The mean extent of IAL≥3 mm of 8% and a mean extent of PD≥4 mm of 2% were the lowest among all 7 periodontal profile classes. PPC-B (Mild Disease) was mainly characterized by a slight increase in IAL≥3 mm and PD≥4 mm mean extent scores, and significant higher BOP (3-fold) and GI (9-fold) when compared to PPC-A. PPC-C (High GI) was notably marked by the highest mean extent GI score among all periodontal profile classes and was seen in 10% of the population. PPC-D (Tooth Loss) was characterized by fewer teeth. PPC-E (Posterior Disease) was marked by a moderate mean extent of IAL≥3 mm of 33% mainly located at the posterior dentition. PPC-F (Severe Tooth Loss) was characterized by the lowest mean number of teeth (8 teeth), where the remaining teeth were mainly mandibular anterior teeth with an edentulous maxilla and reflected 13% of the population. Finally, PPC-G (Severe Disease) was characterized by the highest mean extent of IAL≥3 mm of 54% and PD≥4 mm of 25%. Higher BOP, GI, and PI extent scores were also found in this generalized severe disease profile and was a more severe disease group than the CDC/AAP severe group (data not shown).

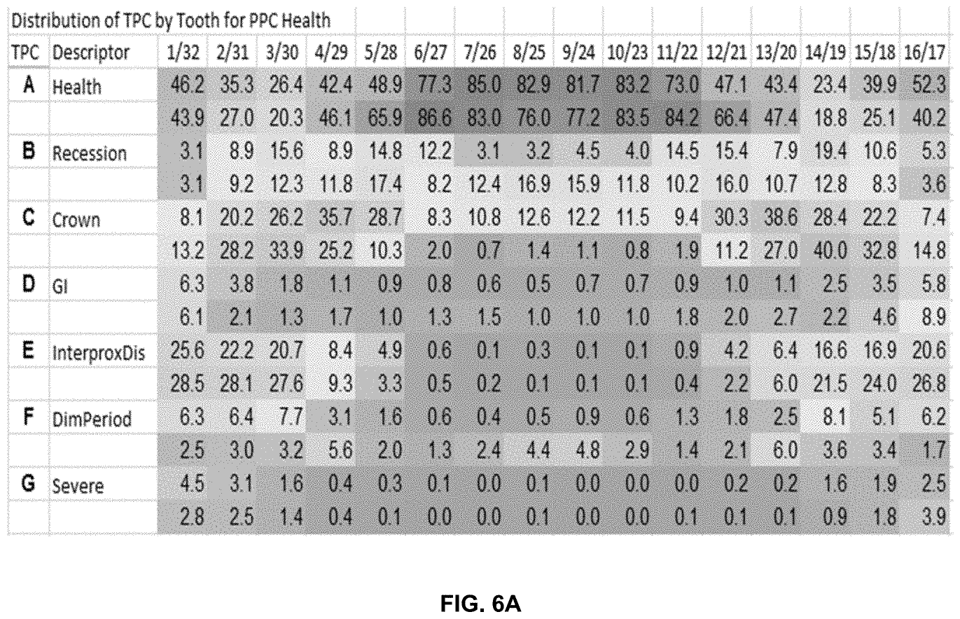

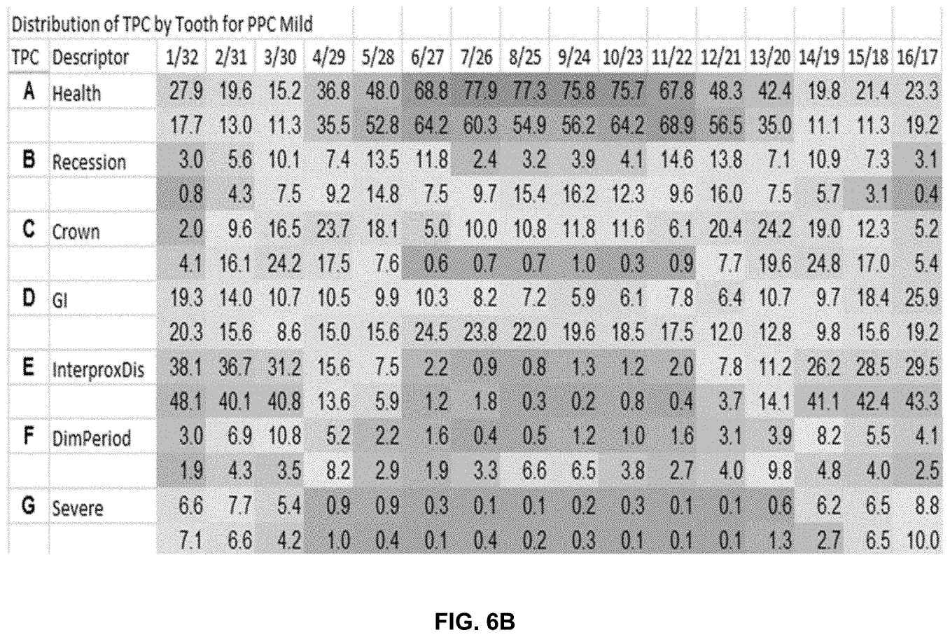

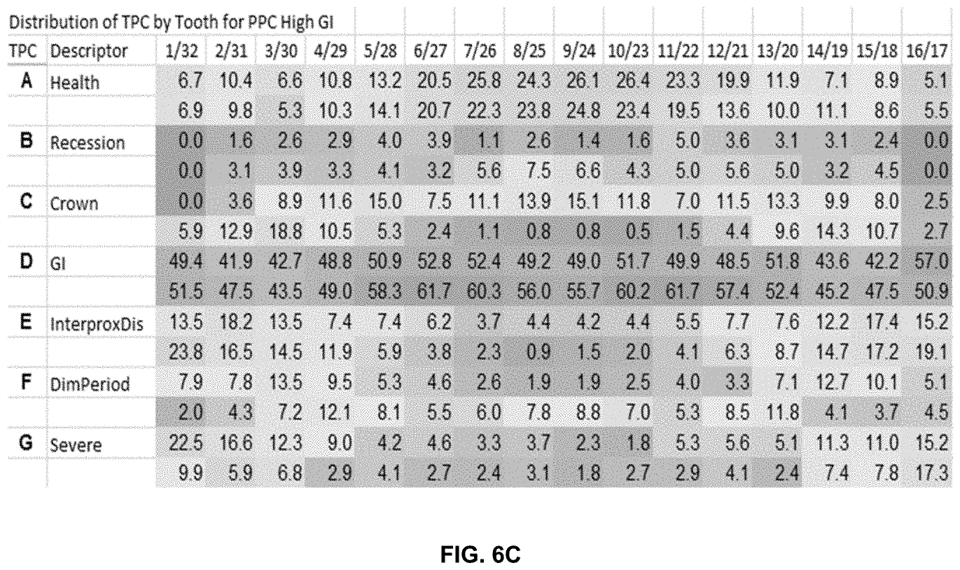

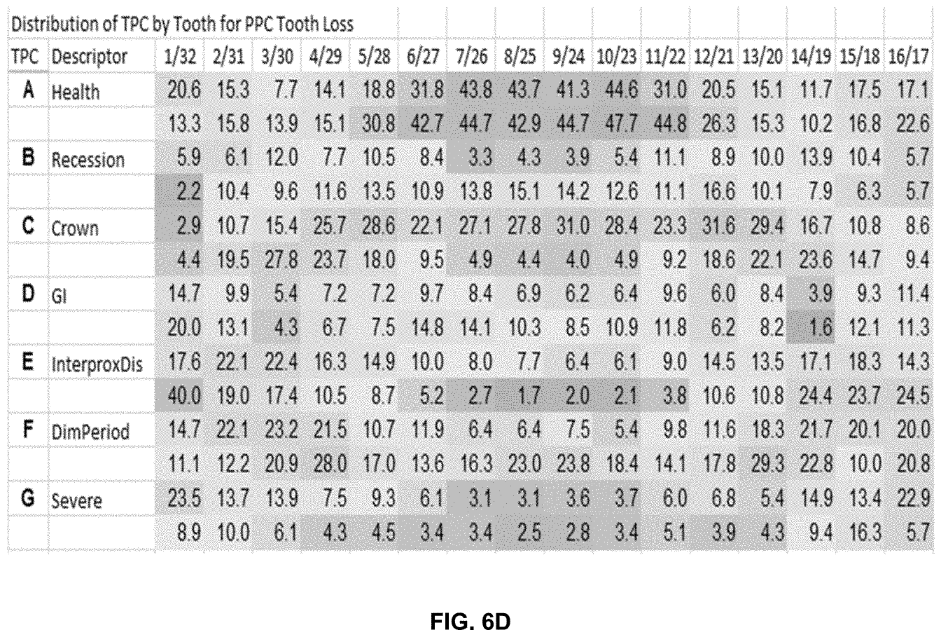

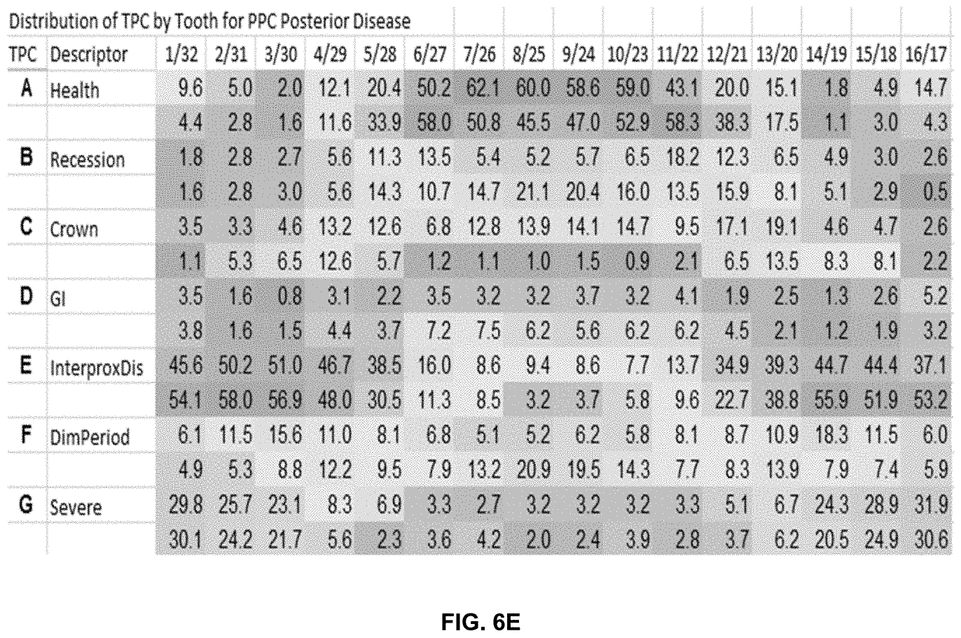

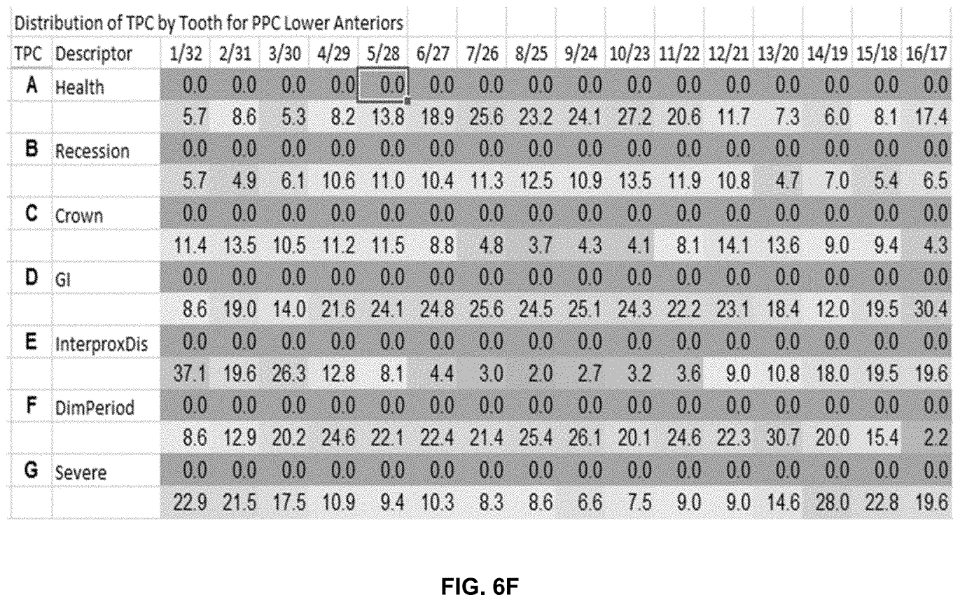

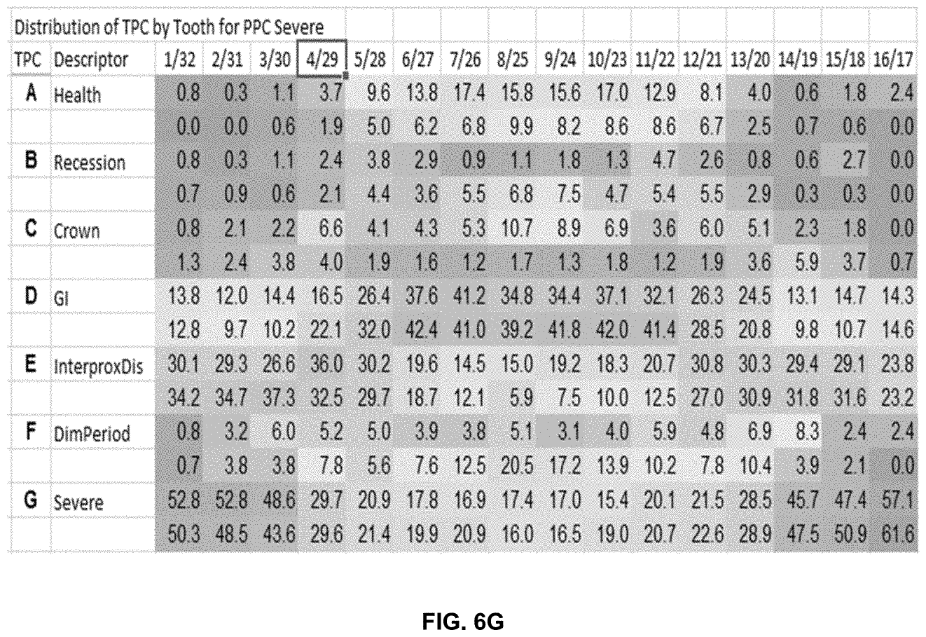

The tooth-level LCA procedure enabled us to identify 7 TPCs (A-G), in the DARIC population. The description of the 14 clinical parameters for each TPC is described in Table 12. As expected, there were significant differences among all seven TPCs, and these values are provided for descriptive and comparative purposes. For example, TPC-A included teeth with the least attachment loss, PD, BOP, recession, GI, PI, caries, and number of crowns. On the other hand, TPC-G included teeth with signs of periodontitis represented by substantial attachment loss, deep PD, high GI and PI. FIGS. 6A-6G shows the distribution of TPCs by tooth and arch for all PPCs. The percentage distribution is shown for each tooth type (1-32) representing both arches graphically in a heatmap for each PPC.

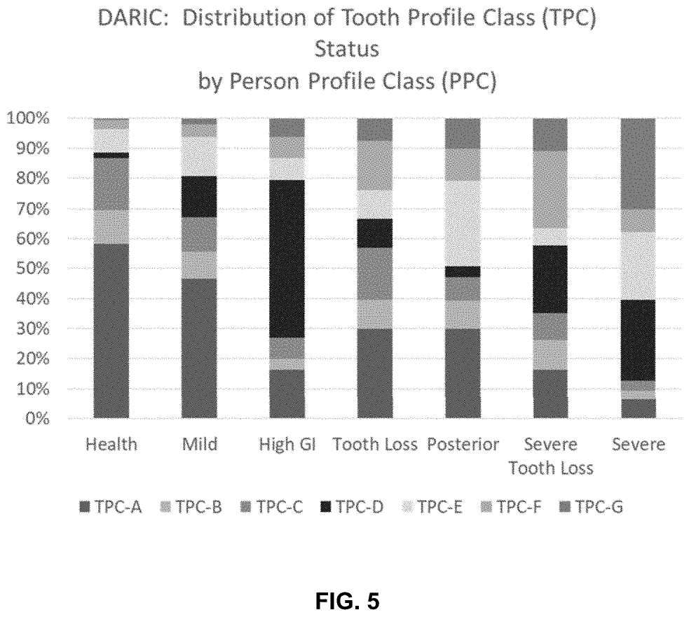

Joint Distribution of Periodontal and Tooth Profile Classes. FIG. 5 shows the distribution of the seven TPCs for each of the seven PPCs. PPC-A (Health) is composed by 59% of teeth classified as TPC-A (Health), 11% as TPC-B (Recession), 17% as TPC-C (Crown), 2% as TPC-D (GI), 8% as TPC-E (Interproximal Disease), 3% as TPC-F (Reduced Periodontium), and less than 1% as TPC-G (Severe). Moreover, PPC-C (High GI) is mostly comprised of TPC-D (GI) teeth (53%). As expected, PPC-G (Severe Disease) is mainly composed of teeth under the TPC-G (Severe) (28%), with the other major classes being TPC-D (GI; 28%) and TPC-E (Interproximal Disease; 23%).

Periodontal Profile Class ReplicationNalidation Among Different Populations. Table 10 presents the results of the person-level LCA DARIC-derived model as applied to or “scored” in the three external population-based cohorts including a total of 7,785 individuals; the NHANES 2009-2010, the NHANES 2011-2012, which are both nationally-representative samples and the PDS. There were remarkable similarities in frequency distributions between the 2 NHANES datasets; the prevalence of each PPC category was either identical or within 2 percentage points. As expected, the older, more diseased and edentate PDS individuals display more disease and higher PPC class assignments.

In contrast to the DARIC population, the PDS and NHANES population datasets did not include GI, PI, BOP, or number of prosthetic crowns. Despite a substantial amount of incomplete data relative to the full-mouth periodontal assessment, the person-level LCA model produced PPCs for each validation dataset with qualitatively similar profiles as in DARIC in terms of CDC/AAP and PPC classifications, extent IAL, extent PD and number of teeth. When indices were omitted singly from the DARIC dataset, the person-level LCA model was able to allocate members into the 7 distinct PPCs with minimal misclassification error, as shown in Table 13. For example, the lowest posterior probability of individual assignment when BOP is missing from the dataset was 0.96 (PPC-B). When GI was excluded from the dataset the lowest posterior probability of individual assignment was 0.95 (PPC-B and PPC-D). The average posterior probability for all classes considering up to four parameters missing is shown in Table 14. It can be observed that even with the lack of 4 clinical parameters, the lowest average posterior probability for correct class assignment was 0.90.

Mean Posterior Probabilities for Periodontal Profile and Tooth Profile Classes. Table 11 presents the mean posterior probabilities of assignment to each PPC or TPC. For example, the mean posterior probability for a person to be assigned into the PPC-A is 0.978 with a chance of 0.022 to be assigned in any other PPC. For all other PPCs the mean posterior probabilities for each person to be assigned in each PPC was extremely high, with PPC-B (Mild Disease) showing the lowest mean posterior probability of 0.96. For TPCs, the lowest mean posterior probability for each tooth to be assigned to a specific TPC was 0.823 (TPC-D). The highest mean posterior probability was 0.953 for TPC-B.

Discussion

This Example describes, inter alia, development and validation of a novel patient stratification system based upon the definition of periodontal and tooth profile classes. There are several strategic advantages of the proposed 7-class person-level LCA model that we are designating the University of North Carolina Periodontal Profile Class (UNC-PPC) classification. It includes tooth-level data on 7 clinical parameters (PD, IAL, BOP, GI, PI, missing teeth and crown restorations) with 7 PPCs that reflect typical tooth loss patterns and disease patterns that mirror what is seen by clinicians. The method does not use any a priori assumptions of disease patterns or characteristics to define disease states and is an agnostic approach to disease definition. For example, it does not require a certain number of teeth or sites with some predefined level of disease for class assignment. Furthermore, the algorithm can be applied robustly to other datasets or individuals for class assignment, even in the presence of partial exams (number of teeth and/or number of indices). In contrast to principal component analyses which define traits within a population²², the LCA method defines distinct categories of members (people or teeth) with previously “hidden” combinations of characteristics, to create mutually exclusive latent classes.

It is significant that this model was developed using the DARIC cohort of 6,793 individuals, but was validated using two cross-sectional NHANES populations and the PDS longitudinal study representing a total of 14,578 individuals. Surprisingly, the effects of partial mouth examinations or missing clinical parameters did not result in significant misclassification error. In contrast to the DARIC population, clinical examinations conducted in the NHANES and PDS studies did not collect data on PI, GI, BOP, number of prosthetic crowns, and third molars. However, additional analyses (Tables 13-14) demonstrated the proportion of individuals misclassified when one or more of the clinical parameters were missing was minimal. Thus, the method appears rather robust as it demonstrates a relative consistency on correctly assigning individuals into classes even with some clinical parameters is completely missing. This suggests that the mapping of existing datasets to these categories to create “harmonized” data could enable a robust disease classification for bioinformatics analytics that can correctly assign individuals into classes even with incomplete clinical data (Table 13).

Although seven distinct PPCs were selected for this classification, the LCA method enabled us to choose the number of classes in the final model. Seven distinct classes were selected that enabled the creation of clinically relevant categories, based on the recommendation of Milligan and Cooper²³, in that an additional eighth class was not clinically distinct from the an class among the existing seven-class model. In addition, the mean posterior probabilities achieved with both person- and tooth-level LCA provided extremely high probability of correct class assignment. The lowest mean posterior probability was 0.823 (TPC-D). According to Nagin, a mean posterior probability >0.7 indicates adequate separation and classification precision.²⁴

As shown in FIGS. 4A-4G, PPC-A was mainly associated with a healthy periodontal phenotype. PPC-B associated with a mild periodontal disease profile. PPC-C had predominantly individuals with mild pocketing and attachment loss but with much higher GI and plaque scores and higher (53%) of the gingivitis TPC-D teeth (FIG. 5). PPC-D predominantly comprised individuals with moderate periodontal disease associated with more missing teeth. PPC-E was characterized by severe molar disease primarily located on posterior teeth. PPC-F was marked by the presence of mainly anterior mandibular teeth to include scattered premolars and an edentulous maxilla. Classifying individuals into distinct classes that include tooth loss and disease patterns is novel to this classification schema. PPC-G was predominantly composed of individuals with generalized severe periodontal disease. Interestingly, the LCA model differentiated individuals into separate clinical phenotypes that would be collapsed under the CDC/AAP classification (Table 8) as well as other common clinical classifications. For example, the CDC/AAP moderate disease group is the largest disease group with approximately 42% of individuals. Table 8 shows that individuals with moderate disease (CDC/AAP) are distributed across all PPCs with approximately 20% following into health (PPC-A) and 6% into severe (PPC-G), suggesting that these individuals with moderate disease have other important hidden or latent characteristics beyond the clinical measures used to define the CDC/AAP moderate disease category (PD, clinical attachment level, and BOP). This means that the LCA-derived definition of periodontal profile classes enables a more detailed and precise stratification than the CDC/AAP classification.

The AAP classification is based upon the presence of attachment/bone loss which reflects history of disease⁴, as is the American Dental Association (ADA/AAP) classification²⁵—both of which are relatively insensitive to changes in individual status, tooth loss or disease activity, but are widely used in healthcare settings. LCA is an increasingly popular statistical modeling technique used to uncover heterogeneity in response patterns or clinical characteristics within a population. LCA usage is common in the social and behavioral sciences and unlike factor analysis, which groups correlated response items, it is a person-centered approach.²⁶ Recently, LCA has been used to analyze data related to HIV¹², mental disorders²⁷, and cancer.²⁸ Finite mixture models, such as LCA, present an opportunity to approach subgroup analysis from a different perspective. These statistical models are appropriate when one posits that a population is comprised of two or more underlying, latent subgroups defined by the intersection of numerous individual characteristics.²⁹ In other words, LCA is a useful tool for identifying a set of underlying subgroups of individuals based on the intersection of multiple observed clinical characteristics. Thus, in this application the LCA successfully classified subjects into 7 periodontal classes with distinct clinical manifestations versus the 3-4 categories associated with other classifications. Admittedly, the rationale underlying nosological strategies fall under two broad philosophies “lumpers vs. splitters”” and this method provides a data-derived splitting classification. It is our contention that this reduction in heterogeneity within each PPC will ultimately enable us to better assess risk, treatment outcomes and design better precision periodontal medicine therapies.

The advantages of the methods described in this Example include, inter alia, an improved stratification model developed on a large population-based sample and validated in three additional large population-based cohorts. Patient stratification based on person-level risk factors has recently been used to evaluate the outcomes of preventive care in dentistry.³⁰ Patient stratification aiming towards the development of personalized dentistry might be an important approach for improving preventive care. The PPC/TPC classifications can offer improvements for 1) combining or “harmonizing” clinical datasets from different studies, 2) developing risk models for attachment and tooth loss and 3) providing sensitive tools for measuring the effects of therapy among differing PPC, and perhaps at a TPC level. Although this Example focuses on older populations, (the mean age of the DARIC and the PDS populations were 62 and 73 years, respectively), nevertheless it appears to perform well among younger populations, as in the two NHANES samples (NHANES 2009-2010: mean age 51 years [range 30-80 years]; NHANES 2011-2012: mean age 52 years [range 30-80 years]). The algorithm could be easily and efficiently made available via a web-based application, and then widely available for analyses and patient class assignment.

This Example can demonstrate how multiple clinical characteristics can be used to identify clinically distinct periodontal and tooth profile classes. Overall, the UNC-PPC/TPC classification represents a novel application of the LCA methodology that is promising for patient stratification and tailoring of treatment, targeting health promotion efforts and optimizing individualized treatment decisions for dental rehabilitation.

### REFERENCES

1. Offenbacher S, Divaris K, Barros S P, et al. Genome-wide association study of biologically-informed periodontal complex traits offers novel insights into the genetic basis of periodontal disease. Hum Mol Genet 2016.

2. Duran-Pinedo A E, Paster B, Teles R, Frias-Lopez J. Correlation network analysis applied to complex biofilm communities. PLoS One 2011; 6:e28438.

3. Ramseier C A, Kinney J S, Herr A E, et al. Identification of pathogen and host-response markers correlated with periodontal disease. Journal of periodontology 2009; 80:436-446.

4. Armitage G C. Development of a classification system for periodontal diseases and conditions. Ann Periodontol 1999; 4:1-6.

5. Eke P I, Page R C, Wei L, Thornton-Evans G, Genco R J. Update of the case definitions for population-based surveillance of periodontitis. Journal of periodontology 2012; 83:1449-1454.

6. Lanza S T, Collins L M, Lemmon D R, Schafer J L. PROC LCA: A SAS Procedure for Latent Class Analysis. Struct Equ Modeling 2007; 14:671-694.

7. Henry K L, Muthen B. Multilevel Latent Class Analysis: An Application of Adolescent Smoking Typologies with Individual and Contextual Predictors. Struct Equ Modeling 2010; 17:193-215.

8. Muthen B, Muthen L K. Integrating person-centered and variable-centered analyses: growth mixture modeling with latent trajectory classes. Alcohol Clin Exp Res 2000; 24:882-891.

9. Xin X, Ming Q, Zhang J, Wang Y, Liu M, Yao S. Four Distinct Subgroups of Self-Injurious Behavior among Chinese Adolescents: Findings from a Latent Class Analysis. PLoS One 2016; 11:e0158609.

10. Hamza C A, Willoughby T. Nonsuicidal self-injury and suicidal behavior: a latent class analysis among young adults. PLoS One 2013; 8:e59955.

11. Bora E, Aydin A, Sarac T, Kadak M T, Kose S. Heterogeneity of subclinical autistic traits among parents of children with autism spectrum disorder: Identifying the broader autism phenotype with a data-driven method. Autism Res 2016.

12. Swartz J A. A Multi-Group Latent Class Analysis of Chronic Medical Conditions Among Men Who Have Sex with Men. AIDS Behav 2016.

13. Granell R, Henderson A J, Sterne J A. Associations of wheezing phenotypes with late asthma outcomes in the Avon Longitudinal Study of Parents and Children: A population-based birth cohort. J Allergy Clin Immunol 2016.

14. Loe H, Silness J. Periodontal Disease in Pregnancy. I. Prevalence and Severity. Acta Odontol Scand 1963; 21:533-551.

15. Silness J, Loe H. Periodontal Disease in Pregnancy. Ii. Correlation between Oral Hygiene and Periodontal Condtion. Acta Odontol Scand 1964; 22:121-135.

16. Beck J D, Elter J R, Heiss G, Couper D, Mauriello S M, Offenbacher S. Relationship of periodontal disease to carotid artery intima-media wall thickness: the atherosclerosis risk in communities (ARIC) study. Arterioscler Thromb Vasc Biol 2001; 21:1816-1822.

17. Eke P I, Dye B A, Wei L, Thornton-Evans G O, Genco R J, Cdc Periodontal Disease Surveillance workgroup: James Beck GDRP. Prevalence of periodontitis in adults in the United States: 2009 and 2010. Journal of dental research 2012; 91:914-920.

18. Eke P I, Dye B A, Wei L, et al. Update on Prevalence of Periodontitis in Adults in the United States: NHANES 2009 to 2012. Journal of periodontology 2015; 86:611-622.

19. Beck J D, Sharp T, Koch G G, Offenbacher S. A study of attachment loss patterns in survivor teeth at 18 months, 36 months and 5 years in community-dwelling older adults. J Periodontal Res 1997; 32:497-505.

20. Beck J D, Koch G G, Rozier R G, Tudor G E. Prevalence and risk indicators for periodontal attachment loss in a population of older community-dwelling blacks and whites. Journal of periodontology 1990; 61:521-528.

21. Beck J D, Koch G G, Offenbacher S. Attachment loss trends over 3 years in community-dwelling older adults. Journal of periodontology 1994; 65:737-743.

22. Jolliffe I T, Cadima J. Principal component analysis: a review and recent developments. Philos Trans A Math Phys Eng Sci 2016; 374:20150202.

23. Milligan G W, Cooper M C. An examination of procedures for determining the number of clusters in a data set. Psychometrika 1985; 50:159-179.

24. NAGIN D. Group-Based Modeling of Development: Harvard University Press; 2009.

25. American Dental Association. Risk Management Series: Diagnosing and Managing the Periodontal Patient. Chicago: American Dental Association; 1986.

26. Lanza S T, Rhoades B L, Nix R L, Greenberg M T, Conduct Problems Prevention Research G. Modeling the interplay of multilevel risk factors for future academic and behavior problems: a person-centered approach. Dev Psychopathol 2010; 22:313-335.

27. Joensuu M, Mattila-Holappa P, Ahola K, et al. Clustering of adversity in young adults on disability pension due to mental disorders: a latent class analysis. Soc Psychiatry Psychiatr Epidemiol 2016; 51:281-287.

28. Ferrat E, Audureau E, Paillaud E, et al. Four Distinct Health Profiles in Older Patients With Cancer: Latent Class Analysis of the Prospective ELCAPA Cohort. J Gerontol A Biol Sci Med Sci 2016.

29. Lanza S T, Rhoades B L. Latent class analysis: an alternative perspective on subgroup analysis in prevention and treatment. Prev Sci 2013; 14:157-168.

30. Giannobile W V, Braun T M, Caplis A K, Doucette-Stamm L, Duff G W, Kornman K S. Patient stratification for preventive care in dentistry. Journal of dental research 2013; 92:694-701.

TABLE 8

| | LCA Classes | | | | | | | | | | | | | | |
|---|---|---|---|---|---|---|---|---|---|---|---|---|---|---|---|
| | PPC-A | | PPC-B | | PPC-C | | PPC-D | | PPC-E | | PPC-F | | PPC-G | | |
| | Class Monikers | | | | | | | | | | | | | | |
| | | | | | | | | | Posterior | | Severe | | Severe | | |
| | Health | | Mild Disease | | High GI | | Tooth Loss | | Disease | | Tooth Loss | | Disease | | |
| | n = 1,845 | | n = 1,047 | | n = 694 | | n = 800 | | n = 999 | | n = 900 | | n = 508 | | |
| | (27%) | | (15%) | | (10%) | | (11%) | | (15%) | | (13%) | | (7%) | | p-value |
| CDC/AAP | 351 | (19.0%) | 93 | (8.9%) | 31 | (4.5%) | 75 | (9.4%) | 0 | (0.0%) | 225 | (25.0%) | 0 | (0.0%) | <0.0001 |
| Health | | | | | | | | | | | | | | | |
| Mild | 867 | (47.0%) | 402 | (38.4%) | 286 | (41.2%) | 204 | (25.5%) | 50 | (5.0%) | 207 | (23.0%) | 19 | (3.7%) | |
| Moderate | 582 | (31.5%) | 486 | (46.4%) | 284 | (40.9%) | 370 | (46.3%) | 573 | (57.4%) | 328 | (36.4%) | 176 | (34.7%) | |
| Severe | 45 | (2.4%) | 66 | (6.3%) | 93 | (13.4%) | 151 | (18.9%) | 376 | (37.6%) | 140 | (15.6%) | 313 | (61.6%) | |
| African | 62 | (3.4%) | 33 | (3.2%) | 502 | (72.3%) | 147 | (18.5%) | 17 | (1.7%) | 308 | (34.3%) | 231 | (45.9%) | <0.0001 |
| American | | | | | | | | | | | | | | | |
| Caucasian | 1,777 | (96.6%) | 1,010 | (96.8%) | 192 | (27.7%) | 647 | (81.5%) | 979 | (98.3%) | 591 | (65.7%) | 272 | (54.1%) | |
| Female | 1,227 | (66.5%) | 489 | (46.7%) | 385 | (55.5%) | 436 | (54.4%) | 445 | (44.5%) | 512 | (56.9%) | 192 | (37.8%) | <0.0001 |
| Male | 618 | (33.5%) | 558 | (53.3%) | 309 | (44.5%) | 364 | (45.5%) | 554 | (55.5%) | 388 | (43.1%) | 316 | (62.2%) | |
| Age, mean | 61.8 | (0.13) | 62.4 | (0.17) | 61.6 | (0.21) | 63.7 | (0.20) | 62.9 | (0.19) | 63.1 | (0.19) | 61.8 | (0.25) | <0.0001 |
| (standard error) | | | | | | | | | | | | | | | |
| Diabetic | 154 | (8.4%) | 126 | (12.1%) | 147 | (21.7%) | 125 | (15.7%) | 108 | (10.8%) | 167 | (18.9%) | 111 | (22.2%) | <0.0001 |
| Non-Diabetic | 1,688 | (91.6%) | 920 | (88.0%) | 531 | (78.3%) | 672 | (84.3%) | 888 | (89.2%) | 719 | (81.2%) | 388 | (77.8%) | |
| Current Heavy | 86 | (4.8%) | 51 | (5.0%) | 43 | (6.7%) | 105 | (13.7%) | 136 | (14.0%) | 141 | (16.7%) | 55 | (11.4%) | |
| Smoker | | | | | | | | | | | | | | | |
| Current Light | 35 | (1.9%) | 8 | (0.8%) | 32 | (5.0%) | 17 | (2.2%) | 21 | (2.2%) | 34 | (4.0%) | 19 | (3.9%) | |
| Smoker | | | | | | | | | | | | | | | |
| Former Heavy | 245 | (13.6%) | 136 | (13.3%) | 67 | (10.5%) | 181 | (23.7%) | 223 | (23.0%) | 188 | (22.3%) | 69 | (14.3%) | <0.0001 |
| Smoker | | | | | | | | | | | | | | | |
| Former Light | 424 | (24.0%) | 251 | (24.6%) | 151 | (23.6%) | 133 | (17.4%) | 227 | (23.4%) | 162 | (19.2%) | 106 | (22.0%) | |
| Smoker | | | | | | | | | | | | | | | |
| Never Smoker | 1008 | (55.8%) | 574 | (56.3%) | 348 | (54.3%) | 329 | (43.0%) | 363 | (37.4%) | 320 | (37.9%) | 234 | (48.5%) | |
| Pack-Years, | 9.1 | (0.47) | 9.5 | (0.62) | 10.3 | (0.80) | 18.2 | (0.72) | 18.7 | (0.64) | 20.3 | (0.69) | 14.0 | (0.91) | <0.0001 |
| mean | | | | | | | | | | | | | | | |
| (standard error) | | | | | | | | | | | | | | | |
| Obese | 438 | (23.8%) | 318 | (30.5%) | 309 | (44.7%) | 285 | (35.7%) | 314 | (31.4%) | 349 | (38.8%) | 208 | (41.0%) | <0.0001 |
| Non-Obese | 1404 | (76.2%) | 726 | (69.5%) | 382 | (55.3%) | 513 | (64.3%) | 685 | (68.6%) | 550 | (61.2%) | 299 | (59.0%) | |
| Episodic DDS | 139 | (7.6%) | 165 | (15.8%) | 358 | (52.0%) | 224 | (28.1%) | 124 | (12.4%) | 524 | (58.7%) | 280 | (55.3%) | <0.0001 |
| User | | | | | | | | | | | | | | | |
| Regular DDS | 1,696 | (92.4%) | 878 | (84.2%) | 331 | (48.0%) | 573 | (71.9%) | 873 | (87.6%) | 368 | (41.3%) | 226 | (44.7%) | |
| User | | | | | | | | | | | | | | | |
| Seen DDS | 152 | (8.3%) | 166 | (15.9%) | 267 | (38.8%) | 156 | (19.6%) | 123 | (12.3%) | 405 | (45.4%) | 231 | (46.0%) | <0.0001 |
| >1 Year | | | | | | | | | | | | | | | |
| <1 year | 1683 | (91.7%) | 876 | (84.1%) | 422 | (61.3%) | 639 | (80.4%) | 874 | (87.7%) | 487 | (54.6%) | 271 | (54.0%) | |
| Income (per yr) | 261 | (14.6%) | 178 | (17.6%) | 280 | (43.2%) | 209 | (27.2%) | 146 | (14.9%) | 386 | (44.5%) | 183 | (38.5%) | <0.0001 |
| <$25K | | | | | | | | | | | | | | | |
| $25k-$50k | 622 | (34.9%) | 379 | (37.5%) | 216 | (33.3%) | 334 | (43.4%) | 373 | (38.2%) | 329 | (37.9%) | 157 | (33.0%) | |
| $50k+ | 901 | (50.5%) | 455 | (45.0%) | 152 | (23.5%) | 226 | (29.4%) | 458 | (46.9%) | 153 | (17.6%) | 136 | (28.6%) | |
| Years of | 100 | (5.4%) | 84 | (8.0%) | 165 | (23.9%) | 131 | (16.4%) | 59 | (5.9%) | 259 | (28.8%) | 120 | (23.6%) | <0.0001 |
| Education, | | | | | | | | | | | | | | | |
| <12 years | | | | | | | | | | | | | | | |
| 12-16 years | 770 | (41.8%) | 472 | (45.2%) | 223 | (32.3%) | 388 | (48.5%) | 445 | (44.6%) | 425 | (47.2%) | 197 | (38.8%) | |
| 17+ years | 972 | (57.2%) | 489 | (46.8%) | 303 | (43.9%) | 281 | (35.1%) | 494 | (49.5%) | 216 | (24.0%) | 191 | (37.6%) | |

TABLE 9
Clinical parameters of the 7 Periodontal Profile Classes (PPC) in the DARIC sample.

| | Periodontal Profiles Classes | | | | | | | | | | | | | | |
|---|---|---|---|---|---|---|---|---|---|---|---|---|---|---|---|
| | PPC-A | | PPC-B | | PPC-C | | PPC-D | | PPC-E | | PPC-F | | PPC-G | | |
| | Class Monikers | | | | | | | | | | | | | | |
| | | | Mild | | | | | | Posterior | | Severe | | Severe | | |
| | Health | | Disease | | High GI | | Tooth Loss | | Disease | | Tooth Loss | | Disease | | p-value |
| Extent IAL | 7.84 | (0.46) | 12.0 | (0.61) | 26.1 | (0.74) | 28.0 | (0.69) | 33.4 | (0.62) | 37.0 | (0.65) | 54.5 | (0.87) | <0.0001 |
| ≥3 mm* | | | | | | | | | | | | | | | |
| Extent PD | 2.07 | (0.23) | 4.27 | (0.31) | 4.52 | (0.38) | 6.61 | (0.35) | 14.2 | (0.31) | 7.41 | (0.33) | 24.6 | (0.44) | <0.0001 |
| ≥4mm* | | | | | | | | | | | | | | | |
| Extent BOP* | 11.7 | (0.47) | 27.7 | (0.62) | 24.2 | (0.77) | 26.4 | (0.71) | 24.3 | (0.64) | 31.7 | (0.67) | 61.5 | (0.90) | <0.0001 |
| Extent GI ≥1* | 2.67 | (0.58) | 27.6 | (0.75) | 92.8 | (0.90) | 30.1 | (0.90) | 5.53 | (0.82) | 61.5 | (0.81) | 82.9 | (1.07) | <0.0001 |
| Extent PQ ≥1* | 9.08 | (0.67) | 53.2 | (0.86) | 75.9 | (1.06) | 32.9 | (1.03) | 28.2 | (0.93) | 69.8 | (0.94) | 81.7 | (1.24) | <0.0001 |
| Mean Number | 26.0 | (0.08) | 26.1 | (0.10) | 20.1 | (0.13) | 16.8 | (0.12) | 25.8 | (0.11) | 7.74 | (0.11) | 24.5 | (0.15) | <0.0001 |
| of Teeth | | | | | | | | | | | | | | | |
| Mean Number | 5.54 | (0.09) | 4.28 | (0.13) | 1.93 | (0.15) | 4.28 | (0.14) | 5.77 | (0.13) | 1.09 | (0.14) | 2.70 | (0.18) | <0.0001 |
| of Crowns | | | | | | | | | | | | | | | |

IAL, interproximal attachment loss;
PD, probing depth;
BOP, bleeding on probing;
GI, gingival index;
PQ, plaque;
*Extent Scores are represented as Mean (standard error), total N = 6,793

TABLE 10
Distribution of periodontal status by Periodontal Profile Class (PPC) for the three validation/replication datasets.

| | | | | | | | | | | | | | | | |
|---|---|---|---|---|---|---|---|---|---|---|---|---|---|---|---|
| CDC/AAP Health | 1,069 | (53.1%) | 0 | (0.0%) | 2 | (0.72%) | 51 | (11.6%) | 0 | (0.0%) | 55 | (22.7%) | 0 | (0.0%) | <0.0001 |
| Mild | 595 | (29.5%) | 81 | (30.7%) | 34 | (12.3%) | 75 | (17.1%) | 13 | (6.91%) | 27 | (11.2%) | 4 | (1.23%) | |
| Moderate | 345 | (17.1%) | 169 | (64.0%) | 204 | (73.7%) | 226 | (51.5%) | 121 | (64.4%) | 134 | (10.3%) | 106 | (32.6%) | |
| Severe | 6 | (0.30%) | 14 | (5.3%) | 37 | (13.4%) | 87 | (19.8%) | 54 | (28.7%) | 26 | (10.7%) | 215 | (66.2%) | |
| Extent IAL ≥3 mm* | 3.57 | (0.38) | 17.2 | (1.06) | 33.5 | (1.04) | 34.4 | (0.82) | 35.9 | (1.26) | 47.4 | (1.11) | 64.7 | (0.96) | <0.0001 |
| Extent PD ≥4 mm* | 0.46 | (0.18) | 5.90 | (0.49) | 2.38 | (0.47) | 7.87 | (0.38) | 13.6 | (0.58) | 5.08 | (0.51) | 30.3 | (0.44) | <0.0001 |
| Number of Teeth | 27.1 | (0.07) | 28.4 | (0.19) | 22.2 | (0.19) | 15.4 | (0.14) | 27.9 | (0.23) | 7.22 | (0.20) | 25.3 | (0.17) | <0.0001 |

NHANES 2011-2012 population

| | | | | | | | | | | | | | | | |
|---|---|---|---|---|---|---|---|---|---|---|---|---|---|---|---|
| N | 1,772 | (53%) | 221 | (6%) | 322 | (9%) | 392 | (12%) | 180 | (5%) | 214 | (6%) | 237 | (7%) | p-value |
| CDC/AAP Health | 563 | (31.8%) | 0 | (0.0%) | 2 | (0.62%) | 22 | (5.61%) | 0 | (0.0%) | 36 | (16.8%) | 0 | (0.0%) | <0.0001 |
| Mild | 849 | (47.9%) | 16 | (7.24%) | 43 | (13.4%) | 76 | (19.4%) | 2 | (1.11%) | 33 | (15.4%) | 0 | (0.0%) | |
| Moderate | 352 | (19.9%) | 175 | (79.2%) | 246 | (19.6%) | 191 | (48.7%) | 99 | (55.0%) | 117 | (54.7%) | 73 | (30.8%) | |
| Severe | 8 | (0.45%) | 30 | (13.6%) | 31 | (9.63%) | 103 | (26.3%) | 79 | (43.9%) | 28 | (13.1%) | 164 | (69.2%) | |
| Extent IAL ≥3 mm* | 6.72 | (0.43) | 25.9 | (1.21) | 42.2 | (1.00) | 43.1 | (0.91) | 51.3 | (1.34) | 59.0 | (1.23) | 75.8 | (1.17) | <0.0001 |
| Extent PD ≥4 mm* | 0.44 | (0.19) | 4.32 | (0.53) | 1.92 | (0.44) | 6.52 | (0.39) | 10.8 | (0.58) | 7.01 | (0.53) | 23.3 | (0.51) | <0.0001 |
| Number of Teeth | 27.2 | (0.07) | 28.2 | (0.21) | 22.7 | (0.17) | 14.8 | (0.16) | 28.4 | (0.21) | 6.91 | (0.21) | 25.1 | (0.20) | <0.0001 |

Piedmont 65+ Dental Study (PDS)

| | | | | | | | | | | | | | | | |
|---|---|---|---|---|---|---|---|---|---|---|---|---|---|---|---|
| N | 135 | (19%) | 31 | (4%) | 187 | (27%) | 159 | (23%) | 6 | (1%) | 131 | (19%) | 48 | (7%) | p-value |
| CDC/AAP Health | 49 | (36.3%) | 0 | (0.0%) | 8 | (4.3%) | 21 | (13.2%) | 0 | (0.0%) | 38 | (29.0%) | 0 | (0.0%) | <0.0001 |
| Mild | 56 | (41.5%) | 9 | (29.0%) | 31 | (16.6%) | 24 | (15.1%) | 0 | (0.0%) | 17 | (13.0%) | 0 | (0.0%) | |
| Moderate | 30 | (22.2%) | 16 | (51.6%) | 110 | (58.8%) | 75 | (47.2%) | 6 | (100%) | 57 | (43.5%) | 14 | (29.2%) | |
| Severe | 0 | (0.0%) | 6 | (19.4%) | 38 | (20.3%) | 39 | (24.5%) | 9 | (0.0%) | 19 | (14.5%) | 34 | (70.8%) | |
| Extent IAL ≥3 mm* | 10.0 | (2.38) | 28.2 | (4.96) | 47.5 | (2.02) | 53.2 | (2.19) | 50.1 | (11.3) | 64.4 | (2.41) | 81.4 | (3.98) | <0.0001 |
| Extent PD ≥4 mm* | 0.81 | (1.05) | 7.61 | (2.20) | 6.47 | (0.90) | 9.54 | (0.97) | 10.6 | (5.00) | 10.9 | (1.07) | 27.7 | (1.77) | <0.0001 |
| Number of Teeth | 25.1 | (0.97) | 27.8 | (0.89) | 19.7 | (0.55) | 12.4 | (0.77) | 26.8 | (1.37) | 6.0 | (0.69) | 26.6 | (0.70) | <0.0001 |

IAL, interproximal attachment loss;
PD, probing depth,
*Extent Scores are represented as mean (standard error)

TABLE 11
Mean posterior probabilities of class assignment for each of the
periodontal and tooth profile classes (PPC/TPC).

| | Post- | Post- | Post- | Post- | Post- | Post- | Post- |
|---|---|---|---|---|---|---|---|
| | Prob- | Prob- | Prob- | Prob- | Prob- | Prob- | Prob- |
| | A | B | C | D | E | F | G |
| PPC-A | 0.978 | 0.007 | 0.000 | 0.006 | 0.010 | 0.000 | 0.000 |
| PPC-B | 0.011 | 0.967 | 0.003 | 0.004 | 0.012 | 0.000 | 0.002 |
| PPC-C | 0.000 | 0.004 | 0.985 | 0.006 | 0.000 | 0.000 | 0.004 |
| PPC-D | 0.016 | 0.004 | 0.006 | 0.968 | 0.006 | 0.000 | 0.001 |
| PPC-E | 0.015 | 0.011 | 0.000 | 0.004 | 0.968 | 0.000 | 0.002 |
| PPC-F | 0.000 | 0.000 | 0.000 | 0.000 | 0.000 | 1.000 | 0.000 |
| PPC-G | 0.000 | 0.007 | 0.009 | 0.002 | 0.002 | 0.000 | 0.981 |
| TPC-A | 0.865 | 0.000 | 0.055 | 0.078 | 0.001 | 0.000 | 0.000 |
| TPC-B | 0.000 | 0.953 | 0.000 | 0.001 | 0.012 | 0.032 | 0.001 |
| TPC-C | 0.013 | 0.004 | 0.934 | 0.011 | 0.037 | 0.000 | 0.000 |
| TPC-D | 0.094 | 0.008 | 0.036 | 0.823 | 0.031 | 0.007 | 0.001 |
| TPC-E | 0.000 | 0.005 | 0.012 | 0.020 | 0.927 | 0.018 | 0.018 |
| TPC-F | 0.000 | 0.010 | 0.000 | 0.002 | 0.014 | 0.928 | 0.046 |
| TPC-G | 0.000 | 0.000 | 0.000 | 0.000 | 0.035 | 0.037 | 0.927 |

Post-Prob, mean posterior probability of individuals or teeth to be assigned to the correct periodontal or tooth profile class.

TABLE 12
Clinical parameters [mean, (standard error)] of the 7 Tooth Profile Classes (TPC) of DARIC population.

| | Tooth Profile Classes | | | | | | | | | | | | | |
|---|---|---|---|---|---|---|---|---|---|---|---|---|---|---|
| | TPC-A | | TPC-B | | TPC-C | | TPC-D | | TPC-E | | TPC-F | | TPC-G | |
| | N = 56,677 | | N = 12,952 | | N = 17,383 | | N = 18,799 | | N = 19,999 | | N = 10,509 | | N = 9,034 | |
| | Class Monikers | | | | | | | | | | | | | |
| | | | | | | | Gingival | | Interproximal | | Reduced | | Severe | |
| | Health | | Recession | | Crown | | Inflammation | | Disease | | Periodontium | | Disease | |
| Mean IAL (mm) | 0.82 | (0.57) | 1.29 | (0.69) | 1.59 | (0.59) | 1.67 | (0.71) | 2.46 | (0.60) | 3.17 | (1.12) | 4.51 | (1.61) |
| Mean DAL (mm) | 0.79 | (0.54) | 2.28 | (0.64) | 1.20 | (0.59) | 1.46 | (0.68) | 1.77 | (0.88) | 3.42 | (1.08) | 3.70 | (1.91) |
| Mean IPD (mm) | 1.64 | (0.52) | 1.83 | (0.57) | 2.08 | (0.49) | 1.95 | (0.52) | 3.14 | (0.54) | 2.12 | (0.75) | 4.18 | (1.14) |
| Mean DPD (mm) | 1.10 | (0.33) | 1.10 | (0.33) | 1.34 | (0.42) | 1.49 | (0.54) | 1.76 | (0.65) | 1.34 | (0.54) | 2.87 | (1.28) |
| Mean IGR (mm) | −0.83 | (0.33) | −0.54 | (0.49) | −0.49 | (0.48) | −0.28 | (0.51) | −0.68 | (0.45) | 1.06 | (0.92) | 0.33 | (1.22) |
| Mean DGR (mm) | −0.31 | (0.52) | 1.18 | (0.58) | −0.13 | (0.47) | −0.03 | (0.44) | 0.01 | (0.76) | 2.06 | (1.00) | 0.83 | (1.45) |
| Mean BOP (%) | 0.11 | (0.20) | 0.14 | (0.22) | 0.24 | (0.29) | 0.31 | (0.32) | 0.41 | (0.33) | 0.25 | (0.31) | 0.64 | (0.33) |
| Mean GI | 0.11 | (0.37) | 0.16 | (0.41) | 0.26 | (0.51) | 0.94 | (0.52) | 0.42 | (0.68) | 0.40 | (0.69) | 0.99 | (0.91) |
| Mean PI | 0.22 | (0.47) | 0.31 | (0.50) | 0.19 | (0.42) | 0.89 | (0.48) | 0.51 | (0.63) | 0.60 | (0.69) | 0.98 | (0.79) |
| Mean DCS | 0.00 | (0.08) | 0.01 | (0.13) | 0.00 | (0.00) | 0.09 | (0.37) | 0.02 | (0.16) | 0.05 | (0.29) | 0.09 | (0.38) |
| Mean FCS | 0.79 | (1.16) | 0.87 | (1.25) | 0.19 | (0.69) | 0.61 | (1.07) | 1.09 | (1.40) | 0.92 | (1.30) | 0.90 | (1.30) |
| Mean DRS | 0.00 | (0.01) | 0.01 | (0.07) | 0.00 | (0.02) | 0.02 | (0.15) | 0.00 | (0.06) | 0.03 | (0.23) | 0.04 | (0.25) |
| Mean FRS | 0.02 | (0.13) | 0.11 | (0.33) | 0.00 | (0.06) | 0.02 | (0.16) | 0.03 | (0.17) | 0.13 | (0.41) | 0.04 | (0.24) |
| Crown (%) | 0.00 | (0.00) | 0.13 | (0.34) | 0.92 | (0.27) | 0.00 | (0.00) | 0.36 | (0.48) | 0.10 | (0.30) | 0.17 | (0.38) |

IAL, interproximal attachment loss;
DAL, direct attachment loss;
IPD, interproximal probing depth;
DPD, direct probing depth;
IGR, interproximal gingival recession;
DGR, direct gingival recession;
BOP, bleeding on probing;
GI, gingival index;
PI, plaque index;
DCS, decayed coronal surface;
FCS, filled coronal surface;
DRS, decayed root surface;
FRS, filled root surface;
total N = 145,353 teeth.

TABLE 13
Mean (StdDev) predicted probability for each Periodontal Profile Class (PPC)
assignment in the presence of missing data.

| | PPC-A | | PPC-B | | PPC-C | | PPC-D | | PPC-E | | PPC-F | | PPC-G | |
|---|---|---|---|---|---|---|---|---|---|---|---|---|---|---|
| No Missing Data | 0.98 | (0.08) | 0.97 | (0.09) | 0.98 | (0.06) | 0.97 | (0.09) | 0.97 | (0.09) | 1.00 | (0.00) | 0.98 | (0.08) |
| Missing Tooth Status | 0.97 | (0.09) | 0.96 | (0.11) | 0.98 | (0.07) | 0.95 | (0.10) | 0.96 | (0.10) | 0.99 | (0.05) | 0.98 | (0.07) |
| Missing Attachment Loss | 0.97 | (0.09) | 0.96 | (0.10) | 0.98 | (0.07) | 0.96 | (0.10) | 0.94 | (0.12) | 1.00 | (0.02) | 0.98 | (0.08) |
| Missing Pocket Depth | 0.97 | (0.09) | 0.96 | (0.09) | 0.98 | (0.08) | 0.96 | (0.10) | 0.95 | (0.11) | 1.00 | (0.00) | 0.98 | (0.08) |
| Missing BOP | 0.97 | (0.09) | 0.96 | (0.09) | 0.97 | (0.10) | 0.97 | (0.08) | 0.97 | (0.10) | 1.00 | (0.00) | 0.98 | (0.08) |
| Missing Gingival Index | 0.97 | (0.09) | 0.95 | (0.11) | 0.96 | (0.10) | 0.95 | (0.11) | 0.96 | (0.10) | 1.00 | (0.00) | 0.98 | (0.08) |
| Missing Plaque Index | 0.97 | (0.09) | 0.96 | (0.10) | 0.98 | (0.08) | 0.96 | (0.10) | 0.96 | (0.11) | 1.00 | (0.01) | 0.98 | (0.07) |
| Missing Crowns | 0.98 | (0.08) | 0.96 | (0.10) | 0.98 | (0.07) | 0.95 | (0.11) | 0.97 | (0.09) | 1.00 | (0.00) | 0.98 | (0.07) |
| Missing Third Molar Data | 0.98 | (0.08) | 0.97 | (0.09) | 0.98 | (0.07) | 0.97 | (0.09) | 0.97 | (0.08) | 1.00 | (0.00) | 0.99 | (0.05) |
| Half Mouth | 0.97 | (0.09) | 0.95 | (0.11) | 0.97 | (0.08) | 0.95 | (0.11) | 0.95 | (0.11) | 1.00 | (0.00) | 0.98 | (0.08) |
| (Opposing Quadrants) | | | | | | | | | | | | | | |
| Half Mouth, No BOP, GI, | 0.84 | (0.15) | 0.75 | (0.17) | 0.74 | (0.18) | 0.83 | (0.17) | 0.90 | (0.15) | 1.00 | (0.02) | 0.90 | (0.16) |
| PI, Crowns | | | | | | | | | | | | | | |

TABLE 14
Mean (StdDev) average posterior probability for all Periodontal Profile Classes considering missing data for each and multiple parameters.

| | | | | | | | | |
|---|---|---|---|---|---|---|---|---|
| Tooth Status | AL | PD | BOP | GI | PI | Crown | 3rd Molar | Half Mouth |

One Index Set Missing

| | | | | | | | | | | | | | | | | | | |
|---|---|---|---|---|---|---|---|---|---|---|---|---|---|---|---|---|---|---|
| 1 Set Missing | 0.97 | (0.09) | 0.97 | (0.09) | 0.97 | (0.09) | 0.97 | (0.08) | 0.97 | (0.09) | 0.97 | (0.09) | 0.98 | (0.08) | 0.98 | (0.08) | 0.97 | (0.09) |

Two Index Sets Missing

| | | | | | | | | | | | | | | | | |
|---|---|---|---|---|---|---|---|---|---|---|---|---|---|---|---|---|
| Tooth Status | 0.96 | (0.10) | 0.96 | (0.10) | 0.96 | (0.10) | 0.96 | (0.11) | 0.96 | (0.10) | 0.97 | (0.09) | 0.97 | (0.09) | 0.95 | (0.11) |
| AL | | | 0.95 | (0.11) | 0.96 | (0.10) | 0.96 | (0.10) | 0.96 | (0.10) | 0.97 | (0.09) | 0.97 | (0.09) | 0.95 | (0.11) |
| PD | | | | | 0.96 | (0.10) | 0.96 | (0.10) | 0.96 | (0.10) | 0.97 | (0.09) | 0.97 | (0.09) | 0.96 | (0.11) |
| BOP | | | | | | | 0.96 | (0.10) | 0.97 | (0.09) | 0.97 | (0.09) | 0.97 | (0.09) | 0.96 | (0.10) |
| GI | | | | | | | | | 0.93 | (0.13) | 0.96 | (0.10) | 0.97 | (0.10) | 0.95 | (0.11) |
| PI | | | | | | | | | | | 0.97 | (0.09) | 0.97 | (0.09) | 0.95 | (0.11) |
| Crown | | | | | | | | | | | | | 0.97 | (0.08) | 0.96 | (0.10) |
| 3^{rd} Molar | | | | | | | | | | | | | | | 0.96 | (0.10) |

Selected Three Index Sets Missing

| | | | | | | | | | | | | | | | | |
|---|---|---|---|---|---|---|---|---|---|---|---|---|---|---|---|---|
| GI, Crown | 0.95 | (0.11) | 0.95 | (0.11) | 0.95 | (0.11) | 0.96 | (0.10) | 0.93 | (0.13) | | | 0.96 | (0.10) | 0.95 | (0.12) |
| GI, PI | 0.92 | (0.14) | 0.92 | (0.13) | 0.92 | (0.13) | 0.91 | (0.14) | | | 0.93 | (0.13) | 0.93 | (0.13) | 0.91 | (0.14) |

Selected Four Index Sets Missinc

| | | | | | | | | | | | | | | | | |
|---|---|---|---|---|---|---|---|---|---|---|---|---|---|---|---|---|
| GI, Crown, 3rds | 0.95 | (0.11) | 0.95 | (0.11) | 0.95 | (0.11) | 0.96 | (0.10) | | | 0.92 | (0.14) | | | 0.95 | (0.12) |
| PI, Crown, Half | 0.94 | (0.12) | 0.94 | (0.12) | 0.94 | (0.12) | 0.95 | (0.11) | 0.90 | (0.15) | | | 0.95 | (0.11) | | |

### EXAMPLE 3

Introduction

One major goal of the profession involves an evidence-based approach to assigning risk of future periodontal disease or disease progression to patients in order to provide individualized treatment (precision dentistry). The practice of precision dentistry will require optimal measures of clinical and biomarker assessments as well as a thoughtful analysis of the etiology and appropriate interventions in order to focus on the risks and treatment needs of the individual patient. The Periodontal Profile Phenotype System (P3) contributes to the practice of precision dentistry by providing a standardized method for classifying a patient based upon history and clinical findings that allows a practitioner to assess risk of future attachment and tooth loss. It does this without the benefit of genetic, biomarker or other patient-specific parameters that will be needed to fully support the practice of precision dentistry.

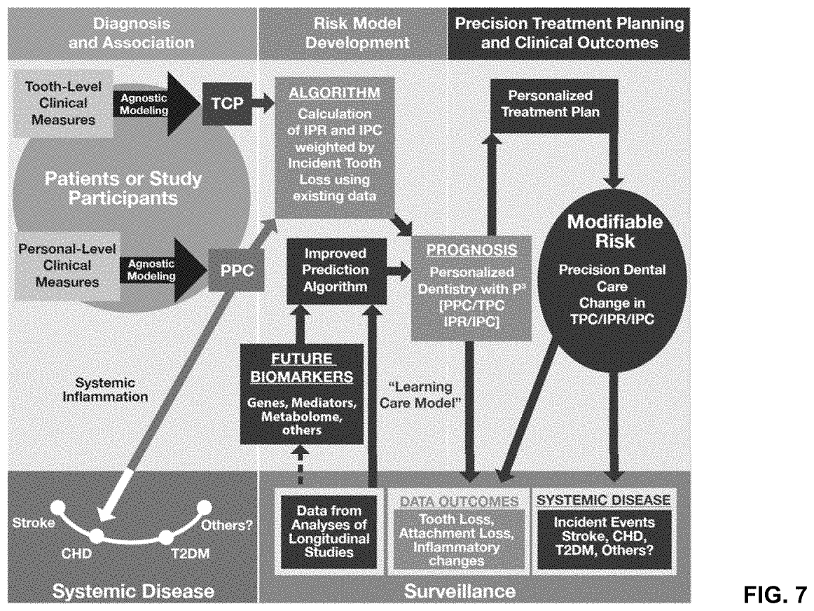

This Example describes, inter alia, the development, validation, testing, and utility for risk profiling for tooth loss and attachment loss for an improved classification of periodontal disease that may have greater utility for personalized dentistry than current case definitions of periodontitis.¹ Importantly, this system uses clinical data to create three person-level measures and one tooth level measure that we refer to as P³ [Periodontal Profile Phenotype] system, presented in FIG. 7. These four key measures generated by the P³ algorithm include:
The Periodontal Profile Class (PPC)], a person-level measure, that provides a clinical, seven-class taxonomic system of the patient's disease status.
The TPC, or Tooth Profile Class, that assigns each tooth present within a patient one of seven possible statuses.
The IPR-[Index of Periodontal Risk] incorporates the individual patient PPC and TPC composition and a longitudinal database of tooth loss and attachment loss to create a point estimate of future disease risk for the individual patient.
The IPC [Index of Periodontal Class] places the IPR risk score of the patient into a population context of low, moderate, or high risk.

The P³ system utilizes these four measures to potentially form the basis of an improved “healthcare Learning System” for precision dentistry. There are four domains outlined in FIG. 7. (1) Creating robust diagnostic “bins” of individuals with the Periodontal Profile Class (PPC). (2) Risk Model Development that can lead to a more precise and probabilistic quantification of prognosis for an individual patient (using IPR for the subject risk and TPC for tooth-level risk. (3) This information will lead to more precise Treatment Planning and provide more sensitive clinical outcome assessments, and (4) by including a Surveillance Platform, the risk modelling can be refined and will accommodate the addition of future biomarker data (e.g. genetics, inflammatory mediator and other “omic” dimensions to optimize precision dentistry.

The aims were: (1) to provide the reader with a more comprehensive understanding of the scope and current status of the P³ project, and (2) to describe in detail the relationships between the PPC and TPC components of the P³ as shown within the orange circle under Diagnosis and Association in FIG. 7. The third aim is to illustrate the unique aspects of this system compared to other commonly used periodontal classification indices by providing a proof-of-principle demonstration for practitioners to eventually use the parts of this system that currently are under development. The P³ System can have utility for establishing more precise dental prognoses, clinical outcome assessments, and potentially stronger associations with systemic diseases.

Materials and Methods

Study Samples. The ARIC study² enrolled 15,792 participants within the age group of 45-64 in four different U.S. communities (Forsyth County [N.C.], city of Jackson [Miss.], suburbs of Minneapolis [Minn.], Washington County [Md.]) . All participants provided written informed consent to a protocol reviewed and approved by the Institutional Review Board on research involving human subjects at the University of North Carolina and at each study performance site. All participants who completed the fourth clinic visit (1996-1998) in ARIC (N=11,656) were eligible for inclusion. Of the 11,656 ARIC participants seen at the fourth clinical visit, we excluded study participants who did not receive a periodontal examination. These exclusions resulted in 6,793 individuals who were included in this study as well as the LCA that resulted in the creation of PPC.³ The National Health and Nutrition Examination Survey (NHANES; 2009-2010, 2011-2012 and 2013-2014) were combined to use as a validation study population. The technical details of the surveys, including sampling design, periodontal data collection protocols, and data availability, are described elsewhere.^{4, 5 6, 7, 8} Briefly, periodontal measurements were collected for 3,750 individuals (NHANES 2009-2010), for 3,338 individuals (NHANES 2011-2012), and 3622 individuals for (NHANES 2013-2014) for a total of 10,710. Periodontal measures were collected on six-sites per tooth for all teeth present in the mouth except third molars.⁸⁻¹⁰

Measurement of Exposures.

Periodontal Profile Class (PPC). The analytical approach implemented person-level LCA to identify discrete classes of individuals using seven tooth-level clinical parameters. These parameters were: ≥1 site with interproximal attachment level (IAL)≥3 mm, site with probing depth (PD) ≥4 mm, extent of bleeding on probing (BOP), dichotomized at 50% or ≥3 sites per tooth), gingival inflammation index¹¹ (GI=0 vs. GI≥1), plaque index¹² (PI=0 vs. PI≥1), the presence/absence of full prosthetic crowns for each tooth, and tooth status (present vs. absent).³

Individuals were classified into mutually exclusive latent classes based upon their responses to a set of observed categorical variables. Criteria used to determine the optimal number of classes included the Akaike Information Criterion (AIC)¹³ and the Bayesian Information Criterion (BIC)¹⁴, while ensuring that clinically relevant categories were maintained. We used Milligan and Cooper's¹⁵ recommendation for the maximum number (n) of classes, suggesting stopping when the newly added class (n+1) is not clinically distinct from the previous number (n) of identified classes. Additionally, it was verified that mean posterior probabilities of correct class assignment were >0.7, which according to Nagan¹⁶ indicates adequate class separation. In the first step, the person-level LCA was used to classify individuals into seven latent classes based on 224 dichotomous variables (derived from seven tooth-level variables, using the clinical parameters referred to above for each of 32 teeth). The class membership probabilities represent the overall, unconditional proportions of individuals in each of seven latent classes. The model parameters from the first step were used to compute the posterior probabilities (the probability of event A occurring given that event B has occurred) of each individual's membership into each class based upon the values of the 224 items, or as many of them as were observed for that individual.³

Since patients with periodontal disease have individual teeth with clinical signs ranging from health to severe disease, we also carried out a tooth-level LCA analysis to capture the distribution of these tooth-specific classes within each PPC subgroup. This tooth-level analysis of each individual's existing complement of teeth produced seven categories of teeth.

CDC/AAP and European Indices. The Centers for Disease Control/American Academy of Periodontology (CDC/AAP) index¹⁷ along with the European Periodontal index¹⁸ may be the most frequently used indices and are a step forward in creating some consistency in periodontal disease case definitions. The CDC/AAP 4-level index (Healthy; Mild; Moderate; and Severe Disease)⁷ was used as it provided separation of the Healthy and Mild groups. The definitions of the levels of disease for both indices appear in Table 15. The European Index has 3-levels (Healthy, Incipient, and Severe).¹⁸

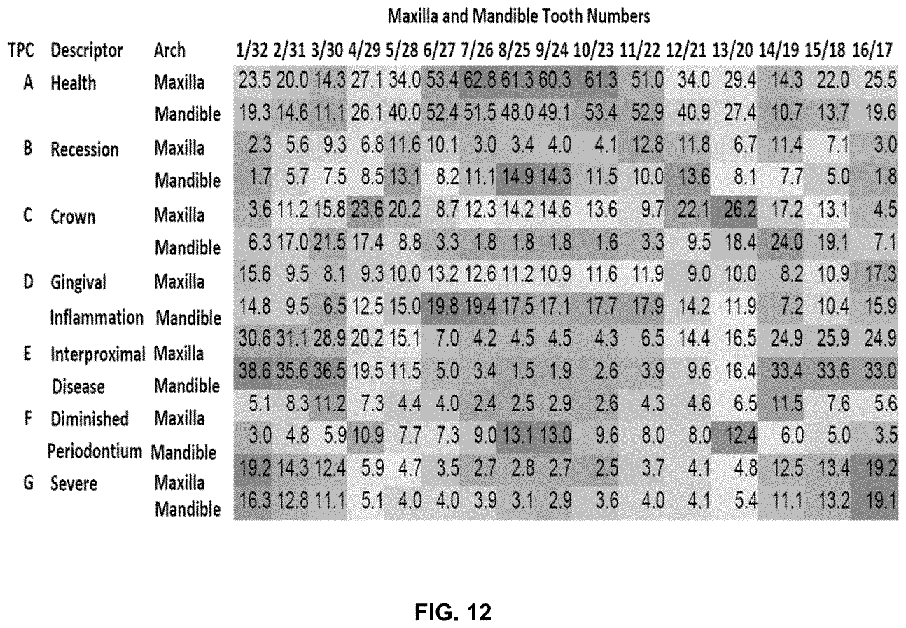

Statistical Analysis. The table and most of the figures in this paper are descriptive in nature and consist of means and proportions. FIG. 12 presents posterior probabilities that were produced as part of the LCA analysis¹³ using the Dental ARIC data set. The average posterior probabilities were calculated for each of the seven person-level and tooth-level LCA classes. The average posterior probabilities for class assignment were calculated within each of the seven indices.

Results

Concordance of PPC, CDC/AAP, and European Classification Systems. Table 15 displays similarities and differences in disease classifications between the PPC and both the CDC/AAP and the European Indexes. The PPC classification creates seven classes that are nominal categories arranged in order of increasing extent interproximal attachment loss 3 mm. The PPC classes were given “monikers or names” based upon the dominant clinical feature of the teeth in that class. Although some of the “monikers” are familiar, the clinical status of the teeth in that class are different than one might expect. The mean clinical characteristics of each class are presented in Table 9 as previously described³ and can demonstrate traditional measures of periodontal disease are represented in each PPC.

For the PPC, 1,845/6793 (27%) of the Dental ARIC participants were PPC Healthy, 15% had Mild disease, 10% had High Gingival Index, 12% had Tooth Loss, 15% had Posterior Disease, 13% had Severe Tooth Loss, and 7% had Severe Disease. By contrast, the CDC/AAP index classified the Dental ARIC participants as 11% Healthy, 30% Mild, 42% Moderate, and 17% Severe and the European system classes were 11% Healthy, 74% Incipient, and 14% Severe. Of participants classified as Healthy by CDC/AAP and European indices, 45% and 47%, respectively, were classified Healthy by the PPC. Of those not classified as Healthy by the PPC, 29% CDC/AAP and 27% European were classified as having “PPC-Severe Tooth Loss”, indicating most subjects were near or completely edentate in the maxillae with only a few lower anterior/premolar teeth that were less diseased³ (Table 9). Of the remaining CDC/AAP and European “Healthy” participants, about 12% were classified as “Mild disease” and 10% were classified as having “Tooth loss” with the remaining 4% being High GI. Of those classified as “Severe” by CDC/AAP, the PPC indicated 32% had “Posterior Disease” and the majority of the remainder had “Tooth Loss” and “Severe Tooth Loss”. Of those classified as “Severe” by the European index, most had “Severe Tooth Loss, Posterior Disease, and Tooth Loss”; 30%, 19% and 15%, respectively. As one examines the extremes of Health vs Severe Disease as identified by the PPC in the Dental ARIC dataset, 45% of the CDC/AAP Healthy are PPC Healthy, while 26% of the CDC/AAP Severe are PPC-Severe. Very similar relationships are evident between the European and PPC indices. Thus, PPC identified four new categories of people with distinct clinical traits in addition to the traditional healthy, mild and severe categories. These new categories are composed of individuals who were in one of the CDC/AAP or European classification categories, but now represent previously “hidden” groupings of individuals with similar within-class clinical presentations.

The age range for the NHANES 2009-2014 sample was 30-85, which made it a younger group than the ARIC sample, where the youngest age was 52. However, the PPCs comparing the CDC to the European classifications showed similar patterns even though this population had less overall disease. For example, in the NHANES studies, about 90% of study participants classified as “Healthy” by both the CDC/AAP and European Indices and classified as “Healthy” by PPC. Of those not classified as “Healthy” by PPC, most were allocated to the two “Tooth Loss” categories. For those classified as “Severe” by CDC/AAP and European indices, 44% and 35%, respectively classified as “Severe” by PPC, while the remainder were spread among the other PPC categories.

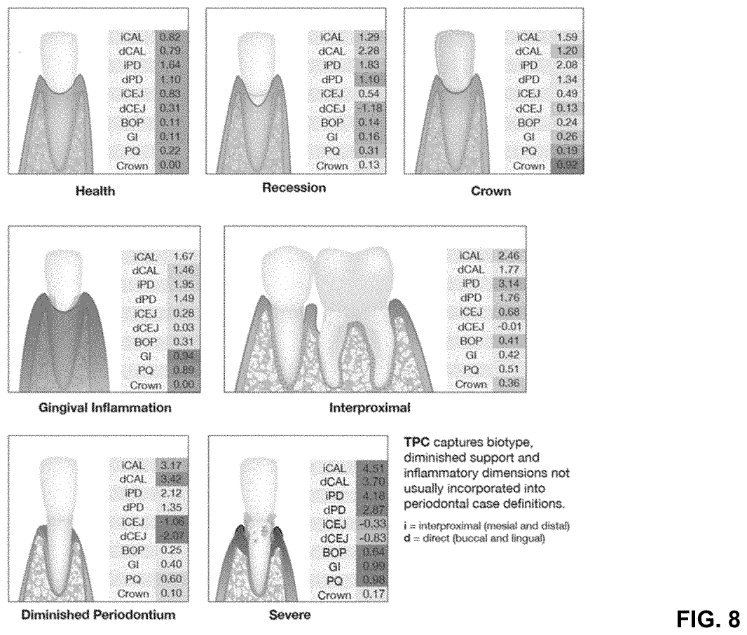

PPC and TPC Distributions. The tooth-based analysis in FIG. 8 presents the mean tooth-level clinical periodontal measures and proportion with crowns within each TPC in the form of multiple heat maps that allow the monikers of the seven TPC classes to become more apparent. For example, the clinical measures are green for Healthy teeth, indicating that there is little disease and no crowns. In contrast, teeth classified as Severe are mostly red indicating that high levels of interproximal and direct CAL and PD as well as high levels of BOP, GI, Plaque, and Recession. As an example, for TPC Recession the mean clinical characteristics of the tooth is direct buccal and/or lingual recession (dCEJ); or circumferential loss of attachment with minimal probing depths to reflect a tooth with diminished periodontium; or high gingival inflammation and plaque that defines a High GI TPC. A profile of a patient can be built by considering the multiple possible combinations of TPC that might arise. The power of this approach is that any given individual can have a unique combination of these TPCs.

For reference purposes, the heat map in FIG. 12 displays another tooth-based analysis of the intraoral distribution of TPCs for all 145,497 teeth expressed as posterior probabilities of having that characteristic. The posterior probability is a conditional probability when all other variables are considered. This provides an intraoral map of probabilities of TPC status for the ARIC cohort.

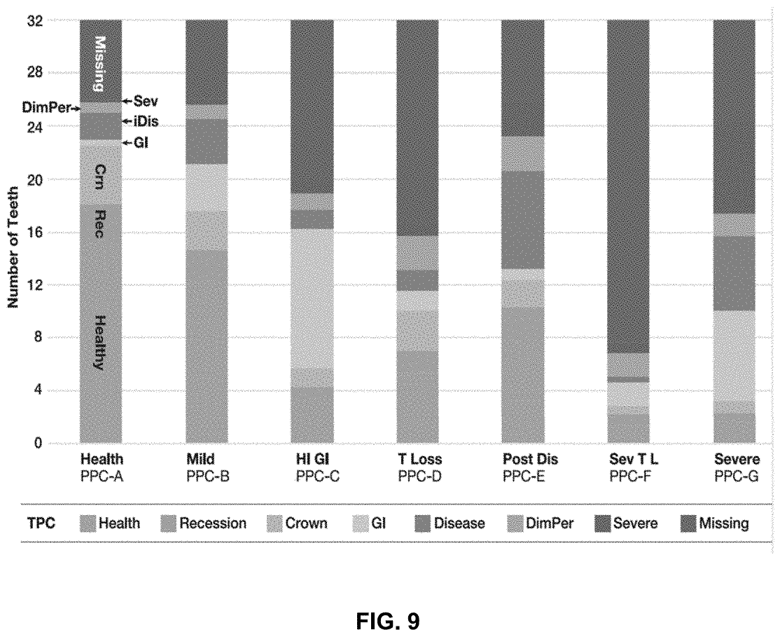

The identification of TPCs in addition to PPCs enables one to assign a tooth class for each existing tooth in a patient, as well as missing teeth. FIG. 9 presents the proportion of each of the seven TPCs and missing teeth within each PPC [0-32 teeth]. This person-based analysis shows that PPC-Healthy individuals have few missing teeth (predominantly 3^{rd} molars) and an average of 15 healthy teeth with very few teeth that are GI or Interproximal attachment loss (iCAL). Severe TPCs are rare, but there are some Recession TPCs (with local attachment loss in that area) and a few crowned teeth can be noted. As one examines the Mild, Posterior, and Severe disease PPCs, those phenotypes are associated with having a high number of diseased TPCs and fewer Healthy TPCs. The key difference here is that most teeth are present in these three PPCs and the Posterior Disease PPC has fewer GI teeth and a greater number of teeth with iAL, along with a few Severe teeth. The Severe Disease PPC has more GI TPCs, more Interproximal Disease iCAL TPCs, more Severe TPC teeth and very few heathy TPC teeth. It is noted that a few severe teeth in the Posterior Disease PPC does not automatically shift the individual to the Severe PPC class, as might be expected with other classifications. Furthermore, the PPC classification algorithm identifies three new PPCs: a High GI that has more missing teeth and teeth with GI scores on the majority of the remaining teeth; Tooth Loss and Severe Tooth Loss. There also is a refinement in what might be considered as moderate periodontitis into Mild Disease and Posterior (interproximal) Disease.

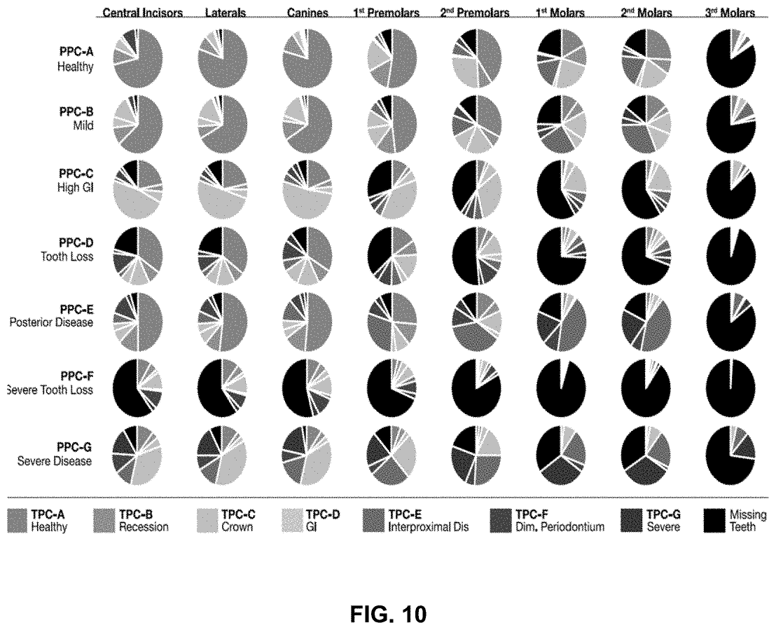

FIG. 10 shows the distribution of TPCs (including missing teeth) within each PPC, stratified by tooth type. This analysis combined all four quadrants of teeth [e.g. central incisors (#s 8+9+24+25)]. The diagram shows the intraoral distribution of TPCs for the various PPC classifications. In other words, the distributions of tooth conditions are displayed considering the periodontal health of the individual (PPC). There are few surprises, in that irrespective of PPC, there is a tendency for more disease and/or missing teeth moving from the anterior to posterior tooth positions. An exception is the High GI individual presents with more GI TPCs in the anterior regions as compared to posterior regions. The Interproximal disease TPCs are mainly on molars in individuals classified as having Mild Disease while the Interproximal disease TPCs extend to the sextant to include the premolars in the individuals with Posterior Disease. FIG. 10 can demonstrate that missing canines are predominantly in the Severe Tooth Loss PPC, consistent with the observation that canines are among the last teeth to be lost in a failing dentition.

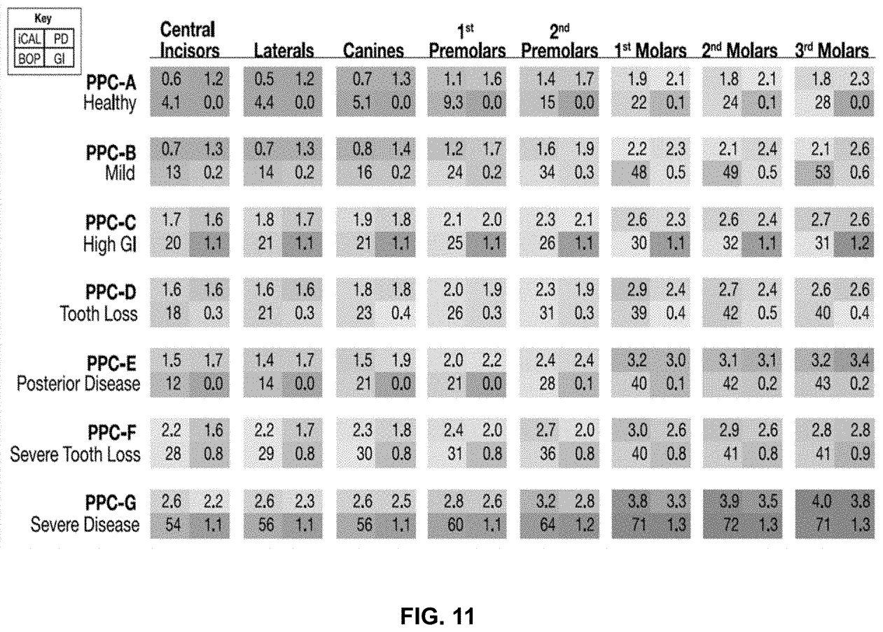

FIG. 11 displays the mean clinical values for interproximal attachment level, probing depths, extent (%) bleeding on probing, and mean gingival index, as a four-celled grid, for the 7×7 table of PPC by tooth type. Worsening mean values are shown in FIG. 11 that generally demonstrates more severe disease from anterior to posterior. It is interesting to note that the High GI PPC and the Severe PPC have multiple GI TPCs throughout the mouth; however, the High GI PPC has low/moderate BOP, whereas the Severe PPC has high BOP. Meanwhile, the Severe TPC also has a high percent of GI and plaque as well as 64% with BOP. In contrast, FIG. 11 shows that among PPC-Mild disease and PPC Posterior disease subjects, the higher extent of BOP scores are in the posterior teeth. However, the BOP scores increase dramatically from 54% to 71% anterior to posterior among Severe PPC, with second molars displaying an average of 3.9 mm iCAL and 3.5 mm Probing Depth. When present, the third molars consistently displayed more disease across all PPCs.

Discussion

Importantly, the reshuffling of subjects across domains of disease by differing classifications is, in itself, not meaningful, unless the new classifications provide additional insight into risk or responses to treatment. Historically, a legion of indices, extent scores, severity scores, clinical measures, and study-specific distributions of attachment loss and probing depth have been used to describe the prevalence and incidence of periodontal diseases and their associations with individual-level and group-level characteristics. These measures also have been useful for diagnosis and treatment planning. The assumption is that these tooth-based measures are as useful for other objectives, such as assigning risk for future disease progression, establishing associations with systemic diseases and conditions, and practicing precision dentistry. It was questioned whether this is a valid assumption. For this reason, all a priori assumptions of what the periodontal phenotype should look like were abandoned in deriving the P³ system.

For many years, literature reviews, position papers, and reports have strongly stated that it is difficult to assess the state of our knowledge, because of the variety of measures used to represent the periodontal phenotype.¹⁹⁻²³ In addition, it is still not known how to value teeth that are lost due to periodontitis, or other causes, when assessing risk for disease progression and tooth loss. For these reasons it was found that the various case status definitions used to describe the periodontal phenotype to be narrowly focused and of limited utility when attempting to generalize across studies or apply to other populations. Perhaps this problem is most profound when trying to establish a relevant case type for intervention studies. Inclusion criteria for case definitions are disparate and responders and non-responders often are thought to be a result of the inclusion criteria. For example, a couple of severe teeth (TPC-Severe) might qualify a subject for study enrollment, but the TPC Severe teeth do not intrinsically have similar risks for attachment loss when compared across PPCs¹.

Table 15 provides a number of insights into how the PPC differs in the way it classified people compared to the CDC/AAP and the European indices. A larger number of study participants are healthy and fewer are classified as Severe compared to the other indices. Of study participants classified as Healthy by the CDC/AAP, only 45% are Healthy by PPC and 12% are Mild. Another 10% are classified as Tooth Loss and another 29% are in the Severe Tooth Loss group. This pattern is similar for individuals classified by the European Index. These patterns show the influence of tooth loss, an event we have not been able to capture as part of a phenotype previously. Admittedly, a number of studies have adjusted for number of teeth in multivariable risk models; however; the PPC captures tooth loss, as well as High GI patterns, as separate sub-classes of the phenotype. The TPC Recession and TPC Diminished Periodontium classes represent special types of attachment loss that likely capture the biotype of the subject. Additional investigation has shown that High GI and the two tooth loss classes are at higher risk for future tooth loss and attachment loss¹. Traditionally, when an individual's periodontium is classified as having periodontitis, the GI status is ignored, i.e. there are noperiodontitis subcategories, such as “periodontitis with extensive inflammation”. The LCA agnostically created this High GI classification and it does perform well in predicting future attachment loss and tooth loss¹. It also is important to note that only slightly more than 25% of individuals classified as having Severe Periodontitis by the CDC/AAP and European indices were classified as Severe by the PPC, because most of the individuals moved to other PPCs that had major tooth loss. Is this separation of the Severe Periodontitis case status meaningful? A companion paper indicates that future tooth loss and attachment loss rates differ by these three groups¹. This pattern could mean that the groups respond differently to treatment, which also has implications for selection of research volunteers in future clinical studies. Among the generally younger individuals in the NHANES studies, similar patterns as 44% CDC/AAP and 35% European classified as Severe Disease were classified as Severe Disease by the PPC were observed. A good proportion (30-45%) of those not classified as Severe by PPC, were allocated to the Tooth Loss and Severe Tooth Loss categories. It should be noted that although the PPC appears to work similarly in the younger NHANES database, these classifications are based on individuals who have chronic periodontitis. However, among subjects of aged 30-35 in the NHANES data there are proportionately higher numbers of PPC-Severe individuals among those with disease (i.e. 22% vs 8.7% for those aged >60, data not shown). Thus, when disease is present among younger individuals, it tends to be more severe in clinical presentation consistent with an aggressive periodontitis classification.

This difference between the PPC and other indices may have implications for clinical studies, trials and treatments, especially those trials involving systemic diseases and chronic conditions. If tooth loss itself conveys part of the risk for prevalent or incident systemic conditions, then current treatments for periodontal disease would not reduce the portion of the risk represented by tooth loss.

The PPC is a more complex phenotype and the fact that a computer algorithm generated it may make some clinicians suspicious of its utility. However, the math has been done to harmonize group-level data to apply to an individual, such that a simple data entry of clinical signs by a practitioner will generate a total TPC profile and assign a PPC for the patient³. FIGS. 8-11 provide information on the more traditional clinical measures that underlie the different PPC and TPC monikers so that the reader can see the patterns and “get a feel” for the underlying clinical structure. For example, FIG. 9 shows for an individual to Healthy, there can be a few teeth with recession (and with concomitant attachment loss), a few missing teeth (mostly 3^{rd} molars), a couple teeth with Interproximal Disease, and on average, less than one tooth with High GI, Diminished Periodontium and Severe Disease; but risk for disease progression is low. However, it is not suggested that individuals classified as “healthy” are not in need of periodontal care—the moniker refers to those individuals with the lowest overall disease state and lowest risk for progression and tooth loss. FIG. 10 shows the influence of the individual's PPC on dental condition for each tooth type. For example, the patterns of Healthy teeth in Healthy individuals differ dramatically for each tooth type compared to individuals classified as High GI. Looking down each column, the influence of the individual's classification on the tooth health (TPC) of each tooth type can be observed. FIG. 11 presents the mean interproximal attachment level, probing depth, sites with BOP, and GI score for each tooth type for each PPC. Clinicians may find that all of these figures show patterns of oral health and disease that are familiar.

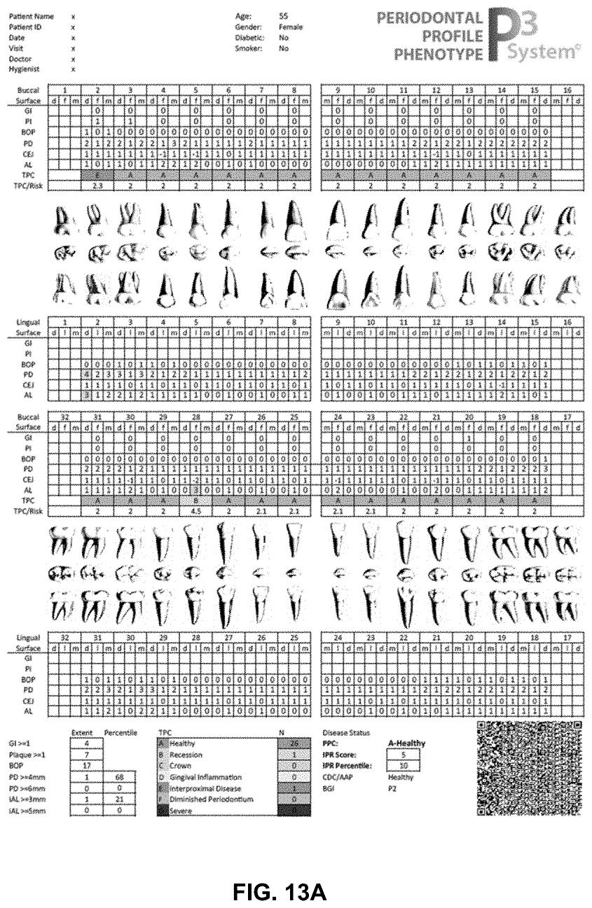

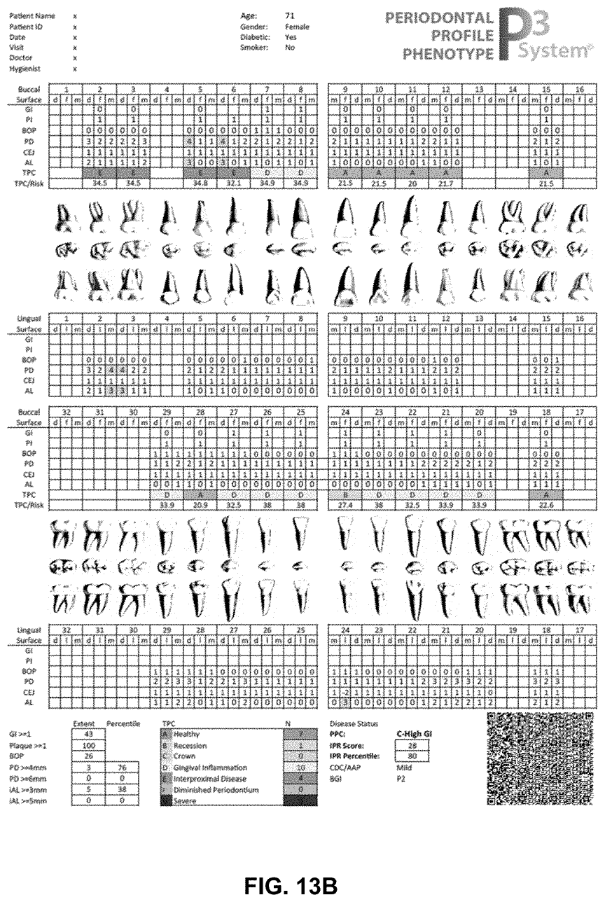

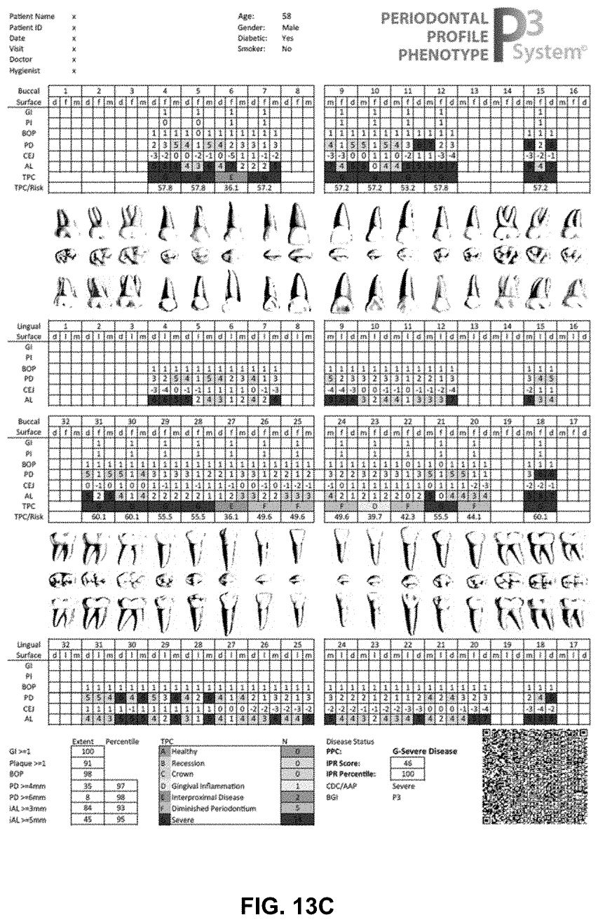

The P³ System was created so that it could be used by practitioners and researchers. A web-based data entry system so that practitioners will be able to submit a patient's clinical data, receive the patient's PPC class along with a risk score for future tooth loss as part of a clinical record. Three example records are displayed as FIGS. 13A, 13B and 13C. These figures show a typical record for a patient classified as PPC Healthy, PPC High GI, and PPC Severe. In FIGS. 13A-13C:

Rows labeled, Buccal or Lingual contain tooth numbers;

Rows labeled Surface are d=distal, f=facial, m=mesial;

GI is Gingival Index; PL is plaque score; BOP is bleeding on probing;

PD is probing depth;

CEJ is cemento-enamel junction; (negative value indicates recession)

AL is attachment level (AL≥3 is highlighted in yellow and AL≥5 is highlighted in red, as computed from PD-CEJ);

TPC is the Tooth Periodontal Class for the designated tooth that is color-coded based on the TPC designations at the bottom of the page; and

TPC/Risk is the 10-year probability of tooth loss for the specified TPC tooth within the patients specific PPC. TPC/Risk for tooth is adjusted for diabetes, gender, race smoking and age as described by Morelli et.al. (in this issue) as person-level, PPC adjustments. In addition, there are further adjustments made for each TPC risk measurement based upon tooth-type, and arch using the Piedmont 65+, 5-year tooth loss data. The person level PPC/TPC risk shown was adjusted at a tooth level using the parameters in Table 16.

### REFERENCES

1. Morelli T M K, Preisser J S, Beck J D, Divaris K, Wu D, Offenbacher S. Periodontal profile classes predict periodontal disease progression and tooth loss. J Periodontol 2017; [Submitted].

2. The Atherosclerosis Risk in Communities (ARIC) Study: design and objectives. The ARIC investigators. Am J Epidemiol 1989; 129:687-702.

3. Morelli T M K, Beck J, Preisser J D, Wu D, Divaris K, Offenbacher S. Derivation and validation of the Periodontal and Tooth Profile Classification System for patient stratification.”. J Periodontol 2017; 88:153-165.

4. Eke P I, Thornton-Evans G O, Wei L, Borgnakke W S, Dye B A. Accuracy of NHANES periodontal examination protocols. J Dent Res 2010; 89:1208-1213.

5. Dye B A, Li X, Lewis B G, lafolla T, Beltran-Aguilar E D, Eke P I. Overview and quality assurance for the oral health component of the National Health and Nutrition Examination Survey (NHANES), 2009-2010. J Public Health Dent 2014; 74:248-256.

6. Eke P I, Dye B A, Wei L, Thornton-Evans G O, Genco R Jat, workgroup. CPDS. Prevalence of periodontitis in adults in the United States: 2009 and 2010. J Dent Res 2012; 91:914-920.

7. Eke P I, Dye B A, Wei L, et al. Update on prevalence of periodontitis in adults in the United States: NHANES 2009 to 2012. J Periodontol 2015; 86:611-622.

8. Beck J D, Koch G G, Rozier R G, Tudor G E. Prevalence and risk indicators for periodontal attachment loss in a population of older community-dwelling blacks and whites. J Periodontol 1990; 61:521-528.

9. Beck J D, Koch G G, Offenbacher S. Attachment loss trends over 3 years in community-dwelling older adults. J Periodontol 1994; 65:737-743.

10. Beck J D, Sharp T, Koch G G, Offenbacher S. A 5-year study of attachment loss and tooth loss in community-dwelling older adults. J Periodontal Res 1997; 32:516-523.

11. Loe H, Silness J. Periodontal Disease in pregnancy. I. prevalence and severity. Acta Odontol Scand 1963; 21:533-551.

12. Silness J, Loe H. Periodontal disease in pregnancy. Ii. correlation between oral hygiene and periodontal condtion. Acta Odontol Scand 1964; 22:121-135.

13. Lanza S T, Collins L M, Lemmon D R, Schafer J L. PROC LCA: A SAS procedure for latent class analysis. Struct Equ Modeling 2007; 14:671-694.

14. Schwarz G. Estimating the dimension of a model. Ann Statist 1978; 6:461-464.

15. Milligan G W CM. An examination of procedures for determining the number of clusters in a data set. Psychometrika 1985; 50:159-179.

16. Nagan D. Group-based modelling of development: Harvard University Press; 2005.

17. Page R C, Eke P I. Case definitions for use in population-based surveillance of periodontitis. J Periodontol 2007; 78:1387-1399.

18. Tonetti M S, Claffey N, European Workshop in Periodontology group C. Advances in the progression of periodontitis and proposal of definitions of a periodontitis case and disease progression for use in risk factor research. Group C consensus report of the 5th European Workshop in Periodontology. J Clin Periodontol 2005; 32 Suppl 6:210-213.

19. Chapple I. L. C GR, and on behalf of working group 2. Diabetes and periodontal diseases: consensus report of the Joint EFP/AAP Workshop on Periodontitis and Systemi Diseases. J Clin Periodontol 2013; 40 S106-S112.

20. Tonetti M S, Van Dyke T E, working group 1 of the joint EFPAAPw. Periodontitis and atherosclerotic cardiovascular disease: consensus report of the Joint EFP/AAP Workshop on Periodontitis and Systemic Diseases. J Periodontol 2013; 84:S24-29.

21. Sanz M K K, on behalf of working group 3 Periodontitis and adverse pregnancy outcomes: consensus report of the Joint EFP/AAP workshop onperiodontitisw and systemic diseases. J Periodontol 2013; 84:S164-S169.

22. Dietrich T S P, Walter C, Weston P, Beck J. The epidemiological evidence behind the association between periodontitis andincident atherosclerotic cardiovascular disease. J Periodontol 2013; 84:S70-S84.

23. AAP. Epidemiology of periodontal diseases: position paper. J Periodontol 2005; 76:1406-1419.

24. Beck J D MK, Morelli T, Offenbacher S. Periodontal Profile Class (PPC) is associated with prevalent diabetes, coronary heart disease, stroke, and systemic markers of C-Reactive Protein and Interleukin 6. J Periodontol 2017; [Submitted].

TABLE 15
Concordance (Percent of Subjects) of PPC Status Classification of Periodontal Disease with CDC/AAP and European
Classifications in the Dental ARIC and NHANES 2009-2014 Studies

| | | | | | | | |
|---|---|---|---|---|---|---|---|
| Study and | PPC-A | PPC-B | PPC-C | PPC-D | PPC-E | PPC-F | PPC-G |
| Classification Used | Healthy | Mild | High GI | Tooth Loss | Posterior Disease | Severe Tooth Loss | Severe Disease |
| PPC Total N = 6,793 | N = 1,845 | N = 1,047 | N = 694 | N = 800 | N = 999 | N = 900 | N = 508 |

Dental ARIC - Number and (Row Percent of Study Participants)

| CDC * | | | | | | | | | | | | | | | |
|---|---|---|---|---|---|---|---|---|---|---|---|---|---|---|---|
| Healthy | N = 775 | 351 | (45%) | 93 | (12%) | 31 | (4%) | 75 | (10%) | 0 | (0%) | 225 | (29%) | 0 | (0%) |
| Mild | N = 2,035 | 867 | (43%) | 402 | (20%) | 286 | (14%) | 204 | (10%) | 50 | (2%) | 207 | (10%) | 19 | (1%) |
| Moderate | N = 2,799 | 582 | (21%) | 486 | (17%) | 284 | (10%) | 370 | (13%) | 573 | (20%) | 328 | (12%) | 176 | (6%) |
| Severe | N = 1,194 | 45 | (4%) | 66 | (6%) | 93 | (8%) | 151 | (19%) | 376 | (32%) | 140 | (12%) | 313 | (26%) |
| Total | 6,793 | | | | | | | | | | | | | | |
| European** | | | | | | | | | | | | | | | |
| Healthy | N = 749 | 379 | (47%) | 98 | (12%) | 28 | (4%) | 75 | (9%) | 0 | (0%) | 219 | (27%) | 0 | (0%) |
| Incipient | N = 5,021 | 1465 | (29%) | 939 | (19%) | 587 | (12%) | 575 | (11%) | 818 | (16%) | 388 | (8%) | 249 | (5%) |
| Severe | N = 973 | 1 | (0%) | 10 | (1%) | 79 | (8%) | 150 | (15%) | 181 | (19%) | 293 | (30%) | 259 | (27%) |
| Total = | 6,793 | | | | | | | | | | | | | | |

NHANES 2009-2014 - Number and (Row Percent of Study Participants)

| PPC Total N = 10,710 | | N = 5,878 | | N = 674 | | N = 962 | | N = 1,197 | | N = 506 | | N = 693 | | N = 800 | |
|---|---|---|---|---|---|---|---|---|---|---|---|---|---|---|---|
| CDC | | | | | | | | | | | | | | | |
| Healthy | N = 2,521 | 2277 | (90%) | 0 | (0%) | 5 | (0%) | 101 | (4%) | 0 | (0%) | 138 | (5%) | 0 | (0%) |
| Mild | N = 3,199 | 2568 | (80%) | 112 | (4%) | 145 | (5%) | 237 | (7%) | 17 | (1%) | 115 | (4%) | 5 | (0%) |
| Moderate | N = 3,791 | 1015 | (27%) | 505 | (13%) | 709 | (19%) | 605 | (16%) | 317 | (8%) | 370 | (10%) | 270 | (7%) |
| Severe | N = 1,199 | 18 | (2%) | 57 | (5%) | 103 | (9%) | 254 | (21%) | 172 | (14%) | 70 | (6%) | 525 | (44%) |
| Total | N = 10,710 | | | | | | | | | | | | | | |
| European | | | | | | | | | | | | | | | |
| Healthy | N = 2,563 | 2350 | (92%) | 0 | (0%) | 4 | (0%) | 95 | (4%) | 0 | (0%) | 114 | (4%) | 0 | (0%) |
| Incipient | N = 6,681 | 3525 | (53%) | 666 | (10%) | 766 | (11%) | 748 | (11%) | 417 | (6%) | 272 | (4%) | 287 | (4%) |
| Severe | N = 1,466 | 3 | (0%) | 8 | (1%) | 192 | (13%) | 354 | (24%) | 89 | (6%) | 307 | (21%) | 513 | (35%) |
| Total = | 10,710 | | | | | | | | | | | | | | |

* The definitions for levels of the CDC/AAP index are:
No periodontitis - No evidence of mild, moderate, or severe periodontitis;
Mild periodontitis - ≥2 interproximal sites with AL of ≥3 mm and ≥2 interproximal sites with PD of ≥4 mm [not on same tooth] or one site with PD ≥5 mm;
Moderate periodontitis - ≥2 interproximal sites with AL of ≥ 4mm [not on same tooth], or ≥2 interproximal sites with PD of ≥5 mm [not on same tooth];
Severe periodontitis - ≥2 interproximal sites with AL of ≥6 mm [not on same tooth]and ≥1 interproximal site with PD ≥5 mm.
**The definitions for levels of the European Index are:
No periodontitis - No evidence of incipient or severe periodontitis;
Incipient Periodontitis - presence of proximal attachment loss of ≥3 mm in ≥2 non-adjacent teeth;
Severe periodontitis - presence of proximal attachment loss of ≥5 mm in ≥30% of teeth.

TABLE 16
Percent Adjustment to TPC Risk for Tooth Type by Arch

| | Maxilla | Mandible |
|---|---|---|
| Incisor | 0% | 9% |
| Canine | −7% | −7% |
| Premolar | 1% | −3% |
| Molar | 0% | 5% |

### EXAMPLE 4

Introduction.

Interest in potential relationships between periodontal disease and systemic diseases remains high among clinicians, researchers, and the public. Over the last twenty years, the majority of human studies of associations between periodontal disease and prevalence of systemic diseases and related conditions have reported significant associations while some studies did not. This has resulted in discussions regarding the inconsistent results of these population-based studies. Many factors could have played a role in the inconsistency of results, such as sample size, the characteristics of the groups studied, examiner differences, and the systemic condition studied. A major factor related to inconsistencies in study results is how the exposure, periodontal disease, is measured.

This Example focuses on, inter ailia, the impact of an investigator's choice of a measure to represent the periodontal phenotype on associations with systemic conditions and inflammation. We have developed a new measure of the periodontal disease phenotype that may have greater utility for personalized dentistry than current case definitions of periodontitis and have validated, tested, and shown utility for risk profiling for tooth loss and attachment loss for that measure. The Periodontal Profile Class (PPC) is a person-level measure that provides a clinical, seven-class taxonomic system for the patient's disease status. The PPC is one component of the Periodontal Profile Phenotype (P³) that is described in another publication¹. This Example can demonstrate the utility of the P³ System by presenting associations between PPC with prevalent diabetes, coronary heart disease (CHD) and stroke, as well as systemic measures of high sensitivity C-Reactive Protein (hs-CRP) and Interleukin-6 (IL-6), adjusting for relevant confounders. In addition, this Example compares the PPC associations with results using traditional measures of periodontal case status.²⁻⁴

Materials and Methods

Study Samples. The ARIC study⁵ enrolled 15,792 participants within the age group of 45-64 in four different U.S. communities (Forsyth County, N.C., Jackson, Miss., suburbs of Minneapolis, Minn., and Washington County, Md.). All participants provided written informed consent to a protocol that was reviewed and approved by the Institutional Review Board on research involving human subjects at the University of North Carolina and/or at each study performance site. All participants who completed the fourth clinic visit (1996-1998) in ARIC (N=11,656) were eligible for inclusion. Of the 11,656 ARIC participants seen at the fourth clinical visit, we excluded study participants who did not receive a periodontal examination. These exclusions resulted in 6,793 individuals who were included in this study as well as the Latent Class Analysis (LCA)⁶ that resulted in the PPC.⁷

Three additional datasets from the National Health and Nutrition Examination Survey (NHANES; 2009-2010, 2011-2012, and 2013-2014) were combined as a validation study population. The technical details of the surveys, including sampling design, periodontal data collection protocols, and data availability, are described elsewhere.8, 9 2, 10 Briefly, periodontal measurements were collected for 3,750 individuals (NHANES 2009-2010), for 3,338 individuals (NHANES 2011-2012), and 3622 individuals for (NHANES 2013-2014) for a total of 10,710. Periodontal measures were collected on six-sites per tooth for all teeth present in the mouth except third molars.¹¹⁻¹³

Measurement of Exposures. Periodontal Profile Class (PPC). The analytical approach implemented person-level LCA to identify discrete classes of individuals using seven tooth-level clinical parameters. These parameters were: site with interproximal attachment level (IAL) ≥3 mm, ≥1 site with probing depth (PD) extent of bleeding on probing (BOP, dichotomized at 50% or ≥3 sites per tooth), gingival inflammation index¹⁴ (GI dichotomized as GI=0 vs. GM), plaque index¹⁵ (PI dichotomized as PI=0 vs. PI≥1), the presence/absence of full prosthetic crowns for each tooth, and tooth status (present vs. absent).⁷

Briefly (see Morelli, et al⁷ for a more complete description), individuals were classified into mutually-exclusive latent classes based upon their responses to a set of observed categorical variables while ensuring that clinically relevant categories were maintained. We used Milligan and Cooper's¹⁶ recommendation for the maximum number (n) of classes, suggesting stopping when the newly added class (n+1) is not clinically distinct from the previous number (n) of identified classes. Additionally, it was verified that mean posterior probabilities of correct class assignment were >0.7, which according to Nagin¹⁷ indicates adequate class separation and membership precision. In the first step of LCA, the person-level LCA classified individuals into seven latent classes based on 224 dichotomous variables (derived from seven tooth-level variables, using the clinical parameters referred to above for each of 32 teeth). The class membership probabilities represent the overall, unconditional proportions of individuals in each of seven latent classes. The model parameters from the first step were then used to compute the posterior probabilities (the probability of event A occurring given that event B has occurred) of each individual's membership into each class conditional upon the values of the 224 items, or as many of them as were measured for that individual.⁷

CDC/AAP and European Indices. The Centers for Disease Control/American Academy of Periodontology (CDC/AAP) index¹⁸ along with the European Periodontal index⁴ may be the most frequently used indices and are a step forward in creating some consistency in periodontal disease case definitions. The CDC/AAP index was developed as a three-level index and later expanded to a four-level index (Healthy; Mild; Moderate; and Severe disease).² We used the four-level index as it provided more separation of the Healthy and Mild groups. The definitions of the levels of disease for both indices appear in Table 17. The European Index has 3-levels (Healthy, Incipient, and Severe)⁴

Prevalent Diseases and Conditions. The dependent variables in this study include three prevalent diseases (diabetes, coronary heart disease, and stroke) and two markers of systemic inflammation (C-reactive protein and Interleukin 6). All prevalent measures of disease gathered at ARIC Visit 1 were updated by incident disease during the nine years of follow-up until the periodontal examination during visit 4. Prevalent coronary heart disease (CHD) was defined as a self-reported history of a physician-diagnosed heart attack; or evidence of an old myocardial infarction by electrocardiogram based on the Minnesota codes; or a history of coronary surgery or coronary angioplasty. Stroke and/or TIA was obtained by an interviewer-administered questionnaire, as a report of stroke or TIA diagnosed by a physician. Angina pectoris and intermittent claudication were measured using the Rose Questionnaire¹⁹. Prevalent diabetes mellitus was classified as a non-fasting serum glucose level of at least 200 mg/dL, a fasting glucose level of at least 140 mg/dL, a history of diabetes, or the current use of diabetes medication.^{20, 21}

Measurement of Serum IL-6 and hsCRP. Interleukin-6 (IL-6) and C-reactive protein (CRP) concentrations from once thawed serum aliquots (frozen at −80 C from collection until December 2009) were all measured by ELISA techniques. Spectrophotometric endpoints were determined on a SpectraMax M2 plate reader (Molecular Probes, Sunnyvale, Calif.) using reagent assay kits from R&D Systems (Minneapolis, Minn.) according to manufacturer's instructions. The Softmax® (v.5.0.1) control software package was used to fit the standard curve data using either 4-PL or 5-PL fitting algorithms to provide a best fit of the seven-point (duplicate) standard curve after subtraction of the mean reagent blank values from all measured optical densities. Standard curve concentrations ranged from 0.156-10 pg/ml for serum IL-6 (using the high sensitivity antibody) and 780-50,000 pg/ml for CRP, Although hs-CRP was measured using ELISA (several years prior to the development of CLIA certified nephelometry methods) the ELISA values have been validated and shown to have high agreement against the current clinical tests (r=0.9, data not shown) with the advantage of having higher sensitivity for values <3 mg/dl.

The NHANES studies^{2,3} collected information on prevalent CHD and Stroke by standardized questions. Responses from three questions were combined to create the CHD variable. The three questions were: Has a doctor ever told you; (1) that you had coronary heart disease; (2) that you had a heart attack, or (3) that you had congestive heart failure? The response to the question; has a doctor ever told you had a stroke is the basis for the stroke variable.

Other Variables of Interest. Age, sex, race, and additional vascular risk factors, such as body mass index and lipid profile, were measured according to published methods. Fasting plasma HDL and LDL cholesterol, triglycerides, and fasting serum glucose, (all in mg/dl) were collected before the clinical examination. Methods for measurement of body mass (BMI) and blood pressure have been described previously^{22, 23} and hypertension was defined as having a systolic blood pressure ≥140 mm/hg or diastolic blood pressure ≥90 mm/hg or taking blood pressure reducing medications^{22,23}.

Participants were defined as never smokers, former smokers, or current smokers by interview. Education level was divided into basic (fewer than 12 years), intermediate (12 to 16 years), or advanced (17 to 21 years), and was included to control for socioeconomic status. Age in years at visit 4 was included and a variable representing race/ethnicity (African-American or White) and ARIC field center was designed to control for the ethnic, regional, and examiner differences in the ARIC study. Hypertension was defined as the average of two blood pressure readings at the visit (with systolic blood pressure having a cutoff point of 140 mm HG or higher and diastolic blood pressure having a cutoff point of 90 mm Hg or higher) or the use of hypertension medication.

Statistical Analysis. Analyses were performed to determine associations between PPC and prevalent diabetes, CHD, stroke, serum CRP, and serum IL-6 for the participants of the Dental ARIC Study using logistic regression to compute odds ratios (OR) and 95% confidence intervals (CI) with adjustment for potential confounders.

Each PPC category was compared to the reference group (Healthy) and used a similar analytic strategy for categories of the CDC/AAP and the European indices and followed the same process for the NHANES study participants, except that the NHANES study did not include a serum IL-6 measure. The Bayesian Information Criterion (BIC)²⁴ was computed to determine the fit of the model and the magnitude of the attributable contribution that periodontal assessment provides beyond traditional risk factors.

The criteria was modified to represent model “improvement” by changing the negative signs to positive signs with the interpretation that the models obtained by adding periodontal disease to the existing traditional variables in the model significantly improved the overall fit of the model. Specific levels of BIC improvement appear in Table 18.

Results

Associations with Prevalent Systemic Diseases and Conditions. Distributions of prevalent CHD according to demographic, systemic health, and periodontal disease case status for ARIC and NHANES 9-14 study participants are shown in Table 17. Similar distributions for diabetes, stroke, CRP and IL-6 are shown in Tables 21-24. Participants in either studies who were Caucasian, male, or hypertensive had a higher CHD prevalence. Higher mean age, BMI, and triglycerides were associated with prevalent CHD as were lower mean scores for LDL, HDL, and total cholesterol. The pattern for education level differed for the two studies with higher prevalence of CHD in those with basic levels of education in ARIC and with intermediate level in NHANES. Patterns also differed for smoking with current smokers in ARIC and former smokers in NHANES with higher CHD prevalence. Participants in both studies classified by the CDC/AAP and European indices as having Severe Periodontitis had higher prevalence of CHD while the pattern differed for the PPC classifications. ARIC participants who had Tooth Loss and Severe Tooth Loss had higher prevalence of CHD closely followed by the High GI and Severe groups. NHANES participants with Severe Tooth Loss had the highest prevalence of CHD followed by the Tooth Loss, High GI, and the Posterior Disease groups. Overall, there appears to be a higher prevalence of CHD in the ARIC participants, who tend to be older. The variables in Table 17 were significantly related to CHD in both studies except for race and BMI in the ARIC study and triglyceride level in NHANES. If a variable was significantly associated with CHD in one of the studies, we included that variable in the model for both studies.

Table 18 presents associations between the PPC and prevalent systemic conditions of diabetes, coronary heart disease (CHD), stroke, C-Reactive Protein (hs-CRP) and serum IL-6 for ARIC study participants. The number of study participants with these diseases and conditions vary and are shown at the top of each column. All models were adjusted for relevant confounders and covariates including race/center, age, gender, BMI, smoking (3-level), education plus lipids and other systemic conditions as relevant to the disease. These models each served as a reference model for computing the BIC—(Baysian Information Criterion) that permitted the comparison of having periodontal disease in the model to not having periodontal disease. It can be seen that BIC improvement scores are above 10 (Very Strong contribution) for the PPC classification with the CDC/AAP and European classifications having lower BIC scores with many below 2.0. For diabetes in Table 18, most classifications show significant odds ratios for the PPC, while nothing was significant for the CDC/AAP, and European indices. The PPC classifications for people with periodontal disease and have retained most of their teeth fall under the Mild, High GI, Posterior Disease and Severe classes. Of these classes, the High GI, and Severe Disease classes are associated with prevalent diabetes. There also are higher odds of having prevalent diabetes with both Tooth Loss classes and Severe Disease. The PPC for Severe Tooth Loss has the highest odds ratio (1.72) for diabetes and the entire PPC model showed the greatest BIC Improvement. All the PPC High GI, Tooth Loss, Severe Tooth Loss, and Severe Disease classes were significant for CHD. Only the Severe Disease class was significant for prevalent stroke. High GI, the two Tooth Loss classes, and Severe Disease were significant for serum CRP and IL-6. The only significant associations for the CDC/AAP and European categories were between Severe Disease and CHD. Cardiovascular disease is divided into coronary heart disease and stroke as they share some risk factors, but have differing mechanisms of pathogenesis. Among the CDC/AAP and European classifications, only the severe categories show significant associations with CHD and none of the associations with stroke were significant. Several of the PPC classes are significantly associated with CHD with High GI being the strongest followed by Mild Disease and the two Tooth Loss classes. However, Severe Disease was not significant. By contrast, only the PPC Severe Disease class was significantly associated with stroke. Hs-CRP and IL-6 demonstrated significant associations with multiple PPC categories while none of the CDC/AAP or European classifications were significantly associated.

The analyses shown in Table 18 appear in Table 19 for the three combined NHANES cohorts (2009-2010, 2011-2012, and 2013-2014). IL-6 information was not available in NHANES, so is absent in this table. BIC Improvement scores for having periodontal disease classifications in the models were very strong for all PPC models, but were weaker for most all CDC/AAP and European-based models. The NHANES results in Table 19 show all PPC classifications except Posterior Disease were significantly associated with Diabetes as were the CDC/AAP Moderate and Severe and the European Incipient and Severe categories. The PPC models show that the Posterior Disease and the Severe Tooth Loss classes are the only ones associated with CHD, while both Tooth Loss and the Posterior Disease classes are associated with Stroke, and CRP. The CDC/AAP and European models were associated with CHD and CRP, but not with Stroke. In the models for CHD, Moderate and Incipient disease were significantly associated with CHD, but not the Severe Disease class. The BIC scores for the PPC and CDC/AAP models indicated that periodontitis made a very strong contribution, while the score for the European model was much weaker.

Discussion

Table 18 showed that study participants with CHD had lower LDL levels than those without CHD. This relationship was supported by additional analysis of ARIC medication data showing that 46% of participants with CHD used lipid-lowering medications compared to 11% without CHD.

In Table 18, the associations between the CDC/AAP, European and PPC indices and the prevalence of three systemic diseases (Diabetes, CHD, and Stroke) were compared along with two markers of systemic inflammation (hs-CRP and serum IL-6) using the ARIC dataset. Without being bound by theory, it was thought that the broader PPC representation of the periodontitis phenotype would have a higher probability of being associated with other person-level oral and systemic conditions. The PPC representation of the periodontitis phenotype produced multiple statistically significant associations with the systemic diseases and conditions along with “strong and very strong” BIC scores indicating that PPC made meaningful additions to the multi-variable models. The only significant associations related to the CDC/AAP and European indices are for prevalent CHD with BIC scores in the “positive and strong” categories, respectively. The associations between Severe Disease and CHD is consistent with a multitude of other studies using a variety of periodontal indices, but the lack of an association between Severe disease and CHD using the PPC could be important as it may indicate that the effects of high levels of tooth loss and High GI underlie traditional associations with prevalent CHD. High GI is significantly associated with all the systemic diseases and conditions except Stroke, which is known to share some common risk factors, but not others. While the High GI class has extensive inflammation with less attachment loss and shallower pockets, it is significantly associated with diabetes, CHD, and the systemic inflammatory markers; whereas other classes with similar levels of periodontal disease (Mild, without extensive inflammation), are not associated with those conditions. The High GI class is a novel feature of the PPC and its association with systemic diseases and inflammatory conditions provides support for the profession's reduction of inflammation as a goal of periodontal treatment.

It is interesting that the Posterior Disease class that is similar to the traditional definitions of moderate and incipient disease, which usually begins in the posterior dentition, is not associated with any of the prevalent diseases or inflammatory biomarkers. Individuals in the Posterior Disease class may have qualified for inclusion in clinical intervention studies or randomized controlled trials to test whether treatment of periodontal disease prevents or reduces these systemic diseases or inflammatory mediators. However, given the lack of significant associations seen in Table 18, we might not expect treatment of this phenotype to affect these conditions.

Table 19 presents the same analyses conducted in Table 18 using the NHANES 2009-2014 dataset except for IL-6 scores, which were not available. This validation sample was larger than the Dental ARIC sample with more cases. It also was a younger sample and likely healthier, since it included study participants as young as 30 years of age. We conducted a second analysis for the NHANES 2009-2014 sample that was restricted to the same age range as the ARIC sample and the patterns of associations were very similar (data not shown). Although the ARIC and NHANES samples differ by age and, likely health, the patterns of associations for the PPC compared to the CDC/AAP indices with prevalent diseases and conditions are very similar. The PPC models generally make stronger contributions to the models and are more likely to show a significant association with prevalent diseases and conditions than the other indices. Additionally, there are some specific patterns of interest between the PPC models for the two datasets. While periodontal disease is associated with prevalent CHD in both datasets, Mild and High GI are not associated with CHD in NHANES, which could be a function of a younger NHANES sample. While only Severe Disease is associated with stroke in ARIC, associations with the two Tooth Loss classes and Posterior Disease in NHANES replace it. Thus, the PPC performs similarly in both datasets, but the strength of the associations may differ among components of PPC.

It is difficult to value the teeth that are lost due to periodontitis or other reasons when assessing risk for disease progression and tooth loss and case status definitions used in the past are narrowly focused when attempting to describe the periodontal phenotype. This difficulty is most profound when trying to establish a relevant case type for intervention studies. Inclusion criteria for case definitions are disparate and responders and non-responders often are thought to be attributable to inclusion criteria. For example, a patient with PPC-G Severe Disease has many teeth at risk for disease progression and is thought to respond better to whole-mouth therapeutic intervention. However, certain PPC-F Severe Tooth Loss patients may have enough teeth and enough disease to qualify for the study, but are at lower risk of progression²⁵ and less likely to respond to the same intervention.

This Example can further support the generation of an index that can represent the periodontal phenotype by including additional commonly collected clinical and person-level characteristics. The P³ system is designed to meet the clinical utility needs of diagnosis, assigning prognosis and risk, as well as measuring clinical outcomes in response to therapy. The diagnostic algorithm is robust and the math has been done so that clinical data can be inputed from a single patient and TPCs, PPC, and risk scores for a given individual can be computed. The assignment of TPCs and PPCs has been demonstrated to be robust using a wide range of datasets and can be used to harmonize different studies, PPC misclassification is not a significant problem, i.e. 85% correct assignment rate under the worst comparisons of two missing clinical indices and less than full-mouth exams. One important caveat in the classification of the TPC and calculation of the IPC is that it includes scoring of coronal and root caries. However, the prevalence of caries in the Dental ARIC sample is very low overall with mean decayed surfaces <1%. Although caries is included in the measures, the contribution of canes to the TPC phenotype is negligible as this condition mostly relates to the probability of tooth loss.

In many trials designed to examine the potential periodontal treatment effects on systemic inflammation and/or systemic disease, criteria for subject eligibility often is predicated upon having a certain minimum number of teeth with pockets and attachment loss but do not consider either a high percentage of GI classified teeth or tooth loss. Since these data demonstrate associations with prevalent outcomes, it suggests the mechanistic importance of having a history of periodontal disease that results in tooth loss as a component of risk for continuing systemic inflammation and risk for systemic disease. However, the current periodontal therapy armamentarium is not able to address the effect of past inflammation implied by a tooth lost to periodontal disease. Increasingly, the evidence that the oral microbiome is highly mobile and can translocate to other tissues and persist in extraoral compartments may provide a link between the history of chronic periodontal disease leading to tooth loss and systemic diseases [for review see Han and Wang²⁶].

These data support the concept that the PPC classification significantly improves the overall risk model of systemic disease association when combined with traditional risk factors.

Summary

The addition of the PPC phenotype to traditional variables associated with prevalent diabetes, stroke, CHD and systemic measures of inflammation resulted in very strong improvement of the overall models. The PPC is consistently associated with systemic diseases and conditions, even though age composition of the sampled populations may differ.The PPC appears to capture the systemic exposure component of the phenotype, as it is more strongly associated with systemic markers of inflammation than comparison indices. The new PPC High GI class is strongly associated with systemic inflammation, prevalent diabetes, and CHD while the Tooth Loss classes show promise for representing the individual's history of inflammation. The components of the PPC provide insight into the aspects of the phenotype that are associated with systemic conditions that can be useful in designing clinical interventions.

### REFERENCES

1. Beck J D, Moss K, Morelli T, Offenbacher S. In search of an appropriate measure of the periodontal phenotype: The periodontal profile class system (PPC). J Periodontol 2017; [Submitted].

2. Eke P I, Dye B A, Wei L, et al. Update on Prevalence of Periodontitis in Adults in the United States: NHANES 2009 to 2012. J Periodontol 2015; 86:611-622.

3. Eke P I, Dye B A, Wei L, Thornton-Evans G O, Genco R J and, CDC Periodontal Disease Surveillance workgroup: Beck J, Douglas G, Page R. Prevalence of periodontitis in adults in the United States: 2009 and 2010. J Dent Res 2012; 91:914-920.

4. Tonetti M S, Claffey N, European Workshop in Periodontology group C. Advances in the progression of periodontitis and proposal of definitions of a periodontitis case and disease progression for use in risk factor research. Group C consensus report of the 5th European Workshop in Periodontology. J Clin Periodontol 2005; 32 Suppl 6:210-213.

5. The Atherosclerosis Risk in Communities (ARIC) Study: design and objectives. The ARIC investigators. Am J Epidemiol 1989; 129:687-702.

6. Lanza S T, Collins L M, Lemmon D R, Schafer J L. PROC LCA: A SAS Procedure for Latent Class Analysis. Struct Equ Modeling 2007; 14:671-694.

7. Morelli T, Moss K, Beck J, Preisser J D, Wu D, Divaris K, Offenbacher S. Derivation and Validation of the Periodontal and Tooth Profile Classification System for Patient Stratification.” J Periodontol 2017; 88:153-165.

8. Eke P I, Thornton-Evans G O, Wei L, Borgnakke W S, Dye B A. Accuracy of NHANES periodontal examination protocols. J Dent Res 2010; 89:1208-1213.

9. Dye B A, Li X, Lewis B G, lafolla T, Beltran-Aguilar E D, Eke P I. Overview and quality assurance for the oral health component of the National Health and Nutrition Examination Survey (NHANES), 2009-2010. J Public Health Dent 2014; 74:248-256.

10. Eke P I, Dye B A, Wei L, Thornton-Evans G O, Genco R J, Cdc Periodontal Disease Surveillance workgroup: James Beck GDRP. Prevalence of periodontitis in adults in the United States: 2009 and 2010. J Dent Res 2012; 91:914-920.

11. Beck J D, Koch G G, Rozier R G, Tudor G E. Prevalence and risk indicators for periodontal attachment loss in a population of older community-dwelling blacks and whites. J Periodontol 1990; 61:521-528.

12. Beck J D, Koch G G, Offenbacher S. Attachment loss trends over 3 years in community-dwelling older adults. J Periodontol 1994; 65:737-743.

13. Beck J D, Sharp T, Koch G G, Offenbacher S. A 5-year study of attachment loss and tooth loss in community-dwelling older adults. J Periodontal Res 1997; 32:516-523.

14. Loe H, Silness J. Periodontal Disease in Pregnancy. I. Prevalence and Severity. Acta Odontol Scand 1963; 21:533-551.

15. Silness J, Loe H. Periodontal Disease in Pregnancy. li. Correlation between Oral Hygiene and Periodontal Condtion. Acta Odontol Scand 1964; 22:121-135.

16. Milligan G W CM. An examination of procedures for determining the number of clusters in a data set. Psychometrika 1985; 50:159-179.

17. Nagan D. Group-Based Modeling of Development: Harvard University Press; 2009.

18. Page R C, Eke P I. Case definitions for use in population-based surveillance of periodontitis. J Periodontol 2007; 78:1387-1399.

19. Rose G A B H, Gillum R F, Prineas R J. Cardiovascular Survey Methods. Switzerland: World Health Organization.

20. Slade G D, Ghezzi E M, Heiss G, Beck J D, Riche E, Offenbacher S. Relationship between periodontal disease and C-reactive protein among adults in the Atherosclerosis Risk in Communities study. Arch Intern Med 2003; 163:1172-1179.

21. Andriankaja O M, Barros S P, Moss K, et al. Levels of serum interleukin (IL)-6 and gingival crevicular fluid of IL-1beta and prostaglandin E(2) among non-smoking subjects with gingivitis and type 2 diabetes. J Periodontol 2009; 80:307-316.

22. University of North Carolina. ARIC Manual of Operations, No. 2: Cohort Component Procedures. Version 1.0.

23. Investigators T A. ARIC Manual of Operations, No. 11: Sitting Blood Pressure. Version 1. Chapel Hill, N.C.: University of North Carolina; 1987.

24. Cavanaugh J E. Model Selection: Lecture VI: The Bayesian Information Criterion” (PowerPoint presentation. In. Iowa City, Iowa: University of Iowa, 2009.

25. Morelli T, Moss K, Preisser J S, Beck J D, Divaris K, Wu D, Offenbacher S. Periodontal profile classes predict periodontal disease progression and tooth loss. J Periodontol 2017; [Submitted].

26. Han Y W W X. Mobile microbiome: oral bacteria in extra-oral infections and inflammation. J Dental Research 2013; 92:485-491.

TABLE 17
ARIC and NHANES: Prevalent CHD by Demographics, General
Health and Periodontal Disease Case Definitions

| Demographic, Health and | Dental ARIC (N = 6,793) | | | | NHANES (N = 10,710) | | | | |
|---|---|---|---|---|---|---|---|---|---|
| Periodontal Disease | CHD | | CHD | | CHD | | CHD | | p-value |
| Variables | N (Yes) | | N (No) | | N (Yes) | | N (No) | | D-ARIC/NHANES |
| African American | 64 | (5.1%) | 1199 | (94.9%) | 66 | (3.0%) | 2162 | (97.0%) | 0.21/<0.0001 |
| Caucasian | 326 | (6.0%) | 5125 | (94.0%) | 183 | (4.0%) | 4397 | (96.0%) | |
| Other | . . . | | . . . | | 81 | (2.1%) | 3810 | (97.9%) | |
| Female | 114 | (3.1%) | 3537 | (96.9%) | 99 | (1.8%) | 5334 | (98.2%) | <0.0001/<0.0001 |
| Male | 278 | (9.0%) | 2810 | (91.0%) | 231 | (4.4%) | 5035 | (95.6%) | |
| Mean(SD) Age | 64.4 | (5.6) | 62.3 | (5.6) | 65.4 | (12.0) | 51.6 | (14.1) | <0.0001/<0.0001 |
| Mean(SD) BMI | 30.0 | (5.4) | 28.7 | (5.4) | 30.5 | (6.6) | 29.3 | (6.7) | 0.14/0.001 |
| Diabetes | 94 | (10.1%) | 837 | (89.9%) | 101 | (7.6%) | 1234 | (92.4%) | <0.0001/<0.0001 |
| No Diabetes | 297 | (5.1%) | 5477 | (94.9%) | 229 | (2.5%) | 9135 | (97.6%) | |
| Hypertensive | 204 | (8.9%) | 2080 | (91.1%) | 248 | (6.2%) | 3760 | (93.8%) | <0.0001/<0.0001 |
| Non-Hypertensive | 179 | (4.0%) | 4249 | (96.0%) | 82 | (1.2%) | 6594 | (98.8%) | |
| Current Smoker | 104 | (10.8%) | 861 | (89.2%) | 74 | (3.7%) | 1932 | (96.3%) | <0.0001/<0.0001 |
| Former Smoke | 257 | (5.2%) | 4723 | (94.8%) | 129 | (4.8%) | 2547 | (95.2%) | |
| Never Smoker | 31 | (3.9%) | 763 | (96.1%) | 127 | (2.1%) | 5885 | (97.9%) | |
| Mean(SD) LDL | 114.4 | (32.1) | 122.5 | (33.1) | 94.1 | (36.8) | 113.4 | (37.2) | <0.0001/<0.0001 |
| Mean(SD) Total | 188.3 | (34.5) | 201.8 | (35.9) | 175.5 | (42.3) | 197.4 | (41.5) | <0.0001/0.0001 |
| Cholesterol | | | | | | | | | |
| Mean(SD) Triglyceride | 154.0 | (91.1) | 142.2 | (83.6) | 171.7 | (115.4) | 159.1 | (145.0) | 0.007/0.13 |
| Mean(SD) HDL | 43.1 | (13.4) | 50.9 | (16.7) | 49.3 | (17.1) | 53.0 | (16.0) | <0.0001/<0.0001 |
| Education | | | | | | | | | |
| Basic | 78 | (8.7%) | 824 | (91.4%) | 88 | (3.5%) | 2409 | (96.5%) | 0.0002/0.0008 |
| Intermediate | 170 | (5.9%) | 2735 | (94.2%) | 92 | (4.0%) | 2208 | (96.0%) | |
| Advanced | 144 | (4.9%) | 2780 | (95.1%) | 149 | (2.5%) | 5740 | (97.5%) | |
| PPC-Health | 61 | (3.3%) | 1774 | (96.7%) | 95 | (1.6%) | 5780 | (98.4%) | <0.0001/<0.0001 |
| PPC-Mild | 57 | (5.5%) | 986 | (94.5%) | 11 | (1.6%) | 663 | (98.4%) | |
| PPC-High GI | 46 | (6.8%) | 634 | (93.2%) | 49 | (5.1%) | 910 | (94.9%) | |
| PPC-Tooth Loss | 62 | (7.8%) | 729 | (92.2%) | 70 | (5.9%) | 1126 | (94.9%) | |
| PPC-Posterior Disease | 62 | (6.2%) | 936 | (93.8%) | 26 | (5.1%) | 480 | (92.5%) | |
| PPC-Severe Tooth Loss | 70 | (7.9%) | 818 | (92.1%) | 52 | (7.5%) | 640 | (92.5%) | |
| PPG-Severe Disease | 34 | (6.8%) | 470 | (93.3%) | 27 | (3.4%) | 770 | (96.6%) | |
| CDC/AAP Healthy | 32 | (4.2%) | 738 | (95.8%) | 29 | (1.2%) | 2491 | (98.9%) | <0.0001/<0.0001 |
| Mild | 88 | (4.4%) | 1932 | (95.6%) | 70 | (2.2%) | 3127 | (97.8%) | |
| Moderate | 168 | (6.1%) | 2603 | (93.9%) | 175 | (4.6%) | 3610 | (95.4%) | |
| Severe | 104 | (8.8%) | 1074 | (91.2%) | 56 | (4.7%) | 1141 | (95.3%) | |
| European Healthy | 31 | (3.9%) | 763 | (96.1%) | 26 | (1.0%) | 2536 | (99.0%) | <0.0001/<0.0001 |
| Incipient | 257 | (5.2%) | 4723 | (94.8%) | 220 | (3.3%) | 6455 | (96.7%) | |
| Severe | 104 | (10.8%) | 861 | (13.6%) | 84 | (5.8%) | 1378 | (94.3%) | |

TABLE 18
Associations between Three Indices of Periodontal Disease Case Status
and Prevalent Systemic Conditions in the Dental ARIC Study.

| | | Diabetes | CHD | Stroke | CRP | IL-6 |
|---|---|---|---|---|---|---|
| | | N Cases/Total | | | | |
| | | 924/6676 | 313/5491 | 91/5503 | 1378/5513 | 1352/5442 |
| Case Status | (N) | OR (95% CI) | OR (95% CI) | OR (95% CI) | OR (95% CI) | OR (95% CI) |
| PPC-Health | (1,845) | Ref | Ref | Ref | Ref | Ref |
| PPC-Mild | (1,047) | 1.21 (0.93-1.58) | 1.49 (0.99-2.26) | 1.63 (0.76-3.53) | 1.20 (0.97-1.49) | 1.25 (1.01-1.55) |
| PPC-High GI | (694) | 1.61 (1.18-2.18) | 2.08 (1.23-3.51) | 1.31 (0.51-3.39) | 1.36 (1.02-1.82) | 1.39 (1.05-1.85) |
| PPC-Tooth Loss | (800) | 1.44 (1.09-1.89) | 1.94 (1.26-2.98) | 1.23 (0.52-2.91) | 1.54 (1.22-1.94) | 1.50 (1.19-1.89) |
| PPC-Posterior Disease | (999) | 1.16 (0.89-1.53) | 1.33 (0.87-2.04) | 0.85 (0.36-2.02) | 1.18 (0.95-1.47) | 1.29 (1.01-1.61) |
| PPC-Severe Tooth Loss | (900) | 1.59 (1.21-2.08) | 1.75 (1.13-2.71) | 1.65 (0.75-3.62) | 1.39 (1.10-1.76) | 1.28 (1.01-1.62) |
| PPG-Severe Disease | (508) | 1.88 (1.38-2.56) | 1.32 (0.75-2.30) | 2.92 (1.24-6.89) | 1.50 (1.11-2.02) | 1.52 (1.15-2.03) |
| Total | (6,793) | | | | | |
| BIC Improvement | | 20.57 | 13.25 | 9.27 | 16.75 | 15.31 |
| CDC-Health | (775) | Ref | Ref | Ref | Ref | Ref |
| CDC-Mild | (2,035) | 0.82 (0.63-1.07) | 1.48 (0.89-2.47) | 0.92 (0.41-2.03) | 1.00 (0.80-1.24) | 1.15 (0.92-1.45) |
| CDC-Moderate | (2,799) | 1.19 (0.92-1.53) | 1.46 (0.89-2.39) | 1.01 (0.48-2.16) | 0.95 (0.77-1.18) | 1.22 (0.97-1.53) |
| CDC-Severe | (1,184) | 1.21 (0.91-1.61) | 1.84 (1.09-3.12) | 1.15 (0.50-2.66) | 1.08 (0.84-1.38) | 1.25 (0.97-1.62) |
| Total | (6,793) | | | | | |
| BIC Improvement | | 17.70 | 5.87 | 0.46 | 1.69 | 3.60 |
| European-Health | (749) | Ref | Ref | Ref | Ref | Ref |
| European-Incipient | (5,021) | 1.07 (0.84-1.36) | 1.43 (0.89-2.30) | 0.92 (0.45-1.90) | 0.98 (0.80-1.19) | 1.08 (0.88-1.34) |
| European-Severe | (973) | 1.20 (0.89-1.61) | 2.20 (1.29-3.74) | 1.43 (0.62-3.28) | 1.11 (0.85-1.44) | 1.38 (1.06-1.79) |
| Total | (6,793) | | | | | |

| | | | | | |
|---|---|---|---|---|---|
| BIC Improvement | 1.61 | 11.27 | 2.50 | 1.68 | 7.47 |

Diabetes adjusted for Race/Center, Age, Gender, BMI, Smoking (3-levels) and Education (3-levels)
CHD and Stroke adjusted for Race/Center, Age, Gender, BMI, Smoking (3-levels), Diabetes, Hypertension), Education (3-levels), HDL, LDL, Triglycerides, Total Cholesterol and Log Serum CRP

TABLE 19
Associations between Three Indices of Periodontal Disease Case Status and Prevalent Systemic
Conditions in the NHANES 2009-2014 Studies.Diabetes adjusted for Race, Age, Gender, BMI, Smoking
(3-levels) and Education (3-levels). Heart Attack and Stroke adjusted for Race, Age, Gender,
BMI, Smoking (3-levels), Diabetes, Hypertension), Education (3-levels), HDL, LDL, Triglycerides
and Total Cholesterol. CRP was only measured at one exam cycle. CRP adjusted for Race, Age,
Gender, BMI, Smoking (3-levels), Diabetes, Hypertension) and Education (3-levels)

| | | Diabetes | CHD | Stroke | CRP |
|---|---|---|---|---|---|
| | | N Cases/Total | | | |
| | | 1320/10632 | 584/10077 | 272/10098 | 899/3575 |
| Case Status | (N) | OR (95% CI) | OR (95% CI) | OR (95% CI) | OR (95% CI) |
| PPC-Health | (5,878) | Ref | Ref | Ref | Ref |
| PPC-Mild | (674) | 1.37 (1.06-1.77) | 1.19 (0.78-1.82) | 0.94 (0.45-1.97) | 1.08 (0.77-1.52) |
| PPC-High GI | (962) | 1.36 (1.10-1.68) | 1.14 (0.84-1.54) | 1.47 (0.95-2.27) | 1.19 (0.85-1.67) |
| PPC-Tooth Loss | (1,197) | 1.51 (1.25-1.82) | 1.19 (0.90-1.57) | 1.99 (1.37-2.89) | 1.57 (1.19-2.07) |
| PPC-Posterior Disease | (506) | 1.17 (0.87-1.58) | 1.83 (1.23-2.72) | 2.07 (1.16-3.71) | 1.75 (1.21-2.53) |
| PPC-Severe Tooth Loss | (693) | 1.72 (1.37-2.16) | 1.41 (1.04-1.91) | 2.18 (1.45-3.28) | 1.61 (1.14-2.29) |
| PPG-Severe Disease | (800) | 1.28 (1.00-1.64) | 1.05 (0.84-2.45) | 1.43 (0.84-2.45) | 1.28 (0.93-1.76) |
| Total | (10,710) | | | | |
| BIC Improvement | | 30.8 | 11.52 | 21.3 | 18.3 |
| CDC-Health | (2,521) | Ref | Ref | Ref | Ref |
| CDC-Mild | (3,199) | 1.12 (0.91-1.37) | 1.22 (0.88-1.70) | 1.02 (0.66-1.57) | 1.21 (0.96-1.54) |
| CDC-Moderate | (3,791) | 1.52 (1.25-1.85) | 1.62 (1.19-2.20) | 1.14 (0.76-1.71) | 1.29 (1.03-1.63) |
| CDC-Severe | (1,199) | 1.45 (1.13-1.85) | 1.34 (0.92-1.96) | 1.15 (0.70-1.88) | 1.58 (1.16-2.15) |
| Total | (10,710) | | | | |
| BIC Improvement | | 24.9 | 12.50 | 0.7 | 9.0 |
| European-Health | (2,563) | Ref | Ref | Ref | Ref |
| European-Incipient | (6,681) | 1.32 (1.09-1.59) | 1.37 (1.02-1.86) | 1.22 (0.81-1.84) | 1.21 (0.98-1.48) |
| European-Severe | (1,466) | 1.53 (1.21-1.94) | 1.37 (0.96-1.97) | 1.41 (0.87-2.28) | 1.77 (1.32-2.39) |
| Total | (10,710) | | | | |

| | | | | |
|---|---|---|---|---|
| BIC Improvement | 13.2 | 4.53 | 2.0 | 14.4 |

*Bayesian Information Criterion (BIC)

TABLE 20
CRP and IL-6 adjusted for Race/Center, Age, Gender, BMI, Smoking
(3-levels), Diabetes, Hypertension) and Education (3-levels)

| BIC Improvement | Evidence for a higher BIC |
|---|---|
| 0 to 2 | Not worth more than a bare mention |
| 2 to 6 | Positive |
| 6 to 10 | Strong |
| >10 | Very Strong |

*Bavesian Information Criterion (BIC)

TABLE 21
RIC and NHANES: Prevalent CHD by Demographics, General
Health and Periodontal Disease Case Definitions

| Demographic, Health and | Dental ARC (N = 6,793) | | | | NHANES (N = 10,710) | | | | |
|---|---|---|---|---|---|---|---|---|---|
| Periodontal Disease | CHD | | CHD | | CHD | | CHD | | p-value |
| Variables | N (Yes) | | N (No) | | N (Yes) | | N (No) | | D-ARIC/NHANES |
| African American | 64 | (5.1%) | 1199 | (94.9%) | 66 | (3.0%) | 2162 | (97.0%) | 0.21/<0.0001 |
| Caucasian | 326 | (6.0%) | 5125 | (94.0%) | 183 | (4.0%) | 4397 | (96.0%) | |
| Other | . . . | | . . . | | 81 | (2.1%) | 3810 | (97.9%) | |
| Female | 114 | (3.1%) | 3537 | (96.9%) | 99 | (1.8%) | 5334 | (98.2%) | <0.0001/<0.0001 |
| Male | 278 | (9.0%) | 2810 | (91.0%) | 231 | (4.4%) | 5035 | (95.6%) | |
| Mean(SD) Age | 64.4 | (5.6) | 62.3 | (5.6) | 65.4 | (12.0) | 51.6 | (14.1) | <0.0001/<0.0001 |
| Mean(SD) BMI | 30.0 | (5.4) | 28.7 | (5.4) | 30.5 | (6.6) | 29.3 | (6.7) | 0.14/0.001 |
| Diabetes | 94 | (10.1%) | 837 | (89.9%) | 101 | (7.6%) | 1234 | (92.4%) | <0.0001/<0.0001 |
| No Diabetes | 297 | (5.1%) | 5477 | (94.9%) | 229 | (2.5%) | 9135 | (97.6%) | |
| Hypertensive | 204 | (8.9%) | 2080 | (91.1%) | 248 | (6.2%) | 3760 | (93.8%) | <0.0001/<0.0001 |
| Non-Hypertensive | 179 | (4.0%) | 4249 | (96.0%) | 82 | (1.2%) | 6594 | (98.8%) | |
| Current Smoker | 104 | (10.8%) | 861 | (89.2%) | 74 | (3.7%) | 1932 | (96.3%) | <0.0001/<0.0001 |
| Former Smoke | 257 | (5.2%) | 4723 | (94.8%) | 129 | (4.8%) | 2547 | (95.2%) | |
| Never Smoker | 31 | (3.9%) | 763 | (96.1%) | 127 | (2.1%) | 5885 | (97.9%) | |
| Mean(SD) LDL | 114.4 | (32.1) | 122.5 | (33.1) | 94.1 | (36.8) | 113.4 | (37.2) | <0.0001/<0.0001 |
| Mean(SD) Total | 188.3 | (34.5) | 201.8 | (35.9) | 175.5 | (42.3) | 197.4 | (41.5) | <0.0001/0.0001 |
| Cholesterol | | | | | | | | | |
| Mean(SD) Triglyceride | 154.0 | (91.1) | 142.2 | (83.6) | 171.7 | (115.4) | 159.1 | (145.0) | 0.007/0.13 |
| Mean(SD) HDL | 43.1 | (13.4) | 50.9 | (16.7) | 49.3 | (17.1) | 53.0 | (16.0) | <0.0001/<0.0001 |
| Education | | | | | | | | | |
| Basic | 78 | (8.7%) | 824 | (91.4%) | 88 | (3.5%) | 2409 | (96.5%) | 0.0002/0.0008 |
| Intermediate | 170 | (5.9%) | 2735 | (94.2%) | 92 | (4.0%) | 2208 | (96.0%) | |
| Advanced | 144 | (4.9%) | 2780 | (95.1%) | 149 | (2.5%) | 5740 | (97.5%) | |
| PPC-Health | 61 | (3.3%) | 1774 | (96.7%) | 95 | (1.6%) | 5780 | (98.4%) | <0.0001/<0.0001 |
| PPC-Mild | 57 | (5.5%) | 986 | (94.5%) | 11 | (1.6%) | 663 | (98.4%) | |
| PPC-High GI | 46 | (6.8%) | 634 | (93.2%) | 49 | (5.1%) | 910 | (94.9%) | |
| PPC-Tooth Loss | 62 | (7.8%) | 729 | (92.2%) | 70 | (5.9%) | 1126 | (94.9%) | |
| PPC-Posterior Disease | 62 | (6.2%) | 936 | (93.8%) | 26 | (5.1%) | 480 | (92.5%) | |
| PPC-Severe Tooth Loss | 70 | (7.9%) | 818 | (92.1%) | 52 | (7.5%) | 640 | (92.5%) | |
| PPG-Severe Disease | 34 | (6.8%) | 470 | (93.3%) | 27 | (3.4%) | 770 | (96.6%) | |
| CDC/AAP Healthy | 32 | (4.2%) | 738 | (95.8%) | 29 | (1.2%) | 2491 | (98.9%) | <0.0001/<0.0001 |
| Mild | 88 | (4.4%) | 1932 | (95.6%) | 70 | (2.2%) | 3127 | (97.8%) | |
| Moderate | 168 | (6.1%) | 2603 | (93.9%) | 175 | (4.6%) | 3610 | (95.4%) | |
| Severe | 104 | (8.8%) | 1074 | (91.2%) | 56 | (4.7%) | 1141 | (95.3%) | |
| European Healthy | 31 | (3.9%) | 763 | (96.1%) | 26 | (1.0%) | 2536 | (99.0%) | <0.0001/<0.0001 |
| Incipient | 257 | (5.2%) | 4723 | (94.8%) | 220 | (3.3%) | 6455 | (96.7%) | |
| Severe | 104 | (10.8%) | 861 | (13.6%) | 84 | (5.8%) | 1378 | (94.3%) | |

TABLE 22
Associations between Three Indices of Periodontal Disease Case Status
and Prevalent Systemic Conditions in the Dental ARIC Study.

| | | Diabetes | CHD | Stroke | CRP | IL-6 |
|---|---|---|---|---|---|---|
| | | N Cases/Total | | | | |
| | | 924/6676 | 313/5491 | 91/5503 | 1378/5513 | 1352/5442 |
| Case Status | (N) | OR (95% CI) | OR (95% CI) | OR (95% CI) | OR (95% CI) | OR (95% CI) |
| PPC-Health | (1,845) | Ref | Ref | Ref | Ref | Ref |
| PPC-Mild | (1,047) | 1.21 (0.93-1.58) | 1.49 (0.99-2.26) | 1.63 (0.76-3.53) | 1.20 (0.97-1.49) | 1.25 (1.01-1.55) |
| PPC-High GI | (694) | 1.61 (1.18-2.18) | 2.08 (1.23-3.51) | 1.31 (0.51-3.39) | 1.36 (1.02-1.82) | 1.39 (1.05-1.85) |
| PPC-Tooth Loss | (800) | 1.44 (1.09-1.89) | 1.94 (1.26-2.98) | 1.23 (0.52-2.91) | 1.54 (1.22-1.94) | 1.50 (1.19-1.89) |
| PPC-Posterior Disease | (999) | 1.16 (0.89-1.53) | 1.33 (0.87-2.04) | 0.85 (0.36-2.02) | 1.18 (0.95-1.47) | 1.29 (1.01-1.61) |
| PPC-Severe Tooth Loss | (900) | 1.59 (1.21-2.08) | 1.75 (1.13-2.71) | 1.65 (0.75-3.62) | 1.39 (1.10-1.76) | 1.28 (1.01-1.62) |
| PPG-Severe Disease | (508) | 1.88 (1.38-2.56) | 1.32 (0.75-2.30) | 2.92 (1.24-6.89) | 1.50 (1.11-2.02) | 1.52 (1.15-2.03) |
| Total | (6,793) | | | | | |
| BIC Improvement | | 20.57 | 13.25 | 9.27 | 16.75 | 15.31 |
| CDC-Health | (775) | Ref | Ref | Ref | Ref | Ref |
| CDC-Mild | (2,035) | 0.82 (0.63-1.07) | 1.48 (0.89-2.47) | 0.92 (0.41-2.03) | 1.00 (0.80-1.24) | 1.15 (0.92-1.45) |
| CDC-Moderate | (2,799) | 1.19 (0.92-1.53) | 1.46 (0.89-2.39) | 1.01 (0.48-2.16) | 0.95 (0.77-1.18) | 1.22 (0.97-1.53) |
| CDC-Severe | (1,184) | 1.21 (0.91-1.61) | 1.84 (1.09-3.12) | 1.15 (0.50-2.66) | 1.08 (0.84-1.38) | 1.25 (0.97-1.62) |
| Total | (6,793) | | | | | |
| BIC Improvement | | 17.70 | 5.87 | 0.46 | 1.69 | 3.60 |
| European-Health | (749) | Ref | Ref | Ref | Ref | Ref |
| European-Incipient | (5,021) | 1.07 (0.84-1.36) | 1.43 (0.89-2.30) | 0.92 (0.45-1.90) | 0.98 (0.80-1.19) | 1.08 (0.88-1.34) |
| European-Severe | (973) | 1.20 (0.89-1.61) | 2.20 (1.29-3.74) | 1.43 (0.62-3.28) | 1.11 (0.85-1.44) | 1.38 (1.06-1.79) |
| Total | 6,793) | | | | | |

| | | | | | |
|---|---|---|---|---|---|
| BIC Improvement | 1.61 | 11.27 | 2.50 | 1.68 | 7.47 |

Diabetes adjusted for Race/Center, Age, Gender, BMI, Smoking (3-levels) and Education (3-levels)
CHD and Stroke adjusted for Race/Center, Age, Gender, BMI, Smoking (3-levels), Diabetes, Hypertension), Education (3-levels), HDL, LDL, Triglycerides, Total Cholesterol and Log Serum CRP
CRP and IL-6 adjusted for Race/Center, Age, Gender, BMI, Smoking (3-levels), Diabetes, Hypertension) and Education (3-levels) *Bayesian Information Criterion (BIC)

TABLE 23
Associations between Three Indices of Periodontal Disease Case Status
and Prevalent Systemic Conditions in the NHANES 2009-2014 Studies.

| | | Diabetes | CHD | Stroke | CRP |
|---|---|---|---|---|---|
| | | N Cases/Total | | | |
| | | 1320/10632 | 584/10077 | 272/10098 | 899/3575 |
| Case Status | (N) | OR (95% CI) | OR (95% CI) | OR (95% CI) | OR (95%C I) |
| PPC-Health | (5,878) | Ref | Ref | Ref | Ref |
| PPC-Mild | (674) | 1.37 (1.06-1.77) | 1.19 (0.78-1.82) | 0.94 (0.45-1.97) | 1.08 (0.77-1.52) |
| PPC-High GI | (962) | 1.36 (1.10-1.68) | 1.14 (0.84-1.54) | 1.47 (0.95-2.27) | 1.19 (0.85-1.67) |
| PPC-Tooth Loss | (1,197) | 1.51 (1.25-1.82) | 1.19 (0.90-1.57) | 1.99 (1.37-2.89) | 1.57 (1.19-2.07) |
| PPC-Posterior Disease | (506) | 1.17 (0.87-1.58) | 1.83 (1.23-2.72) | 2.07 (1.16-3.71) | 1.75 (1.21-2.53) |
| PPC-Severe Tooth Loss | (693) | 1.72 (1.37-2.16) | 1.41 (1.04-1.91) | 2.18 (1.45-3.28) | 1.61 (1.14-2.29) |
| PPG-Severe Disease | (800) | 1.28 (1.00-1.64) | 1.05 (0.84-2.45) | 1.43 (0.84-2.45) | 1.28 (0.93-1.76) |
| Total | (10,710) | | | | |
| BIC Improvement | | 30.8 | 11.52 | 21.3 | 18.3 |
| CDC-Health | (2,521) | Ref | Ref | Ref | Ref |
| CDC-Mild | (3,199) | 1.12 (0.91-1.37) | 1.22 (0.88-1.70) | 1.02 (0.66-1.57) | 1.21 (0.96-1.54) |
| CDC-Moderate | (3,791) | 1.52 (1.25-1.85) | 1.62 (1.19-2.20) | 1.14 (0.76-1.71) | 1.29 (1.03-1.63) |
| CDC-Severe | (1,199) | 1.45 (1.13-1.85) | 1.34 (0.92-1.96) | 1.15 (0.70-1.88) | 1.58 (1.16-2.15) |
| Total | (10,710) | | | | |
| BIC Improvement | | 24.9 | 12.50 | 0.7 | 9.0 |
| European-Health | (2,563) | Ref | Ref | Ref | Ref |
| European-Incipient | (6,681) | 1.32 (1.09-1.59) | 1.37 (1.02-1.86) | 1.22 (0.81-1.84) | 1.21 (0.98-1.48) |
| European-Severe | (1,466) | 1.53 (1.21-1.94) | 1.37 (0.96-1.97) | 1.41 (0.87-2.28) | 1.77(1.32-2.39) |
| Total | (10,710) | | | | |

| | | | | |
|---|---|---|---|---|
| BIC Improvement | 13.2 | 4.53 | 2.0 | 14.4 |

Diabetes adjusted for Race, Age, Gender, BMI, Smoking (3-levels) and Education (3-levels)
Heart Attack and Stroke adjusted for Race, Age, Gender, BMI, Smoking (3-levels), Diabetes, Hypertension), Education (3-levels), HDL, LDL, Triglycerides and Total Cholesterol
CRP was only measured at one exam cycle. CRP adjusted for Race, Age, Gender, BMI, Smoking (3-levels), Diabetes, Hypertension) and Education (3-levels)
*Bayesian Information Criterion (BIC)

TABLE 24
Adjusted CRP and IL-6

| BIC Improvement | Evidence for a higher BIC |
|---|---|
| 0 to 2 | Not worth more than a bare mention |
| 2 to 6 | Positive |
| 6 to 10 | Strong |
| >10 | Very Strong |

### EXAMPLE 5

Introduction

Periodontal disease is a chronic inflammatory disease caused by bacterial colonization that affects the soft and hard structures that support the teeth.^{[1]} The prevalence of periodontal disease is high, with gingivitis or periodontitis affecting up to 90% of the population worldwide. According to recent findings from the Centers for Disease Control and Prevention (CDC), half of Americans aged 30 or older have periodontitis, the more advanced form of periodontal disease.^{[2]} Periodontitis is associated with an increase in systemic inflammation markers, through chronic low-grade exposure to Gram-negative bacteria^{[3,4]}, implicated in the etiology of atherosclerosis and stroke.^{[5]} Observational studies have shown that poor periodontal health status is associated with an increased stroke risk.^{[6-10]} Poor oral hygiene is a major contributor to periodontal disease and thus a potentially modifiable stroke risk factor. An increase in tooth-brushing frequency decreases the concentrations of systemic inflammatory markers levels in the serum.^{[11]} A population based Taiwanese study found that dental prophylaxis or periodontal disease treatment could reduce the incidence of ischemic stroke.^{[12-13]} However, similar data are lacking in the predominantly biracial US population.

Recent reports suggest a rise in the global burden of stroke.^{[14]} Post hoc analyses of prospective—longitudinal and smaller case—control studies have reported an association between periodontal disease and incident stroke.^{[7]} These studies suggest that stroke has a stronger association with periodontal disease than coronary artery disease.^{[15]} Periodontal disease was found to increase the risk stroke by nearly three-fold in a combined analysis of two prospective studies.^{[16]} A more recent meta-analysis of two cohort studies found that periodontal disease increased the risk of incident ischemic strokes by 1.6-fold.^{[17]} Recently, a case—control study confirmed the independent graded association between the severity of periodontal disease and prevalent stroke.^{[18]} If causal, these associations would be of great importance due to the potential that periodontal disease treatment could reduce the stroke risk.

Individual studies have limitations, including the use of many differing definitions of periodontal disease, consideration of potential cofounders such as socioeconomic status, and low statistical power. Additionally, these studies underestimate the prevalence of periodontal disease.^{[19]} Previously, a Latent Class Analysis (LCA) was applied to identify discrete classes of individuals that are discriminated by tooth-level clinical parameters to define seven distinct periodontal profile classes (PPC A-G) and seven distinct tooth profile classes (TPC A-G) ranging from health to severe periodontal disease status,^{[20]} validated in three large cohorts. The periodontal and tooth profile classes using LCA was applied to provide robust periodontal clinical definitions that reflect disease patterns in the population at a subject and tooth level. We examined the relationship between periodontal disease and stroke as well as the ischemic stroke subtypes.

Methods

Study Population. The cohort of the ARIC study recruited in 1987-1989 with an aim of studying the causes of atherosclerosis and clinical sequelae.^{[21]} The study enrolled 15,792 participants within the age group of 45-64 identified by probability sampling in a biracial cohort from four US communities. In addition to follow-up visits every three years, participants have been contacted annually by telephone and queried about hospitalizations. The institutional review boards of all participating institutions approved the study and all participants provided written informed consent. All participants, White or African-Americans, who completed the fourth clinic visit (1996-1998) in ARIC (N=11,656), were included in the current study. Participants (N=1,294) with prevalent stroke or a first ischemic stroke event that occurred before fourth clinical visit were excluded. Thus, 10,362 remaining participants were included for dental care utilization analysis.

Dental ARIC, an ancillary study of the ARIC, was conducted at the fourth clinic visit. Data collection included a comprehensive dental examination, questionnaire, and sample collection. Study participants who were edentulous or those requiring antibiotic prophylaxis for periodontal probing were excluded from the Dental ARIC study. Of the 6,793 Dental ARIC participants that underwent periodontal examination at the fourth visit, a total of 6,736 participants without prior stroke were included for distinct periodontal profile class analysis.^{[20]}

The ARIC Investigators are willing to share the data used in this manuscript with a researcher for the purposes of reproducing the results, subject to completion of a data use agreement ensuring appropriate protection of the confidentiality of ARIC participants' data.

Assessment of Dental Care Utilization. The pattern of dental care utilization or dental visits was classified by patient-reported responses to the Dental History Form questionnaire administered at the fourth clinical ARIC study by trained personnel (1996-1998). Participant dental care utilization was classified as regular use (those who sought routine dental care time(s) a year) or episodic (only when in discomfort, something needed to be fixed, never, or did not receive regular dental care).

Assessment of Periodontal Profile Class. The analytical approach implemented person-level LCA to identify discrete classes of individuals was based upon 7 tooth-level clinical parameters, including: ≥1 site with interproximal attachment level (IAL) ≥3 mm, ≥1 site with probing depth (PD) ≥4 mm, extent of bleeding on probing (BOP, dichotomized at 50% or ≥3 sites per tooth), gingival inflammation index14 (GI, dichotomized as GI=0 vs. GI≥1), plaque index15 (PI, dichotomized as PI=0 vs. PI≥1), the presence/absence of full prosthetic crowns for each tooth, and tooth status presence (present vs. absent).^{[20]} Details of LCA is included in the online supplemental-data.

Adjudication of Stroke Subtypes. Physicians reviewed hospitalization records and stroke diagnoses was made based on a computer algorithm, with any differences adjudicated by a second physician reviewer. All events occurring between the fourth visit (1996-1998) and December 2012 were included as verified ischemic strokes. The study considered incident ischemic strokes and the subjects were censored at the time of the event (recurrent strokes not considered in the analysis). According to criteria adopted from the National Survey of Stroke subtype classification, ischemic strokes were then further classified according to pathogenic subtype as thrombotic brain infarction, lacunar infarction, or cardioembolic stroke. ^{[21-22]} Infarct distribution patterns on neuroimaging were not considered per the algorithm .

Other Variables of Interest. Age, sex, race, and additional vascular risk factors such as body mass index (BMI), waist-to-hip ratio, and lipid profile were assessed according to published methods during the fourth ARIC visit (1996-1998).^{[23]} Hypertension was defined as the average of two blood pressure readings at the visit (systolic blood pressure 140 mm HG or higher and diastolic blood pressure 90 mm Hg or higher) or on hypertension medication. Diabetes was measured by a visit-based definition and an interview-based definition. Visit-based diabetes was defined according to serum glucose measurements (fasting blood glucose >126 mg/dL or >200 mg/dL if not fasting), a self-reported physician diagnosis of diabetes, or on medication. Participants reported their 3-level education status (basic<11 years, intermediate 12-16 years, or advanced 17+ years), smoking status, and alcohol use.^{[24]}

Statistical Analysis. Cox proportional hazards models were used to assess crude and adjusted hazard ratio (HR) and 95% confidence interval to analyze the association among subjects with periodontal disease and incidence of ischemic stroke, in comparison with those with periodontal health. Similar comparisons were made between regular and episodic dental care users. In both analyses, HR was adjusted for race/center, age, gender, BMI, hypertension, diabetes, LDL level, smoking (3-levels), pack years, and educational level (3-levels). Kaplan Meier survival curve and HR analysis were used to evaluate the incidence of each ischemic stroke subtypes in periodontal disease when compared to periodontal health. All data were analyzed using SAS version 9.4 (SAS institute Inc., Cary, N.C.).

Results

In the Dental ARIC study, during the fourth ARIC visit (1996-1998), a subset of 6,736 dentate subjects (mean age±SD=62.3±5.6, 55% female, 81% white and 19% African-American) were assessed for periodontal disease. The subjects excluded from this cohort had a higher rate of incident stroke (Prior stroke 18.5%, edentulous 7.6%, those with other exclusions 9.0%) compared to those included and completed the dental assessment (4.4%). Baseline characteristics of the dental cohort of the ARIC study population, stratified by PPC-A-G on Visit 4, are shown in Table 25. Participants with higher PPC classes (B through G) were similar in age compared with PPC-A (Health). They included a higher proportion of subjects who were of the male gender and the African-American race. Participants with higher PPC classes (B through G) also had a slightly higher BMI, waist-to hip ratio, and higher proportions of hypertension and diabetes, compared with PPC-A. They also had fewer years of education and were less likely to be current or former alcohol users. The PPC classes (C through G) were more likely to be former and current smokers compared with PPC class A and B. The fasting lipid profiles were similar across the groups. Baseline characteristics of the ARIC study population, divided by dental care utilization, are shown in Table 26. The regular dental care users included a higher proportion of female and White subjects compared to the episodic dental care users. They also had a slightly lower BMI, waist-to hip ratio, and lower proportions of hypertension and diabetes. Additionally, they had more years of education and were more likely to be current or former alcohol users. The regular dental care users were less likely to be former and current smokers compared with the episodic dental care users. The fasting lipid profiles were similar across the groups (not shown). The regular dental care users had a higher proportion of low PPC grades compared to the episodic dental care users suggesting that regular dental care utilization was associated with lower burden of periodontal disease.

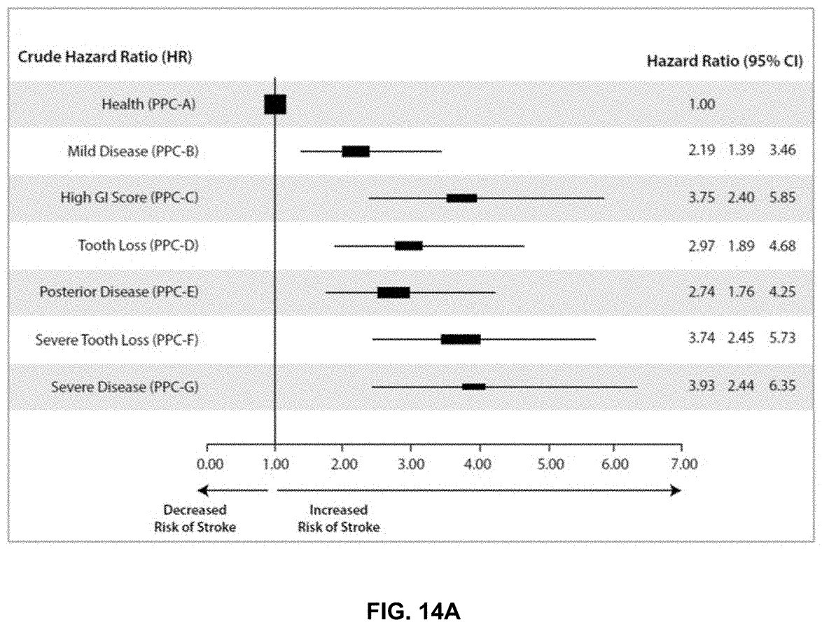

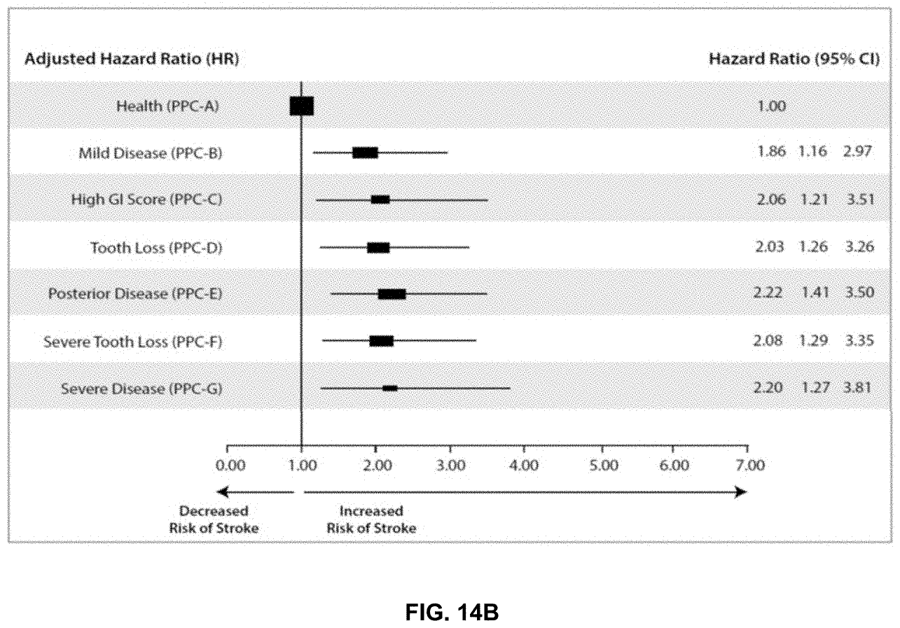

In the Dental ARIC study, during the fourth ARIC visit (1996-1998), a subset of 6,736 dentate subjects (mean age±SD=62.3±5.6, 55% female, 81% white and 19% African-American) were assessed for periodontal disease. A total of 299 incident ischemic strokes occurred over a median of 15-year follow-up period. The retention of ARIC participants during the follow-up period was high (>90%). Compared with the reference healthy group without periodontal disease (PPC-A), mild periodontal disease or PPC-B (Crude HR 2.19 95% CI 1.39-3.46), high GI score or PPC-C (Crude HR 3.75 95% CI 2.40-5.85), tooth loss or PPC-D (Crude HR 2.97 95% CI 1.89-4.68), posterior disease or PPC-E (Crude HR 2.74 95% CI 1.76-4.25), severe tooth loss or PPC-F (Crude HR 3.74 95% CI 2.45-5.73) and severe periodontal disease or PPC-G (Crude HR 3.93 95% CI 2.44-6.35), had a higher risk for incident ischemic stroke depicted in FIG. 14A. After adjustment for race/center, age, gender, BMI, hypertension, diabetes, LDL Level, smoking (3-levels), pack years, and education (3-levels), mild periodontal disease or PPC-B (adjusted HR 1.86 95% CI 1.16-2.97), high GI score or PPC-C (Adjusted HR 2.06 95% CI 1.21-3.51), tooth loss or PPC-D (Adjusted HR 2.03 95% CI 1.26-3.26), posterior disease or PPC-E (Adjusted HR 2.22 95% CI 1.41-3.50), severe tooth loss or PPC-F (Adjusted HR 2.08 95% CI 1.29-3.35) and severe periodontal disease or PPC-G (Adjusted HR 2.20 95% CI 1.27-3.81), had a higher risk for incident ischemic stroke. The incidence rate for ischemic stroke/1,000-person years was 1.29 (95% CI 0.92-1.81) for PPC-A, 2.82 (95% CI 2.08-3.83) for PPC-B, 4.80 (95% CI 3.59-6.43) for PPC-C, 3.81 (95% CI 2.80-5.17) for PPC-D, 3.50 (95% CI 2.64-4.65) for PPC-E, 4.78 (95% CI 3.69-6.20) for PPD-F and 5.03 (3.57-7.07) for PPC-G. The PPC-incident stroke association is depicted in a Kaplan-Meier plot in the online data-supplement. The seven levels of PPC showed a trend towards graded association with incident ischemic stroke (χ² trend p value<0.0001).

As shown in FIG. 14B, all forms of periodontal disease which are characterized by inflammatory changes are at significantly greater risk than health using this 7-level classification. The inflammatory characteristics differ among the 6 disease classes, but are listed in order of increased mean interproximal attachment loss. In supplemental Figure the incident events are stratified by PPC and the highest rate of events was seen among PPC-C (gingival inflammation) and PPC-G (severe disease) which are the most inflamed classes.^{[21]} Thus, inflammation plays a critical role in defining the risk for incident events as compared to PPC-A (health). What emerges from this investigation is that high gingival inflammation in the absence of severe periodontal disease (PPC-C) and the highly inflamed severe periodontitis class (PPC-G) are at higher risk than those with mild, moderate or posterior disease patterns (all have less inflamed periodontal tissues). These data emphasize the importance of inflammation rather than just the level of attachment as being the main determinant of risk.

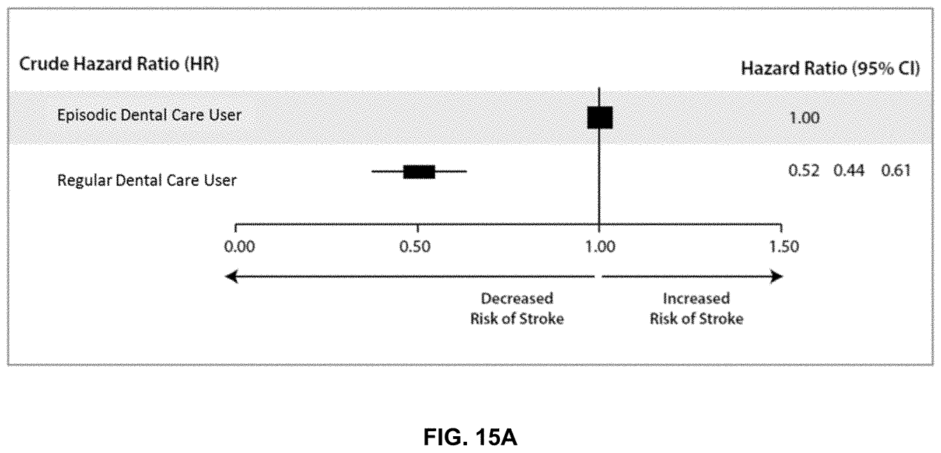

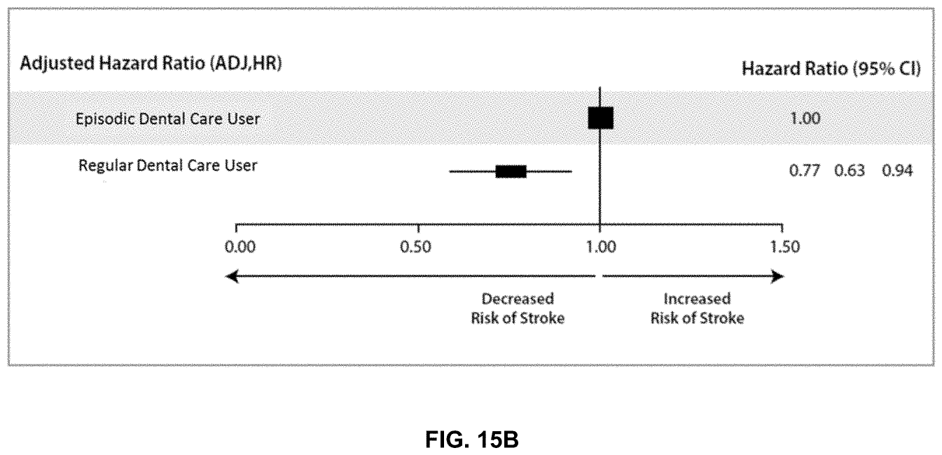

In the main ARIC cohort during the fourth ARIC visit (1996-1998), a total of 11,656 participants (mean age±SD=62.8±5.6, 56% female, 78% White and 22% African-American) were assessed for dental care utilization. Over a 15-year follow-up period, a total of 584 participants had incident ischemic stroke events. Compared with a reference group of episodic dental care users, regular dental care users had a lower risk for ischemic stroke (Crude HR 0.52 95% CI 0.44-0.61). After adjustment for race/center, age, gender, BMI, hypertension, diabetes, LDL level, smoking, and education, regular dental care use continued to be associated with lower rates of ischemic stroke (adjusted HR 0.77, 95% CI 0.63-0.94), as seen in FIGS. 15A-15B.

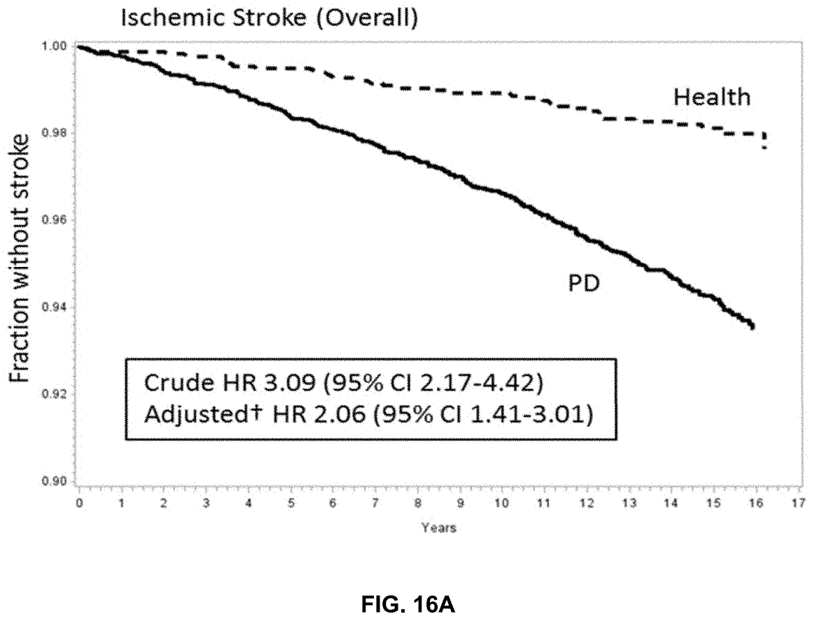

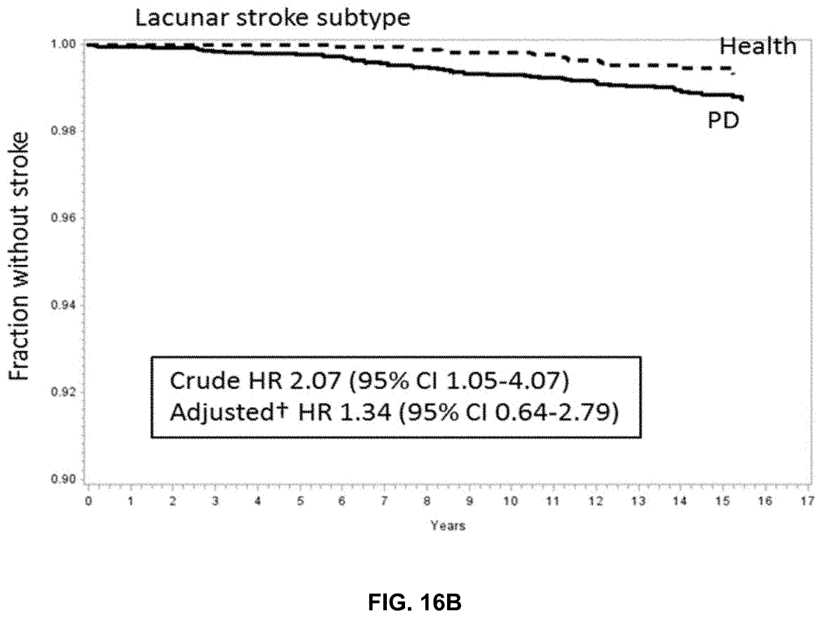

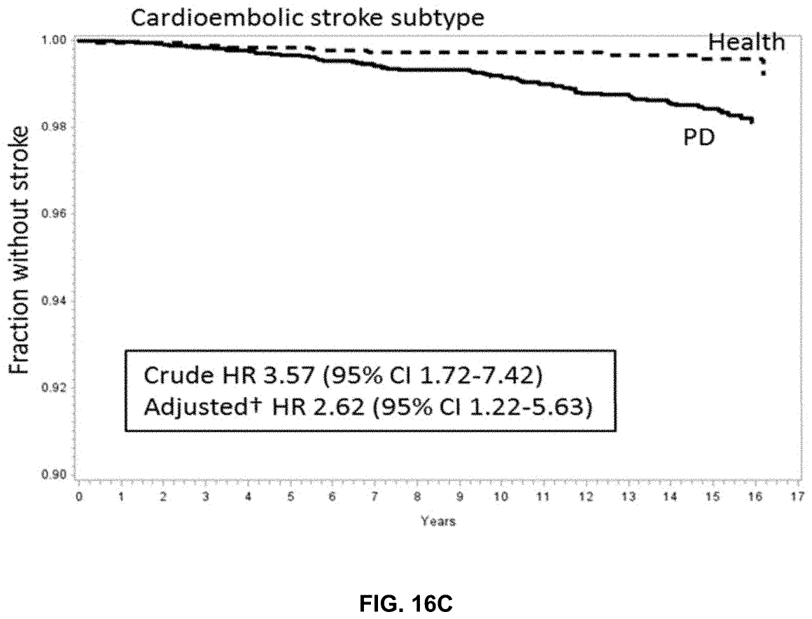

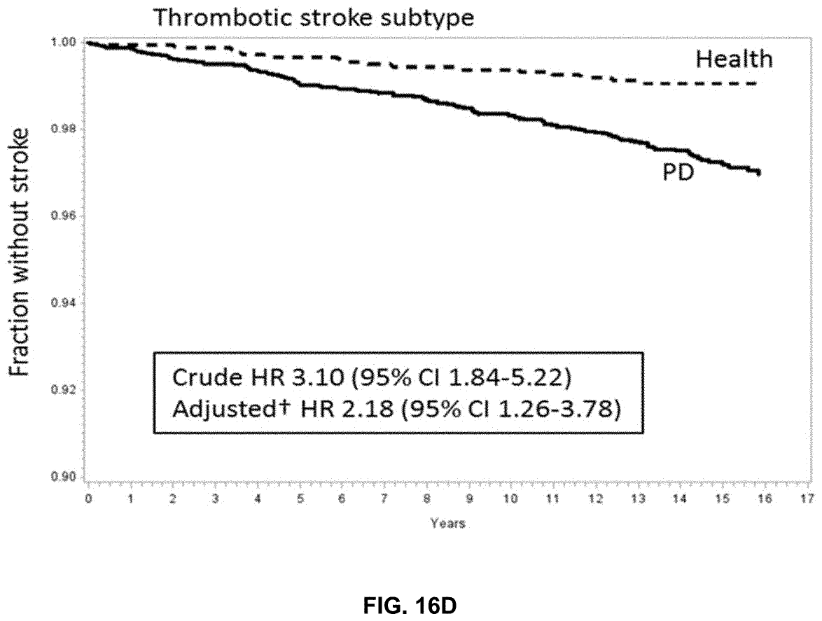

Among the 299 incident ischemic strokes in the dental cohort, 79 were cardioembolic, 140 thrombotic, 61 lacunar, and 19 others. Among the three major stroke subtypes, there was a significant increased hazard of cardioembolic (HR 2.6, 95% CI 1.2-5.6) and thrombotic (HR 2.2, 95% CI 1.3-3.8), but not of lacunar strokes (HR 1.3, 95% CI 0.6-2.8) among study participants with periodontal disease (PPC B-G) compared with those with periodontal health (PPC-A). The association of overall ischemic stroke as well as thrombotic, cardioembolic, or lacunar subtype of stroke and periodontal disease is demonstrated in FIGS. 16A-16B.

Discussion

Periodontal disease is highly prevalent among adults worldwide and is an important public health problem. Assessment of risk profiles for periodontal disease in adults in the United States show male gender, current cigarette smoking, and diabetes are important risk factors for periodontal disease.^{[25]} These findings could enhance recognizable target populations for viable interventions to enhance periodontal health of adults, who may likewise be at an increased risk of ischemic stroke. This Example can demonstrate that periodontal disease is an independent risk factor for incident ischemic stroke. It is significant that the high gingival inflammation group, including gingivitis and mild periodontitis but also highly inflamed, likewise had an increased risk of ischemic stroke. Moreover, we report a trend towards a graded association between periodontal disease and incident ischemic stroke. Individual studies, including post-hoc analyses of prospective—longitudinal studies and case—control studies, have reported an association between periodontal disease and incident stroke.^{[7]} It is possible that an increased risk of thrombotic stroke may be secondary to athero-thrombosis in the cervico-cerebral vasculature. The periodontal disease-cardioembolic stroke association may be due to coronary artery disease or atrial fibrillation related to periodontal disease induced inflammation. Contrary to findings noted in prior case-control studies, periodontal disease was not independently associated with lacunar strokes.^{[26, 27]} Possible factors attributable to this difference may be risk factors for lacunar stroke such as age, hypertension, diabetes and low socioeconomic status, were adjusted for in our final hazards ratio model. The periodontal disease-stroke association, if causal, would be of great importance because of the potential that periodontal treatment could reduce the stroke risk.

Dental care is essential for maintaining good oral health, preventing periodontal disease, and identifying symptoms of systemic conditions that might first manifest in the mouth.^{[28]} During 2013, approximately 42% of the US population reported having a dental visit ^{[29]} with socioeconomic factors serving as a major determinant for not using regular dental care.^{[30]} A population-based, nationwide study in Taiwan identified periodontal disease as an important risk factor for incident ischemic stroke and showed that periodontal treatment lowered risk of stroke, significantly among young adults.^{[13]} This Example can demonstrate an independent role of regular dental care in prevention of incident ischemic stroke in a relatively elderly population. This can be validated by the fact that dental care was associated with lower burden of periodontal disease.

Important limitations of this study include the reliance on single periodontal disease assessment, a limited number of incident stroke subtypes, and owing to the observational nature of our investigation, the possibility of residual confounding cannot be eliminated. Socioeconomic factors such as access to care, income, and health-care behaviors may be potential confounders. However, education levels were adjusted that in these data serve as a surrogate for the socioeconomic status. Despite these potential limitations, this effort is one of the largest, US-based community studies of periodontal disease, dental care utilization, and ischemic stroke. Subjects excluded raise the possibility of selection bias. However as noted these subjects had a higher rate of incident ischemic stroke compared to those included, suggesting that they are unlikely to negatively influence the study results. Major strengths of this ARIC ancillary study were the use of comprehensive periodontal assessment classified into validated class, adjudication of incident ischemic stroke, stroke subtypes, and rigorous measurement of confounders.

Treatment of periodontitis in pregnant women improved periodontal disease and was safe, but did not significantly alter rates of pregnancy or fetal outcomes.^{[31]} Evidence from randomized controlled trials have established that intensive periodontal treatment improves systemic inflammation, high blood pressure, improves lipid profile^{[32]}, and endothelial dysfunction.^{[33]} Therefore, the treatment of PD could plausibly reduce stroke incidence. To the best of our knowledge this hypothesis has not been tested in a randomized clinical trial. The effect of PD treatment on recurrent vascular events in stroke/TIA patients is currently being investigated in PREMIERS trial (NCT02541032). Results may further help verify if periodontal treatment may reduce stroke risk.

### REFERENCES

1. Pihlstrom, B L, Michalowicz B S, Johnson N W. Periodontal diseases. Lancet 2005; 366: 1809-1820.
2. Eke P I, Jaramillo F, Thornton-Evans G O, Borgnakke W S. Dental visits among adult Hispanics—BRFSS 1999 and 2006. J Public Health Dent 2011; 71: 252-256.
3. Mustapha I Z, Debrey S, Oladubu M, Ugarte R. Markers of systemic bacterial exposure in periodontal disease and cardiovascular disease risk: a systematic review and meta-analysis. J Periodontol 2007; 78: 2289-2302.
4. Romagna C, Dufour L, Troisgros O, Lorgis L, Richard C, Buffet P, et al. Periodontal disease: a new factor associated with the presence of multiple complex coronary lesions. J Clin Periodontol 2012; 39: 38-44.
5. You Z, Cushman M, Jenny N S, Howard G. Tooth loss, systemic inflammation, and prevalent stroke among participants in the Reasons for Geographic and Racial Difference in Stroke (REGARDS) study. Atherosclerosis 2009; 203: 615-619.
6. Desvarieux, M, Demmer, R T, Rundek T, Boden-Albala B, Jacobs D R, Sacco R L, et al. Periodontal microbiota and carotid intima-media thickness: the Oral Infections and Vascular Disease Epidemiology Study (INVEST). Circulation. 2005; 111:576-582.
7. Scannapieco F A, Bush R B, Paju S. Associations between periodontal disease and risk for atherosclerosis, cardiovascular disease, and stroke. A systemic review. Ann Periodonol. 2003; 8:38-53.
8. Beck J, Garcia R, Heiss G, Vokonas P S, Offenbacher S. Periodontal disease and cardiovascular disease. J Periodontol. 1996; 67:1123-1137.
9. Syrjanen J, Peltola J, Valtonen V, livanainen M, Kaste M, Huttunen J K. Dental infections in association with cerebral infarction in young and middle-aged men. J Intern Med. 1989; 225:179-184.
10. Loesche W J, Schork A, Terpenning M S, Chen Y M, Kerr C, Dominguez B L. The relationship between dental disease and cerebral vascular accident in elderly United States veterans. Ann Periodontol. 1998; 3:161-174.
11. de Oliveira C, Watt R, Hamer M. Toothbrushing, inflammation, and risk of cardiovascular disease: results from Scottish Health Survey. BMJ. 2010; 340:c2451.
12. Chen Z Y, Chiang C H, Huang C C, Chung C M, Chan W L, Huang P H, et al. The association of tooth scaling and decreased cardiovascular disease: a nationwide population-based study. Am J Med. 2012; 125:568-575.
13. Lee Y L, Hu H Y, Huang N, Hwang D K, Chou P, Chu D. Dental prophylaxis and periodontal treatment are protective factors to ischemic stroke. Stroke 2013; 44: 1026-1030.
14. Feigin V L, Krishnamurthi R, Parmar P, Norrving B, Mensah G A, Bennett D A, et al. UPDATE ON THE GLOBAL BURDEN OF ISCHAEMIC AND HAEMORRHAGIC STROKE IN 1990-2013: THE GBD 2013 STUDY. Neuroepidemiology. 2015; 45:161-176.
15. Beck J D, Offenbacher S. Systemic effects of periodontitis: epidemiology of periodontal disease and cardiovascular disease. J Periodontol 2005; 76:2089-2100.
16. Janket S J, Baird A E, Chuang S K, Jones J A. Meta-analysis of periodontal disease and risk of coronary heart disease and stroke. Oral Surg Oral Med Oral Pathol Oral Radiol Endod 2003; 95:559-569.
17. Lafon A, Pereira B, Dufour T, Rigouby V, Giroud M, Bejot Y, et al. Periodontal disease and stroke: a meta-analysis of cohort studies. Eur J Neurol 2014; 21: 1155-1161.
18. Grau A J, Becher H, Ziegler C M, Lichy C, Buggle F, Kaiser C, et al. Periodontal disease as a risk factor for ischemic stroke. Stroke 2004; 35:496-501.
19. Albandar J M. Underestimation of Periodontitis in NHANES Surveys. Journal of Periodontology, 2011; 82: 337-341.
20. Morelli, T., Moss K L, Beck J, Preisser J S, Wu D, Divaris K, et al. Derivation and Validation of the Periodontal and Tooth Profile Classification System for Patient Stratification. J Periodontol 2017; 88:153-165.
21. Rosamond, W D, Folsom A R, Chambless L E, Wang C H, McGovern P G, Howard G, et al. Stroke Incidence and Survival among Middle-Aged Adults 9-Year Follow-Up of the Atherosclerosis Risk in Communities (ARIC) Cohort. Stroke 1999; 30: 736-743.
22. Autenrieth C S, Evenson K R, Yatsuya H, Shahar E, Baggett C, Rosamond W. Association between physical activity and risk of stroke subtypes: the atherosclerosis risk in communities study. Neuroepidemiology 2013; 40:109-116.
23. ARIC Investigators. The Atherosclerosis Risk in Community (ARIC) study: design and objectives. Am J Epidemiol 1989; 129:687-702.
24. Selvin E, Steffes M W, Zhu H, Matsushita K, Wagenknecht L, Pankow J, et al. Glycated hemoglobin, diabetes, and cardiovascular risk in nondiabetic adults. N Engl J Med 2010; 362: 800-811.
25. Eke P I, Dye B A, Wei L, Slade G D, Thornton-Evans G O, Borgnakke W S, et al. Update on Prevalence of Periodontitis in Adults in the United States: NHANES 2009 to 2012. J Periodontol 2015; 86: 611-622.
26. Leira Y, Lopez-Dequidt I, Arias S, Rodriguez-Yanez M, Leira R, Sobrino T, et al. Chronic periodontitis is associated with lacunar infarct: a case-control study. Eur J Neurol 2016; 23: 1572-1579.
27. Taguchi A, Miki M, Muto A, Kubokawa K, Migita K, Higashi Y, et al. Association between oral health and the risk of lacunar infarction in Japanese adults. Gerontology 2013; 59: 499-506.
28. Institute of Medicine. Improving access to oral health care for vulnerable and underserved populations. Washington (DC): National Academies Press; 2011.
29. Chad D. Meyerhoefer, Irina Panovska and Richard J. Manski. Projections of Dental Care Use Through 2026: Preventive Care to Increase While Treatment WII Decline. Health Affairs 2016; 35: 2183-2189.
30. Manski R J, Hyde J S, Chen H, Moeller J F. Differences Among Older Adults in the Types of Dental Services Used in the United States. 2016;Inquiry 53.
31. Michalowicz B S, Hodges J S, DiAngelis A J, Lupo V R, Novak M J, Ferguson J E, et al. Treatment of periodontal disease and the risk of preterm birth. N Engl J Med. 2006 Nov. 2; 355:1885-94
32. D'Aiuto F, Parkar M, Nibali L, Suvan J, Lessem J, Tonetti M S. Periodontal infections cause changes in traditional and novel cardiovascular risk factors: results from a randomized controlled clinical trial. Am Heart J 2006. 151: 977-984.
33. Tonetti M S, D'Aiuto F, Nibali L, Donald A, Storry C, Parkar M, et al. Treatment of periodontitis and endothelial function. N Engl J Med 2007. 356: 911-920.

TABLE 25
Selected Characteristics of the Study Participants, According to Periodontal Profile Class. *

| | Periodontal Disease class at baseline | | | | | | |
|---|---|---|---|---|---|---|---|
| | PPC-A | PPC-B | PPC-C | PPC-D | PPC-E | PPC-F | PPC-G |
| Value | (n = 1837) | (n = 1036) | (n = 689) | (n = 793) | (n = 993) | (n = 890) | (n = 498) |
| Age (yr) | 61.7 ± 5.5 | 62.3 ± 5.8 | 61.5 ± 5.5 | 63.7 ± 5.6 | 62.8 ± 5.6 | 63.0 ± 5.5 | 618 ± 5.7 |
| Sex (%) | | | | | | | |
| Female | 66.5 | 47.2 | 55.4 | 54.9 | 44.6 | 56.9 | 38.4 |
| Male | 33.5 | 52.8 | 44.6 | 45.1 | 55.4 | 43.1 | 61.6 |
| Race (%) | | | | | | | |
| African- | 3.4 | 3.2 | 72.3 | 18.2 | 1.6 | 34.2 | 45.8 |
| American | | | | | | | |
| White | 96.6 | 96.8 | 27.7 | 81.8 | 98.4 | 65.8 | 54.2 |
| Body Mass | 27.4 ± 4.8 | 28.3 ± 4.9 | 30.4 ± 6.2 | 28.9 ± 5.3 | 28.2 ± 5.0 | 29.7 ± 6.1 | 29.5 ± 5.9 |
| Index† | | | | | | | |
| Waist-to-hip | 0.92 ± 0.08 | 0.95 ± 0.07 | 0.94 ± 0.07 | 0.96 ± 0.07 | 0.95 ± 0.07 | 0.96 ± 0.07 | 0.96 ± 0.07 |
| Ratio | | | | | | | |
| Hypertension | 26.0 | 32.1 | 47.4 | 38.3 | 28.1 | 40.0 | 41.1 |
| (%) | | | | | | | |
| Diabetes (%) | 8.4 | 12.1 | 21.7 | 15.7 | 10.9 | 18.7 | 21.7 |
| Education (%) | | | | | | | |
| Basic | 5.4 | 8.1 | 23.8 | 16.4 | 5.7 | 28.5 | 23.3 |
| Intermediate | 41.8 | 45.3 | 32.2 | 48.5 | 44.7 | 47.3 | 39.0 |
| Advanced | 52.8 | 46.6 | 44.0 | 35.1 | 49.6 | 24.2 | 37.8 |
| Smoking (%) | | | | | | | |
| Never | 54.6 | 55.2 | 51.0 | 41.2 | 36.4 | 35.8 | 46.8 |
| Former | 38.7 | 38.6 | 36.2 | 42.5 | 46.9 | 42.9 | 37.7 |
| Current | 6.8 | 6.2 | 12.8 | 16.3 | 16.7 | 21.3 | 15.6 |
| Alcohol (%) | | | | | | | |
| Never | 16.0 | 18.8 | 29.7 | 23.8 | 8.4 | 25.0 | 24.1 |
| Former | 21.0 | 25.0 | 34.6 | 28.6 | 21.0 | 35.0 | 32.8 |
| Current | 16.0 | 18.8 | 29.7 | 23.8 | 8.4 | 25.0 | 24.1 |
| Cholesterol | 203.5 ± 34.6 | 200.5 ± 35.5 | 199.0 ± 37.5 | 199.4 ± 36.7 | 201.2 ± 35.2 | 202.1 ± 37.1 | 198.5 ± 37.8 |
| (mg/dl) | | | | | | | |
| LDL | 120.7 ± 32.2 | 121.6 ± 31.9 | 121.9 ± 35.2 | 121.3 ± 33.2 | 123.3 ± 32.4 | 124.2 ± 34.8 | 124.5 ± 33.8 |
| HDL | 54.1 ± 17.0 | 48.5 ± 16.2 | 52.1 ± 17.2 | 48.1 ± 15.6 | 49.5 ± 16.9 | 48.8 ± 16.1 | 48.3 ± 15.7 |
| Triglycerides | | | | | | | |
| (mg/dl) | | | | | | | |
| Median | 125 | 130 | 104 | 127 | 125 | 126 | 110 |
| 25th-75th | 89-174 | 93-186 | 77-144 | 92-179 | 90-171 | 92-177 | 80-150 |
| percentile | | | | | | | |

* Plus-minus values are means ± standard deviation
†The body-mass index = weight (kilogram)/height (meter)2

TABLE 26
Selected Characteristics of the Study Participants.
Accordina to Frequency of Dental Care Utilization

Dental Care Utilization

| | Regular DDS User | Episodic DDS User |
|---|---|---|
| Value | (n = 6670) | (n = 3692) |
| Age (yr) | 62.7 ± 5.6 | 63.0 ± 5.7 |
| Sex (%) | | |
| Female | 57.8 | 52.9 |
| Male | 42.2 | 47.1 |
| Race (%) | | |
| African-American | 8.5 | 39.3 |
| White | 91.5 | 60.7 |
| Body Mass Index† | 28.1 ± 5.1 | 29.9 ± 6.2 |
| Waist-to-hip Ratio | 0.94 ± 0.07 | 0.96 ± 0.07 |
| Hypertension (%) | 33.0 | 44.3 |
| Diabetes (%) | 11.9 | 23.3 |
| Education (%) | | |
| Basic | 8.5 | 34.4 |
| Intermediate | 43.5 | 41.7 |
| Advanced | 48.0 | 23.9 |
| Smoking (%) | | |
| Never | 47.7 | 40.8 |
| Former | 41.3 | 39.4 |
| Current | 11.0 | 19.8 |
| Alcohol (%) | | |
| Never | 17.1 | 26.5 |
| Former | 24.7 | 37.5 |
| Current | 58.2 | 35.0 |
| Periodontal status | | |
| PPC-A | 34.4 | 7.8 |
| PPC-B | 17.7 | 9.1 |
| PPC-C | 6.7 | 19.8 |
| PPC-D | 11.6 | 12.3 |
| PPC-E | 17.7 | 6.8 |
| PPC-F | 7.4 | 28.9 |
| PPC-G | 4.5 | 15.4 |

*Plus-minus values are means ± standard deviation
†The body-mass index = weight (kilogram)/height (meter)²

### EXAMPLE 6

This Example reviews aspects of current efforts to allow precision dentistry to be realized and focus on one of the major innovations that may help allow precision dentistry to be practiced by periodontists.¹ That recent innovation is the World Workshop Model (WW17). The other approach to enabling precision dentistry is the disclosed Periodontal Profile Class System (PPC).² These two approaches represent examples of Supervised and Unsupervised Learning systems, respectively. This Example compares and contrasts these two learning systems for their ability to successfully classify patients into homogeneous disease and risk groups, as well as their feasibility at achieving the goals of enabling precision dentistry.

During the development of the PPC system, the World Workshop for Disease Classification 2017 released its recommendation for Stages and Grades. As shown in Table 27, PPC classes can be renamed and reordered to PPC-Stages to harmonize PPC-Stages with the World Workshop recommendations. It is important to note the people assigned to each designation do not change. Only the names and order of the PPC to PPC-Stages were changed.

TABLE 27
Harmonization of PPC with World Workshop Stages and Grades

| PPC-Designation | WW Compliant Designation |
|---|---|
| PPC-A Health | PPC-Stage I Health/lncidental |
| PPC-B Mild | PPC-Stage II Mild |
| PPC-C High GI | PPC-Stage V Mild TL/High GI |
| PPC-D Tooth Loss | PPC-Stage VI Mod TL/Reduced |
| | Periodontium |
| PPC-E Posterior Disease | PPC-Stage III Moderate |
| PPC-F Severe Tooth Loss | PPC-Stage VII Severe TL |
| PPC-G Severe Disease | PPC-Stage IV Severe Disease |

As disclosed herein, the concept of Stages and Grades works as expected in that periodontal status appears to be more serious in each successive Stage. In addition, the seriousness and complexity of the disease is greater as Grade is higher within each Stage. Stages and Grades are important for precision dentistry because they consider risk of future disease and prognosis, and enable practitioners to use more signs, symptoms and other associated factors when placing a patient in a diagnostic category. As disclosed herein, the assignment of Stages and Grades using Unsupervised Learning systems is superior to Supervised Learning systems for prediction of 10-year tooth loss and attachment loss progression. In addition, the disclosed Unsupervised learning approach (PPC Stages) results in stronger associations between the periodontal phenotypes and systemic diseases and conditions (prevalent diabetes, C-reactive protein, and incident stroke). This likely occurs because an unsupervised learning model produces more data-driven, mutually exclusive, homogeneous groups than a supervised learning model.

What is Precision Medicine?

Precision medicine can be generally thought of as a collection of health care strategies that take individual variability into account.³ It is a contemporary, multifaceted approach to care involving the consideration of “individual differences due to people's genetic makeups, environments and lifestyles”.⁴ Precision medicine uses personal and disease-related information to classify individuals with similar characteristics into groups with the goal of improving treatment recommendations.⁵ The notion of customizing care according to individuals' characteristics has been referred to by many names, including individualized medicine,⁶ stratified medicine,⁷ network medicine,⁸ predictive medicine,⁵ personalized medicine,⁹ precision medicine¹⁰ and P4 medicine.¹¹

These terms have been used interchangeably and for diverse definitions ranging from “large ‘omics’ data to variations of common laboratory measurements—in order to provide better applications and dosing of drugs and improved clinical stratification of patients, as well as to identify biomarkers that indicate susceptibility to or severity of disease”.¹² Personalized medicine is similar to precision medicine because it is said to individualize care by using a person's unique genetic, environmental and clinical profile.¹³ However, the term ‘personalized medicine’ has also been thought to imply the creation of new technologies or treatments for an individual based on their unique biological fingerprint.¹⁴

Precision medicine is rather the ability to group people based on similar risk factors and response to treatment.¹⁰ The term, precision medicine, reflects the emphasis on more precise accounting for individual variability than used previously.¹⁵ Various groups have been exploring the application of precision medicine in oral health care, known as, ‘Precision Dentistry’.^{4,16} We will refer specifically to precision medicine throughout this paper, which will primarily focus on the applications of Precision Oral Health related to Periodontal disease.

The concept of personalized care is not new. Throughout history, physicians have collected information from patients (e.g. medical and dental history and clinical signs and symptoms) in order to better address their unique health needs. Clinicians have also demonstrated personalized care while classifying alike groups of people to better tailor treatments. For example, the long-standing practice of identifying and treating individuals based on their blood type.³

The concept of precision medicine is well-known in the field of Oncology. Patients with the same type of cancer have often failed to respond to standard treatments, which has been attributed to the idea that cancer in one person may be very different than cancer in another.¹⁷ Decades of research have been done to determine why people with the same condition respond differently.

Clinical outcomes have been associated with molecular subtypes of tumors, irrespective of their organ of origin.¹⁷ This means that some forms of breast cancer may be more similar to a subtype of stomach cancer than other types of breast cancer. These two types of cancer could receive the same treatment protocol because the label (e.g. ‘stomach cancer’ or ‘breast cancer’) doesn't matter in terms of the effectiveness of therapy. Thus, cancer treatments have shifted away from broadly-acting cytotoxic drugs to tumor-specific drugs that in some instances may require the addition of radiation or surgery. This treatment approach is changing the face of cancer care.^{3,9}

A precision approach to oncology uses baseline information to predict patients' response to treatment in order to tailor treatment decisions.⁷

A similar phenomenon has been observed with periodontal disease. Periodontal diseases are highly prevalent, affecting between 50-90% of the population.¹⁸ Dramatic differences can occur between individuals with very similar clinical presentations in addition to individuals with identical clinical presentations responding differently to the same therapy.¹⁹ Standard methods to assess Periodontal disease severity include evaluating clinical signs and symptoms, along with medical and dental history, and have failed to reliably predict treatment response.²⁰ While much work on Precision Medicine involves pharmacogenomics and gene-specific therapies in cancer care, precision medicine is also being explored for applications in complex, chronic diseases.¹²

The larger goals of precision medicine are to inform and improve health care, including the treatment of chronic diseases.^{3,4} Chronic diseases account for over 70% of care in the United States, with a projected increase as this population ages.¹² Addressing complex chronic diseases with a precision medicine approach is in its infancy. Much of the work in precision medicine has focused on single gene targets, so it may prove challenging to apply this work to chronic conditions that are influenced by hundreds of genes.^{5,12} For example, Periodontal disease is a chronic condition believed to be influenced by hundreds of genes and numerous environmental factors. Non-genetic factors (e.g. social, behavioral and environmental) predominate over genetic factors in terms of disease risk and development.⁵ For example, over 90% of diabetes mellitus²¹ and more than 80% of coronary heart disease²² can be prevented through healthy lifestyle habits and behaviors (e.g. balanced diet, maintaining normal Body Mass Index, smoking avoidance, etc.). Non-genetic factors like smoking, diabetes, and nutrition, also play a large role in Periodontitis.¹⁸ Improved phenotyping (e.g. categorizing patients into groups based on similar risk factors and disease characteristics) and increased utilization of the Electronic Health Record are two key factors to employing a precision approach to chronic disease.¹² Thus, statistical models and improvements to health information technology at the individual level—those that can account for both genomic and non-genomic factors—are particularly well suited for a precision medicine approach to chronic disease.

As technologies have evolved, more has been learned about patients—the complexity of human physiology and how the world we live in affects people.¹³ High-throughput technologies have contributed to an individualized, data-driven approach to care. Physicians are now able to collect and monitor personal profiles of “omics” (i.e. the genome, the epigenome, the transcriptome, the proteome, and the metabolome),²³ which are being used to identify a patient's physiological or pathological state at the time of the examination, as well as create a more comprehensive picture about each patient's health.

While researchers and clinicians are beginning to understand the multifactorial nature of health and disease, the traditional approach to care remains focused on diagnosing a condition or disease using clinical signs and symptoms, and only a few risk factors and biomarkers. For example, low-density lipoprotein (LDL) levels are associated with cardiovascular disease risk.

While some individuals are genetically predisposed to certain diseases, social, behavioral and environmental risk factors also play a large role in disease development, and are important components of the Precision Medicine initiative.²⁴ Periodontitis is often represented by one or two variables characterizing the phenotype, e.g. clinical attachment loss, probing depth.¹⁹

New multivariate classification systems—capable of accounting for the complexity of disease risk, development and progression—have been called for to better identify disease-susceptible and treatment—non-responsive individuals. These new systems would replace current classifications that are heavily influenced by a few clinical signs or risk factors (e.g. with Periodontitis, probing and attachment level measurements).^{19,25}

According to the National Research Council, Precision Medicine refers to the tailoring of medical treatment to the individual characteristics of each patient.¹⁰ While some people have interpreted that this involves the creation of drugs or medical devices that are unique to a patient, this is not a reasonable undertaking. Clearly, trying to treat each person based on their unique biological characteristics is infeasible for a number of reasons. It is also unnecessary because groups of patients with complex chronic diseases, such as periodontitis often share similar phenotypes, or alike risk factors, and response patterns to disease.^{5,16} Thus, patients can be categorized into discrete subpopulations or groups based on phenotypes, and still receive specific treatment for their disease.¹⁶ The goal behind creating these discrete, non-overlapping groups or phenotypes, is that the more homogeneous the group, the more likely the treatment will be effective for all the members of the group.

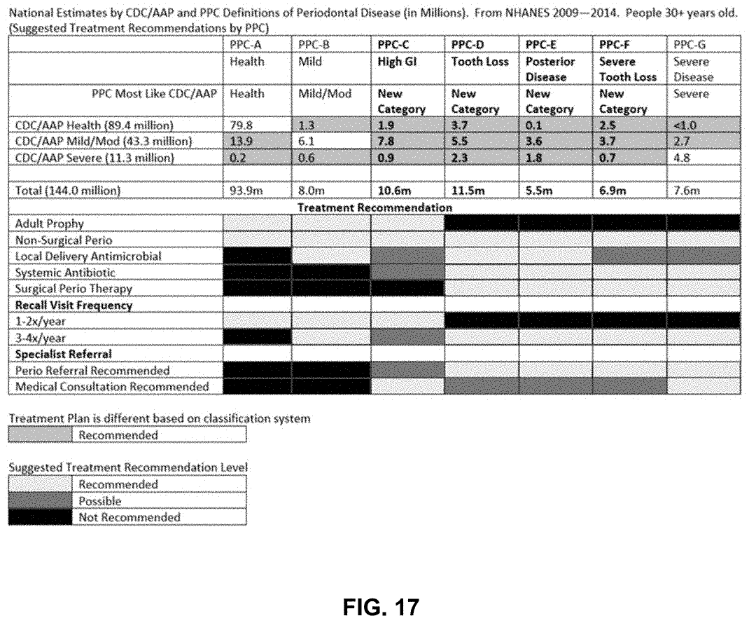

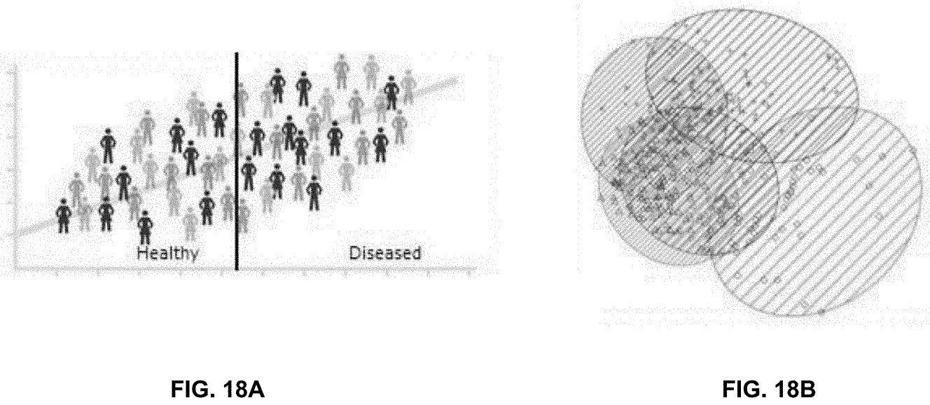

Through precision medicine, the opportunity to use data-informed phenotypes of disease redefines the way we look at disease. Why is it necessary to look for a new method of defining disease? Perhaps Henry Ford (the founder of Ford Motor Company) said it best: “If you always do what you've always done, you'll always get what you've always got.” For example, traditional definitions of Periodontal disease have served well for decades. These definitions are typically based on one (or a few) periodontal variables where one or more experts draw ‘lines in the sand’ to differentiate health from disease (FIG. 18A). Further, this approach lends to one-size-fits-all treatments, which often fail.²⁶

It has been suggested that the current diagnostic process is based on symptoms, test results, or other factors.²⁷ However, once diagnosed with a disease, patients may be assigned a stage based on another set of human-defined rules. A major limitation of using traditional definitions of disease is that one can only find patterns of disease that they choose to look for.²⁸

The future of precision medicine relies on transitioning away from predefined clinical disease categories to allow the data to speak for themselves.²⁸ In addition, traditional definitions of disease picked by experts (i.e. lines in the sand) can be good at finding associations where enough is known to describe the risk exposure and disease outcome, but can be limited where the exposure or outcome are not well-defined. Clinical data can be used to search for ‘patterns in the sand’ instead of experts predefining ‘lines in the sand’.

The methods used to create data-informed diagnostic groups generally are referred to as Unsupervised Learning (i.e. patterns in the sand) to contrast them from the more traditional Supervised Learning of experts in the field drawing ‘lines in the sand’ to create categories of health and disease (FIG. 18A). Another type of Supervised Learning is creating clusters of people based on their disease and personal characteristics, e.g. Principal Component Analysis (FIG. 18B).

This method does create different groups, but they are overlapping, which means some people can be in more than one group. In addition, the analyses are often done on a group basis and the level of risk is applied to everyone in that group. This is problematic if the groups are created based on a small number of disease and personal characteristics related to disease progression.

If this occurs, some individuals will receive unneeded treatment and others may not respond to the treatment protocol.

The Unsupervised approach is supported by recent results indicating that long-recognized diseases such as Asthma or Heart Failure are not really single entities, but instead are collections of many different phenotypes that may or may not coincide with historical disease boundaries.^{29.31} So, while Supervised Learning is good at finding patterns that explain phenotypes enough is known to label in advance, it is unsuited to the scenario in which enough is not known to label the phenotypes, and there is a desire to discover them from the data.²⁸

The Importance of Latent Class Analysis for Facilitating Precision Oral Health and Treatment

There are several types of statistical models that would fit under the category of Unsupervised Learning. One such application is Deep Learning, a class of machine learning algorithms based on neural networks.²⁷ Another method is k-means clustering based on vectors. Latent Class Analysis (LCA) is a type of model that finds latent (“hidden”) homogeneous classes of people based on input variables according to their maximum likelihood class membership.

Latent Class Analysis (LCA) is a statistical model of Unsupervised Learning that is rather new to dentistry, but has been used in medicine and other disciplines for some time. The fundamental assumption underlying latent class models is that of local independence that states that objects (i.e. persons, cases) in the same latent class share a common joint probability distribution among the observed variables. Since persons in the same latent class (cluster) cannot be distinguished from each other based on their observed responses, they are similar to each other (homogeneous) with respect to these observed variables. Persons are classified into that class having the highest posterior membership probability of belonging within the set of responses for that case.³² Posterior probability is the probability of something occurring given other factors in the model.³³

This approach is important because, (1) LCA can take into account multiple characteristics of the phenotype, rather than just one or two as used in other approaches, (2) LCA classification is based on individuals who are placed together in mutually exclusive classes based on multiple, similar characteristics, rather than pre-defined groups; (3) The individuals in each class are more homogeneous than in most other approaches; (4) LCA modeling creates latent (hidden) classes that may not be obvious from looking at the clinical signs themselves; and (5) The probability of being placed in a specific class based on their characteristics are extremely high, e.g., misclassification is extremely low.²

LCA has been used to identify phenotypes or subgroups of similar patients in order to modify treatment in several areas of medicine, including cancer, arthritis, psoriasis, psychology and cardiovascular health. For example, Ferrat, et al. conducted a study of older adults in order to identify profiles that would help physicians better select cancer treatments and other geriatric interventions (2016).³⁴ They found four distinct profiles in these patients.³⁴ Another study conducted a latent class analysis of 227 older women with self-reported arthritis.³⁵ Classes of women were identified based upon the multi-dimensional nature of their pain experience (e.g. sensory, affective, and cognitive dimensions).³⁵ De Luca, et al., found three distinct subgroups of pain profiles (2017).³⁵ Women had very different experiences of pain, and class membership impacted significantly on health-related quality of life.³⁵ These preliminary findings provide a stronger understanding of profiles of pain and may contribute to the development of tailored treatment options in arthritis.³⁵

LCA has also been used to categorize patients with psoriasis. Psoriasis is associated with much comorbidity. An understanding of these comorbidity patterns can help foster better care of patients with psoriasis. LCA was used to empirically identify psoriasis comorbidity patterns in a nationwide sample of 110,729 incident cases of psoriasis (2002-2012) from the National Health Insurance database in Taiwan.³⁶

Additionally, LCA has been examined in post-Myocardial Infarction (MI) patients with psychological depression.³⁷ This investigation focused on whether different patterns of post-MI depressive symptoms can be identified and their associations with cardiac events determined.

Five distinct patterns were found: no depressive symptoms (56.4%), mild depressive symptoms (25.7%), moderate and increasing depressive symptoms (9.3%), significant but decreasing depressive symptoms (4.6%), and significant and increasing depressive symptoms (4.0%).37 LCA revealed a subgroup of depressed, post Myocardial Infarction subjects, who developed significant ongoing depressive symptoms, and exhibited increasing risk of new cardiac events.³⁷ This analysis led researchers to question the effects of antidepressant treatment on cardiac prognosis.

Precision Oral Health/Dentistry

Traditionally a patient is classified based on how well she or he meets established disease classifications. However, one goal of precision dentistry is to classify a patient based on their risk for disease progression and tooth loss. This new objective requires a different approach.

Until now, the profession has been using Supervised Learning methods to achieve the goals. While current supervised methods of Periodontal disease classification are based on decades of data-based experience, patients with a diagnosis of some category of periodontitis (e.g. incipient, mild, moderate, severe) respond differently to the same treatment—meaning that current classifications of periodontal phenotypes do not contain patients who are homogeneous in their makeup. This is likely because precision care requires that we classify a patient based on the goal of placing each individual into a group where all members of the group are homogeneous as far as the condition is concerned. The idea is that members of that group have similar risk for disease development, progression or response to treatment. If this requirement is met, then the label or name we give to the group of individuals is not important (e.g. groups could be labeled A, B, C, D, or 1, 2, 3, 4). Alternatively, they could be given a name that is somewhat descriptive of the clinical status of the group members.

It may be useful to consider Precision Oral Health as requiring a paradigm shift in our thinking about disease classification. Precision care requires that we classify a patient based on the goal of assigning an individual into a group where all members of the group are homogeneous as far as the condition is concerned. Consequently, disease classification should no longer be thought of as accurately placing a patient into a specific disease category. Instead, the goal is to use data to place each individual into a mutually exclusive, homogeneous, unbiased group that represents the phenotype of interest with the idea that members of that group have similar risk for disease development, progression or response to treatment. Instead of thinking “What is the proper diagnostic category for this patient?” the thought needs to be “What is this patient's risk of disease progression, tooth loss, or poor treatment response?”

There are many advantages for using Precision Dentistry as a custom dental management model. According to Bartold (2017), it aligns the field of dentistry with current medical practice, avoiding a tendency to administer an ‘average’ treatment for all patients with a certain condition.³⁸ Instead, it provides patient-centered treatment that will likely be cost effective as well as improve health outcomes.^{26,38} According to Schwendicke (2018), this ‘tailored dentistry’ is not only advantageous, but necessary for the future of dentistry.³⁰

Various studies have been exploring the application of precision medicine concepts to dentistry.^{4,16,33,39} A few examples of these research topics include genomics and oral cancer, orofacial pain, and periodontal disease.¹³ It has been suggested that a precision oral health approach would benefit the diagnosis and treatment of periodontal disease, although genetic evidence to support this is scarce.¹⁶ Precision dentistry models are also being used to predict risk of early childhood caries⁴⁰ as well as adult caries.⁴¹

An example of where this approach is being investigated is in the area of Temporomandibular Joint Dysfunction. Three subgroups of patients with Temporomandibular Joint Dysfunction pain were identified using supervised cluster analysis (referred to as the adaptive, pain-sensitive, and global symptoms clusters).⁴² Compared with the adaptive cluster, participants in the pain-sensitive cluster showed heightened sensitivity to experimental pain, and participants in the global symptoms cluster showed both greater pain sensitivity and greater psychological distress.⁴² This group continues work in this area by treating these patients based on their subcategory.

Another research area is head and neck squamous cell carcinoma. Recent successes in the use of immunity-inducing antibodies have stimulated increased interest in the use of precision immunotherapy of head and neck squamous cell carcinoma.⁴³

Comparing Supervised Learning and Unsupervised Learning Models for Classifying Risk for Periodontal Disease, Disease Progression and Tooth Loss

The role of doctors has become almost analogous with that of investigators, in that both groups gather information to determine how to appropriately diagnose and treat. However, all too often there is an incomplete clinical picture that produces inherent guesswork in the practice of medicine and dentistry. It is necessary to develop tools that eliminate this uncertainty and standardize clinical practice.¹⁶ Such tools would be able to classify patients with similar characteristics into groups that have known risks for disease and disease progression, allowing clinicians to select a more specific treatment plans for that individual, and hopefully improve clinical outcomes.

Several tools are being developed and proposed to classify individuals' risk for developing periodontal disease. This Examples compares and contrasts these tools and how they could help achieve a precision approach to oral health. One of these is the World Workshop Model (WW17)1 and the other is the disclosed Periodontal Profile Classes (PPC Stages) Model.² The WW17 and PPC Stages models have common goals. The WW17 model represents a Supervised Learning approach while the PPC Stages model is representative of an Unsupervised model.

World Workshop Model

As part of the World Workshop on Periodontal Disease Classification (WW17), a seminal paper by Tonetti, Greenwell, and Kornmanl introduced a multidimensional staging and grading system as a framework for reclassifying chronic periodontitis. This schema is similar to the method used in oncology or rheumatology, in which ‘staging’ is based upon the severity of disease and complexity of case management while ‘grading’ speaks to biological features, such as the predicted rate of disease progression, which also relates to the risk for tooth loss and for becoming edentulous. Furthermore, grading should reflect the impact of person-level risk factors and potential threats to general health. Specifically, the authors proposed a new nosological structure (i.e. classification of diseases) that included distinct classes of disease that reflect a diagnosis. These classes are referred to as Stages, but there also is a designation of Grade of disease for each stage that reflects risk for tooth loss as well as other risk factors, such as smoking and diabetes. Furthermore, the proposed staging and grading was developed with the requirement that it should reflect prognosis, as well as the complexity of treatment needs, including recommendations for referral if needed to assist the clinician in case management.

This system, which represents an example of a Supervised Learning model, has great potential for disease classification that could support the concept of precision dentistry, since it uses multiple variables to represent the phenotype and requires that the provider considers prognosis as well as risk for disease progression and tooth loss. The WW17 model is an important next step to help enable precision dentistry.

Periodontal Profile Classes (PPC) Stages Model

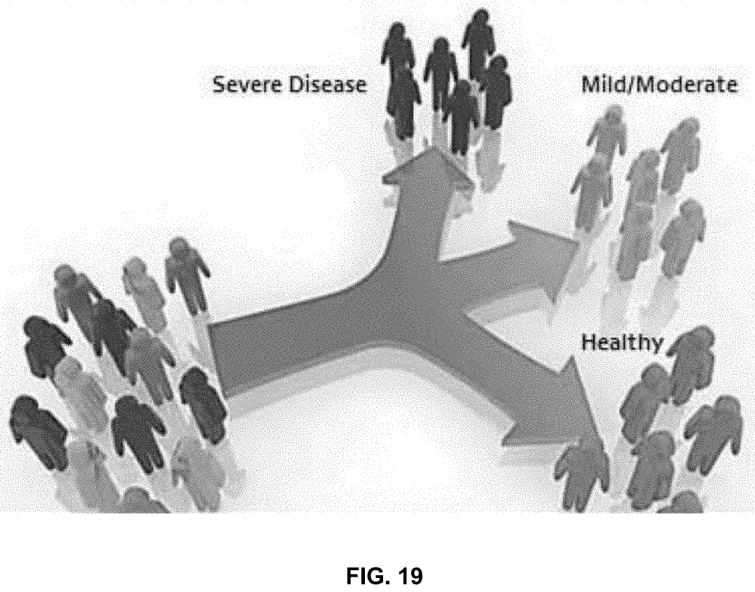

The disclosed Periodontal Profile Classes (PPC) model is an example of an unsupervised learning model that uses an approach to classification called Latent Class Analysis. LCA takes a group of people and places each individual into mutually exclusive classes (bins) based on their characteristics as illustrated in FIG. 19. To accomplish this, Latent Class Analysis takes a different approach to classification (e.g. creating multidimensional disease patterns using a data-driven, probabilistic modeling approach) than traditional methods of predefining disease classification (e.g. drawing lines in the sand).

An agnostic, data-based approach to define classes of periodontal conditions has been developed, which includes the spectrum of clinical presentations of health and disease as well as missing teeth. Seven classes of periodontal disease were identified using Latent Class Analysis. They included three new classes associated with mild tooth loss and very high Gingival Index (GI) scores, moderate tooth loss with reduced periodontium, and severe tooth loss. This group of seven periodontal disease conditions was designated as ‘Periodontal Profile Classes’ and the individual diagnoses were named Health, Mild Disease, High Gingival Inflammation (High GI), Tooth Loss, Moderate (Posterior) Disease, Severe Tooth Loss, and Severe Disease.²

A similar classification based on the teeth within each individual (Tooth Profile Class=TPC), was also derived using LCA.² Fundamentally, the model builds upon seven types of periodontal status seen around individual teeth as determined agnostically, plus missing teeth. These seven TPCs include teeth that are: Healthy, have Recession, have Crowns, have a High GI, have Interproximal Attachment Loss, have a Reduced Periodontium, and Severe disease (deep pockets and attachment loss).²

The IPR—(Index of Periodontal Risk) incorporates the individual patient PPC and TPC composition and a longitudinal database of tooth loss and attachment loss to create a point estimate of future disease risk for each individual patient.² The Dental Atherosclerosis Risk In Communities Study (ARIC) 10-year tooth loss data were used to compute the risk of tooth loss for each tooth profile class within each periodontal profile class assignment. A composite risk score for each individual was then calculated based on tooth loss risks; this continuous score is the IPR (range 4-46). The analytical approach used to calculate IPR was based on a 7×7 table (PPC×TPC) of predicted probabilities for 10-year tooth loss. First, each participant was assigned to one of the 7 PPCs and then, each tooth was classified to one of the 7 TPCs. The IPR was then calculated as the mean predicted probability for 10-year tooth loss across all teeth present for each individual. The development of the IPR included traditional risk factors for periodontal disease, such as age, sex, race, diabetes, and smoking status.

The probabilities of being assigned to a particular PPC or TPC are extremely high. The posterior probabilities (the conditional probability when all other variables are considered) of being assigned to a particular PPC ranged from 0.967 for Mild Disease to 1.00 for Severe Tooth Loss. The posterior probabilities for assignment to a particular TPC ranged from 0.823 for Gingival Inflammation to 0.953 for Recession.²

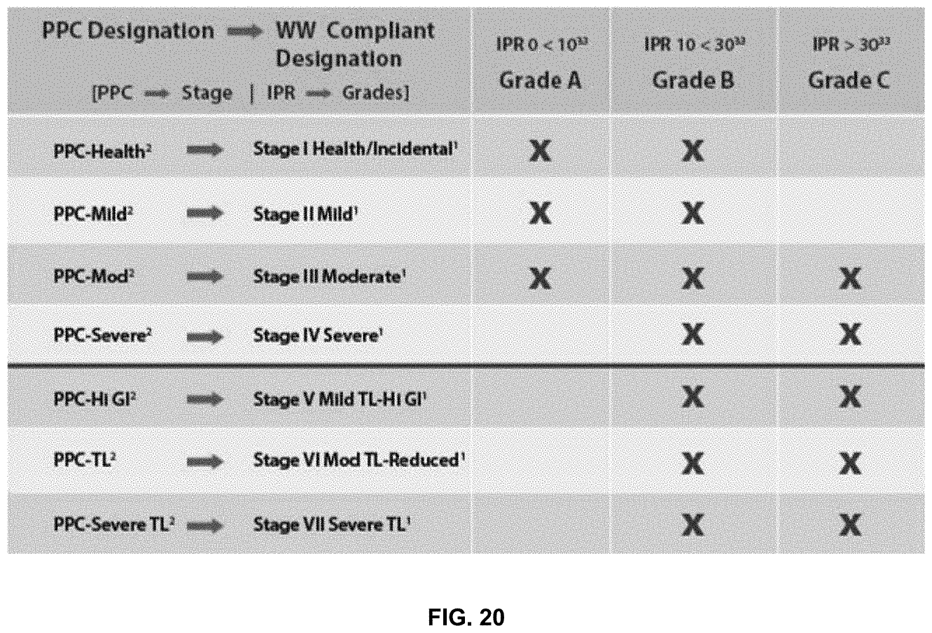

An Unsupervised Learning system has the goal of placing individuals into mutually independent, homogeneous risk groups in relation to a disease or condition. Once the groups are established, it doesn't matter what the group is named. A specific nomenclature was used during the development of the PPC model.^{1,2,33,39,44} The seven groups were first labeled as Groups A through G. Since periodontal disease has characteristics not usually found in a chronic disease, (multiple teeth and sites that can have different patterns of disease or disease progression within an individual), each group was labeled based on the patterns of different clinical conditions (ranging from health to severe disease) in the teeth of individuals in each group. Those labels were Healthy, Mild, High GI, Tooth Loss, Posterior Disease, High Tooth Loss, and Severe Disease. While some of these labels sound like traditional periodontal disease categories, they do not contain all of the same individuals as a similarly sounding traditional category because they were developed with a different goal, which was to place individuals into groups with a similar risk for tooth loss. Therefore, it was relatively simple to change the original PPC categories to be consistent with Stages and Grades (FIG. 20).

The transition to harmonize with the WW17 nomenclature of Stages and Grades is described briefly here. The previous seven PPC disease states become Stages I-VII and the Grades of disease were defined using the following IPR cut-off-values. Grade A=IPR<10, Grade B=IPR 10 to <30, Grade C=30 or greater. Stages were assigned first, and then using IPR as a risk indicator, the Grade was assigned for those within each Stage. For clarity the adaptation of PPC nomenclature^{1,2,33} to the WW17 Stages & Grades are shown in FIG. 20 and described in the Legend. Stages are grouped to show four Stages of periodontal status among those with a largely intact dentition (Health/Incidental, Mild Disease, Moderate Disease and Severe Disease) and three Stages that accommodate patterns of tooth loss, severe inflammation and a reduced periodontium (Mild Tooth Loss/High Gingival Inflammation, Moderate Tooth Loss/Reduced Periodontium, and Severe Tooth Loss). The Health/Incidental label for Stage I was created because the vast majority of individuals in this group have healthy teeth and gingiva, but some may also have a few pockets or sites with ≥3 mm CAL that may be a result of past inflammation or third molar extractions or some other etiology as indicated by Lang and Bartold (2018).⁴⁵ Lang and Bartold state that, “thus, probing pocket depth or probing attachment levels alone should not be used as evidence of gingival health or disease (2018).⁴⁵ They must be considered in conjunction with other important clinical parameters such as bleeding on probing, as well as modifying and predisposing factors.”⁴⁵ The risk for tooth loss or disease progression is the lowest of all the other Stages.

Importantly, classifications such as periodontitis associated with systemic disease, e.g. diabetes, were not used, but diabetes was considered as a subject-level risk for higher IPR and higher-Grade assignment on an individual basis-like a propensity risk score -that assigns risk at an individual level rather than adjusting the overall population risk estimates. Furthermore, the agnostic LCA assignments had no a priori assumptions of health or gingivitis, but rather were applied to the entire ARIC test population to permit the algorithm to assign traits that best described Stage and Grade strata.

The WW17 model states that after Stage is established for a patient, then Grade should be established based on an estimate of periodontitis progression. They suggest that clinicians start with the assumption that the patient is Grade B and then look for evidence that would modify that assumption. The first step is to look at longitudinal evidence of worsening clinical attachment loss (CAL) or radiographic bone loss (percent of bone loss/age) over a 5-year period.

If there is no progression over 5 years, the individual is classified as Grade A. If there is progression of less than 2 mm over 5 years they are classified as Grade B and if the progression is two or more mm over five years the individual is a Grade C. If these direct measures are not available, the clinician can use indirect measures, such as percent bone loss/age or the relationship between level of biofilm deposits and level of destruction. Once the Grade level is established the clinician should consider known risk factors, specifically whether the individual smokes, and if so, the number of cigarettes per day. The other risk factor is having diabetes and if they do, HbA1c levels. The clinician also can consider any systemic risk due to periodontitis that can be inferred from High sensitivity C-reactive protein levels. Other biomarkers that are indicators of CAL/bone loss can also be useful. The patient data that was used to model WW17 Grades does not contain longitudinal measures of CAL or radiographic bone loss, nor HbA1c information. There was information on biofilm deposits and CAL measures as well as hsCRP along with information on smoking and intensity of smoking. However, since the primary direct evidence required to create WW17 Grades was not available, grades are not presented.

However, data are available for PPC-derived Stages and Grades so the reader can evaluate how well the WW17 Stages and Grades system relates to risk for tooth loss due to periodontal disease.

Comparing WW17 and PPC Stages and Grades

If you follow the WW17 instructions for deciding on a Stage, what proportion of people fall into each Stage?

The WW17 instructions indicate that their Stages and Grades assume that everyone has Periodontitis or they would not be seeing a Periodontist. Periodontitis is defined as “Interdental clinical attachment loss (CAL) detectable at ≥2 non-adjacent teeth, or buccal or oral CAL≥3 mm with pocketing>3 mm is detectable at ≥2 teeth.”1

Dental ARIC data were applied to the WW17 model in order to create the distribution of participants for each Stage according the WW17 specifications. In the study sample, everyone met the criteria for periodontitis, except four individuals who only had one tooth. The initial stage should be determined using clinical attachment level; if not available, then radiographic bone loss (RBL) should be used.

As indicated in Table 27, six percent of the participants were classified as Stage I, forty-seven percent fell into Stage II, forty-seven percent were Stage III, and zero percent were classified as Stage IV using CAL as the clinical measure. It is possible that a younger population would have more individuals in Stage I. The cell for Stage IV was empty, because Stages III and IV have the same criterion for CAL of ≥5 mm. All were placed in Stage III until additional criteria were evaluated to determine who met the Stage IV criteria.

After deciding on a Stage, what proportion of people move to a different Stage based on WW17 measures of complexity?

WW17 measures of complexity include information on tooth loss that can be attributed primarily to periodontitis, which if available, may modify stage definition even in the absence of complexity factors. Complexity factors may shift the stage to a higher level, for example furcation II or III would shift to either stage III or IV irrespective of clinical attachment loss (CAL).

The distinction between stage III and stage IV is primarily based on complexity factors. For example, a high level of tooth mobility and/or posterior bite collapse would indicate a stage IV diagnosis. For any given case only some, not all, complexity factors may be present, however, in general it only takes one complexity factor to shift the diagnosis to a higher stage. It should be emphasized that these case definitions are guidelines that should be applied using sound clinical judgment to arrive at the most appropriate clinical diagnosis. For post-treatment patients CAL and radiographic bone loss (RBL) are still the primary stage determinants. If a stage-shifting complexity factor(s) is eliminated by treatment, the stage should not retrogress to a lower stage since the original stage complexity factor should always be considered in maintenance phase management.

Table 28 shows that there is Stage Modification from tooth loss due to periodontal disease. For those classified as Stage I, <1 percent moved to Stage III and 39 percent moved to Stage IV. For those in Stage II, 1 percent moved to Stage III and 24 percent moving to Stage IV. For those initially classified as Stage III, 38 percent moved to Stage IV due to tooth loss. Thus, 2136 individuals moved from Stage III to Stage IV based on both having both CAL and tooth loss due to periodontal disease. There also was Stage modification due to probing depth. For those classified as Stage I, <1 percent moved to Stage II and for those originally classified as Stage II, one percent moved to Stage III. Thus, tooth loss and probing depth in addition to the initial classification based on interdental CAL modified the final classification of individuals into Stages. The consideration of tooth loss and probing depth reduced the percent of participants in Stage I from 6 percent to 4 percent or a loss of 155 participants in Stage I. The same considerations for Stage II also reduced the percent of participants from 47 percent to 35 percent or a loss of 996. Stage III was reduced from 47 percent to 30 percent of the participants or a loss of 1,185 participants. Stage IV had zero occupants based on CAL measures, but ended up containing 31 percent of the participants, a gain of 2,136 participants.

TABLE 28
Classification of Dental ARIC Participants According
to World Workshop 2017 (WW17) Stages. (N = 6793)

| WW17 Periodontitis stage | | Stage I | Stage II | Stage III | Stage IV |
|---|---|---|---|---|---|
| Severity | Interdental CAL | 1 to 2 mm | 3 to 4 mm | ≥5 mm | ≥5 mm |
| | at site of | | | | |
| | greatest loss | | | | |
| | Initial Stage | 393 (6%) | 3186 (47%) | 3214 (47%) | 0 (0%) |
| | Assignment | | | | |
| | Distribution | | | | |
| | Radiographic | Coronal | Coronal | Extending to | Extending to |
| | bone loss | third | third | mid-third of | mid-third of |
| | | (<15%) | (15% to 33%) | root and | root and |
| | | | | beyond | beyond |
| | Tooth loss | No tooth loss due | | Tooth loss due | Tooth loss due |
| | | to periodontitis | | to periodontitis ≤4 | to periodontitis |
| | | | | teeth | of ≥5 teeth |
| Stage Modification based on | | <1% shift | 1% shift | 38% shift | |
| Tooth loss due to periodontitis | | to Stage III | to Stage III | to Stage IV | |
| | | 39% shift | 24% shift | | |
| | | to Stage IV | to Stage IV | | |
| Stage Assignment After Stage | | 239 (4%) | 2408 (35%) | 2010 (30%) | 2136 (31%) |
| Modification based on TL due to | | | | | |
| Periodontitis | | | | | |
| Complexity | Local | Maximum | Maximum | In addition | In addition |
| | | probing | probing | to stage II | to stage III |
| | | depth <=4 mm | depth <=5 mm | complexity: | complexity: |
| | | Mostly | Mostly | Probing | Need for |
| | | horizontal | horizontal | depth >=6 mm | complex |
| | | bone loss | bone loss | Vertical bone | rehabilitation |
| | | | | loss >=3 mm | due to: |
| | | | | Furcation | Masticatory |
| | | | | involvement | dysfunction |
| | | | | Class II or III | Secondary |
| | | | | Moderate | occlusal |
| | | | | ridge defect | trauma (tooth |
| | | | | | mobility |
| | | | | | degree >=2) |
| | | | | | Severe ridge |
| | | | | | defect |
| | | | | | Bite collapse, |
| | | | | | drifting, flaring |
| | | | | | Less than 20 |
| | | | | | remaining |
| | | | | | teeth (10 |
| | | | | | opposing pairs) |

| | | |
|---|---|---|
| Stage Modification based on | <1% shift | 1% shift |
| Probing depth | to Stage II | to Stage III |

| | | |
|---|---|---|
| Extent and | Add to stage as | For each stage, describe extent as localized (<30% of teeth |
| distribution | descriptor | involved), generalized or molar/incisor pattern. |

| | | | | |
|---|---|---|---|---|
| Modified Stage Assignment | 238 (4%) | 2390 (35%) | 2029 (30%) | 2136 (31%) |
| based on Interdental CAL, Tooth | | | | |
| loss and Probing depth | | | | |
| Modified Stage Assignment | 219 (4%) | 2056 (42%) | 1585 (32%) | 1085 (22%) |
| based on Interdental CAL, Tooth | | | | |
| loss and Probing depth for | | | | |
| Regular Dental Users | | | | |
| Modified Stage Assignment | 18 (1%) | 329 (18%) | 432 (24%) | 1035 (57%) |
| based on Interdental CAL, Tooth | | | | |
| loss and Probing depth for | | | | |
| Episodic Dental Users | | | | |

Information on individuals who visit the dentist on a regular basis can provide a sense of disease progression and tooth loss in regular dental attenders. The last two rows of Table 27 show the distribution across Stages for individuals who are regular dental patients and those who visit the dentist on an episodic basis. These data show that episodic users are less likely to be classified as Stage I, II, or III and more likely to be Stage IV than regular users or for the combined groups.

Regular users are just as likely to be classified as Stage 1. This is likely to be due to regular users making up 73% of the total number of individuals in Stage 1. Regular users are more likely to be in Stage II than the group as a whole and less likely to be in Stage IV. Episodic users are more likely to be assigned to Stages III and IV. This pattern could be due to a variety of reasons, including that they are resistant to periodontal treatment.

As expected, there was a difference in stage distribution between regular and episodic dental users, with episodic dental users more likely to be in Stages III and IV than regular users. When comparing regular dental users with the entire group, the distributions among Stages are quite similar, except regular dental users are eight percent more likely to be in Stage II and nine percent less likely to be in Stage IV. This indicates that the Stages concept does represent differences between regular and episodic dental attenders.

Pattern of Clinical Measures for 10-Year Tooth Loss

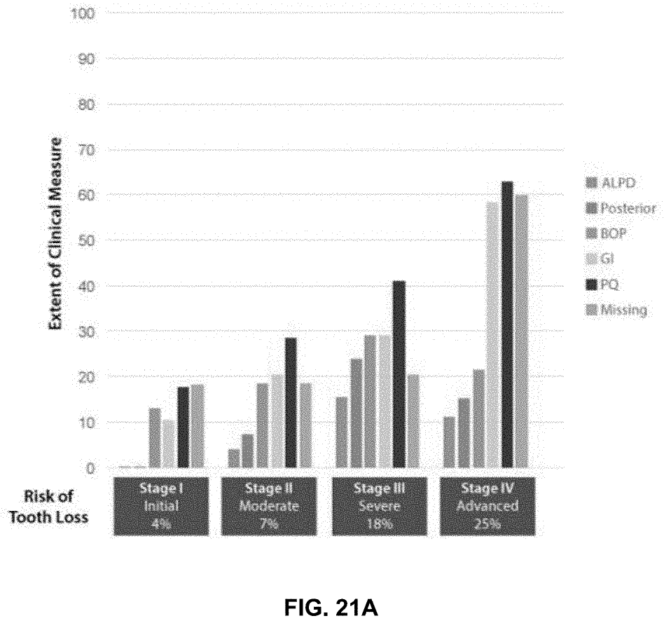

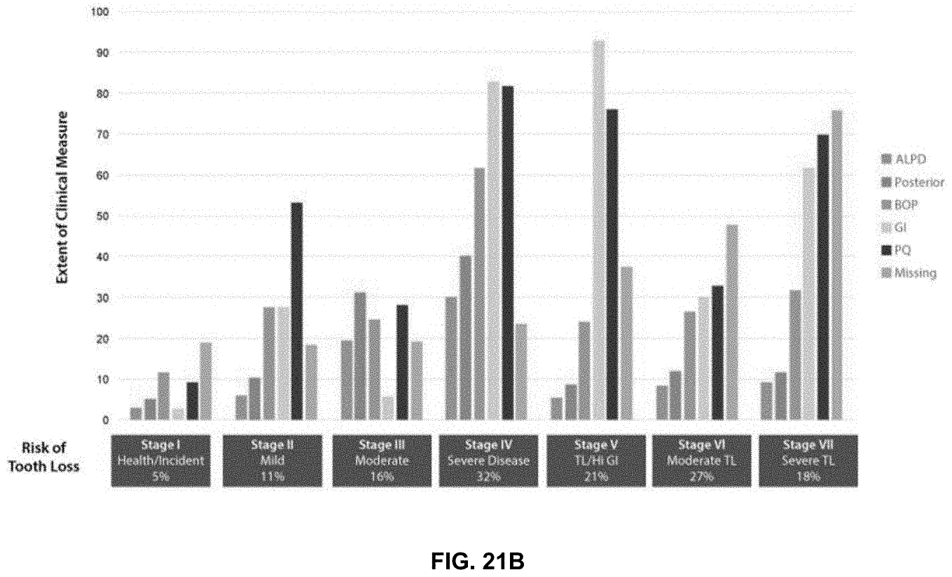

FIG. 21A shows the risk for 10-year tooth loss associated with each Stage and the relative level of the clinical measures within Stages. There is a trend for all clinical measures being higher going from Stage I to IV. For example Attachment Loss Pocket Depth (ALPD; defined as extent of CAL≥3 mm&PD4≥mm) is low in Stage I, increases in Stage II, and is much higher in Stage III. It is lower in Stage IV, likely due to the greater number of missing teeth. The extent of plaque and GI is greater as Stage increases as might be expected. In Stage III, extent of ALPD and Posterior disease are at their highest level. Stage IV is predominantly composed of missing teeth, PQ, GI, and BOP, but there is less posterior interproximal PD, and ALPD than in Stage III.

FIG. 21B presents the same clinical measures for the PPC Stages. The patterns for Stages I to IV are somewhat similar. The biggest difference between the two classification procedures is that missing teeth are a big component of the WW17 Stage 4 and the other clinical measures are a smaller component in comparison to the PPC Stage 4. Missing teeth are a small component of the PPC Stage 4 compared to the other clinical measures. PPC Stages V-VII have increasing higher levels of tooth loss. Notably, PPC Stage V includes individuals with mild tooth loss with a remarkably high amount of gingival inflammation that is associated with extensive GI scores.

This is the dominant clinical trait of this Stage that notably has tooth loss that is greater than the severe periodontitis in PPC Stage III. This High GI group of individuals has a little periodontal attachment loss and pocketing, but the gingival inflammation scores are overall very high.

Interestingly, these individuals are distinct from PPC Stage 4—Severe Periodontitis in that within the PPC Stage 4, the BOP scores are high reflecting deeper ulceration of the attachment apparatus that is more consistent with active periodontitis, but these high BOP scores are much lower in the PPC Stage VI group, indicating a more superficial and distinct gingival inflammation pattern. PPC Stage VI is characterized as having a moderate level of tooth loss (missing almost half of their teeth) with multiple teeth exhibiting a reduced periodontium (Moderate Tooth Loss/Reduced Periodontium). Stage VI individuals are missing an average of 24 teeth, but have lower plaque levels, bleeding, and GI scores than the Moderate Tooth Loss group. The severity of disease increases according to Stages V-VII. Stage VII individuals typically have a pattern of an edentulous maxilla with only lower premolars, canines and incisors remaining.

Patterns of clinical measures across Stages I-IV appear to be as expected for both models (see FIG. 21). These trends include: 1) a greater expression of the dominant clinical trait and 2) a greater prevalence of missing teeth with fewer healthy teeth.

Which Risk Factors are Associated with 10-Year Tooth Loss Within the Different Stages and are the Patterns of Risk Factors Different Between WW17 and PPC Stages?

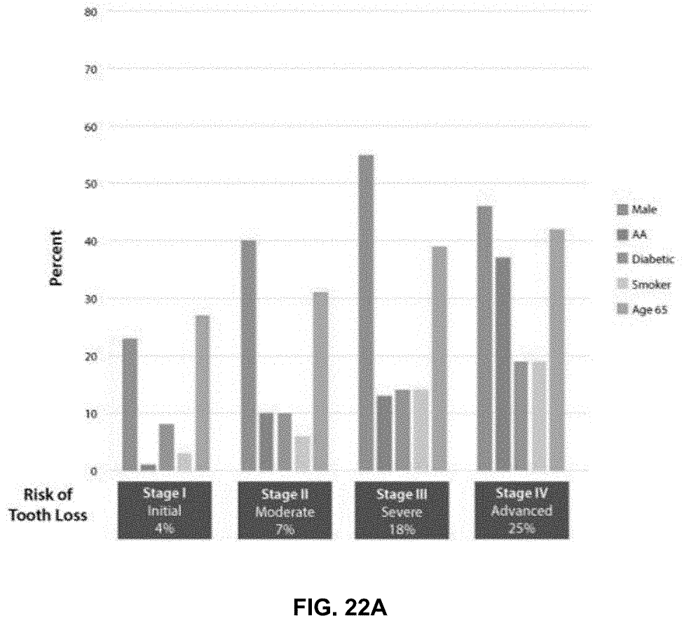

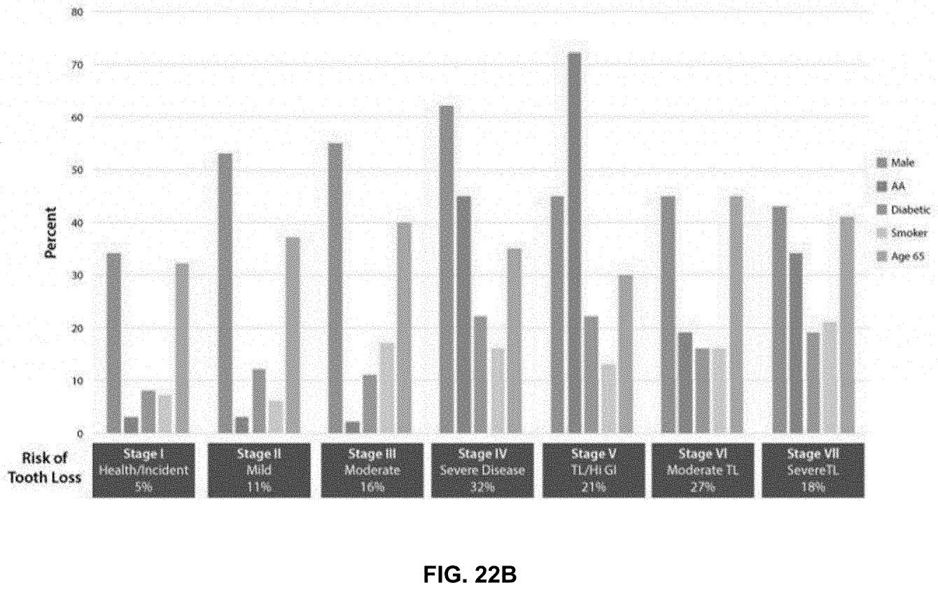

While there are multiple individual characteristics that are thought to be risk factors for tooth loss, there is some general consensus that smoking and diabetes are the major risk factors. There also are demographic characteristics that are often positively associated with tooth loss. Three such characteristics are Male sex, African American race, and increased age. Since the study participants were a middle-aged group, the threshold for age was set at 65 years and older. The patterns of these three demographic risk factors along with cigarette smoking and diabetes status are shown in FIGS. 22A and 22B for the WW17 and PPC Stages, respectively.

In the WW17 model, lower stages tend to have fewer individuals with risk factors, while higher stages have more individuals with risk factors (e.g. older males aged 65+ with diabetes and who smoke). However, the distribution of individuals with risk factors was more complex in the PPC Stage model. The proportion of Males increased from Stage I to Stage IV, but the proportion of participants over age 65 increased only through Stage III and then was lower in Stage IV. The proportions of African-Americans, smokers, and with diabetes were reasonably low (generally less than 10%) in Stages I and II, but the proportion of smokers was higher in Stage III. Stage IV, which has the highest risk for tooth loss, is predominately male and African-American and has more participants with diabetes than the earlier Stages. Stages V, VI, and VII, which exhibit successively higher levels of missing teeth, are characterized by having higher proportions of African-Americans with Stage V in excess of 70%. Comparing the patterns of risk factors associated with Stages IV through VII with the earlier Stages, it appears that the later Stages are more likely to be composed of individuals who are African-American, smokers, and older.

When considering differences that occur moving across from Stage I to Stage IV (see FIGS. 22A and 22B), there is a major trend shared by both models, which is a greater prevalence of concomitant risk factors, such as smoking, age, race, sex and diabetes. This finding would be supported by the concept of Stages, indicating levels of disease.

The PPC Stages that already have tooth loss (V, VI, and VII) continue a pattern of increased risk of tooth loss, even though there are already a decreased number of teeth at risk. This bimodal pattern is most noticeable for Stage VII individuals, who on average have eight remaining teeth. The trend is that the more teeth already lost is predictive of a higher risk for continuing to lose teeth.

PPC Stage V stands out as being highly inflammatory and is predominantly composed of males and African Americans. This important information was generated by an unsupervised model, which has a history of identifying hidden (latent) phenotypes.

Is Being Classified into a Higher Grade Within a Stage Associated with Higher Risk for 10-Year Tooth Loss?

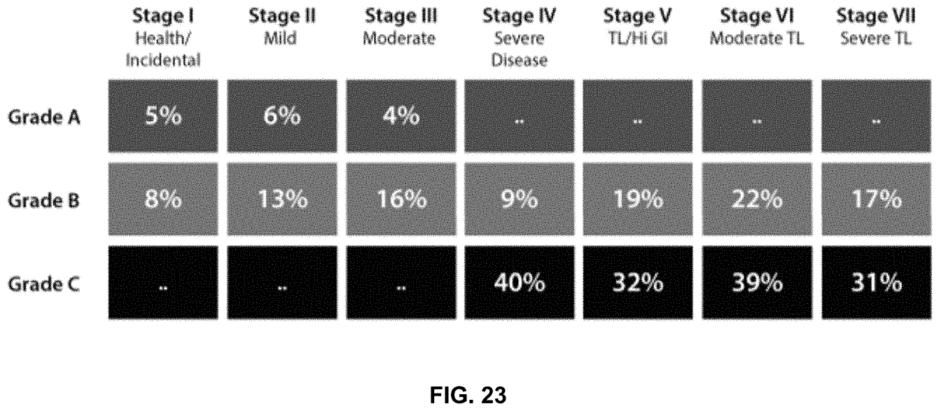

Since the WW17 Grades could not be created using existing data, only the PPC Stages and Grades are presented in order to determine whether the expected patterns of risk increases both by Stage and Grade within each Stage. FIG. 23 presents the risk for 10-year tooth loss using the PPC Stage and Grade system. First, the pattern of blank cells is appropriate as one might not expect a Grade A in Stages IV-VII, nor would one expect the earlier Stages to have the complexity in treatment represented by Grade C. Thus, for each Stage, being classified as a higher Grade is associated with greater risk for tooth loss over a 10-year period. Remembering that Stages V-VII have successively greater tooth loss, the drop in risk for tooth loss in Stage VII might be expected, since the individuals in that Stage have an average of eight teeth present in the mouth.

The data available in the PPC model indicate that there is a higher prevalence of 10-year tooth loss as Grade goes from A to C as might be expected. This information supports the WW17 proposed model of Stages and Grades. More information is needed to verify this finding using the supervised learning model.

Does Being Classified into a Higher Stage Imply also Imply Higher Risk for Clinical Attachment Loss?

In the WW17 model, the primary outcome is based on attachment loss over five years. The Dental ARIC dataset did not have attachment loss information. The Piedmont 65+ Dental Study (PDS)⁴⁶ contains information on periodontal progression rates for attachment loss (10% of sites increasing by 3+mm over three years). While the PDS contains longitudinal data, the sample size is much smaller than the Dental ARIC study and reflects an older cohort.

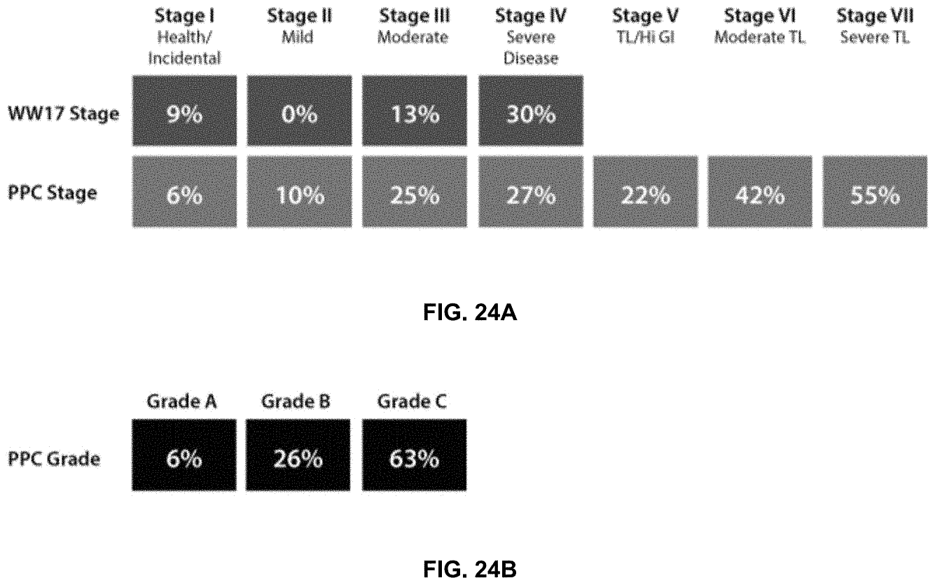

FIG. 24A shows that the higher the WW17 Stage, the greater the risk for having at least 10% of sites with attachment loss during the next 3 years. The exception was WW17 Stage II, because there were not enough people in that Stage. The pattern was similar for PPC Stages I to IV. In FIG. 24B, the smaller sample size did not allow calculation of Grades within each Stage so only the pattern for Grade alone is presented. For each successive Grade, there was a much higher risk of attachment loss occurring. Thus, it appears that both the WW17 and PPC Stage methods work similarly for tooth loss and attachment loss.

In FIG. 24A the highest risk for disease progression is among those in PPC Stages VI and VII. This suggests that capturing numbers of missing teeth is critical for assigning risk for future disease.

Stages, Grades and Systemic Disease

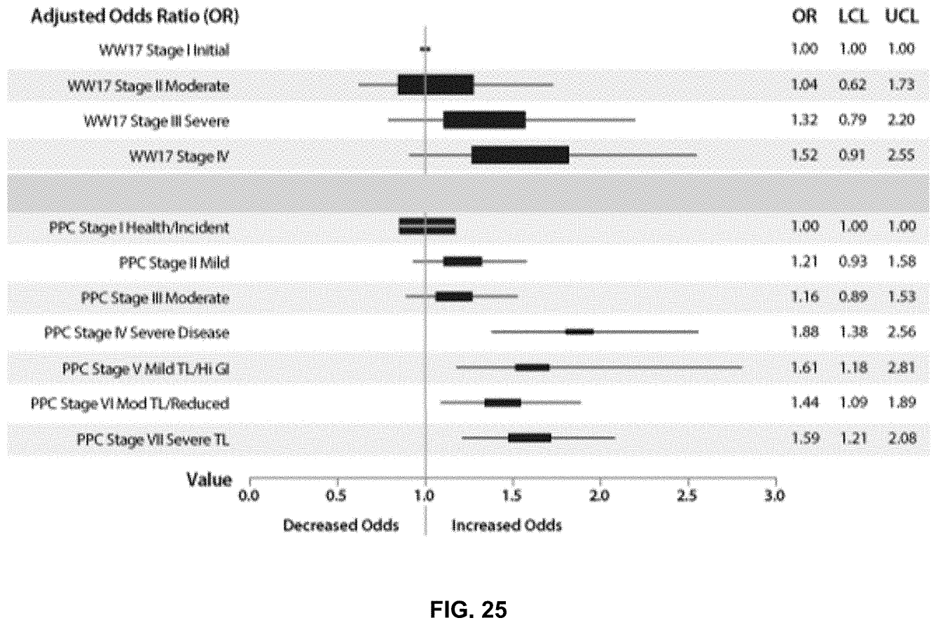

The World Workshop paper also suggests that potential relationship with systemic conditions should be considered as part of patient classification, because the systemic conditions add to the complexity of treatment Previously, the PPC categories were shown to be associated with prevalent diabetes and coronary heart disease, as well as high sensitivity C-reactive protein (hs-CRP) and Interleukin-6 (IL-6), which are systemic measures of inflammation often associated with systemic diseases.³⁹ FIG. 25 presents the associations between the WW17 and the PPC Stages with prevalent diabetes.³⁹

Since PPC Stages are derived from the original PPC designations, it was expected that the PPC Stages would be significantly associated with prevalent diabetes, but the relationships between the WW17 Stages and diabetes are still unknown. FIG. 25 shows that compared to individuals classified as WW17 Stage I, none of the other Stages are significantly associated with increased odds of having diabetes. A similar analysis using PPC Stages indicates that while individuals in Stages II and III do not have increased odds of having diabetes, individuals classified to Stages IV-VII all have increased odds of having diabetes with Stages IV and V having the highest odds of diabetes.

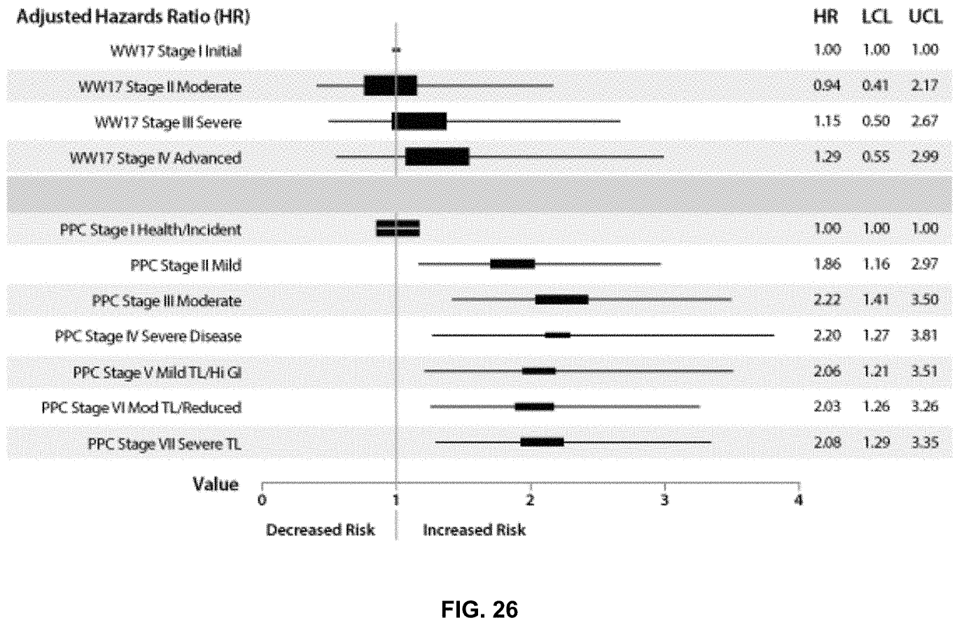

FIG. 26 presents relationships between being classified into different stages using the WW17 and the PPC Stages approaches and risk of a stroke in the future.⁴⁴ The results shown in FIG. 26 are similar to those in FIG. 26. Compared to Stage I, none of the other Stages convey excess risk for a stroke using the WW17 approach to classification. Using the PPC Stage approach, compared to Stage I, all of the other Stages appear to convey excess risk for a stroke. This excess risk was present after adjusting for race/examination center, age, sex, BMI, hypertension, diabetes, LDL level, smoking (3-levels), pack years, and education (3-levels).

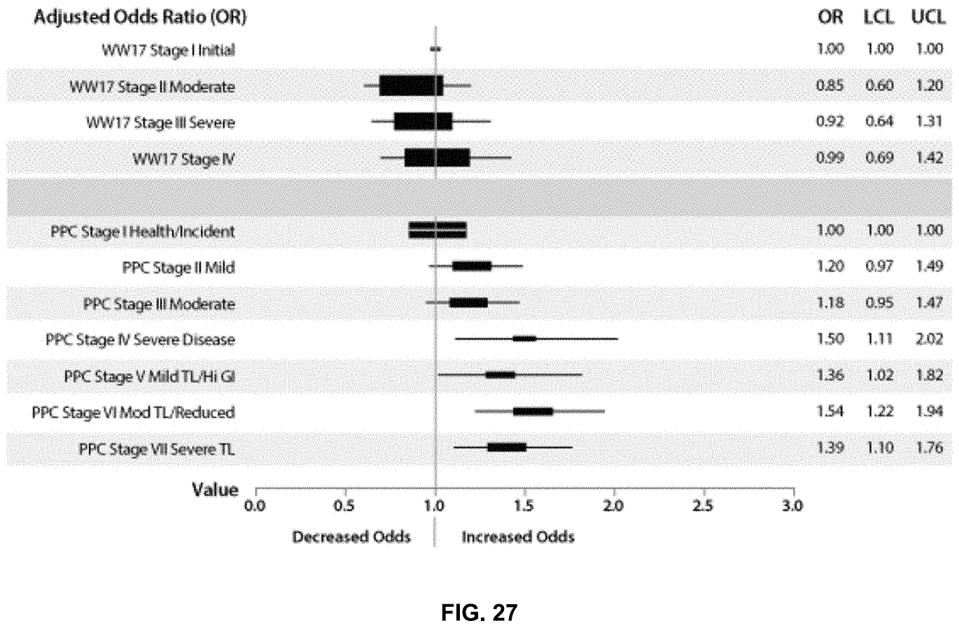

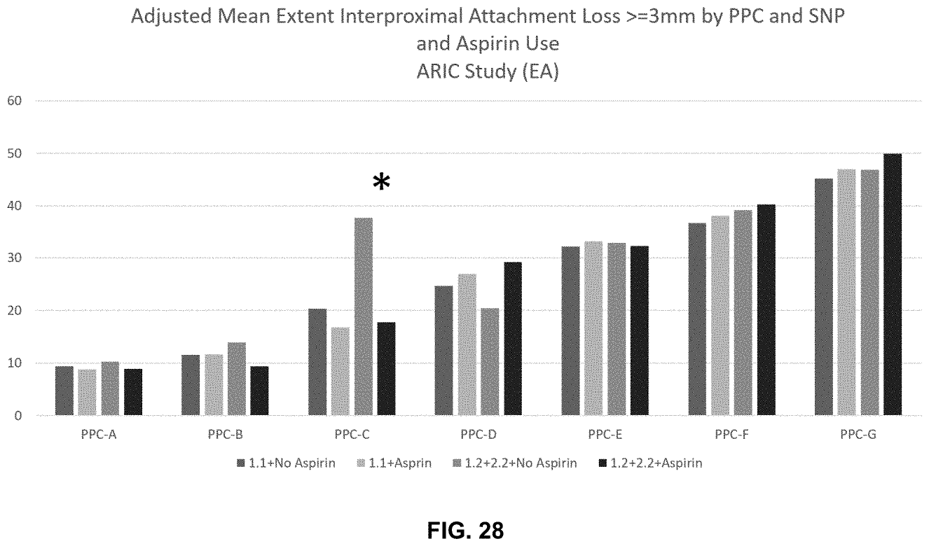

The relationships between WW17 and PPC Stages with having a CRP level in the highest quartile are shown in FIG. 27. Again, the WW17 Stages did not show any significant increase in odds for having high CRP according to Stage and the pattern for the PPC Stages showed that Stages II and III were not more likely to have high CRP levels than Stage I. Stages IV-VII had significantly greater odds for having high CRP levels.

Why does PPC Stage IV (Periodontitis) have a significant association with diabetes and high CRP when the WW17 Stage IV does not? This is likely due to the PPC Stages being more homogeneous classifications. For example, PPC Stage IV contains a smaller number of missing teeth than the WW17 Stage IV. Of note, PPC Stage V individuals have greater odds of having diabetes and high CRP and this Stage has an extensive number of sites with gingival inflammation. These excess odds were present after adjusting for race/examination center, age, sex, BMI, hypertension, diabetes, LDL level, smoking (3-levels), pack years, and education (3-levels).

While the hazards ratios in FIG. 26 indicate that individuals classified in a stage share a level of risk for incident stroke, they do not indicate which individuals in that Stage will have a stroke. The odds ratios for associations between Stages and prevalent diabetes and high CRP levels are interesting, but they do not answer the question as to whether individuals classified into a particular stage are at greater risk of developing the systemic condition in the future. Longitudinal studies are needed.

The unsupervised learning approach (PPC Stages) appears to result in stronger associations between the periodontal phenotype and systemic diseases and conditions. This likely occurs because an unsupervised learning model produces more mutually exclusive, homogeneous groups than a supervised learning model.

Supervised Learning vs. Unsupervised Learning

Up to this point, the WW17 model has been compared to the PPC model on a variety of features and attributes related to enabling precision dentistry. The reason these two models have been compared is because WW17 represents a supervised learning model and the PPC represents an unsupervised learning model. The issues about Stages and Grades are common to both models. Below is a short description of the Bayesian Information Criterion (BIC) and its application to test superiority of the two learning approaches.

The Bayesian Information Criterion (BIC) was computed to test whether the Supervised Learning or Unsupervised Learning approach made a greater contribution to explaining an oral or systemic outcome.^{39,47,48} A lower BIC score indicates better performance in terms of fit of the model and the contribution to the outcome of interest. Between-model difference in BIC is (BIC1-BIC2), which is used to compare the performance of each model. The larger the delta BIC score (i.e. when the lower BIC score is subtracted from the higher BIC score) indicates the following: 0-2=not worth more than a bare mention, 2-6=Positive, 6-10=Strong, 10+=Very Strong.

For example, determining whether having the Supervised or Unsupervised approach in a model to explain 10-year tooth loss in a model that also contains Race/Center, Age, Sex, BMI, Smoking, Education, Diabetes and Hypertension; the Supervised Learning approach had a BIC Score of 2997.7 and the Unsupervised Learning approach had a BIC score of 2978.4. Subtracting the latter score from the former resulted in a delta score of 19.3, meaning that the Unsupervised model was a better fit than the Supervised model and the resulting delta score of 19.3 indicates that the level of improvement in the model was Very Strong.

For 3-year Attachment Loss progression adjusting for Race/Center, Age, Gender, BMI, Smoking, Education, Diabetes and Hypertension; the Unsupervised Learning approach did a better job with a delta BIC score of 20.3 (Very Strong).

For Prevalent Diabetes adjusting for Race/Center, Age, Gender, BMI, Smoking, Education, and Hypertension; the Unsupervised Learning approach did a better job with a delta BIC score of 4.9 (Positive).

For C-reactive Protein adjusting for Race/Center, Age, Gender, BMI, Smoking, Education, Diabetes and Hypertension; the Unsupervised Learning approach did a better job with a delta BIC score of 13.2 (Very Strong).

For Incident Stroke adjusting for Race/Center, Age, Gender, BMI, Smoking, Education, Diabetes and Hypertension; the Unsupervised Learning approach did a better job with a delta BIC score of 12.9 (Very Strong).

Summary and Conclusions

Various groups have been exploring the application of precision medicine concepts to dentistry. Research advances are being made in areas of genomics and oral cancer, head and neck cancer, orofacial pain, TMD, caries, periodontal disease and other conditions. It has been suggested that a precision oral health approach would benefit the diagnosis and treatment of periodontal disease, although genetic work in this area is in its infancy. Discovery in all of these areas can provide information leading to new treatments for various periodontal disease phenotypes that can then be used by dentists to practice precision dentistry.

The move to precision dentistry is a new way of approaching health care that should be thought of as a paradigm shift because precision dentistry is not just placing an individual into a diagnostic category. Instead, it includes placing alike people into mutually exclusive, homogeneous categories of risk, specific to a particular disease or condition. This means that instead of using an “average treatment” for all people with a particular diagnosis, precision dentistry requires that the people in each diagnostic subgroup be homogeneous so that a specific treatment will be more effective for people in that subgroup. Additional studies are needed to determine whether this can be achieved by using a method or model that places each individual in a subgroup where each member is the same as every other member in relation to the disease of interest.

Currently, a variety of dental conditions are being approached with precision dentistry in mind. Models are being developed to place individuals with a disease into similar groups. These models use different approaches and have been characterized as being either Supervised Learning or Unsupervised Learning. Experts in the field who have predefined diagnostic categories based on studies that have indicated representative aspects of a phenotype build Supervised models. Unsupervised models employ analytic procedures, such as Latent Class Analysis or Deep Learning Models, that use data to place each individual together with alike individuals into multiple groups that represent risk for disease development, progression, or response to treatment. The medical literature has shown that the use of Latent Class Analysis has been useful in creating these mutually exclusive groups that respond well to treatment.

The World Workshop on Periodontal Disease Classification has created a new model that uses Stages and Grades of Disease. The model itself appears to work well in creating Stages that exhibit increasing risk for tooth loss and for Grades that also show increasing risk for tooth loss within each Stage. Data presented in this Example indicate that the concept of Stages and Grades works as expected in that periodontal disease appears to be more serious in each successive Stage. In addition, except in illogical situations (Grade C within earlier Stages and Grade A within higher Stages), the seriousness and complexity of the disease is greater as grade is higher within each Stage. The concept of Stages and Grades is integral for precision dentistry because it accounts for the risk of future disease and prognosis as well as enabling the practitioner to use more signs, symptoms and other associated factors when placing a patient in a diagnostic category.

However, a major issue in creating Stages and Grades is how the information that goes into them is constructed. Procedures to construct Stages and Grades have been classified into two categories: 1) Supervised Learning System (World Workshop Model or WW17) and 2) Unsupervised Learning System (Periodontal Profile Class or PPC). Both systems use the same dataset to compare the two Learning styles in this paper. A weakness of the approach followed in this paper is that not all the data to create WW17 Stages and Grades were available. Thus, additional studies need to be done to provide this information across a larger number of people.

BIC scores were used to test whether using a Supervised or Unsupervised Learning System to create Stages and Grades made a difference on how well these Stages and Grades predicted outcomes of interest. The Unsupervised Learning System appears to be more useful for creating Stages and Grades. For example, using both approaches to predict 10-year tooth loss (adjusting for Race/Center, Age, Sex, BMI, Smoking, Education, Diabetes and Hypertension), the BIC score for Unsupervised Learning was stronger, indicating that this model had better fit than its counterpart. The same was true for attachment loss progression (adjusting for Race/Center, Age, Sex, BMI, Smoking, Education, Diabetes and Hypertension). In addition, the unsupervised learning approach (PPC Stages) resulted in stronger associations between the periodontal phenotypes and systemic diseases and conditions (prevalent diabetes, C-reactive protein, and incident stroke).

The PPC identified three new periodontal disease stages that are not recognized in the WW17 model. These categories were hidden in the WW17 model because it was a supervised approach, but when an unsupervised approach was used, these new categories arose from a more complete examination of patient risk factors (224 characteristics). These new stages are important because they allow more homogeneity within the diagnostic groups. Clearly, an Unsupervised learning model follows a more well-defined manner of considering the multitude of risk factors.

While the WW17 focused on the ways systemic conditions affected periodontitis, this paper focused on ways periodontitis may affect systemic conditions. The purpose here was to present additional information on the how Supervised and Unsupervised approaches result in different periodontitis phenotypes that, in turn, affect relationships between periodontitis and systemic diseases.

### REFERENCES

1. Tonetti M, Greenwell H, Kornman K. Periodontitis case definition: Framework for staging and grading the individual periodontitis case. Journal of Clinical Periodontology. 2018; 45:S149-S161.

2. Morelli T, Moss K L, Beck J, et al. Derivation and validation of the periodontal and tooth profile classification system for patient stratification. Journal of periodontology. 2017; 88(2):153-165.

3. Collins F S, Varmus H. A new initiative on precision medicine. New England Journal of Medicine. 2015; 372(9):793-795.

4. Divaris K. Precision dentistry in early childhood: the central role of genomics. Dental Clinics. 2017; 61(3):619-625.

5. Horesh Bergquist S, Lobelo F. The Limits and Potential Future Applications of Personalized Medicine to Prevent Complex Chronic Disease. Public Health Reports. 2018; 133(5):519-522.

6. Shastry B. Pharmacogenetics and the concept of individualized medicine. The pharmacogenomics journal. 2006; 6(1):16.

7. Hingorani A D, van der Wndt D A, Riley R D, et al. Prognosis research strategy (PROGRESS) 4: stratified medicine research. Bmj. 2013; 346:e5793.

8. Silverman E K, Loscalzo J. Developing new drug treatments in the era of network medicine. Clinical Pharmacology & Therapeutics. 2013; 93(1):26-28.

9. Van't Veer L J, Bernards R. Enabling personalized cancer medicine through analysis of gene-expression patterns. Nature. 2008; 452(7187):564.

10. Council N R. Toward precision medicine: building a knowledge network for biomedical research and a new taxonomy of disease. National Academies Press; 2011.

11. Tian Q, Price N D, Hood L. Systems cancer medicine: towards realization of predictive, preventive, personalized and participatory (P4) medicine. Journal of internal medicine. 2012; 271(2):111-121.

12. Radder J E, Shapiro S D, Berndt A. Personalized medicine for chronic, complex diseases: chronic obstructive pulmonary disease as an example. Personalized medicine. 2014; 11(7):669-679.

13. Garcia I, Kuska R, Somerman M. Expanding the foundation for personalized medicine: implications and challenges for dentistry. Journal of dental research. 2013; 92(7_suppl):S3-S10.

14. McAlister F A, Laupacis A, Armstrong P W. Finding the right balance between precision medicine and personalized care. CMAJ: Canadian Medical Association Journal. 2017; 189(33):E1065.

15. Green E. Presentation entitled “From the Human Genome Project to Precision Medicine: A Journey To Advance Human Health”. Advances in Precision Oral Health. Bethesda, Md.: National Human Genome Research Institute at the National Institutes of Health; 2018.

16. Kornman KS. Contemporary approaches for identifying individual risk for periodontitis. Periodontology 2000. 2018; 78(1):12-29.

17. Biankin A V, Piantadosi S, Hollingsworth S J. Patient-centric trials for therapeutic development in precision oncology. Nature. 2015; 526(7573):361.

18. Pihlstrom B L, Michalowicz B S, Johnson N W. Periodontal diseases. The lancet. 2005; 366(9499):1809-1820.

19. Offenbacher S, Barros S P, Beck J D. Rethinking periodontal inflammation. Journal of periodontology. 2008; 79(8S):1577-1584.

20. Offenbacher S, Barros S, Singer R, Moss K, Williams R, Beck J. Periodontal disease at the biofilm—gingival interface. Journal of periodontology. 2007; 78(10):1911-1925.

21. Hu F B, Manson J E, Stampfer M J, et al. Diet, lifestyle, and the risk of type 2 diabetes mellitus in women. New England journal of medicine. 2001; 345(11):790-797.

22. Stampfer M J, Hu F B, Manson J E, Rimm E B, Willett W C. Primary prevention of coronary heart disease in women through diet and lifestyle. New England Journal of Medicine. 2000; 343(1):16-22.

23. Rosenblum D, Peer D. Omics-based nanomedicine: the future of personalized oncology. Cancer letters. 2014; 352(1):126-136.

24. Riley W T, Nilsen W J, Manolio T A, Masys D R, Lauer M. News from the NIH: potential contributions of the behavioral and social sciences to the precision medicine initiative. Translational behavioral medicine. 2015; 5(3):243-246.

25. Baelum V, Lopez R. Defining and classifying periodontitis: need for a paradigm shift? European journal of oral sciences. 2003; 111(1):2-6.

26. Schwendicke F. Tailored Dentistry: From “One Size Fits All” to Precision Dental Medicine? Operative dentistry. 2018; 43(5):451-459.

27. Ching T, Himmelstein D S, Beaulieu-Jones B K, et al. Opportunities and obstacles for deep learning in biology and medicine. Journal of The Royal Society Interface. 2018; 15(141):20170387.

28. Lasko T A, Denny J C, Levy M A. Computational phenotype discovery using unsupervised feature learning over noisy, sparse, and irregular clinical data. PloS one. 2013; 8(6):e66341.

29. Wenzel S E. Asthma phenotypes: the evolution from clinical to molecular approaches. Nature medicine. 2012; 18(5):716.

30. De Keulenaer G W B D. The heart failure spectrum: time for a phenotype-oriented approach. Circulation. 2009; 119(24):3044-3046.

31. De Keulenaer G W, Brutsaert D L. Systolic and Diastolic Heart Failure Are Overlapping Phenotypes Within the Heart Failure Spectrum Response to De Keulenaer and Brutsaert. Circulation. 2011; 123(18):1996-2005.

32. Magidson J, Vermunt J K. Latent class modeling as a probabilistic extension of K-means clustering. Quirk's Marketing Research Review. 2002; 20:77-80.

33. Morelli T, Moss K L, Preisser J S, et al. Periodontal profile classes predict periodontal disease progression and tooth loss. Journal of periodontology. 2018; 89(2):148-156.

34. Ferrat E, Audureau E, Paillaud E, et al. Four distinct health profiles in older patients with cancer: Latent class analysis of the prospective ELCAPA cohort. Journals of Gerontology Series A: Biomedical Sciences and Medical Sciences. 2016; 71(12):1653-1660.

35. de Luca K, Parkinson L, Downie A, Blyth F, Byles J. Three subgroups of pain profiles identified in 227 women with arthritis: a latent class analysis. Clinical rheumatology. 2017; 36(3):625-634.

36. Wu C-Y, Hu H-Y, Li C-P, Chou Y-J, Chang Y-T. Comorbidity profiles of psoriasis in Taiwan: A latent class analysis. PloS one. 2018; 13(2):e0192537.

37. Kaptein K I, De Jonge P, Van Den Brink R H, Korf J. Course of depressive symptoms after myocardial infarction and cardiac prognosis: a latent class analysis. Psychosomatic Medicine. 2006; 68(5):662-668.

38. Bartold P M. Personalized/Precision Dentistry—The Future of Dentistry? Australian dental journal. 2017; 62(3):257-257.

39. Beck J D, Moss K L, Morelli T, Offenbacher S. Periodontal profile class is associated with prevalent diabetes, coronary heart disease, stroke, and systemic markers of C-reactive protein and interleukin-6. Journal of periodontology. 2018; 89(2):157-165.

40. Divaris K. Predicting dental caries outcomes in children: a “risky” concept. Journal of dental research. 2016; 95(3):248-254.

41. Weber M, Søvik J B, Mulic A, et al. Redefining the Phenotype of Dental Caries. Caries research. 2018; 52(4):263-271.

42. Bair E, Gaynor S, Slade G D, et al. Identification of clusters of individuals relevant to temporomandibular disorders and other chronic pain conditions: the OPPERA study. Pain. 2016; 157(6):1266.

43. Polverini P, D'Silva N, Lei Y. Precision therapy of head and neck squamous cell carcinoma. Journal of dental research. 2018; 97(6):614-621.

44. Sen S, Giamberardino L D, Moss K, et al. Periodontal disease, regular dental care use, and incident ischemic stroke. Stroke. 2018; 49(2):355-362.

45. Lang N P, Bartold P M. Periodontal health. Journal of periodontology. 2018; 89:S9-S16.

46. Beck J D, Koch G G, Offenbacher S. Attachment loss trends over 3 years in communitydwelling older adults. Journal of periodontology. 1994; 65(8):737-743.

47. Lanza S T, Rhoades B L. Latent class analysis: An alternative perspective on subgroup analysis in prevention and treatment. Prevention Science. 2013; 14(2):157-168.

48. Schwarz G. Estimating the dimension of a model The Annals of Statistics. 1978; 6(2):461-464.

### EXAMPLE 7

Table 1 is a list of SNPs associated with each PPC stage. And their corresponding genes. Table 29 identifies drugs that target the genes identified in Table 1. Therapeutic Agents were identified using two approaches. The first approach was to take all genes (using the nearest gene to the SNP) identified in Table 1 and search Drug-Gene Interactions using DGldb.com. The second approach was to take all genes (using the nearest gene to the SNP) identified in Table 1 and search GUILDify v2.0. to search for therapeutic agents that target genes and gene networks for each PPC class.

TABLE 29
Drugs that directly target genes identified in each PPC-Stage

PPC-A
None
PPC-B
DEXTROSE, PYROGLUTAMIC ACID, STREPTOL, VANDETANIB, PROGESTERONE, ACARBOSE, MIGLITOL, CELGOSIVIR,
DUVOGLUSTAT HYDROCHLORIDE, DUVOGLUSTAT, PHORBOL MYRISTATE ACETATE, LEUCOVORIN, IRINOTECAN
PPC-C
VANTICTUMAB, FLANVOTUMAB, DOCETAXEL, PACLITAXEL, ACALABRUTINIB, IBRUTINIB, INOSITOL, RETINOL,
GENISTEIN, LEUPROLIDE ACETATE, DEXAMETHASONE, HALOFUGINONE, DIHYDROSPINGOSINE, SPHINGOSINE,
NIFEDIPINE, CHEMBL1179605, MEFENAMIC ACID, PIOGLITAZONE, ROSIGLITAZONE, TROGLITAZONE, CHEMBL169233,
ASPIRIN, CHEMBL1161866, STANOLONE, CHEMBL566340, COLFORSIN, DAROTROPIUM BROMIDE, TRIDIHEXETHYL
CHLORIDE, TOLTERODINE TARTRATE, PROPANTHELINE BROMIDE, OXYPHENONIUM BROMIDE, ARIPIPRAZOLE,
OLANZAPINE, METHIXENE, TERFENADINE, CLOZAPINE, OXYPHENCYCLIMINE, PROCYCLIDINE, LOXAPINE, PROMAZINE,
HYOSCYAMINE, DARIFENACIN, TRIDIHEXETHYL, ANISOTROPINE METHYLBROMIDE, DIPHEMANIL METHYLSULFATE,
SCOPOLAMINE, BENZQUINAMIDE, PROPIOMAZINE, TROPICAMIDE, BROMPHENIRAMINE, GLYCOPYRROLATE,
TOLTERODINE, PILOCARPINE, MIVACURIUM, DIPHENIDOL, CHLORPROTHIXENE, PIPECURONIUM,
LEVOMEPROMAZINE, ISOPROPAMIDE, MEPENZOLATE, FESOTERODINE, METHACHOLINE, ACLIDINIUM, UMECLIDINIUM,
ACETYLCHOLINE, ARECAIDINE PROPARGYL ESTER, ARECOLINE, BETHANECHOL, FURTRETHONIUM, 5-
METHYLFURMETHIODIDE, CHEMBL99521, ERIBAXABAN, OXOTREMORINE, CHEMBL130715, CHEMBL74300,
MILAMELINE, SABCOMELINE, XANOMELINE, ALCURONIUM, BRUCINE, CHEMBL343796, STRYCHNINE, CHEMBL2206331,
CHEMBL343357, VINBURNINE, VINCAMINE, CHEMBL139677, CHEMBL523685, CHEMBL1256845, 4-DAMP, CHEMBL279453,
AMITRIPTYLINE, BENZTROPINE MESYLATE, ATROPINE, BIPERIDEN (CHEMBL1101), CLIDINIUM, DICYCLOMINE,
DOTHIEPIN (CHEMBL1492500), CHEMBL580785, HEXOCYCLIUM, HIMBACINE, CHEMBL1256682, IPRATROPIUM,
METHOCTRAMINE, HYDROCHLORIC ACID, OTENZEPAD, OXYBUTYNIN, PIRENZEPINE, PROPANTHELINE, SOLIFENACIN,
COENZYME A, QUINUCLIDINYL BENZILATE, TIOTROPIUM (CHEMBL1900528), TRIPITRAMINE, CHEMBL1233686,
CHEMBL1628667, REVATROPATE, UMECLIDINIUM BROMIDE, LAS190792, Afacifenacin, Tarafenacin, SOLIFENACIN
SUCCINATE, ACLIDINIUM BROMIDE, METHACHOLINE CHLORIDE, TIOTROPIUM BROMIDE, ACETYLCHOLINE CHLORIDE,
CARBACHOL (CHEMBL14), PILOCARPINE HYDROCHLORIDE, CEVIMELINE HYDROCHLORIDE, SUXAMETHONIUM,
BATEFENTEROL, MEPENZOLATE BROMIDE, HEXOCYCLIUM METHYLSULFATE, DICYCLOMINE HYDROCHLORIDE,
BETHANECHOL CHLORIDE, ATROPINE SULFATE, OXYBUTYNIN CHLORIDE, DARIFENACIN HYDROBROMIDE,
IPRATROPIUM BROMIDE HYDRATE, METHSCOPOLAMINE BROMIDE, OXYPHENCYCLIMINE HYDROCHLORIDE,
GLYCOPYRROLATE BROMIDE, ISOPROPAMIDE IODIDE, TROSPIUM CHLORIDE, FESOTERODINE FUMARATE,
CYCLOPENTOLATE HYDROCHLORIDE, ASM-024, AZD8683, CLIDINIUM BROMIDE, THIETHYLPERAZINE, CEVIMELINE,
DOXEPIN, PROMETHAZINE, ITOPRIDE, HOMATROPINE METHYLBROMIDE, ANACETRAPIB, DALCETRAPIB,
TORCETRAPIB, CHEMBL67129, EVACETRAPIB, CERIVASTATIN, TAMOXIFEN, CEP-2563, TICLOPIDINE, GSK-690693,
SOTRASTAURIN, (7S)-HYDROXYL-STAUROSPORINE, MIDOSTAURIN, QUERCETIN, SOTRASTAURIN ACETATE,
STAUROSPORINE, DEXFOSFOSERINE, INGENOL MEBUTATE, CHEMBL369507, BRYOSTATIN, MEDRONIC ACID, INSULIN
HUMAN, VANDETANIB, CHEMBL552425, SULFASALAZINE, RIDOGREL, DAZOXIBEN, PHORBOL MYRISTATE ACETATE,
BELIMUMAB, BRIOBACEPT, ATACICEPT, TABALUMAB, BLISIBIMOD, DIOXANE, ETOPOSIDE, CITRIC ACID
PPC-D
ISRADIPINE, NIMODIPINE, NISOLDIPINE, VERAPAMIL, FELODIPINE, NITRENDIPINE, NIFEDIPINE, MIBEFRADIL,
NILVADIPINE, SALSALATE, BEPRIDIL HYDROCHLORIDE, PREGABALIN, GABAPENTIN, GABAPENTIN ENACARBIL,
IMAGABALIN, ATAGABALIN, MAGNESIUM SULFATE, AMLODIPINE, CELECOXIB, DRONEDARONE, CHEMBL566340, GO-
6976, QUERCETIN, GSK-690693, (7S)-HYDROXYL-STAUROSPORINE, CEP-2563, MIDOSTAURIN, SOTRASTAURIN,
BRYOSTATIN, RESVERATROL, VASOPRESSIN, BOSUTINIB, CHEMBL359482, PAROXETINE, OCRIPLASMIN,
HYDROCHLOROTHIAZIDE, CHEMBL384759, INSULIN HUMAN, BUSULFAN, ZONISAMIDE, FRUCTOSE, TRICHOSTATIN,
ASCORBATE, OCRIPLASMIN, GUANIDINE HYDROCHLORIDE, DALFAMPRIDINE, TEDISAMIL, NERISPIRDINE, TRETINOIN,
ETRETINATE, ZOLEDRONIC ACID, CHEMBL300914, PYRIDOXAL PHOSPHATE, THREONINE, NIROGACESTAT, REGN-421,
CYCLOPHOSPHAMIDE
PPC-E
QUERCETIN, METAPROTERENOL SULFATE, COPANLISIB, ALPELISIB, PICTILISIB, APITOLISIB, PF-04691502,
GEDATOLISIB, SONOLISIB, SF-1126, VOXTALISIB, PILARALISIB (CHEMBL3218575), DACTOLISIB, PI-103, GSK-2636771,
BUPARLISIB, CHEMBL1229535, HYDROGEN PEROXIDE, CLAVULANIC ACID, AMOXICILLIN, ME-344, NV-128, METFORMIN
HYDROCHLORIDE, CHEMBL1161866, CAPSAICIN, CHEMBL1213492, NINTEDANIB ESYLATE, SU-014813, KRN-633, L-
21649, TAK-593, AG-13958, BMS-690514, FORETINIB, MGCD-265, BRIVANIB ALANINATE, SEMAXANIB, CEDIRANIB,
SORAFENIB, Anlotinib, CC-223, DOVITINIB, Fruquintinib, LENVATINIB, LINIFANIB, MK-2461, MOTESANIB, NINTEDANIB,
PAZOPANIB, SUNITINIB, TESEVATINIB, TIVOZANIB, VATALANIB, AXITINIB, REGORAFENIB, LENVATINIB MESYLATE,
ILORASERTIB, CEP-11981, CEP-7055, Chiauranib, JNJ-26483327, OSI-930, RG-1530, TELATINIB, Famitinib, XL-999,
BRIVANIB, SUNITINIB MALATE, VANDETANIB, SORAFENIB TOSYLATE, PAZOPANIB HYDROCHLORIDE, LUCITANIB,
ENMD-2076, ORANTINIB, X-82, XL-820, CEP-5214, SU-14813, CP-459632, Sulfatinib, 4SC-203, CHEMBL313417, IMC-3C5,
TABERMINOGENE VADENOVEC, TG100-801, IMC-1C11, PEGPLERANIB SODIUM, CHEMBL384759, GUANOSINE
TRIPHOSPHATE, GLYCINE, TEZAMPANEL, BUTABARBITAL, BUTALBITAL, TALBUTAL, SECOBARBITAL, METHARBITAL,
THIOPENTAL, PRIMIDONE, MEPHOBARBITAL, PHENOBARBITAL, CHEMBL301536, 2S.4R-4-METHYLGLUTAMATE,
DOMOIC ACID, KAINIC ACID, MESALAMINE, METHYPRYLON, AMOBARBITAL, APROBARBITAL, BUTETHAL,
HEPTABARBITAL, HEXOBARBITAL, BARBITAL, Selurampanel, TOPIRAMATE, PENTOBARBITAL, QUISQUALATE, L-
GLUTAMATE, PROTIRELIN, TALTIRELIN, GANITUMAB, PICROPODOPHYLLOTOXIN, BMS-754807, LINSITINIB, AEW-541,
DALOTUZUMAB, INSULIN GLARGINE, EMACTUZUMAB, AZD-4547, CHEMBL401930, BRIGATINIB, CERITINIB,
CHEMBL464552, CHEMBL458997, DIMETHISTERONE, XL-228, BIIB-022, AVE-1642, CIXUTUMUMAB, INSM-18, KW-2450,
FIGITUMUMAB, ROBATUMUMAB, MECASERMIN, PL-225B, TEPROTUMUMAB, M ECASERM IN RINFABATE,
CHEMBL1230989, ACETYLCYSTEINE, THROMBIN, INSULIN HUMAN, CHEMBL263143, CHEMBL397666, RALOXIFENE,
ERLOTINIB, RINFABATE, STAUROSPORINE, HYDROCHLOROTHIAZIDE
PPC-F
DANAZOL, CHEMBL195368, CHEMBL1234621, CONBERCEPT, SORAFENIB TOSYLATE, LENALIDOMIDE, BEVACIZUMAB,
NIMODIPINE, PROGESTERONE, SPIRONOLACTONE, EPLERENONE, FELODIPINE, DESOXYCORTICOSTERONE
PIVALATE, DROSPIRENONE, ALDOSTERONE, HYDROCORTISONE, DESOXYCORTICOSTERONE, DEXAMETHASONE,
FLUDROCORTISONE, PREDNISOLONE, FINERENONE, ONAPRISTONE, PF-03882845, OXPRENOATE POTASSIUM, XL550,
MT-3995, LY2623091, DESOXYCORTICOSTERONE ACETATE, FLUDROCORTISONE ACETATE, CORTICOSTERONE,
RIZATRIPTAN, CHEMBL482796, LIPOXIN A4, LITHIUM, GADOBENATE DIMEGLUMINE, CHEMBL185515, AMILORIDE,
PYRIMETHAMINE, NAFAMOSTAT, PREGNENOLONE, GENISTEIN, LEUPROLIDE ACETATE, DEXAMETHASONE,
HALOFUGINONE, DAVALINTIDE, PRAMLINTIDE, PRAMLINTIDE ACETATE, CALCITONIN SALMON RECOMBINANT,
DOXORUBICIN, CHEMBL574817, CLOZAPINE, ODANACATIB, PALMITIC ACID, FLANVOTUMAB, INGENOL MEBUTATE,
ELLAGIC ACID, APRINOCARSEN SODIUM, CHEMBL1236539, BALANOL, ENZASTAURIN, GO-6976, SEBACIC ACID,
RUBOXISTAURIN, SOTRASTAURIN, MIDOSTAURIN, QUERCETIN, SOTRASTAURIN ACETATE, GSK-690693, CEP-2563,
(7S)-HYDROXYL-STAUROSPORINE, BRYOSTATIN, TAMOXIFEN, DEXFOSFOSERINE, VITAMIN E, FRUCTOSE,
TRICHOSTATIN, SACITUZUMAB GOVITECAN, TEGLARINAD CHLORIDE, FLUOROURACIL, OCRIPLASMIN, HALOTHANE,
PROXYPHYLLINE, LISOFYLLINE, CAFFEINE, TRETINOIN, UROKINASE, CARBOQUONE, INSULIN HUMAN, MESALAMINE,
L-GLUTAMATE, PYRIMETHAMINE, PYRIDOSTIGMINE, PYRILAMINE, PEGINTERFERON LAMBDA-1A, RINTATOLIMOD,
PYROXAMIDE, HYDROXYCHLOROQUINE, ELEDOISIN, KASSININ, NEUROKININ A, NEUROKININ B, CHEMBL69367,
SENKTIDE, SUBSTANCE P, ISOFLURANE, AZD2624, CHEMBL480249, CHEMBL221445, OSANETANT, CHEMBL44229,
SAREDUTANT, CHEMBL9843, CHEMBL1991816, AMCINONIDE, TALNETANT, SB-222200, CHEMBL275544,
PYRIMETHAMINE, COBALT (II) ION, VERAPAMIL, VASOPRESSIN TANNATE, FAMOXADONE, AZOXYSTROBIN, Coenzyme
Q2, PROXYPHYLLINE, CHOLIC ACID, AFIMOXIFENE (CHEMBL489), DIARYLPROPIONITRILE, DIETHYLSTILBESTROL,
CHLOROTRIANISENE, ESTROGENS, CONJUGATED, ETONOGESTREL, DESOGESTREL, LEVONORGESTREL,
PROGESTERONE, TOREMIFENE, MEDROXYPROGESTERONE ACETATE, ESTRONE, TAMOXIFEN, DIENESTROL,
FULVESTRANT, NORGESTIMATE, ETHINYL ESTRADIOL, MELATONIN, TRILOSTANE, FLUOXYMESTERONE,
ESTRAMUSTINE, ESTRIOL, PRINABEREL, PROPYLPYRAZOLETRIOL, RALOXIFENE, CHEMBL282489, CHEMBL188528,
LASOFOXIFENE, BAZEDOXIFENE, CLOMIPHENE, CHEMBL201013, HEXESTROL, CHEMBL520107, MESTRANOL,
DANAZOL, ALLYLESTRENOL, PRASTERONE, ESTROPIPATE, QUINESTROL, OSPEMIFENE, TIBOLONE, ESTROGENS,
CONJUGATED SYNTHETIC A, SYNTHETIC CONJUGATED ESTROGENS, B, ESTRADIOL, MITOTANE, SR16234
(CHEMBL3545210), FISPEMIFENE, LY2245461, IDOXIFENE, GTx-758, AFIMOXIFENE (CHEMBL10041), DROLOXIFENE,
ACOLBIFENE, TAMOXIFEN CITRATE, ESTRADIOL VALERATE, CLOMIPHENE CITRATE, ESTRADIOL CYPIONATE,
ESTROGENS, ESTERIFIED, DIETHYLSTILBESTROL DIPHOSPHATE, TOREMIFENE CITRATE, BAZEDOXIFENE ACETATE,
CHF4227, GDC-0810, ESTRADIOL ACETATE (CHEMBL1200430), POLYESTRADIOL PHOSPHATE, MK-6913, IODINE,
VINTAFOLIDE, CUSTIRSEN, CHEMBL304552, EVEROLIMUS, CHEMBL181936, CHEMBL391910, PERTUZUMAB,
CHEMBL180300, DIENOGEST, ESTROGEN, RAD1901, CHEMBL222501, ARZOXIFENE, CHEMBL180071, CHEMBL193676,
ETHYNODIOL DIACETATE, GENISTEIN, RALOXIFEN, Ribociclib, RALOXIFENE CORE, TRASTUZUMAB, CHEMBL236718,
ABEMACICLIB, 2-AMINO-1-METHYL-6- PHENYLIMIDAZO[4,5- B]PYRIDINE, ERTEBEREL, EXEMESTANE, ENDOXIFEN,
MEGESTROL ACETATE, SIVIFENE, PALBOCICLIB, LEFLUNOMIDE, ESTRONE SODIUM SULFATE, LETROZOLE,
CHEMBL236086, CHEMBL184151, CHEMBL223026, LAPATINIB, CHEMBL180517, NORELGESTROMIN, ANASTROZOLE,
CARBOQUONE, GONADORELIN, CHEMBL1213270, NORGESTREL, MEDRONIC ACID
PPC-G
TEMAZEPAM, ADINAZOLAM, HALAZEPAM, DIAZEPAM, OXAZEPAM, TRIAZOLAM, ESTAZOLAM, BROMAZEPAM,
CLOTIAZEPAM, FLUDIAZEPAM, KETAZOLAM, PRAZEPAM, QUAZEPAM, CINOLAZEPAM, NITRAZEPAM, CLORAZEPATE
DIPOTASSIUM, CHLORDIAZEPOXIDE, ESZOPICLONE, MEPROBAMATE, CLOBAZAM, ALPRAZOLAM, BUTALBITAL,
CLONAZEPAM, DESFLURANE, TALBUTAL, BUTABARBITAL SODIUM, LORAZEPAM, METHARBITAL, METHYPRYLON,
MIDAZOLAM HYDROCHLORIDE, PRIMIDONE, PROPOFOL, PENTOBARBITAL SODIUM, SECOBARBITAL SODIUM,
SEVOFLURANE, THIAMYLAL SODIUM, FLURAZEPAM HYDROCHLORIDE, HALOTHANE, ISOFLURANE, ADIPIPLON,
Loredipion, RESEQUINIL, PF-06372865, METHOHEXITAL SODIUM, THIOPENTAL SODIUM, CHLORDIAZEPOXIDE
HYDROCHLORIDE, PENTOBARBITAL, ETHCHLORVYNOL, GLUTETHIMIDE, FLUMAZENIL, CLORAZEPIC ACID,
METHOXYFLURANE, MIDAZOLAM, TRICLOFOS SODIUM, TOPIRAMATE, GABOXADOL, ETOMIDATE, ACAMPROSATE
CALCIUM, FLURAZEPAM, OCINAPLON, ENFLURANE, ETAZOLATE, CHEMBL2325441, PREGNENOLONE, S-
ADENOSYLHOMOCYSTEINE, ODANACATIB, PROSCILLARIDIN, CYPROHEPTADINE, CYCLOPENTOLATE, ELETRIPTAN,
METHYSERGIDE, ZOLMITRIPTAN, ERGOTAMINE, RIZATRIPTAN, MIANSERIN, ELETRIPTAN HYDROBROMIDE,
ALMOTRIPTAN MALATE, LASMIDITAN, CHEMBL266591, SEROTONIN, 5-MEO-DMT, 5-METHOXYTRYPTAMINE, 8-OH-DPAT,
CHEMBL1256797, CLOZAPINE, DIHYDROERGOTAMINE, CHEMBL1332062, DONITRIPTAN, CHEMBL101690,
CHEMBL3186179, NARATRIPTAN, OLANZAPINE, QUETIAPINE, SUMATRIPTAN, TRYPTAMINE, XANOMELINE, BRL-15,572,
GR-127935, CHEMBL1256701, CHEMBL277120, METERGOLINE, METITEPINE, METHYLERGONOVINE, RISPERIDONE,
SERTINDOLE, YOHIMBINE, ADENOSINE TRIPHOSPHATE, AZD-5438, PHA-793887, AT-7519, Roniciclib, COLFORSIN,
PAPAVERINE HYDROCHLORIDE, METHICILLIN, ADENOSINE PHOSPHATE, HESPERIDIN, CHEMBL578514, LIPOIC ACID,
METHACHOLINE CHLORIDE, LEUCINE, CHEMBL506495, GALMIC, GALNON, CHEMBL541253, CHEMBL450441,
METHYLENE BLUE, BRETYLIUM TOSYLATE, CHEMBL604991, METHOXAMINE, TEMAZEPAM, GALANIN, DIPHEMANIL,
GALANTIDE, ANDROSTENEDIONE, TESTOSTERONE, VANDETANIB, SORAFENIB, CABOZANTINIB, REGORAFENIB,
PONATINIB, AST-487, CEP-11981, LINIFANIB, LUCITANIB, QUIZARTINIB, SUNITINIB, TAMATINIB, AT-9283, MOTESANIB,
AMUVATINIB, LENVATINIB, ALECTINIB HYDROCHLORIDE, CEP-32496, LESTAURTINIB, SORAFENIB TOSYLATE,
SUNITINIB MALATE, CEP-2563, CETUXIMAB, CHEMBL1213492, INK-128, EVEROLIMUS, DEXAMETHASONE, AZD-1480,
CHEMBL306380, DOVITINIB, GENISTEIN, IMATINIB, CHEMBL126955, ENMD-2076, ALECTINIB, PHORBOL MYRISTATE
ACETATE, XL-999, TRETINOIN, NINTEDANIB, PYRIMETHAMINE, COBALT (II) ION, VERAPAMIL, TETANUS TOXOID,
CERITINIB, MEDRONIC ACID, QUERCETIN, METAPROTERENOL SULFATE, COPANLISIB, ALPELISIB, PICTILISIB,
APITOLISIB, PF-04691502, GEDATOLISIB, SONOLISIB, SF-1126, VOXTALISIB, PILARALISIB (CHEMBL3218575),
DACTOLISIB, PI-103, GSK-2636771, BUPARLISIB, CHEMBL1229535, ARSENIC TRIOXIDE, MOTEXAFIN GADOLINIUM,
CHEMBL449269, FLAVIN ADENINE DINUCLEOTIDE, SPERMIDINE, FOTEMUSTINE, CERIVASTATIN, PSEUDOEPHEDRINE
HYDROCHLORIDE, BOSUTINIB, DASATINIB, HYDROCORTISONE, LITHIUM CARBONATE, LITHIUM CITRATE HYDRATE,
ACETYLCYSTEINE, SUNITINIB, ARIPIPRAZOLE, NAPHTHALENE, CHEMBL435278, CHEMBL122264, CHEMBL17639,
CHEMBL21283, MONOETHANOLAMINE, QUERCETIN, PREDNISOLONE, PUROMYCIN

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating and/or preventing periodontal disease and/or tooth loss in a subject, the method comprising:
classifying a subject into one of seven Periodontal Profile Classes (PPCs) based on information on each tooth present in the subject obtained via a dental examination, wherein
PPC-A designates a healthy subject,
PPC-B designates a subject with mild periodontal disease,
PPC-C designates a subject with high gingival inflammation index (GI),
PPC-D designates a subject with about 50% tooth loss,
PPC-E designates a subject with posterior disease,
PPC-F indicates a subject with at least 75% tooth loss, and
PPC-G indicates a subject with severe disease; and
treating the subject based on their PPC classification.

2. The method of claim 1, wherein the subject treated with an effective amount of a therapeutic agent that targets one or more genes or gene networks associated with the classified PPC.

3. The method of claim 1, wherein the subject is classified as PPC-B, and wherein the subject is treated with adult prophy, non-surgical periodontal therapy, local delivery of antibiotic, or a combination thereof.

4. The method of claim 1, wherein the subject is classified as PPC-B, and wherein the subject is treated with a therapeutic agent selected from the group consisting of dextrose, pyroglutamic acid, streptol, vandetanib, progesterone, acarbose, miglitol, celgosivir, duvoglustat hydrochloride, duvoglustat, phorbol myristate acetate, leucovorin, and irinotecan.

5. The method of claim 1, wherein the subject is classified as PPC-C, and wherein the subject is treated with non-surgical periodontal therapy, local delivery of antibiotic, or a combination thereof.

6. The method of claim 1, wherein the subject is classified as PPC-C, and wherein the subject is treated with a therapeutic agent selected from the group consisting of VANTICTUMAB, FLANVOTUMAB, DOCETAXEL, PACLITAXEL, ACALABRUTINIB, IBRUTINIB, INOSITOL, RETINOL, GENISTEIN, LEUPROLIDE ACETATE, DEXAMETHASONE, HALOFUGINONE, DIHYDROSPINGOSINE, SPHINGOSINE, NIFEDIPINE, CHEMBL1179605, MEFENAMIC ACID, PIOGLITAZONE, ROSIGLITAZONE, TROGLITAZONE, CHEMBL169233, ASPIRIN, CHEMBL1161866, STANOLONE, CHEMBL566340, COLFORSIN, DAROTROPIUM BROMIDE, TRIDIHEXETHYL CHLORIDE, TOLTERODINE TARTRATE, PROPANTHELINE BROMIDE, OXYPHENONIUM BROMIDE, ARIPIPRAZOLE, OLANZAPINE, METHIXENE, TERFENADINE, CLOZAPINE, OXYPHENCYCLIMINE, PROCYCLIDINE, LOXAPINE, PROMAZINE, HYOSCYAMINE, DARIFENACIN, TRIDIHEXETHYL, ANISOTROPINE METHYLBROMIDE, DIPHEMANIL METHYLSULFATE, SCOPOLAMINE, BENZQUINAMIDE, PROPIOMAZINE, TROPICAMIDE, BROMPHENIRAMINE, GLYCOPYRROLATE, TOLTERODINE, PILOCARPINE, MIVACURIUM, DIPHENIDOL, CHLORPROTHIXENE, PIPECURONIUM, LEVOMEPROMAZINE, ISOPROPAMIDE, MEPENZOLATE, FESOTERODINE, METHACHOLINE, ACLIDINIUM, UMECLIDINIUM, ACETYLCHOLINE, ARECAIDINE PROPARGYL ESTER, ARECOLINE, BETHANECHOL, FURTRETHONIUM, 5-METHYLFURMETHIODIDE, CHEMBL99521, ERIBAXABAN, OXOTREMORINE, CHEMBL130715, CHEMBL74300, MILAMELINE, SABCOMELINE, XANOMELINE, ALCURONIUM, BRUCINE, CHEMBL343796, STRYCHNINE, CHEMBL2206331, CHEMBL343357, VINBURNINE, VINCAMINE, CHEMBL139677, CHEMBL523685, CHEMBL1256845, 4-DAMP, CHEMBL279453, AMITRIPTYLINE, BENZTROPINE MESYLATE, ATROPINE, BIPERIDEN (CHEMBL1101), CLIDINIUM, DICYCLOMINE, DOTHIEPIN (CHEMBL1492500), CHEMBL580785, HEXOCYCLIUM, HIMBACINE, CHEMBL1256682, IPRATROPIUM, METHOCTRAMINE, HYDROCHLORIC ACID, OTENZEPAD, OXYBUTYNIN, PIRENZEPINE, PROPANTHELINE, SOLIFENACIN, COENZYME_A, QUINUCLIDINYL BENZILATE, TIOTROPIUM (CHEMBL1900528), TRIPITRAMINE, CHEMBL1233686, CHEMBL1628667, REVATROPATE, UMECLIDINIUM BROMIDE, LAS190792, Afacifenacin, Tarafenacin, SOLIFENACIN SUCCINATE, ACLIDINIUM BROMIDE, METHACHOLINE CHLORIDE, TIOTROPIUM BROMIDE, ACETYLCHOLINE CHLORIDE, CARBACHOL (CHEMBL14), PILOCARPINE HYDROCHLORIDE, CEVIMELINE HYDROCHLORIDE, SUXAMETHONIUM, BATEFENTEROL, MEPENZOLATE BROMIDE, HEXOCYCLIUM METHYLSULFATE, DICYCLOMINE HYDROCHLORIDE, BETHANECHOL CHLORIDE, ATROPINE SULFATE, OXYBUTYNIN CHLORIDE, DARIFENACIN HYDROBROMIDE, IPRATROPIUM BROMIDE HYDRATE, METHSCOPOLAMINE BROMIDE, OXYPHENCYCLIMINE HYDROCHLORIDE, GLYCOPYRROLATE BROMIDE, ISOPROPAMIDE IODIDE, TROSPIUM CHLORIDE, FESOTERODINE FUMARATE, CYCLOPENTOLATE HYDROCHLORIDE, ASM-024, AZD8683, CLIDINIUM BROMIDE, THIETHYLPERAZINE, CEVIMELINE, DOXEPIN, PROMETHAZINE, ITOPRIDE, HOMATROPINE METHYLBROMIDE, ANACETRAPIB, DALCETRAPIB, TORCETRAPIB, CHEMBL67129, EVACETRAPIB, CERIVASTATIN, TAMOXIFEN, CEP-2563, TICLOPIDINE, GSK-690693, SOTRASTAURIN, (7S)-HYDROXYL-STAUROSPORINE, MIDOSTAURIN, QUERCETIN, SOTRASTAURIN ACETATE, STAUROSPORINE, DEXFOSFOSERINE, INGENOL MEBUTATE, CHEMBL369507, BRYOSTATIN, MEDRONIC ACID, INSULIN HUMAN, VANDETANIB, CHEMBL552425, SULFASALAZINE, RI DOGREL, DAZOXIBEN, PHORBOL MYRISTATE ACETATE, BELIMUMAB, BRIOBACEPT, ATACICEPT, TABALUMAB, BLISIBIMOD, DIOXANE, ETOPOSIDE, and CITRIC ACID.

7. The method of claim 1, wherein the subject is classified as PPC-D, and wherein the subject is treated with non-surgical periodontal therapy, local delivery of antibiotic, or a combination thereof.

8. The method of claim 1, wherein the subject is classified as PPC-D, and wherein the subject is treated with a therapeutic agent selected from the group consisting of ISRADIPINE, NIMODIPINE, NISOLDIPINE, VERAPAMIL, FELODIPINE, NITRENDIPINE, NIFEDIPINE, MIBEFRADIL, NILVADIPINE, SALSALATE, BEPRIDIL HYDROCHLORIDE, PREGABALIN, GABAPENTIN, GABAPENTIN ENACARBIL, IMAGABALIN, ATAGABALIN, MAGNESIUM SULFATE, AMLODIPINE, CELECOXIB, DRONEDARONE, CHEMBL566340, GO-6976, QUERCETIN, GSK-690693, (7S)-HYDROXYL-STAUROSPORINE, CEP-2563, MIDOSTAURIN, SOTRASTAURIN, BRYOSTATIN, RESVERATROL, VASOPRESSIN, BOSUTINIB, CHEMBL359482, PAROXETINE, OCRIPLASMIN, HYDROCHLOROTHIAZIDE, CHEMBL384759, INSULIN HUMAN, BUSULFAN, ZONISAMIDE, FRUCTOSE, TRICHOSTATIN, ASCORBATE, OCRIPLASMIN, GUANIDINE HYDROCHLORIDE, DALFAMPRIDINE, TEDISAMIL, NERISPIRDINE, TRETINOIN, ETRETINATE, ZOLEDRONIC ACID, CHEMBL300914, PYRIDOXAL PHOSPHATE, THREONINE, NIROGACESTAT, REGN-421, and CYCLOPHOSPHAMIDE.

9. The method of claim 1, wherein the subject is classified as PPC-E, and wherein the subject is treated with non-surgical periodontal therapy, local delivery of antibiotic, or a combination thereof.

10. The method of claim 1, wherein the subject is classified as PPC-E, and wherein the subject is treated with a therapeutic agent selected from the group consisting of QUERCETIN, METAPROTERENOL SULFATE, COPANLISIB, ALPELISIB, PICTILISIB, APITOLISIB, PF-04691502, GEDATOLISIB, SONOLISIB, SF-1126, VOXTALISIB, PILARALISIB (CHEMBL3218575), DACTOLISIB, PI-103, GSK-2636771, BUPARLISIB, CHEMBL1229535, HYDROGEN PEROXIDE, CLAVULANIC ACID, AMOXICILLIN, ME-344, NV-128, METFORMIN HYDROCHLORIDE, CHEMBL1161866, CAPSAICIN, CHEMBL1213492, NINTEDANIB ESYLATE, SU-014813, KRN-633, L-21649, TAK-593, AG-13958, BMS-690514, FORETINIB, MGCD-265, BRIVANIBALANINATE, SEMAXANIB, CEDIRANIB, SORAFENIB, Anlotinib, CC-223, DOVITINIB, Fruquintinib, LENVATINIB, LINIFANIB, MK-2461, MOTESANIB, NINTEDANIB, PAZOPANIB, SUNITINIB, TESEVATINIB, TIVOZANIB, VATALANIB, AXITINIB, REGORAFENIB, LENVATINIB MESYLATE, ILORASERTIB, CEP-11981, CEP-7055, Chiauranib, JNJ-26483327, OSI-930, RG-1530, TELATINIB, Famitinib, XL-999, BRIVANIB, SUNITINIB MALATE, VANDETANIB, SORAFENIB TOSYLATE, PAZOPANIB HYDROCHLORIDE, LUCITANIB, ENMD-2076, ORANTINIB, X-82, XL-820, CEP-5214, SU-14813, CP-459632, Sulfatinib, 4SC-203, CHEMBL313417, IMC-3C5, TABERMINOGENE VADENOVEC, TG100-801, IMC-1C11, PEGPLERANIB SODIUM, CHEMBL384759, GUANOSINE TRIPHOSPHATE, GLYCINE, TEZAMPANEL, BUTABARBITAL, BUTALBITAL, TALBUTAL, SECOBARBITAL, METHARBITAL, THIOPENTAL, PRIMIDONE, MEPHOBARBITAL, PHENOBARBITAL, CHEMBL301536, 2S,4R-4-METHYLGLUTAMATE, DOMOIC ACID, KAINIC ACID, MESALAMINE, METHYPRYLON, AMOBARBITAL, APROBARBITAL, BUTETHAL, HEPTABARBITAL, HEXOBARBITAL, BARBITAL, Selurampanel, TOPIRAMATE, PENTOBARBITAL, QUISQUALATE, L-GLUTAMATE, PROTIRELIN, TALTIRELIN, GANITUMAB, PICROPODOPHYLLOTOXIN, BMS-754807, LINSITINIB, AEW-541, DALOTUZUMAB, INSULIN GLARGINE, EMACTUZUMAB, AZD-4547, CHEMBL401930, BRIGATINIB, CERITINIB, CHEMBL464552, CHEMBL458997, DIMETHISTERONE, XL-228, BUB-022, AVE-1642, CIXUTUMUMAB, INSM-18, KW-2450, FIGITUMUMAB, ROBATUMUMAB, MECASERMIN, PL-225B, TEPROTUMUMAB, MECASERMIN RINFABATE, CHEMBL1230989, ACETYLCYSTEINE, THROMBIN, INSULIN HUMAN, CHEMBL263143, CHEMBL397666, RALOXIFENE, ERLOTINIB, RINFABATE, STAUROSPORINE, and HYDROCHLOROTHIAZIDE.

11. The method of claim 1, wherein the subject is classified as PPC-F, and wherein the subject is treated with non-surgical periodontal therapy, local delivery of antibiotic, or a combination thereof.

12. The method of claim 1, wherein the subject is classified as PPC-F, and wherein the subject is treated with with a therapeutic agent selected from the group consisting of DANAZOL, CHEMBL195368, CHEMBL1234621, CONBERCEPT, SORAFENIB TOSYLATE, LENALIDOMIDE, BEVACIZUMAB, NIMODIPINE, PROGESTERONE, SPIRONOLACTONE, EPLERENONE, FELODIPINE, DESOXYCORTICOSTERONE PIVALATE, DROSPIRENONE, ALDOSTERONE, HYDROCORTISONE, DESOXYCORTICOSTERONE, DEXAMETHASONE, FLUDROCORTISONE, PREDNISOLONE, FINERENONE, ONAPRISTONE, PF-03882845, OXPRENOATE POTASSIUM, XL550, MT-3995, LY2623091, DESOXYCORTICOSTERONE ACETATE, FLUDROCORTISONE ACETATE, CORTICOSTERONE, RIZATRIPTAN, CHEMBL482796, LIPDXIN A4, LITHIUM, GADOBENATE DIMEGLUMINE, CHEMBL185515, AMILORIDE, PYRIMETHAMINE, NAFAMOSTAT, PREGNENOLONE, GENISTEIN, LEUPROLIDE ACETATE, DEXAMETHASONE, HALOFUGINONE, DAVALINTIDE, PRAMLINTIDE, PRAMLINTIDE ACETATE, CALCITONIN SALMON RECOMBINANT, DOXORUBICIN, CHEMBL574817, CLOZAPINE, ODANACATIB, PALMITIC ACID, FLANVOTUMAB, INGENOL MEBUTATE, ELLAGIC ACID, APRINOCARSEN SODIUM, CHEMBL1236539, BALANOL, ENZASTAURIN, GO-6976, SEBACIC ACID, RUBOXISTAURIN, SOTRASTAURIN, MIDOSTAURIN, QUERCETIN, SOTRASTAURIN ACETATE, GSK-690693, CEP-2563, (7S)-HYDROXYL-STAUROSPORINE, BRYOSTATIN, TAMOXIFEN, DEXFOSFOSERINE, VITAMIN E, FRUCTOSE, TRICHOSTATIN, SACITUZUMAB GOVITECAN, TEGLARINAD CHLORIDE, FLUOROURACIL, OCRIPLASMIN, HALOTHANE, PROXYPHYLLINE, LISOFYLLINE, CAFFEINE, TRETINOIN, UROKINASE, CARBOQUONE, INSULIN HUMAN, MESALAMINE, L-GLUTAMATE, PYRIMETHAMINE, PYRIDOSTIGMINE, PYRILAMINE, PEGINTERFERON LAMBDA-1A, RINTATOLIMOD, PYROXAMIDE, HYDROXYCHLOROQUINE, ELEDOISIN, KASSININ, NEUROKININ A, NEUROKININ B, CHEMBL69367, SENKTIDE, SUBSTANCE P, ISOFLURANE, AZD2624, CHEMBL480249, CHEMBL221445, OSANETANT, CHEMBL44229, SAREDUTANT, CHEMBL9843, CHEMBL1991816, AMCINONIDE, TALNETANT, SB-222200, CHEMBL275544, PYRIMETHAMINE, COBALT (II) ION, VERAPAMIL, VASOPRESSIN TANNATE, FAMOXADONE, AZOXYSTROBIN, Coenzyme Q2, PROXYPHYLLINE, CHOLIC ACID, AFIMOXIFENE (CHEMBL489), DIARYLPROPIONITRILE, DIETHYLSTILBESTROL, CHLOROTRIANISENE, ESTROGENS, CONJUGATED, ETONOGESTREL, DESOGESTREL, LEVONORGESTREL, PROGESTERONE, TOREMIFENE, MEDROXYPROGESTERONE ACETATE, ESTRONE, TAMOXIFEN, DIENESTROL, FULVESTRANT, NORGESTIMATE, ETHINYL ESTRADIOL, MELATONIN, TRILOSTANE, FLUOXYMESTERONE, ESTRAMUSTINE, ESTRIOL, PRINABEREL, PROPYLPYRAZOLETRIOL, RALOXIFENE, CHEMBL282489, CHEMBL188528, LASOFOXIFENE, BAZEDOXIFENE, CLOMIPHENE, CHEMBL201013, HEXESTROL, CHEMBL520107, MESTRANOL, DANAZOL, ALLYLESTRENOL, PRASTERONE, ESTROPIPATE, QUINESTROL, OSPEMIFENE, TIBOLONE, ESTROGENS, CONJUGATED SYNTHETIC A, SYNTHETIC CONJUGATED ESTROGENS, B, ESTRADIOL, MITOTANE, SR16234 (CHEMBL3545210), FISPEMIFENE, LY2245461, IDOXIFENE, GTx-758, AFIMOXIFENE (CHEMBL10041), DROLOXIFENE, ACOLBIFENE, TAMOXIFEN CITRATE, ESTRADIOL VALERATE, CLOMIPHENE CITRATE, ESTRADIOL CYPIONATE, ESTROGENS, ESTERIFIED, DIETHYLSTILBESTROL DIPHOSPHATE, TOREMIFENE CITRATE, BAZEDOXIFENE ACETATE, CHF4227, GDC-0810, ESTRADIOL ACETATE (CHEMBL1200430), POLYESTRADIOL PHOSPHATE, MK-6913, IODINE, VINTAFOLIDE, CUSTIRSEN, CHEMBL304552, EVEROLIMUS, CHEMBL181936, CHEMBL391910, PERTUZUMAB, CHEMBL180300, DIENOGEST, ESTROGEN, RAD1901, CHEMBL222501, ARZOXIFENE, CHEMBL180071, CHEMBL193676, ETHYNODIOL DIACETATE, GENISTEIN, RALOXIFEN, Ribociclib, RALOXIFENE CORE, TRASTUZUMAB, CHEMBL236718, ABEMACICLIB, 2-AMINO-1-METHYL-6-PHENYLIMIDAZO[4,5-B]PYRIDINE, ERTEBEREL, EXEMESTANE, ENDOXIFEN, MEGESTROLACETATE, SIVIFENE, PALBOCICLIB, LEFLUNOMIDE, ESTRONE SODIUM SULFATE, LETROZOLE, CHEMBL236086, CHEMBL184151, CHEMBL223026, LAPATINIB, CHEMBL180517, NORELGESTROMIN, ANASTROZOLE, CARBOQUONE, GONADORELIN, CHEMBL1213270, NORGESTREL, and MEDRONIC ACID.

13. The method of claim 1, wherein the subject is classified as PPC-G, and wherein the subject is treated with non-surgical periodontal therapy, local delivery of antibiotic, or a combination thereof.

14. The method of claim 1, wherein the subject is classified as PPC-G, and wherein the subject is treated with a therapeutic agent selected from the group consisting of TEMAZEPAM, ADINAZOLAM, HALAZEPAM, DIAZEPAM, OXAZEPAM, TRIAZOLAM, ESTAZOLAM, BROMAZEPAM, CLOTIAZEPAM, FLUDIAZEPAM, KETAZOLAM, PRAZEPAM, QUAZEPAM, CINOLAZEPAM, NITRAZEPAM, CLORAZEPATE DIPOTASSIUM, CHLORDIAZEPDXIDE, ESZOPICLONE, MEPROBAMATE, CLOBAZAM, ALPRAZOLAM, BUTALBITAL, CLONAZEPAM, DESFLURANE, TALBUTAL, BUTABARBITAL SODIUM, LORAZEPAM, METHARBITAL, METHYPRYLON, MIDAZOLAM HYDROCHLORIDE, PRIMIDONE, PROPOFOL, PENTOBARBITAL SODIUM, SECOBARBITAL SODIUM, SEVOFLURANE, THIAMYLAL SODIUM, FLURAZEPAM HYDROCHLORIDE, HALOTHANE, ISOFLURANE, ADIPIPLON, Lorediplon, RESEQUINIL, PF-06372865, METHOHEXITAL SODIUM, THIOPENTAL SODIUM, CHLORDIAZEPDXIDE HYDROCHLORIDE, PENTOBARBITAL, ETHCHLORVYNOL, GLUTETHIMIDE, FLUMAZENIL, CLORAZEPIC ACID, METHOXYFLURANE, MIDAZOLAM, TRICLOFOS SODIUM, TOPIRAMATE, GABOXADOL, ETOMIDATE, ACAMPROSATE CALCIUM, FLURAZEPAM, OCINAPLON, ENFLURANE, ETAZOLATE, CHEMBL2325441, PREGNENOLONE, S-ADENOSYLHOMOCYSTEINE, ODANACATIB, PROSCILLARIDIN, CYPROHEPTADINE, CYCLOPENTOLATE, ELETRIPTAN, METHYSERGIDE, ZOLMITRIPTAN, ERGOTAMINE, RIZATRIPTAN, MIANSERIN, ELETRIPTAN HYDROBROMIDE, ALMOTRIPTAN MALATE, LASMIDITAN, CHEMBL266591, SEROTONIN, 5-MEO-DMT, 5-METHOXYTRYPTAMINE, 8-OH-DPAT, CHEMBL1256797, CLOZAPINE, DIHYDROERGOTAMINE, CHEMBL1332062, DONITRIPTAN, CHEMBL101690, CHEMBL3186179, NARATRIPTAN, OLANZAPINE, QUETIAPINE, SUMATRIPTAN, TRYPTAMINE, XANOMELINE, BRL-15,572, GR-127935, CHEMBL1256701, CHEMBL277120, METERGOLINE, METITEPINE, METHYLERGONOVINE, RISPERIDONE, SERTINDOLE, YOHIMBINE, ADENOSINE TRI PHOSPHATE, AZD-5438, PHA-793887, AT-7519, Roniciclib, COLFORSIN, PAPAVERINE HYDROCHLORIDE, METHICILLIN, ADENOSINE PHOSPHATE, HESPERIDIN, CHEMBL578514, LIPOIC ACID, METHACHOLINE CHLORIDE, LEUCINE, CHEMBL506495, GALMIC, GALNON, CHEMBL541253, CHEMBL450441, METHYLENE BLUE, BRETYLIUM TOSYLATE, CHEMBL604991, METHOXAMINE, TEMAZEPAM, GALANIN, DIPHEMANIL, GALANTIDE, ANDROSTENEDIONE, TESTOSTERONE, VANDETANIB, SORAFENIB, CABOZANTINIB, REGORAFENIB, PONATINIB, AST-487, CEP-11981, LINIFANIB, LUCITANIB, QUIZARTINIB, SUNITINIB, TAMATINIB, AT-9283, MOTESANIB, AMUVATINIB, LENVATINIB, ALECTINIB HYDROCHLORIDE, CEP-32496, LESTAURTINIB, SORAFENIB TOSYLATE, SUNITINIB MALATE, CEP-2563, CETUXIMAB, CHEMBL1213492, INK-128, EVEROLIMUS, DEXAMETHASONE, AZD-1480, CHEMBL306380, DOVITINIB, GENISTEIN, IMATINIB, CHEMBL126955, ENMD-2076, ALECTINIB, PHORBOL MYRISTATE ACETATE, XL-999, TRETINOIN, NINTEDANIB, PYRIMETHAMINE, COBALT (II) ION, VERAPAMIL, TETANUS TOXOID, CERITINIB, MEDRONIC ACID, QUERCETIN, METAPROTERENOL SULFATE, COPANLISIB, ALPELISIB, PICTILISIB, APITOLISIB, PF-04691502, GEDATOLISIB, SONOLISIB, SF-1126, VOXTALISIB, PILARALISIB (CHEMBL3218575), DACTOLISIB, PI-103, GSK-2636771, BUPARLISIB, CHEMBL1229535, ARSENIC TRIOXIDE, MOTEXAFIN GADOLINIUM, CHEMBL449269, FLAVIN ADENINE DINUCLEOTIDE, SPERMIDINE, FOTEMUSTINE, CERIVASTATIN, PSEUDOEPHEDRINE HYDROCHLORIDE, BOSUTINIB, DASATINIB, HYDROCORTISONE, LITHIUM CARBONATE, LITHIUM CITRATE HYDRATE, ACETYLCYSTEINE, SUNITINIB, ARI PIPRAZOLE, NAPHTHALENE, CHEMBL435278, CHEMBL122264, CHEMBL17639, CHEMBL21283, MONOETHANOLAMINE, QUERCETIN, PREDNISOLONE, PUROMYCIN
